(19) United States
(12) Patent Application Publication
(10) Pub. No.: US 2024/0286998 A1
(43) Pub. Date: Aug. 29, 2024

(54) FLUORINATED TRYPTAMINE COMPOUNDS, ANALOGUES THEREOF, AND METHODS USING SAME

(71) Applicants: Saint Joseph's University, Philadelphia, PA (US); Compass Pathfinder Ltd., Altrincham Cheshire (GB)

(72) Inventors: Jason Wallach, Lansdowne, PA (US); Michael Dybek, Philadelphia, PA (US)

(21) Appl. No.: 18/565,565

(22) PCT Filed: Jun. 2, 2022
(86) PCT No.: PCT/US2022/032000

§ 371 Date: Nov. 30, 2023

Related U.S. Application Data
(60) Provisional application No. 63/195,943, filed on Jun. 2, 2021.
(60) Provisional application No. 63/288,313, filed on Dec. 10, 2021.

(51) Int. Cl. C07D 209/16; A61K 31/4045; A61K 45/06; A61P 25/00; C07D 209/26; C07D 401/04; C07D 403/04; C07D 403/06; C07D 409/12

(52) U.S. Cl. CPC C07D 209/16; A61K 31/4045; A61K 45/06; A61P 25/00; C07D 209/26; C07D 401/04; C07D 403/04; C07D 403/06; C07D 409/12

(57) ABSTRACT

The present disclosure relates in one aspect to fluorinated tryptamine compounds, or analogues thereof, and compositions comprising the same. Compounds of the disclosure can be used to treat, prevent, and/or ameliorate a psychiatric disease or disorder in a subject.

### CROSS-REFERENCE TO RELATED APPLICATIONS

The present application claims priority under 35 U.S.C. § 119(e) to U.S. Provisional Application No. 63/195,943, filed Jun. 2, 2021, and U.S. Provisional Application No. 63/288,313, filed Dec. 10, 2021, all of which applications are hereby incorporated herein by reference in their entireties.

### BACKGROUND OF THE INVENTION

It is estimated that 51.5 million adults aged 18 or older in the United States suffer with at least one psychiatric disease and/or disorder, representing approximately 20.6% of all U.S. adults. The impact of such psychiatric diseases and/or disorders may vary, ranging from mild to severe impairment, i.e., severe mental illness, which substantially interferes with or limits one or more major life activities. It is estimated that there are 13.1 million U.S. adults (5.2%) with severe mental illness.

There is thus a need in the art for compositions and methods of treating, preventing, and/or ameliorating a psychiatric disease and/or disorder in a subject. The present disclosure addresses this need.

### BRIEF SUMMARY OF THE INVENTION

The present disclosure provides certain compounds of formula (I), formula (II), and formula (III), or a salt, prodrug, solvate, isotopologue, or stereoisomer thereof, wherein the substituents in (I), (II), and (III) are defined elsewhere herein:

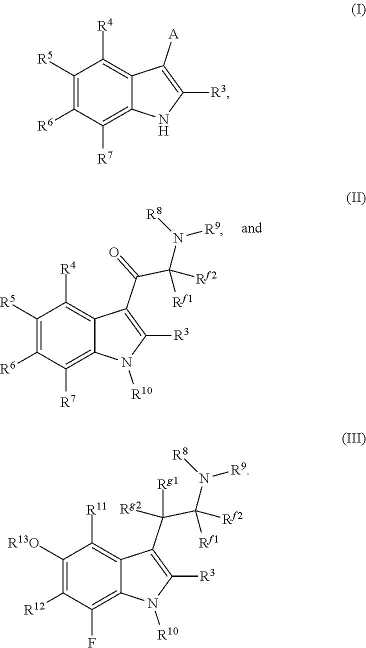

The present disclosure further provides pharmaceutical compositions comprising at least one compound of the present disclosure and a pharmaceutically acceptable carrier.

The present disclosure further provides methods of treating, preventing, and/or ameliorating a psychiatric disease or disorder in a subject in need thereof. In certain embodiments, the method comprises administering to the subject a therapeutically effective amount of at least one compound of the present disclosure and/or at least one pharmaceutical composition of the present disclosure. In certain embodiments, the psychiatric disease or disorder is selected from the group consisting of depressive disorder, anxiety disorder, and eating disorder.

In certain embodiments, the subject is further administered at least one additional agent useful for treating, preventing, and/or ameliorating the psychiatric disease or disorder. In certain embodiments, the at least one additional agent is selected from the group consisting of a selective serotonin reuptake inhibitor, triple reuptake inhibitor, serotonin and norepinephrine reuptake inhibitor, tricyclic antidepressant, tetracyclic antidepressant, dopamine reuptake inhibitor, mood stabilizer, anticonvulsant, antipsychotic, anxiolytics, benzodiazepines, monoamine releasers, dopamine receptor agonist, cannabinoids, triptans, anti-migraine medications, analgesics, anti-inflammatory, immune modulator, 5-HT_{1A} receptor antagonist, 5-HT₂ receptor antagonist, 5-HT₃ receptor antagonist, monoamine oxidase inhibitor, and noradrenergic antagonist.

### BRIEF DESCRIPTION OF THE FIGURES

The drawings illustrate generally, by way of example, but not by way of limitation, various embodiments of the present application.

FIG. 1 shows the Head-Twitch Response (HTR) dose response curve of compound 4.

FIG. 2 shows the Head-Twitch Response (HTR) dose response curve of compound 9.

FIG. 3 shows the Head-Twitch Response (HTR) dose response curve for non-psychedelic 6-fluoro-N,N-diethyltryptamine (6-F-DET).

FIG. 4 shows the predicted binding pose of compound 51 to 5-HT_{2A}.

FIG. 5 shows Head-Twitch Response (HTR) in mice as a function of time with administration of compound 88 at 2 mg/kg and 6 mg/kg as compared to a control (vehicle).

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

5-HT_{1A} agonism is being investigated in numerous therapeutic areas including pain, seizure, depression, etc. 5-HT_{1A} partial agonists, like buspirone, are approved for the treatment of anxiety. At the cellular and organismal level, without wishing to be bound by theory, 5-HT_{1A} agonism may counter 5-HT_{2A} activation reducing the intensity of 5-HT_{2A} signaling and psychedelic action. For example, blocking 5-HT_{1A} using a weak partial agonist (e.g., pindolol) was reported to potentiate the psychoactive effects of dimethyl tryptamine (DMT) in human volunteers. This action of 5-HT_{1A} on 5-HT_{2A} signaling, can be desirable or undesirable depending on the therapeutic indication.

5-HT_{1A} agonism may also contribute to the psychoactive subjective experience of a 5-HT_{2A} agonist in other ways. For example, the potent psychedelic tryptamine 5-methoxy-dimethyltryptamine (5-MeO-DMT) lacks a strong visual component, in contrast to many other psychedelic 5-HT_{2A} agonists like DMT, but often leads to intense tactile effects and a near death like experience. It is speculated that the strong 5-HT_{1A} agonism is responsible for at least some of the aforementioned differences. 5-HT_{2C} agonism is known to cause an anorectic effect (i.e., loss of appetite), as well as effects on mood including anxiety. With respect to psychedelic drug profile, such effects could be desirable or undesirable depending on indication. In some embodiments it is desirable to minimize 5-HT_{2C} agonism, potentially leading to a psychedelic experience with an attenuated or reduced chance of anxiety relative to existing compounds. In some instances, some degree of anxiety may actually raise the probability of having a transformative experience that could contribute to a desirable therapeutic outcome. Likewise strong 5-HT_{2C} agonism may lead to long term (weeks to months) changes in 5-HT_{2C} receptor expression following the administration of a 5-HT_{2A}/5-HT_{2C} agonist, which could confer or contribute to a lasting anxiolytic and antidepressant effect.

It has been further hypothesized that changes in 5-HT_{2C} expression (e.g., downregulation) that may follow agonist exposure could have unique benefits in anorexia. 5-HT_{2C} antagonism or knock down leads to weight gain in many models. Supporting this, chronic treatment with 5-HT_{2C} agonists has led to significant rebound weight gain in animal models.

The compounds described herein may agonize one or more receptors selected from the group consisting of 5-HT_{2A}, 5-HT_{2B}, 5-HT_{2C}, and 5-HT_{1A}, or any mixtures thereof. In certain embodiments, the compounds described herein are selective for 5-HT_{2A} over 5-HT_{2B}. In certain embodiments, the compounds described herein are selective for 5-HT_{2A} over 5-HT_{2C}. In certain embodiments, the compounds described herein are selective for 5-HT_{2A} over 5-HT_{1A}. In certain embodiments, the compounds described herein are selective for 5-HT_{2A} over 5-HT_{2B}, 5-HT_{2C}, and 5-HT_{1A}. In certain embodiments, the compounds of the present disclosure selectively agonize 5-HT_{2A} and 5-HT_{1A} over 5-HT_{2B} and 5-HT_{2C}. In certain embodiments, the compounds of the present disclosure selective agonize 5-HT_{2A}, 5-HT_{2C}, and 5-HT_{1A} over 5-HT_{2B}. In certain embodiments, the compounds of the present disclosure selectively agonize 5-HT_{1A} over 5-HT_{2A}, 5-HT_{2B}, and 5-HT_{2C}.

In certain embodiments, selectivity for 5-HT_{2A} over 5-HT_{2B} is desirable. In certain embodiments, selectivity for 5-HT_{2A} over 5-HT_{2C} is desirable. In certain embodiments, selectivity for 5-HT_{2A} over 5-HT_{1A} is desirable.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms.

Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like.

Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

As used herein, the term “aromatic” refers to a carbocycle or heterocycle with one or more polyunsaturated rings and having aromatic character, i.e., having (4n+2) delocalized π (pi) electrons, where ‘n’ is an integer.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups.

Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach.

The co-administered compound and/or composition may be formulated in any kind of combinations as mixtures of solids and liquids under a variety of solid, gel, and liquid formulations, and as a solution.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “half-maximal inhibitory concentration” or “IC₅₀” as used herein refers to the concentration of a compound or composition required to obtain a 50% decrease in a biological or biochemical process with administration of a compound and/or composition.

The term “half-maximal effective concentration” or “EC₅₀” as used herein refers to the concentration of a compound or composition required to obtain a 50% increase in a biological or biochemical process with administration of a compound and/or composition.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Non-limiting examples include: —OCH₂CH₂CH₃, —CH₂CH₂CH₂OH, —CH₂CH₂NHCH₃, —NHCH₂CH₂CH₃, —CH₂CH₂CH₂NH₂, —SCH₂CH₂CH₃, —CH₂SCH₂CH₃, CH₂CH₂CH₂SH, and —CH₂CH₂S(═O)CH₃. Up to two heteroatoms may be consecutive, such as, for example, —CH₂NH—OCH₃, or —CH₂CH₂SSCH₃.

The term “heteroarylalkyl” as used herein refers to alkyl groups as defined herein in which a hydrogen or carbon bond of an alkyl group is replaced with a bond to a heteroaryl group as defined herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth.

Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “median effective dose” or “ED₅₀” as used herein refers to the dose or concentration of a compound and/or composition that produces a therapeutic effect in 50% of the population administered the dose.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body.

Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

The term “prodrug” as used herein refers to a biologically inactive compound which is metabolized to a biologically active compound in vivo. Non-limiting examples of metabolic reactions include hydrolysis and reduction. It is understood that a prodrug of a compound of the present invention may comprise substitution of any heteroatom with any of a number of unique prodrug moieties, including but not limited to an ester, amide, carbamate, carbonate ester, urea, imine, enamine, phosphate ester, thioester, sulfate, sulfonamide, acyloxyalkyl ester, disulfide, and N-sulfonyl imidate.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The terms “treat,” “treating” and “treatment,” as used herein, means reducing the frequency or severity with which symptoms of a disease or condition are experienced by a subject by virtue of administering an agent or compound to the subject.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

### Description

There is an urgent unmet need for short acting 5-HT_{2A} agonists. The current gold standard 5-HT_{2A} agonist in clinical development is Psilocybin, which has a duration of 5-8 hours. The psychoactive experience (acute subjective effects) induced by 5-HT_{2A} agonists like Psilocybin (via its active metabolite psilocin) can be an intense psychological experience for patients. This duration risks limiting the access of this medication to patients and puts restrictions on the number of patients a clinician can treat. Furthermore, a longer duration is unlikely to be necessary for the clinical benefits in most therapeutic indications. For example, ketamine, an atypical rapid acting antidepressant, also induces potent psychoactive effects but with a one hour duration of action. Thus, a 5-8 hour experience is likely unnecessary for clinical outcome. Likewise, because the psychoactive effects of psilocybin and other 5-HT_{2A} agonists can be intense, patients need to be monitored by a physician or other caregiver during administration. Short duration 5-HT_{2A} agonists that retain desired clinical activity can offer greater convenience for patients and their caregivers.

Numerous previously studied fluorinated tryptamines have been shown to lack or reduce the classic psychoactivity associated with 5-HT_{2A} agonists. The clearest and strongest example of this is with 6-fluoro-N,N-diethyltryptamine (6-F-DET), which has been tested in humans and found to lack the characteristic psychoactive effects of hallucinogenic 5-HT_{2A} agonists, such as psilocybin. As such, 6-F-DET has been used as an inactive control in clinical research since it induces some of the physiological effects but not the potent psychological effects seen with psychedelic 5-HT_{2A} agonists. Others have described fluorinated dimethyl tryptamine (DMT) analogs as non-hallucinogenic synaptogenesis modulators. The duration of DMT analogs is generally short, under thirty minutes, which may be too brief for long term efficacy in disease states like major depressive disorder, post-traumatic stress disorder and substance use disorders. DMT and many of its analogs also lack oral activity. The fluorinated compounds of the present invention act as 5-HT_{2A} receptor agonists and induce psychoactive effects comparable to longer acting 5-HT_{2A} agonists like psilocybin, but with a reduced duration of action. Fluorine substitution at key sites of the indole ring can maintain high 5-HT_{2A} efficacy and provide potent psychoactive effects.

In addition to the importance of the added fluorine, the N-alkyl amine substituents discovered here uniquely modulate the pharmacokinetics of the compounds allowing tailoring of the desired pharmacological activities including binding profiles, bioavailability, half-life, and thus efficacy, side effect profile, and duration of the classic 5-HT_{2A} mediated psychoactive effects. In several cases, asymmetric N,N-dialkyl amine substitutions, completely novel to the 5-HT_{2A} tryptamine SAR, offer a unique strategy to optimize the balance between pharmacodynamic activity and desired pharmacokinetics, such as duration of action.

### Compounds

In one aspect, the present disclosure provides a compound of formula (I), or a salt, prodrug, solvate, isotopologue, or stereoisomer thereof:

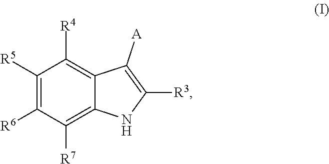

wherein:
   A is R²,

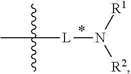

wherein * indicates the bond between L and N(R¹)(R²);
L is

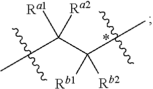

R^{a1} and R^{a2} are each independently selected from the group consisting of H, halogen, C₁-C₆ alkoxy, and C₁-C₆ alkyl,
   or R^{a1} and R^{a2} may combine to form a carbonyl (C═O);
R^{b1} and R^{b2} are each independently selected from the group consisting of H and C₁-C₆ alkyl;
R¹ and R² are each independently selected from the group consisting of optionally substituted C₁-C₈ alkyl, optionally substituted C₃-C₈ cycloalkyl, optionally substituted C₁-C₈ heteroalkyl, optionally substituted C₂-C₈ heterocycloalkyl, optionally substituted C₂-C₈ alkenyl, and optionally substituted C₂-C₈ alkynyl,
   wherein R¹ and R² are not identical, and
   wherein one of R¹ and R² may combine with one of R^{a1}, R^{a2}, R^{b1}, and R^{b2} to form an optionally substituted C₂-C₈ heterocyclyl,
   with the proviso that, if one of R¹ and R² combines with R^{b1} or R^{b2} to form a 5 membered ring and R⁵ is F, then at least one of R⁴, R⁶, and R⁷ is not H, or if one of R¹ and R² combine with R^{b1} or R^{b2} to form a stereocenter then the compound consists essentially of one stereoisomer;
R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
   wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl);
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl; and
wherein the isotopologue does not comprise F¹⁸ in R¹ or R².

In certain embodiments, the compound of formula (I) is not N-ethyl-N-(2-(4-fluoro-1H-indol-3-yl)ethyl)propan-1-amine. In certain embodiments, the compound of formula (I) is not N-ethyl-N-(2-(5-fluoro-1H-indol-3-yl)ethyl)propan-1-amine. In certain embodiments, the compound of formula (I) is not N-ethyl-N-(2-(6-fluoro-1H-indol-3-yl)ethyl)propan-1-amine. In certain embodiments, the compound of formula (I) is not N-ethyl-N-(2-(7-fluoro-1H-indol-3-yl)ethyl)propan-1-amine. In certain embodiments, the compound of formula (I) is not N-ethyl-2-(4-fluoro-1H-indol-3-yl)-N-methylethan-1-amine. In certain embodiments, the compound of formula (I) is not N-ethyl-2-(5-fluoro-1H-indol-3-yl)-N-methylethan-1-amine. In certain embodiments, the compound of formula (I) is not N-ethyl-2-(6-fluoro-1H-indol-3-yl)-N-methylethan-1-amine. In certain embodiments, the compound of formula (I) is not N-ethyl-2-(7-fluoro-1H-indol-3-yl)-N-methylethan-1-amine. In certain embodiments, the compound of formula (I) is not (R)-4-fluoro-3-((1-(methyl-d3)pyrrolidin-2-yl)methyl-d2)-1H-indole. In certain embodiments, the compound of formula (I) is not (R)-4-fluoro-3-((1-(methyl-d3)pyrrolidin-2-yl)methyl)-1H-indole. In certain embodiments, the compound of formula (I) is not (R)-4-fluoro-3-(pyrrolidin-2-ylmethyl-d2)-1H-indole. In certain embodiments, the compound of formula (I) is not (S)-4-fluoro-3-((1-(methyl-d3)pyrrolidin-2-yl)methyl-d2)-1H-indole.

In certain embodiments, if one of the following applies:
(a) R¹ is ethyl and R² is n-propyl,
(b) R¹ is n-propyl and R² is ethyl,
(c) R¹ is methyl and R² is ethyl, or
(d) R¹ is ethyl and R² is methyl, then no more than two of R⁴, R⁵, R⁶, and R⁷ are H.

In certain embodiments, if R¹ is n-butyl, then R² is not 4-fluorobutyl. In certain embodiments, if R² is n-butyl, then R¹ is not 4-fluorobutyl. In certain embodiments, if R¹ is n-butyl, then R² is not a C₄ fluoroalkyl. In certain embodiments, if R² is n-butyl, then R¹ is not a C₄ fluoroalkyl.

In certain embodiments, at least two of R⁴, R⁵, R⁶, and R⁷ are F.

In certain embodiments, the compound of formula (I) is a compound of formula (I′), or a salt, prodrug, solvate, isotopologue, or stereoisomer thereof:

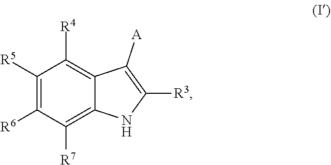

wherein:
   A is

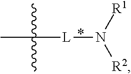

wherein * indicates the bond between L and N(R¹)(R²);
L is

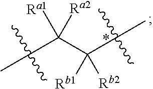

R^{a1} and R^{a2} are each independently selected from the group consisting of H, halogen, C₁-C₆ alkoxy, and C₁-C₆ alkyl,
   or R^{a1} and R² may combine to form a carbonyl (C═O);
R^{b1} and R^{b2} are each independently selected from the group consisting of H and C₁-C₆ alkyl;
R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
   wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl);
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl; and
wherein one of the following applies:
(a) R¹ is iso-propyl and R² is ethyl; (b) R¹ is iso-propyl and R² is methyl; (c) R¹ is sec-butyl and R² is methyl; (d) R¹ is iso-propyl and R² is n-propyl; (e) R¹ is iso-propyl and R² is allyl; (f) R¹ is methyl and R² is n-propyl; (g) R¹ is ethyl and R² is sec-butyl; (h) R¹ is n-propyl and R² is sec-butyl; (i) R¹ is iso-propyl and R² is sec-butyl; (j) R¹ is allyl and R² is sec-butyl; (k) R¹ is allyl and R² is ethyl; (l) R¹ is allyl and R² is n-propyl; (m) R¹ is isopropyl and R² is cyanomethyl; (n) R¹ is iso-propyl and R² is propargyl; (o) R¹ is methyl and R² is 2-cyclopropyleth-2-yl; (p) R¹ is methyl and R² is 3-thietanyl; (q) R¹ is ethyl and R² is cyclobutyl; (r) R¹ is n-propyl and R² is cyclobutyl; (s) R¹ is iso-propyl and R² is cyclobutyl; (t) R¹ is allyl and R² is cyclobutyl; (u) R¹ is methyl and R² is iso-butyl; (v) R¹ is cyclobutyl and R² is methyl; (w) R¹ is methyl and R² is allyl; (x) R¹ is iso-propyl and R² is iso-butyl; (y) R¹ is methyl and R² is cyclobutyl; (z) R¹ is ethyl and R² is cyclobutyl; (aa) R¹ is propyl and R² is cyclobutyl; (ab) R¹ is isopropyl and R² is cyclobutyl; (ac) R¹ is 1,2-dimethylpropyl and R² is cyclobutyl; (ad) R¹ is cyclopropyl and R² is cyclobutyl; (ae) R¹ is 1-methylpropyl and R² is cyclobutyl; (af) R¹ is isopropyl and R² is methylcyclobutyl; (ag) R¹ is methyl and R² is 1,2-dimethylpropyl; (ah) R¹ is methyl and R² is 1-methylallyl; (ai) R¹ is propargyl and R² is propyl; (aj) R¹ is propargyl and R² is 1-methylpropyl; (ak) R¹ is cyanomethyl and R² is propyl; (al) R¹ is cyanomethyl and R² is 1-methylpropyl; (am) R¹ is methyl, and R² is cyclopropylmethyl; (an) R¹ is methyl, and R² is cyanomethyl; (ao) R¹ is methyl, and R² is propargyl; (ap) R¹ is ethyl, and R² is propyl; (aq) R¹ is ethyl, and R² is iso-butyl; (ar) R¹ is ethyl, and R² is 3-thietanyl; (as) R¹ is ethyl, and R² is cyclopropyleth-2-yl; (at) R¹ is ethyl, and R² is cyclopropylmethyl; (au) R¹ is ethyl, and R² is 1,2-dimethylpropyl; (av) R¹ is ethyl, and R² is cyanomethyl; (aw) R¹ is propyl, and R² is iso-butyl; (ax) R¹ is propyl, and R² is 3-thietanyl; (ay) R¹ is propyl, and R² is cyclopropyleth-2-yl; (az) R¹ is propyl, and R² is cyclopropylmethyl; (ba) R¹ is propyl, and R² is 1,2-dimethylpropyl; (bb) R¹ is iso-propyl, and R² is 3-thietanyl; (bc) R¹ is iso-propyl, and R² is cyclopropyleth-2-yl; (bd) R¹ is but-2-yl, and R² is cyclopropyl; (be) R¹ is but-2-yl, and R² is iso-butyl; (bf) R¹ is but-2-yl, and R² is 3-thietanyl; (bg) R¹ is but-2-yl, and R² is cyclopropyleth-2-yl; (bh) R¹ is but-2-yl, and R² is cyclopropylmethyl; (bi) R¹ is but-2-yl, and R² is 1,2-dimethylpropyl; (bj) R¹ is but-2-yl, and R² is 1-methylallyl; (bk) R¹ is cyclopropyl, and R² is allyl; (bl) R¹ is cyclopropyl, and R² is iso-butyl; (bm) R¹ is cyclopropyl, and R² is 3-thietanyl; (bn) R¹ is cyclopropyl, and R² is cyclopropylmethyl; (bo) R¹ is cyclopropyl, and R² is cyclopropyleth-2-yl; (bp) R¹ is cyclopropyl, and R² is 1,2-dimethylpropyl; (bq) R¹ is cyclopropyl, and R² is 1-methylallyl; (br) R¹ is cyclopropyl, and R² is cyanomethyl; (bs) R¹ is cyclopropyl, and R² is propargyl; (bt) R¹ is cyclobutyl, and R² is allyl; (bu) R¹ is cyclobutyl, and R² is iso-butyl; (by) R¹ is cyclobutyl, and R² is 3-thietanyl; (bw) R¹ is cyclobutyl, and R² is cyclopropylmethyl; (bx) R¹ is cyclobutyl, and R² is cyclopropyleth-2-yl; (by) R¹ is cyclobutyl, and R² is 1,2-dimethylpropyl; (bz) R¹ is cyclobutyl, and R² is 1-methylallyl; (ca) R¹ is cyclobutyl, and R² is cyanomethyl; (cb) R¹ is cyclobutyl, and R² is propargyl; (cc) R¹ is iso-butyl, and R² is allyl; (cd) R¹ is iso-butyl, and R² is 3-thietanyl; (ce) R¹ is iso-butyl, and R² is cyclopropylmethyl; (cf) R¹ is iso-butyl, and R² is cyclopropyleth-2-yl; (cg) R¹ is iso-butyl, and R² is 1,2-dimethylpropyl; (ch) R¹ is iso-butyl, and R² is 1-methylallyl; (ci) R¹ is iso-butyl, and R² is cyanomethyl; (cj) R¹ is iso-butyl, and R² is propargyl; (ck) R¹ is allyl, and R² is iso-butyl; (cl) R¹ is allyl, and R² is 3-thietanyl; (cm) R¹ is allyl, and R² is cyclopropylmethyl; (en) R¹ is allyl, and R² is cyclopropyleth-2-yl; (co) R¹ is allyl, and R² is 1,2-dimethylpropyl; (cp) R¹ is allyl, and R² is 1-methylallyl; (cq) R¹ is allyl, and R² is cyanomethyl; (cr) R¹ is allyl, and R² is propargyl; (cs) R¹ is 3-thietanyl, and R² is cyclopropylmethyl; (ct) R¹ is 3-thietanyl, and R² is cyclopropyleth-2-yl; (cu) R¹ is 3-thietanyl, and R² is 1,2-dimethylpropyl; (cv) R¹ is 3-thietanyl, and R² is 1-methylallyl; (cw) R¹ is 3-thietanyl, and R² is cyanomethyl; (cx) R¹ is 3-thietanyl, and R² is propargyl; (cy) R¹ is cyclopropyleth-2-yl, and R² is cyclopropylmethyl; (cz) R¹ is cyclopropyleth-2-yl, and R² is 1,2-dimethylpropyl; (da) R¹ is cyclopropyleth-2-yl, and R² is 1-methylallyl; (db) R¹ is cyclopropyleth-2-yl, and R² is cyanomethyl; (dc) R¹ is cyclopropyleth-2-yl, and R² is propargyl; (dd) R¹ is cyclopropylemthyl, and R² is cyanomethyl; (de) R¹ is cyclopropylmethyl, and R² is propargyl; (df) R¹ is 1,2-dimethylpropyl, and R² is 1-methylallyl; (dg) R¹ is 1,2-dimethylpropyl, and R² is propargyl; (dh) R¹ is 1,2-dimethylpropyl, and R² is cyanomethyl; (di) R¹ is 1-methylallyl, and R² is cyanomethyl; (dj) R¹ is 1-methylallyl, and R² is propargyl; or (dk) R¹ is propargyl, and R² is cyanomethyl.

In certain embodiments, the compound of formula (I) is a compound of formula (I″), or a salt, prodrug, solvate, isotopologue, or stereoisomer thereof:

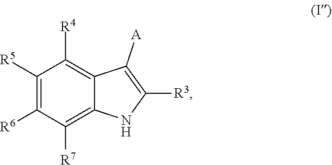

wherein:
   A is selected from the group consisting of

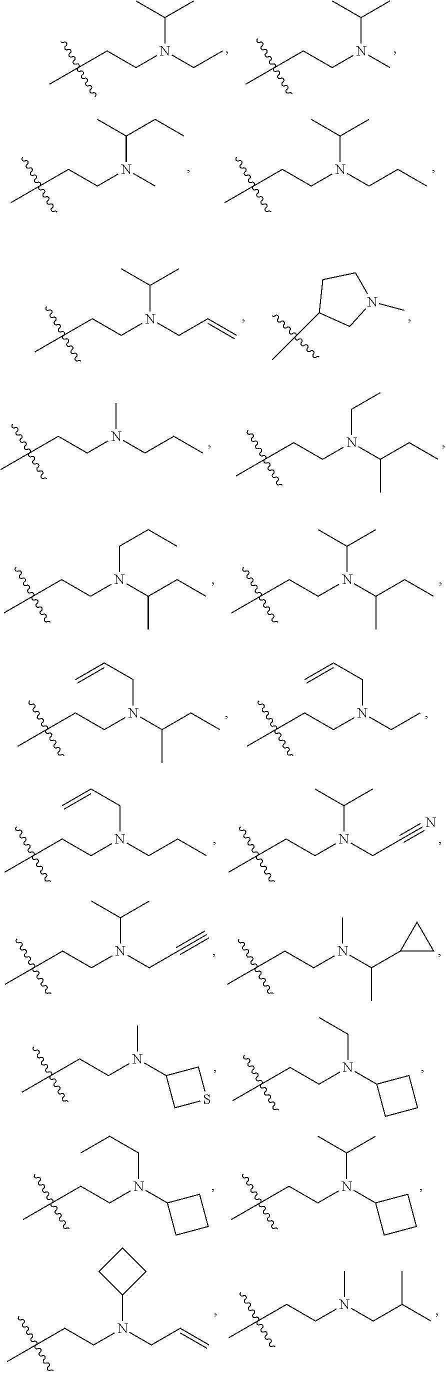

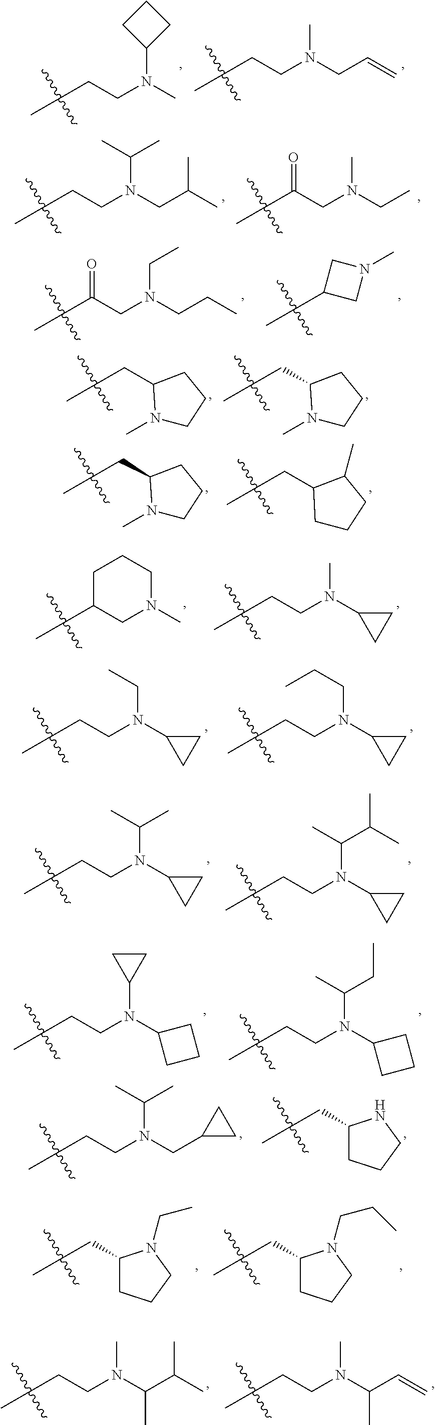

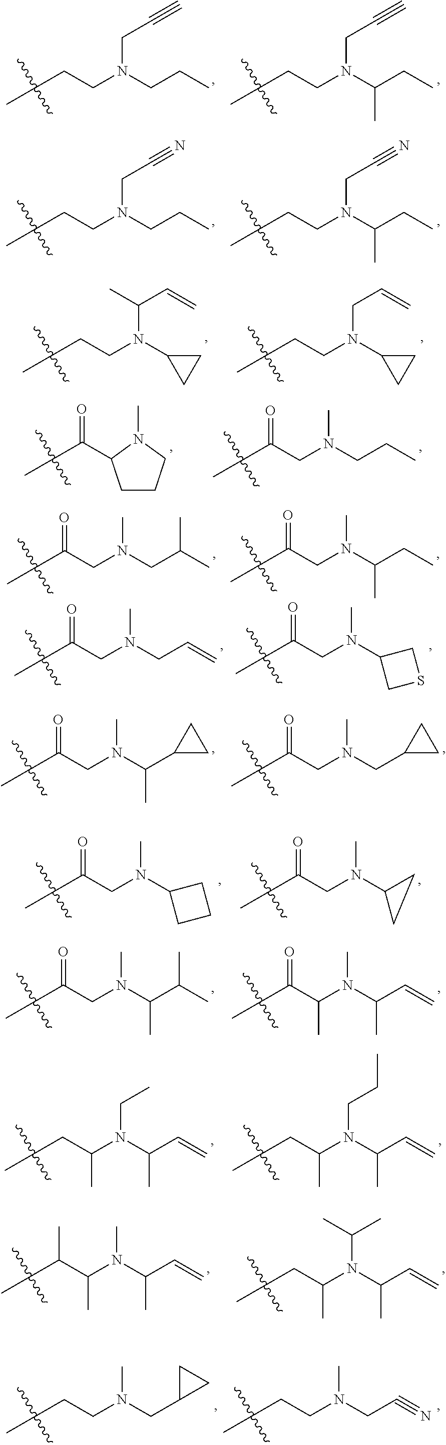

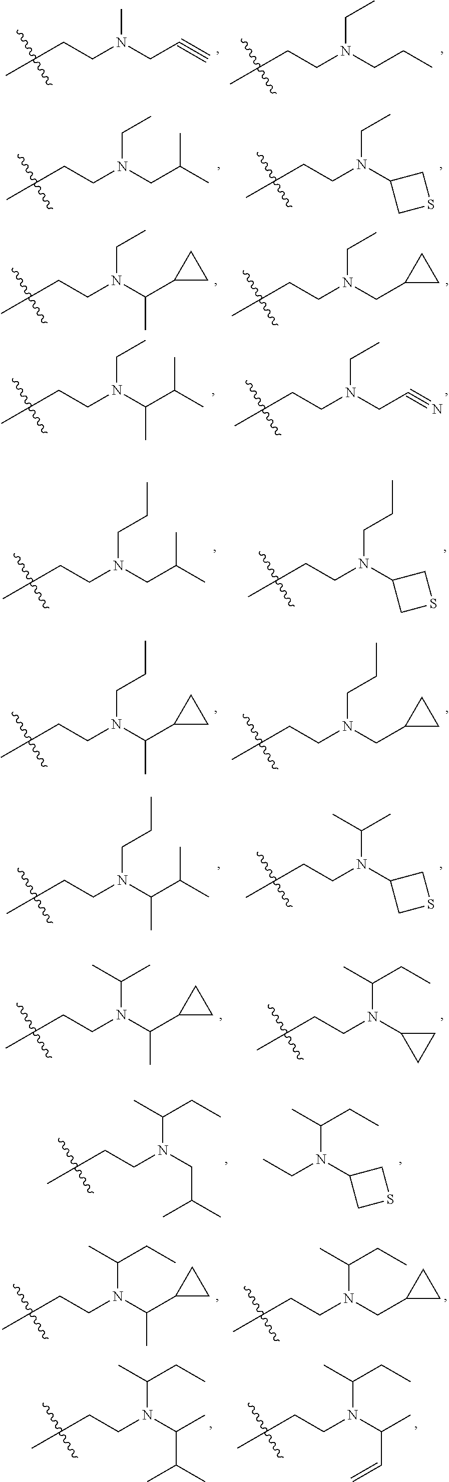

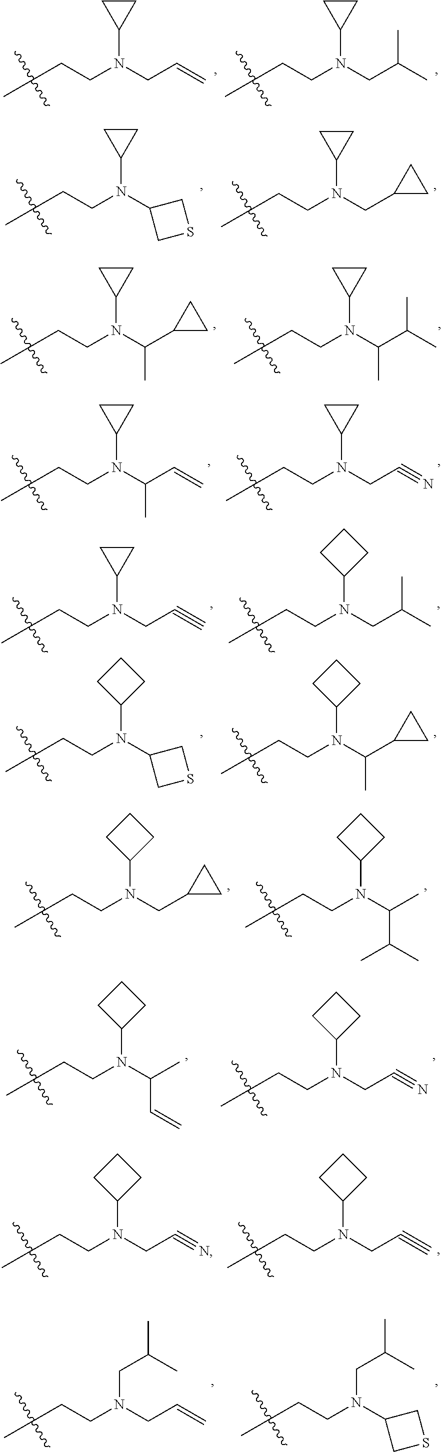

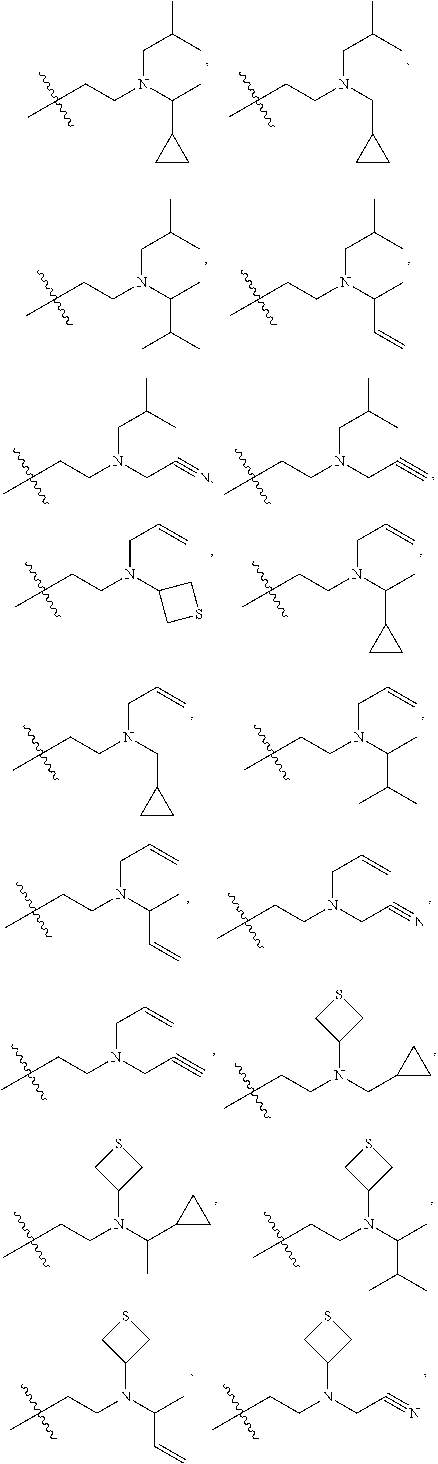

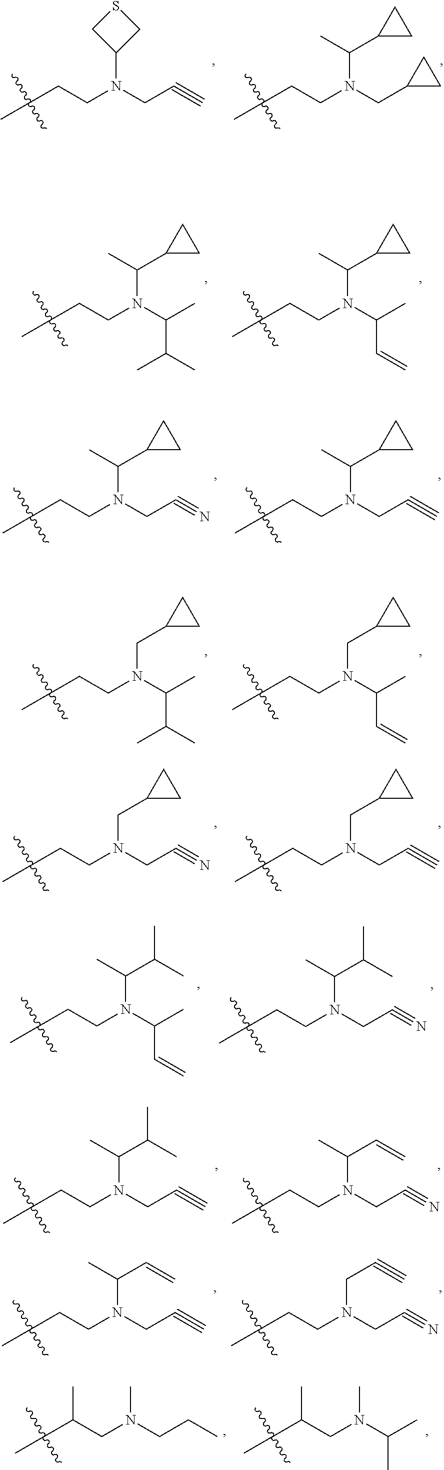

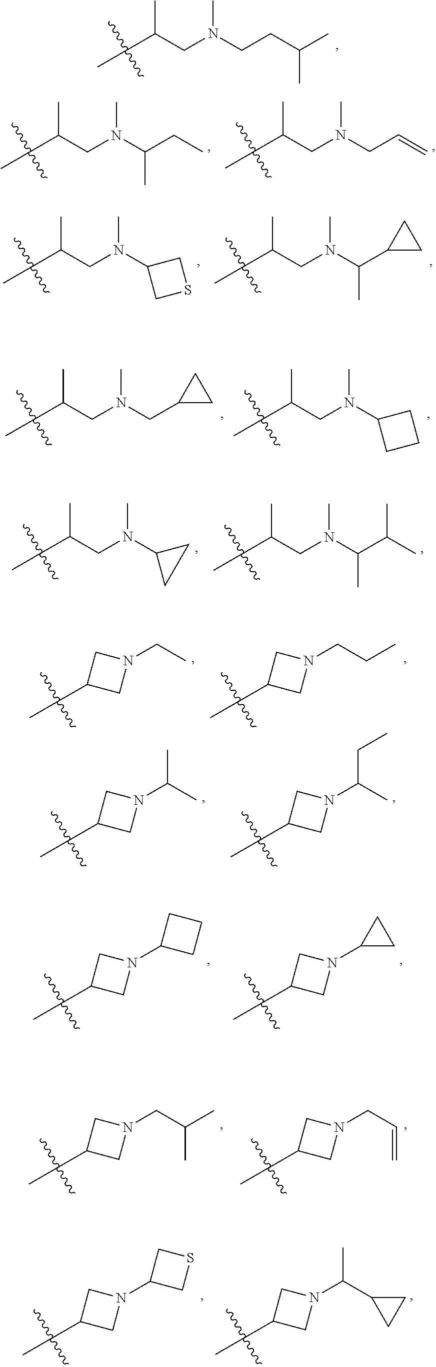

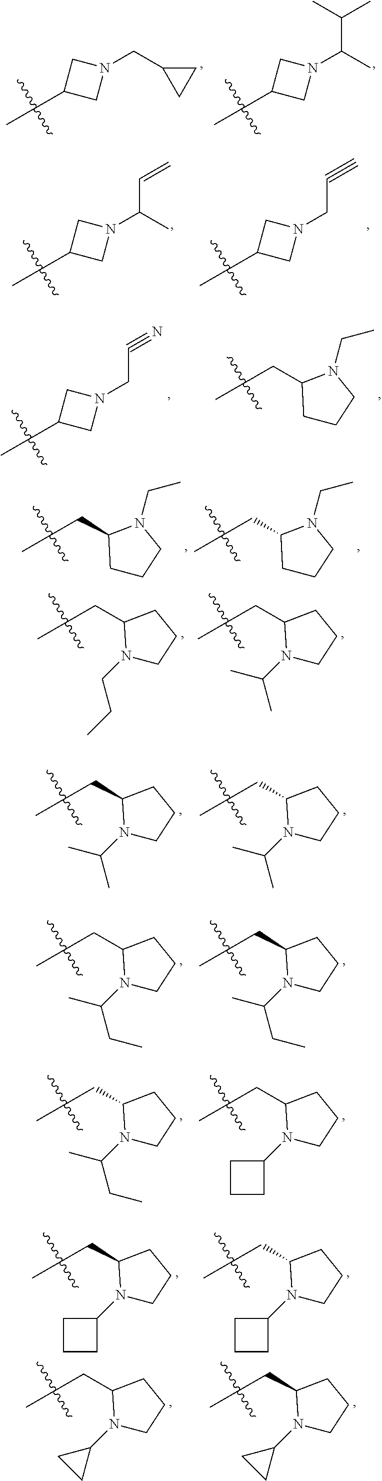

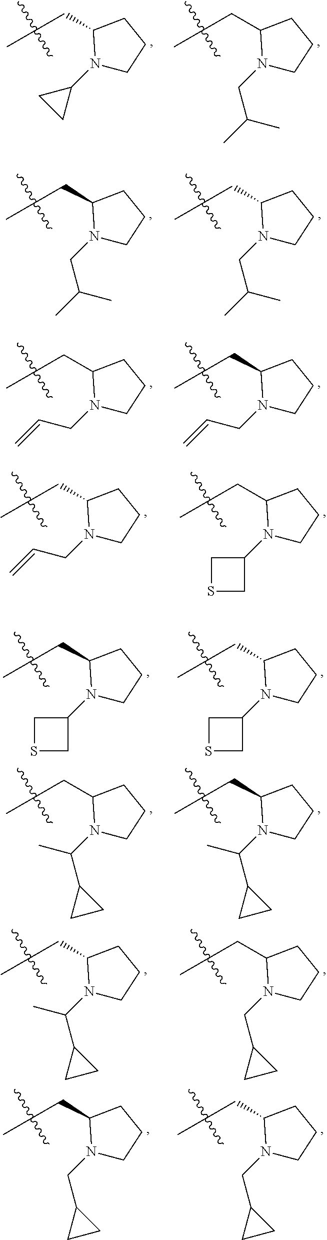

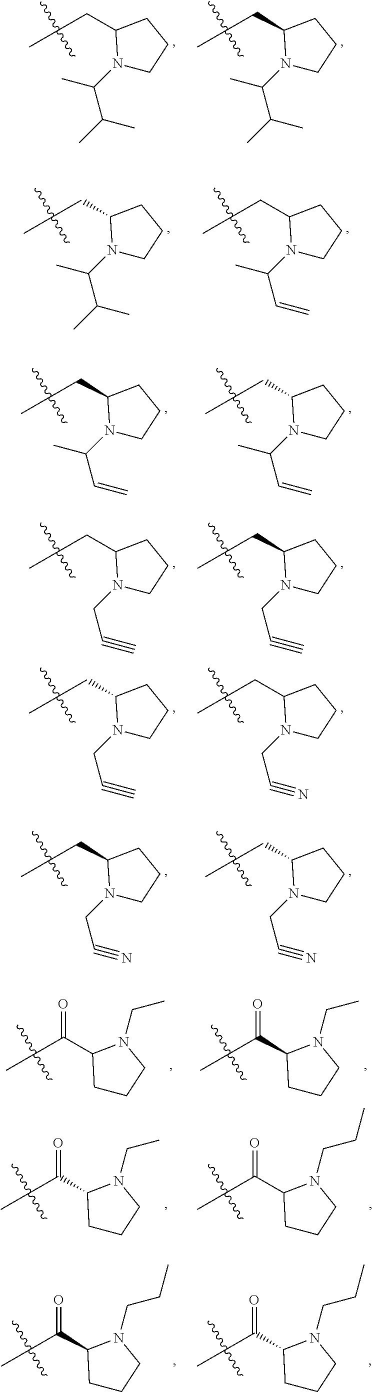

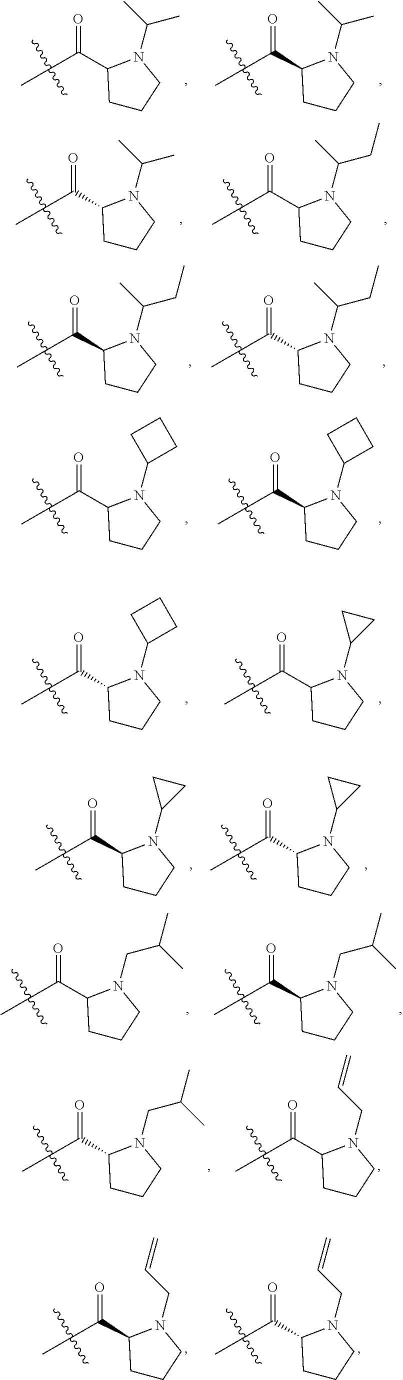

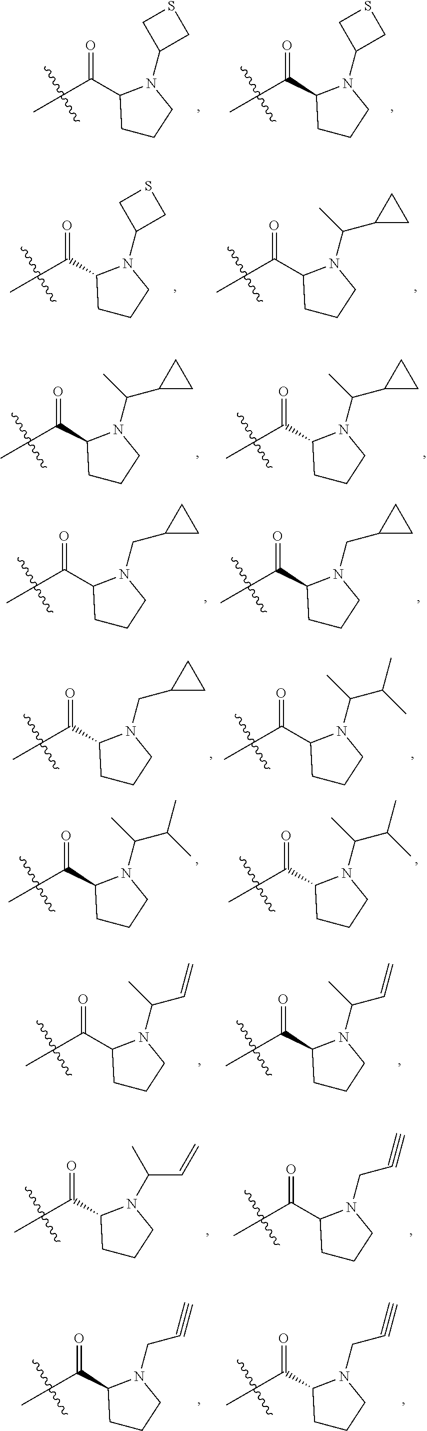

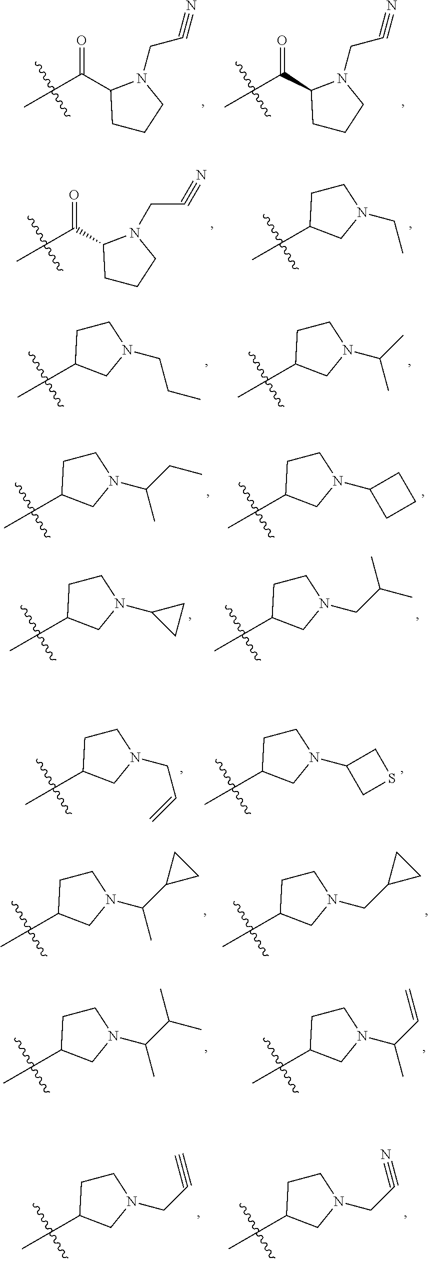

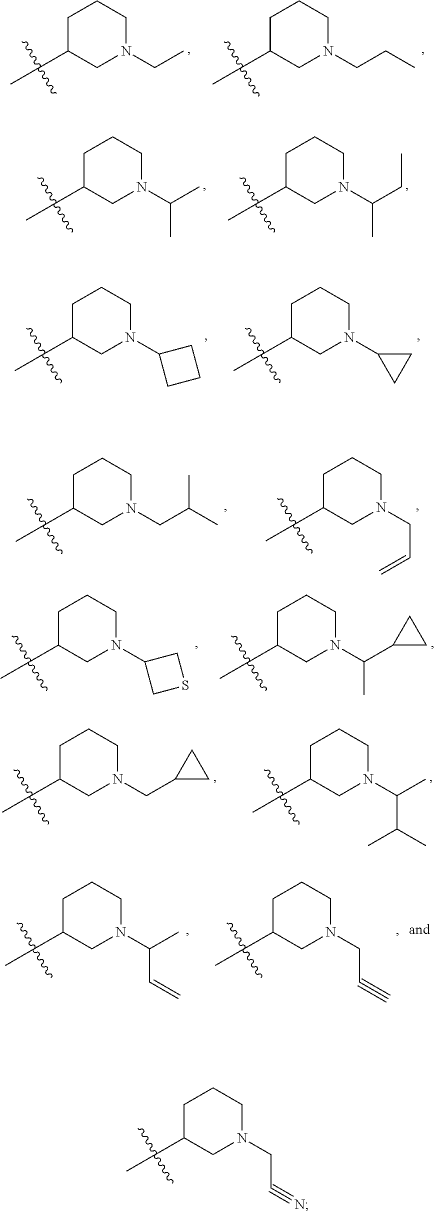

R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
   wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl);
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

In certain embodiments, the compound of formula (I) is a compound of formula (I″), or a salt, prodrug, solvate, isotopologue, or stereoisomer thereof:

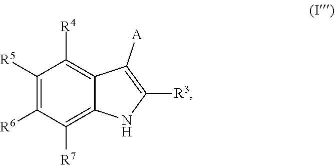

wherein:
   A is selected from the group consisting of

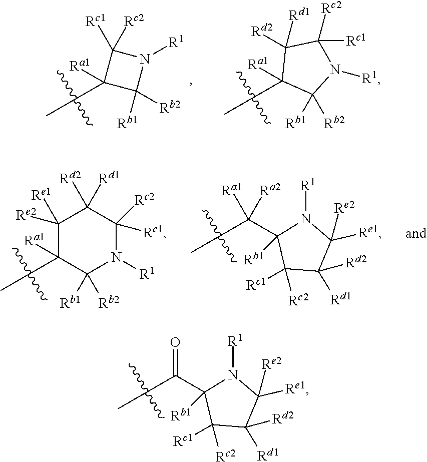

R^{a1} and R^{a2}, if present, are each independently selected from the group consisting of H, halogen, C₁-C₆ alkoxy, and C₁-C₆ alkyl,
R^{b1} and R^{b2}, if present, are each independently selected from the group consisting of H and C₁-C₆ alkyl;
R^{c1}, R^{c2}, R^{d1}, R^{d2}, R^{c1}, and R^{c2}, if present, are each independently selected from the group consisting of H, C₁-C₃ alkyl, and C₁-C₃ haloalkyl;
R¹ is selected from the group consisting of optionally substituted C₁-C₈ alkyl, optionally substituted C₃-C₈ cycloalkyl, optionally substituted C₁-C₈ heteroalkyl, optionally substituted C₂-C₈ alkenyl, and optionally substituted C₂-C₈ alkynyl,
R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
   wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂₀(C₆-C₁₀ aryl), —S(═O)₂₀(C₁-C₆ alkyl), —S(═O)₂₀(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

In certain embodiments, R¹ is optionally substituted C₁-C₃ alkyl and R² is optionally substituted, branched C₃-C₈ alkyl. In certain embodiments, R¹ is selected from the group consisting of methyl, allyl, and n-propyl and R² is optionally substituted, branched C₃-C₈ alkyl.

In certain embodiments, R¹ is C₁-C₃ alkyl and R² is selected from the group consisting of iso-propyl, sec-butyl, iso-butyl, 1,2-dimethylpropyl, methylallyl, and 2-methylallyl. In certain embodiments, R¹ is selected from the group consisting of methyl, allyl, and n-propyl and R² is selected from the group consisting of iso-propyl, sec-butyl, iso-butyl, 1,2-dimethylpropyl, methylallyl, and 2-methylallyl.

In certain embodiments, R¹ is optionally substituted C₁-C₃ alkyl and R² is optionally substituted C₃-C₈ cycloalkyl. In certain embodiments, R¹ is selected from the group consisting of methyl, allyl, and n-propyl and R² is optionally substituted C₃-C₈ cycloalkyl. In certain embodiments, R¹ is optionally substituted C₁-C₃ alkyl, and R² is selected from the group consisting of cyclopropyl and cyclobutyl. In certain embodiments, R¹ is selected from the group consisting of methyl, allyl, and n-propyl and R² is selected from the group consisting of cyclopropyl and cyclobutyl.

In certain embodiments, R¹ is optionally substituted alkoxy. In certain embodiments, R¹ is optionally substituted C₁-C₈ haloalkyl. In certain embodiments, R¹ is methyl. In certain embodiments, R¹ is ethyl. In certain embodiments, R¹ is n-propyl. In certain embodiments, R¹ is iso-propyl. In certain embodiments, R¹ is sec-butyl. In certain embodiments, R¹ is iso-butyl. In certain embodiments, R¹ is n-butyl. In certain embodiments, R¹ is cyclopropyl. In certain embodiments, R¹ is cyclopropylmethyl. In certain embodiments, R¹ is methylcyclopropyl. In certain embodiments, R¹ is cyclopropylethyl. In certain embodiments, R¹ is 2-cyclopropyleth-2-yl. In certain embodiments, R¹ is cyclobutyl. In certain embodiments, R¹ is 2-thietanyl. In certain embodiments, R¹ is 3-thietanyl. In certain embodiments, R¹ is allyl. In certain embodiments, R¹ is methylallyl. In certain embodiments, R¹ is 2-methylallyl. In certain embodiments, R¹ is 3-methylallyl. In certain embodiments, R¹ is allylmethyl. In certain embodiments, R¹ is propargyl.

In certain embodiments, R¹ is cyanomethyl. In certain embodiments, R¹ is 2-hydroxyethyl. In certain embodiments, R¹ is and 2-methoxyethyl. In certain embodiments, R¹ is 1,2-dimethylpropyl.

In certain embodiments, R² is optionally substituted alkoxy. In certain embodiments, R² is optionally substituted C₁-C₈ haloalkyl. In certain embodiments, R² is methyl. In certain embodiments, R² is ethyl. In certain embodiments, R² is n-propyl. In certain embodiments, R² is iso-propyl. In certain embodiments, R² is sec-butyl. In certain embodiments, R² is iso-butyl. In certain embodiments, R² is n-butyl. In certain embodiments, R² is cyclopropyl. In certain embodiments, R² is cyclopropylmethyl. In certain embodiments, R² is methylcyclopropyl. In certain embodiments, R² is cyclopropylethyl. In certain embodiments, R¹ is 2-cyclopropyleth-2-yl. In certain embodiments, R² is cyclobutyl. In certain embodiments, R¹ is 2-thietanyl. In certain embodiments, R¹ is 3-thietanyl. In certain embodiments, R² is allyl. In certain embodiments, R² is methylallyl. In certain embodiments, R² is 2-methylallyl. In certain embodiments, R² is 3-methylallyl. In certain embodiments, R² is allylmethyl. In certain embodiments, R² is propargyl. In certain embodiments, R² is cyanomethyl. In certain embodiments, R² is 2-hydroxyethyl. In certain embodiments, R² is and 2-methoxyethyl. In certain embodiments, R² is 1,2-dimethylpropyl.

In certain embodiments, R³ is H. In certain embodiments, R³ is methyl. In certain embodiments, R³ is phenyl. In certain embodiments, R³ is benzyl.

In certain embodiments, R⁴ is selected from the group consisting of H, C₁, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl, and R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
wherein at least one of R⁵, R⁶, and R⁷ is F.

In certain embodiments, R⁴ is H, and R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
wherein at least one of R⁵, R⁶, and R⁷ is F.

In certain embodiments, R⁴ is C₁-C₆ haloalkyl. In certain embodiments, R⁴ is H. In certain embodiments, R⁴ is F. In certain embodiments, R⁴ is OH. In certain embodiments, R⁴ is OMe. In certain embodiments, R⁴ is C₁.

In certain embodiments, R⁵ is C₁-C₆ haloalkyl. In certain embodiments, R⁵ is H. In certain embodiments, R⁵ is F. In certain embodiments, R⁵ is selected from the group consisting of OR^{A}, N(R^{A})(R^{B}), and SR^{A}. In certain embodiments, R⁵ is OH. In certain embodiments, R⁵ is OMe. In certain embodiments, R⁵ is C₁. In certain embodiments, R⁵ is Me.

In certain embodiments, R⁶ is C₁-C₆ haloalkyl. In certain embodiments, R⁶ is H. In certain embodiments, R⁶ is F. In certain embodiments, R⁶ is OH. In certain embodiments, R⁶ is OMe. In certain embodiments, R⁶ is Cl. In certain embodiments, R⁶ is Me.

In certain embodiments, R⁷ is C₁-C₆ haloalkyl. In certain embodiments, R⁷ is H. In certain embodiments, R⁷ is F. In certain embodiments, R⁷ is OH. In certain embodiments, R⁷ is OMe. In certain embodiments, R⁷ is Cl. In certain embodiments, R⁷ is Br. In certain embodiments, R⁷ is Me.

In certain embodiments, R⁴ is F, and each of R⁵, R⁶, and R⁷ is H. In certain embodiments, R⁵ is F, and each of R⁴, R⁶, and R⁷ is H. In certain embodiments, R⁷ is F, and each of R⁴, R⁵, and R⁶ is H. In certain embodiments, each of R⁴ and R⁵ is F, and each of R⁶ and R⁷ is H. In certain embodiments, each of R⁴ and R⁶ is F, and each of R⁵ and R⁷ is H. In certain embodiments, each of R⁴ and R⁷ is F, and each of R⁵ and R⁶ is H. In certain embodiments, each of R⁵ and R⁶ is F, and each of R⁴ and R⁷ is H. In certain embodiments, each of R⁵ and R⁷ is F, and each of R⁴ and R⁶ is H. In certain embodiments, each of R⁶ and R⁷ is F, and each of R⁴ and R⁵ is H. In certain embodiments, R⁵ is OMe, R⁷ is F, and each of R⁴ and R⁶ is H. In certain embodiments, R⁵, R⁶, and R⁷ are each F, and R⁴ is H.

In certain embodiments, each of R⁴ and R⁶ is H.

In certain embodiments, L is

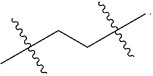 [SMILES: *CC*]

In certain embodiments, L is

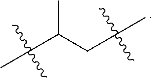 [SMILES: *CC(*)C]

In certain embodiments, L is

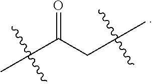 [SMILES: *CC(*)=O]

In certain embodiments, A is selected from the group consisting of

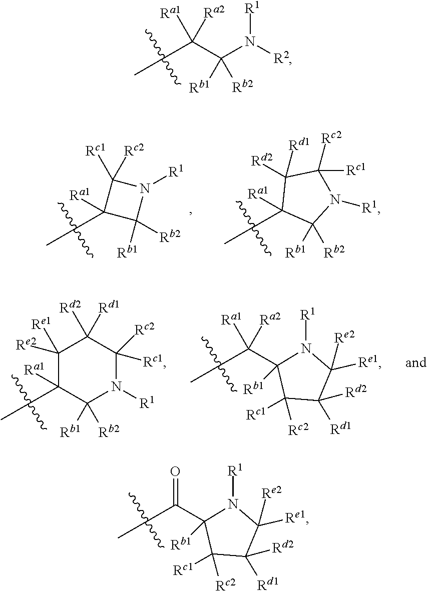

and
wherein:
   R^{c1}, R^{c2}, R^{d1}, R^{d2}, R^{c1}, and R^{c2}, if present, are each independently selected from the group consisting of H, C₁-C₃ alkyl, and C₁-C₃ haloalkyl.

In certain embodiments, each of R^{a1}, R^{a2}, R^{b1}, R^{b2}, R^{c1}, R^{c2}, R^{d1}, R^{d2}, R^{c1}, R^{c2}, R^{f1}, and R^{f2}, if present, is H.

In certain embodiments, R¹ is iso-propyl and R² is ethyl. In certain embodiments, R¹ is iso-propyl and R² is methyl. In certain embodiments, R¹ is sec-butyl and R² is methyl. In certain embodiments, R¹ is iso-propyl and R² is n-propyl. In certain embodiments, R¹ is iso-propyl and R² is allyl. In certain embodiments, R¹ is methyl and R² is ethyl. In certain embodiments, R¹ is methyl and R² is n-propyl. In certain embodiments, R¹ is ethyl and R² is n-propyl. In certain embodiments, R¹ is ethyl and R² is sec-butyl. In certain embodiments, R¹ is n-propyl and R² is sec-butyl. In certain embodiments, R¹ is iso-propyl and R² is sec-butyl. In certain embodiments, R¹ is allyl and R² is sec-butyl. In certain embodiments, R¹ is allyl and R² is ethyl. In certain embodiments, R¹ is allyl and R² is n-propyl. In certain embodiments, R¹ is isopropyl and R² is cyanomethyl. In certain embodiments, R¹ is iso-propyl and R² is propargyl. In certain embodiments, R¹ is methyl and R² is 2-cyclopropyleth-2-yl. In certain embodiments, R¹ is methyl and R² is 3-thietanyl. In certain embodiments, R¹ is ethyl and R² is cyclobutyl. In certain embodiments, R¹ is n-propyl and R² is cyclobutyl. In certain embodiments, R¹ is iso-propyl and R² is cyclobutyl. In certain embodiments, R¹ is allyl and R² is cyclobutyl. In certain embodiments, R¹ is methyl and R² is iso-butyl. In certain embodiments, R¹ is cyclobutyl and R² is methyl. In certain embodiments, R¹ is methyl and R² is allyl. In certain embodiments, R¹ is iso-propyl and R² is iso-butyl. In certain embodiments, R¹ is methyl and R² is cyclobutyl. In certain embodiments, R¹ is ethyl and R² is cyclobutyl. In certain embodiments, R¹ is propyl and R² is cyclobutyl. In certain embodiments, R¹ is isopropyl and R² is cyclobutyl. In certain embodiments, R¹ is 1,2-dimethylpropyl and R² is cyclobutyl. In certain embodiments, R¹ is cyclopropyl and R² is cyclobutyl. In certain embodiments, R¹ is 1-methylpropyl and R² is cyclobutyl. In certain embodiments, R¹ is isopropyl and R² is methylcyclobutyl. In certain embodiments, R¹ is methyl and R² is 1,2-dimethylpropyl. In certain embodiments, R¹ is methyl and R² is 1-methylallyl. In certain embodiments, R¹ is propargyl and R² is propyl. In certain embodiments, R¹ is propargyl and R² is 1-methylpropyl. In certain embodiments, R¹ is cyanomethyl and R² is propyl. In certain embodiments, R¹ is cyanomethyl and R² is 1-methylpropyl. In certain embodiments, R¹ is methyl, and R² is cyclopropylmethyl. In certain embodiments, R¹ is methyl, and R² is cyanomethyl. In certain embodiments, R¹ is methyl, and R² is propargyl. In certain embodiments, R¹ is ethyl, and R² is propyl. In certain embodiments, R¹ is ethyl, and R² is iso-butyl. In certain embodiments, R¹ is ethyl, and R² is 3-thietanyl. In certain embodiments, R¹ is ethyl, and R² is cyclopropyleth-2-yl. In certain embodiments, R¹ is ethyl, and R² is cyclopropylmethyl. In certain embodiments, R¹ is ethyl, and R² is 1,2-dimethylpropyl. In certain embodiments, R¹ is ethyl, and R² is cyanomethyl. In certain embodiments, R¹ is propyl, and R² is iso-butyl. In certain embodiments, R¹ is propyl, and R² is 3-thietanyl. In certain embodiments, R¹ is propyl, and R² is cyclopropyleth-2-yl. In certain embodiments, R¹ is propyl, and R² is cyclopropylmethyl. In certain embodiments, R¹ is propyl, and R² is 1,2-dimethylpropyl. In certain embodiments, R¹ is iso-propyl, and R² is 3-thietanyl. In certain embodiments, R¹ is iso-propyl, and R² is cyclopropyleth-2-yl. In certain embodiments, R¹ is but-2-yl, and R² is cyclopropyl. In certain embodiments, R¹ is but-2-yl, and R² is iso-butyl.

In certain embodiments, R¹ is but-2-yl, and R² is 3-thietanyl. In certain embodiments, R¹ is but-2-yl, and R² is cyclopropyleth-2-yl. In certain embodiments, R¹ is but-2-yl, and R² is cyclopropylmethyl. In certain embodiments, R¹ is but-2-yl, and R² is 1,2-dimethylpropyl. In certain embodiments, R¹ is but-2-yl, and R² is 1-methylallyl In certain embodiments, R¹ is cyclopropyl, and R² is allyl. In certain embodiments, R¹ is cyclopropyl, and R² is iso-butyl. In certain embodiments, R¹ is cyclopropyl, and R² is 3-thietanyl. In certain embodiments, R¹ is cyclopropyl, and R² is cyclopropylmethyl. In certain embodiments, R¹ is cyclopropyl, and R² is cyclopropyleth-2-yl. In certain embodiments, R¹ is cyclopropyl, and R² is 1,2-dimethylpropyl. In certain embodiments, R¹ is cyclopropyl, and R² is 1-methylallyl. In certain embodiments, R¹ is cyclopropyl, and R² is cyanomethyl. In certain embodiments, R¹ is cyclopropyl, and R² is propargyl. In certain embodiments, R¹ is cyclobutyl, and R² is allyl. In certain embodiments, R¹ is cyclobutyl, and R² is iso-butyl. In certain embodiments, R¹ is cyclobutyl, and R² is 3-thietanyl. In certain embodiments, R¹ is cyclobutyl, and R² is cyclopropylmethyl. In certain embodiments, R¹ is cyclobutyl, and R² is cyclopropyleth-2-yl. In certain embodiments, R¹ is cyclobutyl, and R² is 1,2-dimethylpropyl. In certain embodiments, R¹ is cyclobutyl, and R² is 1-methylallyl. In certain embodiments, R¹ is cyclobutyl, and R² is cyanomethyl. In certain embodiments, R¹ is cyclobutyl, and R² is propargyl. In certain embodiments, R¹ is iso-butyl, and R² is allyl. In certain embodiments, R¹ is iso-butyl, and R² is 3-thietanyl. In certain embodiments, R¹ is iso-butyl, and R² is cyclopropylmethyl. In certain embodiments, R¹ is iso-butyl, and R² is cyclopropyleth-2-yl. In certain embodiments, R¹ is iso-butyl, and R² is 1,2-dimethylpropyl. In certain embodiments, R¹ is iso-butyl, and R² is 1-methylallyl. In certain embodiments, R¹ is iso-butyl, and R² is cyanomethyl. In certain embodiments, R¹ is iso-butyl, and R² is propargyl. In certain embodiments, R¹ is allyl, and R² is iso-butyl. In certain embodiments, R¹ is allyl, and R² is 3-thietanyl. In certain embodiments, R¹ is allyl, and R² is cyclopropylmethyl. In certain embodiments, R¹ is allyl, and R² is cyclopropyleth-2-yl. In certain embodiments, R¹ is allyl, and R² is 1,2-dimethylpropyl. In certain embodiments, R¹ is allyl, and R² is 1-methylallyl. In certain embodiments, R¹ is allyl, and R² is cyanomethyl. In certain embodiments, R¹ is allyl, and R² is propargyl. In certain embodiments, R¹ is 3-thietanyl, and R² is cyclopropylmethyl. In certain embodiments, R¹ is 3-thietanyl, and R² is cyclopropyleth-2-yl. In certain embodiments, R¹ is 3-thietanyl, and R² is 1,2-dimethylpropyl. In certain embodiments, R¹ is 3-thietanyl, and R² is 1-methylallyl. In certain embodiments, R¹ is 3-thietanyl, and R² is cyanomethyl. In certain embodiments, R¹ is 3-thietanyl, and R² is propargyl. In certain embodiments, R¹ is cyclopropyleth-2-yl, and R² is cyclopropylmethyl. In certain embodiments, R¹ is cyclopropyleth-2-yl, and R² is 1,2-dimethylpropyl. In certain embodiments, R¹ is cyclopropyleth-2-yl, and R² is 1-methylallyl. In certain embodiments, R¹ is cyclopropyleth-2-yl, and R² is cyanomethyl. In certain embodiments, R¹ is cyclopropyleth-2-yl, and R² is propargyl. In certain embodiments, R¹ is cyclopropylmethyl, and R² is cyanomethyl. In certain embodiments, R¹ is cyclopropylmethyl, and R² is propargyl. In certain embodiments, R¹ is 1,2-dimethylpropyl, and R² is 1-methylallyl. In certain embodiments, R¹ is 1,2-dimethylpropyl, and R² is propargyl. In certain embodiments, R¹ is 1,2-dimethylpropyl, and R² is cyanomethyl. In certain embodiments, R¹ is 1-methylallyl, and R² is cyanomethyl. In certain embodiments, R¹ is 1-methylallyl, and R² is propargyl. In certain embodiments, R¹ is propargyl, and R² is cyanomethyl.

In certain embodiments, A is

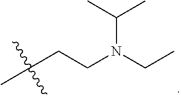 [SMILES: *CCN(CC)C(C)C]

In certain embodiments, A is

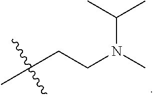 [SMILES: *CCN(C)C(C)C]

In certain embodiments, A is

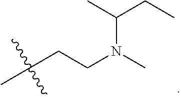 [SMILES: *CCN(C)C(C)CC]

In certain embodiments, A is

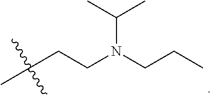 [SMILES: *CCN(CCC)C(C)C]

In certain embodiments, A is

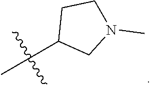 [SMILES: *C1CCN(C)C1]

In certain embodiments, A is

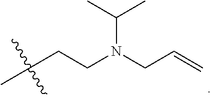 [SMILES: *CCN(CC=C)C(C)C]

In certain embodiments, A is

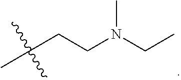 [SMILES: *CCN(C)CC]

In certain embodiments, A is

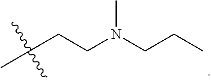 [SMILES: *CCN(C)CCC]

In certain embodiments, A is

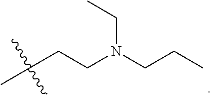 [SMILES: *CCN(CC)CCC]

In certain embodiments, A is

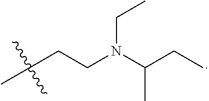 [SMILES: *CCN(CC)C(C)CC]

In certain embodiments, A is

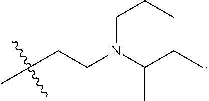 [SMILES: *CCN(CCC)C(C)CC]

In certain embodiments, A is

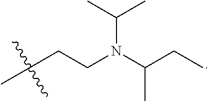 [SMILES: *CCN(C(C)C)C(C)CC]

In certain embodiments, A is

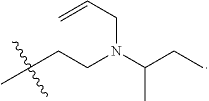 [SMILES: *CCN(CC=C)C(C)CC]

In certain embodiments, A is

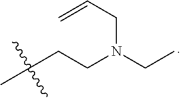 [SMILES: *CCN(CC)CC=C]

In certain embodiments, A is

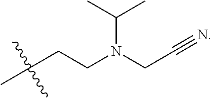 [SMILES: *CCN(CC#N)C(C)C]

In certain embodiments, A is

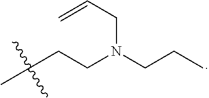 [SMILES: *CCN(CC=C)CCC]

In certain embodiments, A is

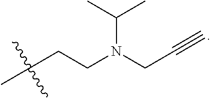 [SMILES: *CCN(CC#C)C(C)C]

In certain embodiments, A is

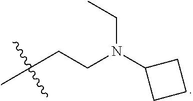 [SMILES: *CCN(CC)C1CCC1]

In certain embodiments, A is

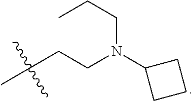 [SMILES: *CCN(CCC)C1CCC1]

In certain embodiments, A is

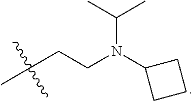 [SMILES: *CCN(C(C)C)C1CCC1]

In certain embodiments, A is

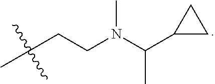 [SMILES: *CCN(C)C(C)C1CC1]

In certain embodiments, A is

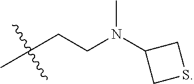 [SMILES: *CCN(C)C1CSC1]

In certain embodiments, A is

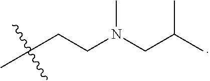 [SMILES: *CCN(C)CC(C)C]

In certain embodiments, A is

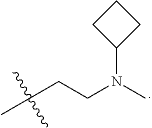 [SMILES: *CCN(C)C1CCC1]

In certain embodiments, A is

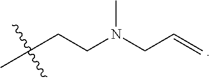 [SMILES: *CCN(C)CC=C]

In certain embodiments, A is

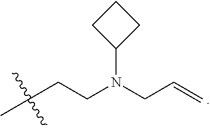 [SMILES: *CCN(CC=C)C1CCC1]

In certain embodiments, A is

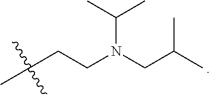 [SMILES: *CCN(CC(C)C)C(C)C]

In certain embodiments, A is

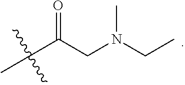 [SMILES: *C(=O)CN(C)CC]

In certain embodiments, A is

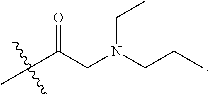 [SMILES: *C(=O)CN(CC)CCC]

In certain embodiments, A is

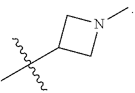 [SMILES: *C1CN(C)C1]

In certain embodiments, A is

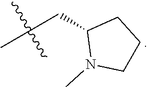 [SMILES: *C[C@@H]1CCCN1C]

In certain embodiments, A is

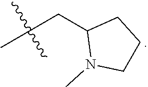 [SMILES: *CC1CCCN1C]

In certain embodiments, A is

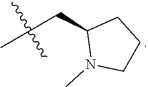 [SMILES: *C[C@H]1CCCN1C]

In certain embodiments, A is

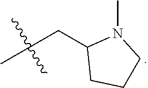 [SMILES: *CC1CCCN1C]

In certain embodiments, A is

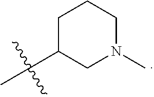 [SMILES: *C1CCCN(C)C1]

In certain embodiments, A is

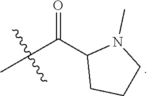 [SMILES: *C(=O)C1CCCN1C]

In certain embodiments, A is

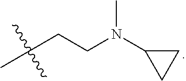 [SMILES: *CCN(C)C1CC1]

In certain embodiments, A is

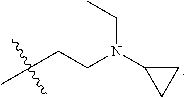 [SMILES: *CCN(CC)C1CC1]

In certain embodiments, A is

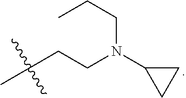 [SMILES: *CCN(CCC)C1CC1]

In certain embodiments, A is

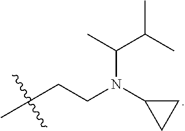 [SMILES: *CCN(C1CC1)C(C)C(C)C]

In certain embodiments, A is

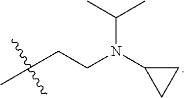 [SMILES: *CCN(C(C)C)C1CC1]

In certain embodiments, A is

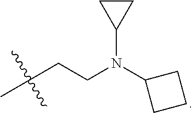 [SMILES: *CCN(C1CCC1)C1CC1]

In certain embodiments, A is

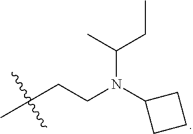 [SMILES: *CCN(C(C)CC)C1CCC1]

In certain embodiments, A is

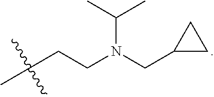 [SMILES: *CCN(CC1CC1)C(C)C]

In certain embodiments, A is

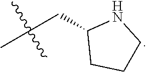 [SMILES: *C[C@H]1CCCN1]

In certain embodiments, A is

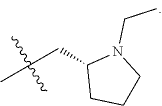 [SMILES: *C[C@H]1CCCN1CC]

In certain embodiments, A is

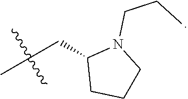 [SMILES: *C[C@H]1CCCN1CCC]

In certain embodiments, A is

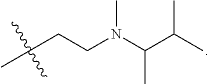 [SMILES: *CCN(C)C(C)C(C)C]

In certain embodiments, A is

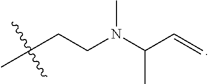 [SMILES: *CCN(C)C(C)C=C]

In certain embodiments, A is

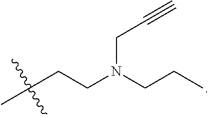 [SMILES: *CCN(CC#C)CCC]

In certain embodiments, A is

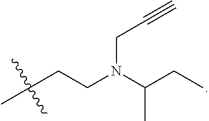 [SMILES: *CCN(CC#C)C(C)CC]

In certain embodiments, A is

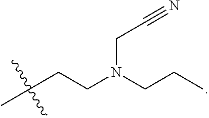 [SMILES: *CCN(CC#N)CCC]

In certain embodiments, A is

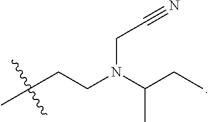 [SMILES: *CCN(CC#N)C(C)CC]

In certain embodiments, A is

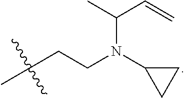 [SMILES: *CCN(C(C)C=C)C1CC1]

In certain embodiments, A is

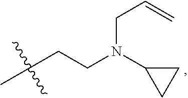 [SMILES: *CCN(CC=C)C1CC1]

In certain embodiments, A is

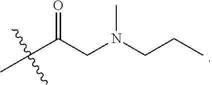 [SMILES: *C(=O)CN(C)CCC]

In certain embodiments, A is

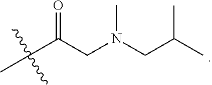 [SMILES: *C(=O)CN(C)CC(C)C]

In certain embodiments, A is

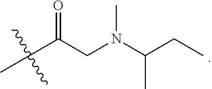 [SMILES: *C(=O)CN(C)C(C)CC]

In certain embodiments, A is

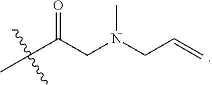 [SMILES: *C(=O)CN(C)CC=C]

In certain embodiments, A is

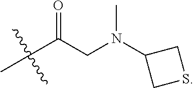 [SMILES: *C(=O)CN(C)C1CSC1]

In certain embodiments, A is

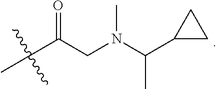 [SMILES: *C(=O)CN(C)C(C)C1CC1]

In certain embodiments, A is

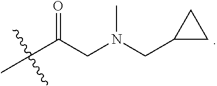 [SMILES: *C(=O)CN(C)CC1CC1]

In certain embodiments, A is

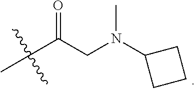 [SMILES: *C(=O)CN(C)C1CCC1]

In certain embodiments, A is

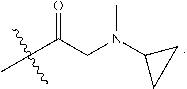 [SMILES: *C(=O)CN(C)C1CC1]

In certain embodiments, A is

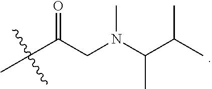 [SMILES: *C(=O)CN(C)C(C)C(C)C]

In certain embodiments, A is

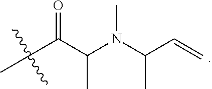 [SMILES: *C(=O)C(C)N(C)C(C)C=C]

In certain embodiments, A is

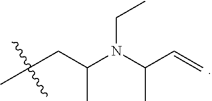 [SMILES: *CC(C)N(CC)C(C)C=C]

In certain embodiments, A is

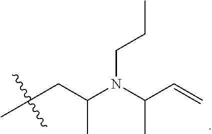 [SMILES: *CC(C)N(CCC)C(C)C=C]

In certain embodiments, A is

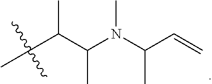 [SMILES: *C(C)C(C)N(C)C(C)C=C]

In certain embodiments, A is

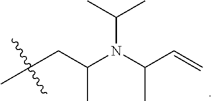 [SMILES: *CC(C)N(C(C)C)C(C)C=C]

In certain embodiments, A is

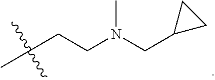 [SMILES: *CCN(C)CC1CC1]

In certain embodiments, A is

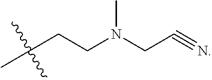 [SMILES: *CCN(C)CC#N]

In certain embodiments, A is

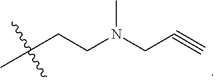 [SMILES: *CCN(C)CC#C]

In certain embodiments, A is

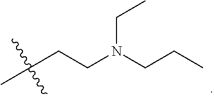 [SMILES: *CCN(CC)CCC]

In certain embodiments, A is

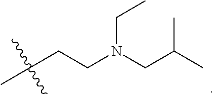 [SMILES: *CCN(CC)CC(C)C]

In certain embodiments, A is

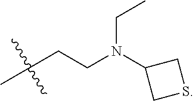 [SMILES: *CCN(CC)C1CSC1]

In certain embodiments, A is

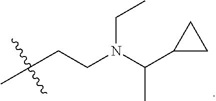 [SMILES: *CCN(CC)C(C)C1CC1]

In certain embodiments, A is

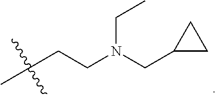 [SMILES: *CCN(CC)CC1CC1]

In certain embodiments, A is

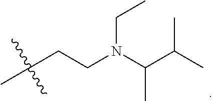 [SMILES: *CCN(CC)C(C)C(C)C]

In certain embodiments, A is

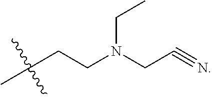 [SMILES: *CCN(CC)CC#N]

In certain embodiments, A is

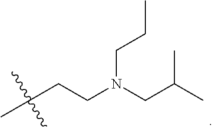 [SMILES: *CCN(CCC)CC(C)C]

In certain embodiments, A is

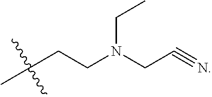 [SMILES: *CCN(CC)CC#N]

In certain embodiments, A is

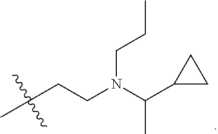 [SMILES: *CCN(CCC)C(C)C1CC1]

In certain embodiments, A is

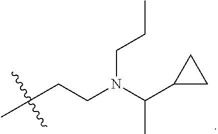 [SMILES: *CCN(CCC)C(C)C1CC1]

In certain embodiments, A is

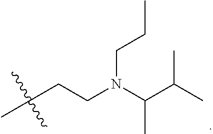 [SMILES: *CCN(CCC)C(C)C(C)C]

In certain embodiments, A is

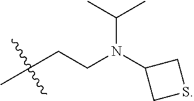 [SMILES: *CCN(C(C)C)C1CSC1]

In certain embodiments, A is

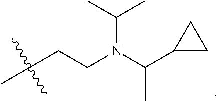 [SMILES: *CCN(C(C)C)C(C)C1CC1]

In certain embodiments, A is

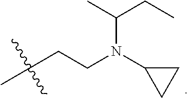 [SMILES: *CCN(C(C)CC)C1CC1]

In certain embodiments, A is

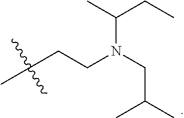 [SMILES: *CCN(CC(C)C)C(C)CC]

In certain embodiments, A is

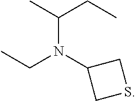

In certain embodiments, A is

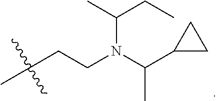 [SMILES: *CCN(C(C)CC)C(C)C1CC1]

In certain embodiments, A is

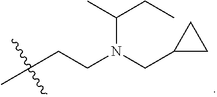 [SMILES: *CCN(CC1CC1)C(C)CC]

In certain embodiments, A is

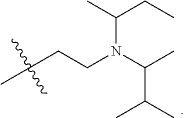 [SMILES: *CCN(C(C)CC)C(C)C(C)C]

In certain embodiments, A is

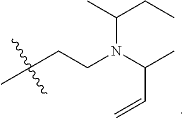 [SMILES: *CCN(C(C)C=C)C(C)CC]

In certain embodiments, A is

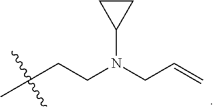 [SMILES: *CCN(CC=C)C1CC1]

In certain embodiments, A is

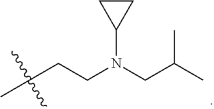 [SMILES: *CCN(CC(C)C)C1CC1]

In certain embodiments, A is

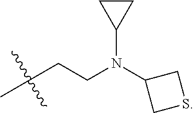 [SMILES: *CCN(C1CC1)C1CSC1]

In certain embodiments, A is

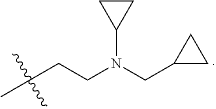 [SMILES: *CCN(CC1CC1)C1CC1]

In certain embodiments, A is

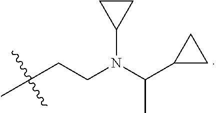 [SMILES: *CCN(C1CC1)C(C)C1CC1]

In certain embodiments, A is

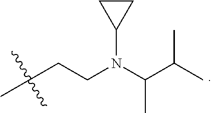 [SMILES: *CCN(C1CC1)C(C)C(C)C]

In certain embodiments, A is

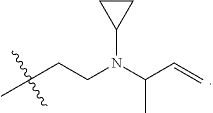 [SMILES: *CCN(C(C)C=C)C1CC1]

In certain embodiments, A is

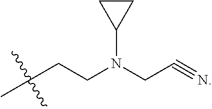 [SMILES: *CCN(CC#N)C1CC1]

In certain embodiments, A is

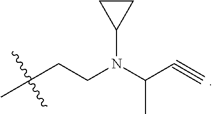 [SMILES: *CCN(C(C)C#C)C1CC1]

In certain embodiments, A is

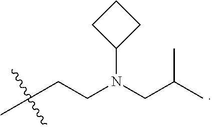 [SMILES: *CCN(CC(C)C)C1CCC1]

In certain embodiments, A is

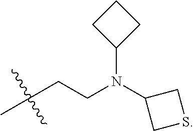 [SMILES: *CCN(C1CCC1)C1CSC1]

In certain embodiments, A is

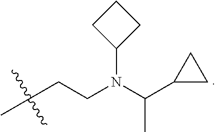 [SMILES: *CCN(C1CCC1)C(C)C1CC1]

In certain embodiments, A is

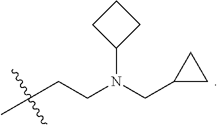 [SMILES: *CCN(CC1CC1)C1CCC1]

In certain embodiments, A is

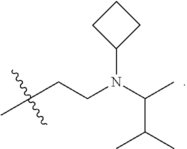 [SMILES: *CCN(C1CCC1)C(C)C(C)C]

In certain embodiments, A is

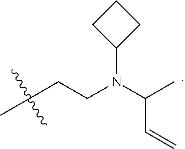 [SMILES: *CCN(C(C)C=C)C1CCC1]

In certain embodiments, A is

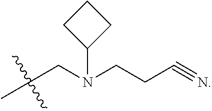 [SMILES: *CN(CCC#N)C1CCC1]

In certain embodiments, A is

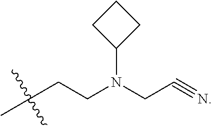 [SMILES: *CCN(CC#N)C1CCC1]

In certain embodiments, A is

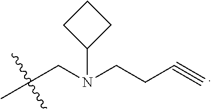 [SMILES: *CN(CCC#C)C1CCC1]

In certain embodiments, A is

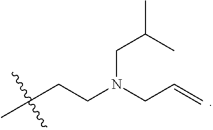 [SMILES: *CCN(CC=C)CC(C)C]

In certain embodiments, A is

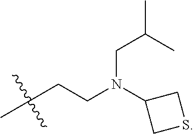 [SMILES: *CCN(CC(C)C)C1CSC1]

In certain embodiments, A is

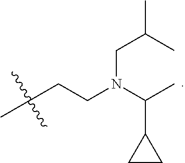 [SMILES: *CCN(CC(C)C)C(C)C1CC1]

In certain embodiments, A is

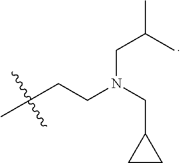 [SMILES: *CCN(CC(C)C)CC1CC1]

In certain embodiments, A is

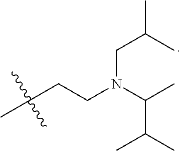 [SMILES: *CCN(CC(C)C)C(C)C(C)C]

In certain embodiments, A is

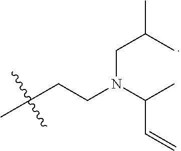 [SMILES: *CCN(CC(C)C)C(C)C=C]

In certain embodiments, A is

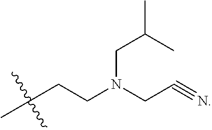 [SMILES: *CCN(CC#N)CC(C)C]

In certain embodiments, A is

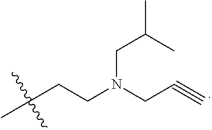 [SMILES: *CCN(CC#C)CC(C)C]

In certain embodiments, A is

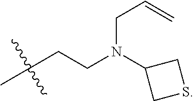 [SMILES: *CCN(CC=C)C1CSC1]

In certain embodiments, A

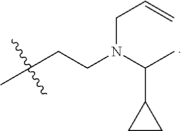 [SMILES: *CCN(CC=C)C(C)C1CC1]

In certain embodiments, A is

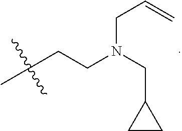 [SMILES: *CCN(CC=C)CC1CC1]

In certain embodiments, A is

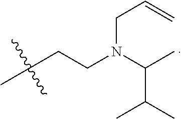 [SMILES: *CCN(CC=C)C(C)C(C)C]

In certain embodiments, A is

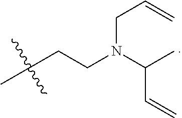 [SMILES: *CCN(CC=C)C(C)C=C]

In certain embodiments, A is

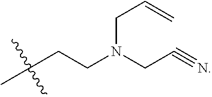 [SMILES: *CCN(CC#N)CC=C]

In certain embodiments, A is

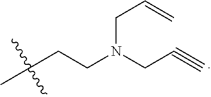 [SMILES: *CCN(CC#C)CC=C]

In certain embodiments, A is

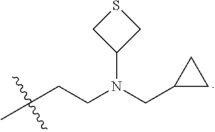 [SMILES: *CCN(CC1CC1)C1CSC1]

In certain embodiments, A is

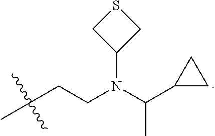 [SMILES: *CCN(C1CSC1)C(C)C1CC1]

In certain embodiments, A is

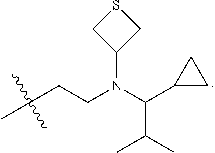 [SMILES: *CCN(C1CSC1)C(C(C)C)C1CC1]

In certain embodiments, A is

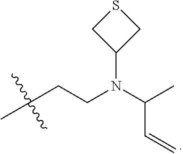 [SMILES: *CCN(C(C)C=C)C1CSC1]

In certain embodiments, A is

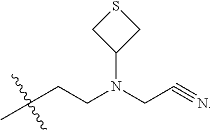 [SMILES: *CCN(CC#N)C1CSC1]

In certain embodiments, A is

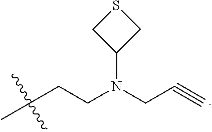 [SMILES: *CCN(CC#C)C1CSC1]

In certain embodiments, A is

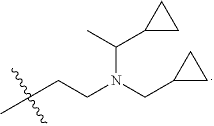 [SMILES: *CCN(CC1CC1)C(C)C1CC1]

In certain embodiments, A is

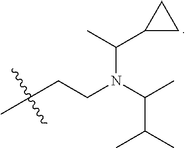 [SMILES: *CCN(C(C)C(C)C)C(C)C1CC1]

In certain embodiments, A is

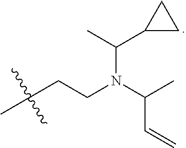 [SMILES: *CCN(C(C)C=C)C(C)C1CC1]

In certain embodiments, A is

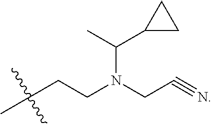 [SMILES: *CCN(CC#N)C(C)C1CC1]

In certain embodiments, A is

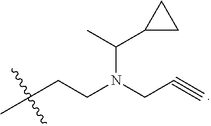 [SMILES: *CCN(CC#C)C(C)C1CC1]

In certain embodiments, A is

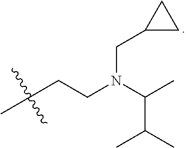 [SMILES: *CCN(CC1CC1)C(C)C(C)C]

In certain embodiments, A is

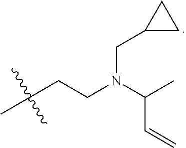 [SMILES: *CCN(CC1CC1)C(C)C=C]

In certain embodiments, A is

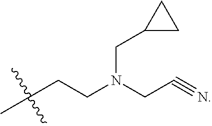 [SMILES: *CCN(CC#N)CC1CC1]

In certain embodiments, A is

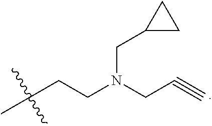 [SMILES: *CCN(CC#C)CC1CC1]

In certain embodiments, A is

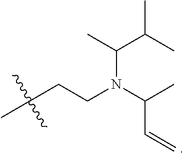 [SMILES: *CCN(C(C)C=C)C(C)C(C)C]

In certain embodiments, A is

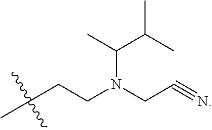 [SMILES: *CCN(CC#N)C(C)C(C)C]

In certain embodiments, A is

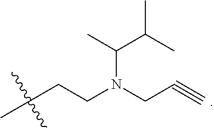 [SMILES: *CCN(CC#C)C(C)C(C)C]

In certain embodiments, A is

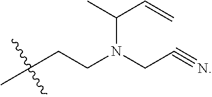 [SMILES: *CCN(CC#N)C(C)C=C]

In certain embodiments, A is

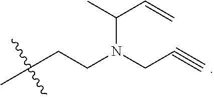 [SMILES: *CCN(CC#C)C(C)C=C]

In certain embodiments, A is

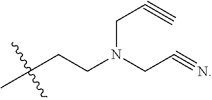 [SMILES: *CCN(CC#C)CC#N]

In certain embodiments, A is

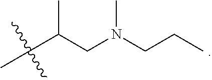 [SMILES: *C(C)CN(C)CCC]

In certain embodiments, A is

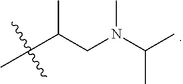 [SMILES: *C(C)CN(C)C(C)C]

In certain embodiments, A is

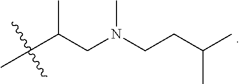 [SMILES: *C(C)CN(C)CCC(C)C]

In certain embodiments, A is

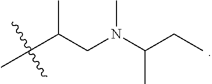 [SMILES: *C(C)CN(C)C(C)CC]

In certain embodiments, A is

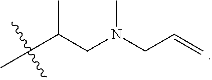 [SMILES: *C(C)CN(C)CC=C]

In certain embodiments, A is

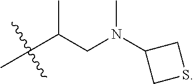 [SMILES: *C(C)CN(C)C1CSC1]

In certain embodiments, A is

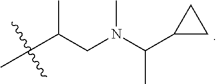 [SMILES: *C(C)CN(C)C(C)C1CC1]

In certain embodiments, A is

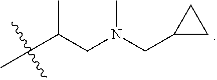 [SMILES: *C(C)CN(C)CC1CC1]

In certain embodiments, A is

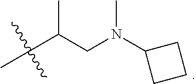 [SMILES: *C(C)CN(C)C1CCC1]

In certain embodiments, A is

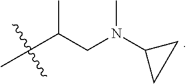 [SMILES: *C(C)CN(C)C1CC1]

In certain embodiments, A is

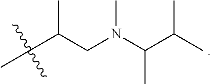 [SMILES: *C(C)CN(C)C(C)C(C)C]

In certain embodiments, A is

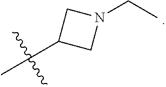 [SMILES: *C1CN(CC)C1]

In certain embodiments, A is

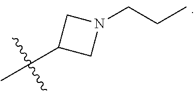 [SMILES: *C1CN(CCC)C1]

In certain embodiments, A is

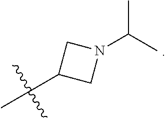 [SMILES: *C1CN(C(C)C)C1]

In certain embodiments, A is

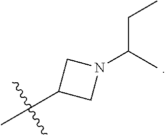 [SMILES: *C1CN(C(C)CC)C1]

In certain embodiments, A is

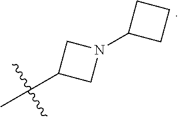 [SMILES: *C1CN(C2CCC2)C1]

In certain embodiments, A is

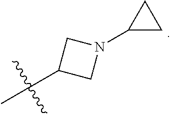 [SMILES: *C1CN(C2CC2)C1]

In certain embodiments, A is

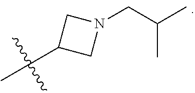 [SMILES: *C1CN(CC(C)C)C1]

In certain embodiments, A is

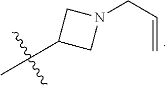 [SMILES: *C1CN(CC=C)C1]

In certain embodiments, A is

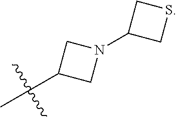 [SMILES: *C1CN(C2CSC2)C1]

In certain embodiments, A is

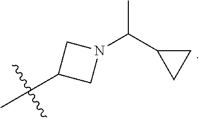 [SMILES: *C1CN(C(C)C2CC2)C1]

In certain embodiments, A is

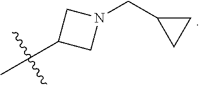 [SMILES: *C1CN(CC2CC2)C1]

In certain embodiments, A is

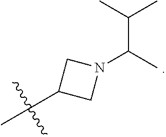 [SMILES: *C1CN(C(C)C(C)C)C1]

In certain embodiments, A is

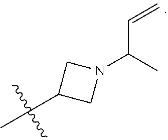 [SMILES: *C1CN(C(C)C=C)C1]

In certain embodiments, A is

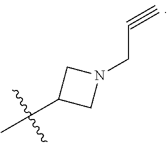 [SMILES: *C1CN(CC#C)C1]

In certain embodiments, A is

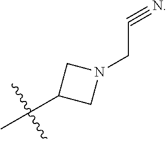 [SMILES: *C1CN(CC#N)C1]

In certain embodiments, A is

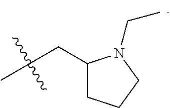 [SMILES: *CC1CCCN1CC]

In certain embodiments, A is

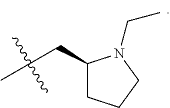 [SMILES: *C[C@@H]1CCCN1CC]

In certain embodiments, A is

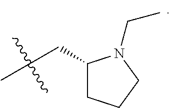 [SMILES: *C[C@H]1CCCN1CC]

In certain embodiments, A is

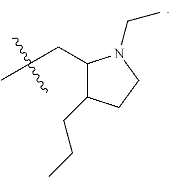 [SMILES: *CC1C(CCC)CCN1CC]

In certain embodiments, A is

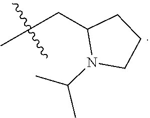 [SMILES: *CC1CCCN1C(C)C]

In certain embodiments, A is

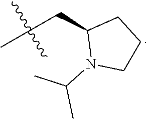 [SMILES: *C[C@H]1CCCN1C(C)C]

In certain embodiments, A is

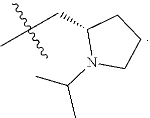 [SMILES: *C[C@@H]1CCCN1C(C)C]

In certain embodiments, A is

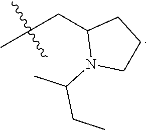 [SMILES: *CC1CCCN1C(C)CC]

In certain embodiments, A is

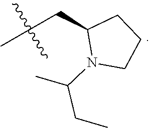 [SMILES: *C[C@H]1CCCN1C(C)CC]

In certain embodiments, A is

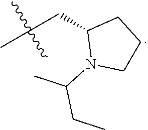 [SMILES: *C[C@@H]1CCCN1C(C)CC]

In certain embodiments, A is

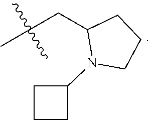 [SMILES: *CC1CCCN1C1CCC1]

In certain embodiments, A is

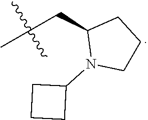 [SMILES: *C[C@H]1CCCN1C1CCC1]

In certain embodiments, A is

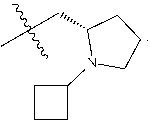 [SMILES: *C[C@@H]1CCCN1C1CCC1]

In certain embodiments, A is

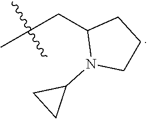 [SMILES: *CC1CCCN1C1CC1]

In certain embodiments, A is

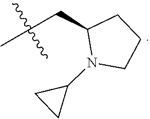 [SMILES: *C[C@H]1CCCN1C1CC1]

In certain embodiments, A is

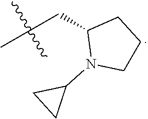 [SMILES: *C[C@@H]1CCCN1C1CC1]

In certain embodiments, A is

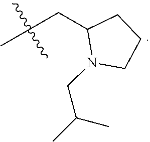 [SMILES: *CC1CCCN1CC(C)C]

In certain embodiments, A is

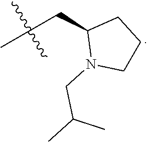 [SMILES: *C[C@H]1CCCN1CC(C)C]

In certain embodiments, A is

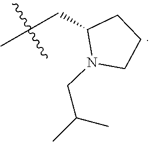 [SMILES: *C[C@@H]1CCCN1CC(C)C]

In certain embodiments, A is

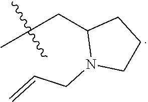 [SMILES: *CC1CCCN1CC=C]

In certain embodiments, A is

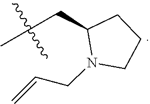 [SMILES: *C[C@H]1CCCN1CC=C]

In certain embodiments, A is

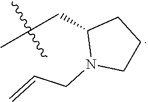 [SMILES: *C[C@@H]1CCCN1CC=C]

In certain embodiments, A is

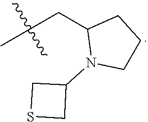 [SMILES: *CC1CCCN1C1CSC1]

In certain embodiments, A is

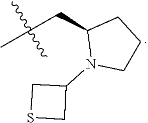 [SMILES: *C[C@H]1CCCN1C1CSC1]

In certain embodiments, A is

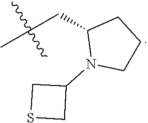 [SMILES: *C[C@@H]1CCCN1C1CSC1]

In certain embodiments, A is

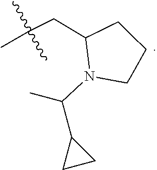 [SMILES: *CC1CCCCN1C(C)C1CC1]

In certain embodiments, A is

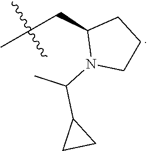 [SMILES: *C[C@H]1CCCN1C(C)C1CC1]

In certain embodiments, A is

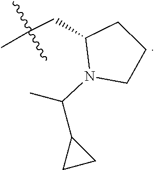 [SMILES: *C[C@@H]1CCCCN1C(C)C1CC1]

In certain embodiments, A is

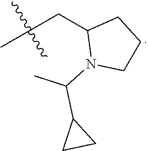 [SMILES: *CC1CCCN1C(C)C1CC1]

In certain embodiments, A is

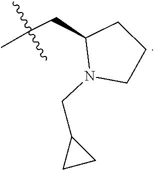 [SMILES: *C[C@H]1CCCCN1CC1CC1]

In certain embodiments, A is

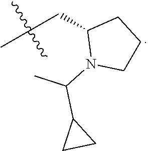 [SMILES: *C[C@@H]1CCCN1C(C)C1CC1]

In certain embodiments, A is

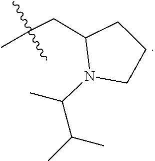 [SMILES: *CC1CCCCN1C(C)C(C)C]

In certain embodiments, A is

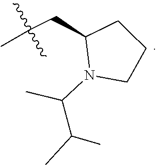 [SMILES: *C[C@H]1CCCCN1C(C)C(C)C]

In certain embodiments, A is

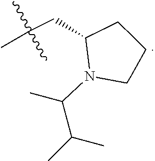 [SMILES: *C[C@@H]1CCCCN1C(C)C(C)C]

In certain embodiments, A is

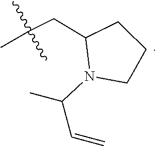 [SMILES: *CC1CCCCN1C(C)C=C]

In certain embodiments, A is

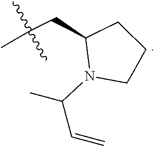 [SMILES: *C[C@H]1CCCCN1C(C)C=C]

In certain embodiments, A is

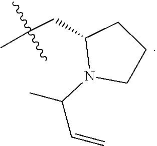 [SMILES: *C[C@@H]1CCCCN1C(C)C=C]

In certain embodiments A is

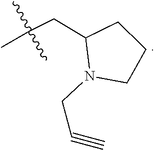 [SMILES: *CC1CCCCN1CC#C]

In certain embodiments A is

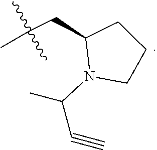 [SMILES: *C[C@H]1CCCCN1C(C)C#C]

In certain embodiments, A is

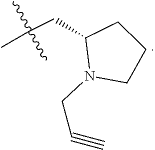 [SMILES: *C[C@@H]1CCCCN1CC#C]

In certain embodiments, A is

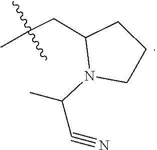 [SMILES: *CC1CCCCN1C(C)C#N]

In certain embodiments, A is

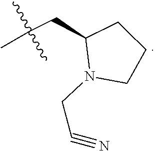 [SMILES: *C[C@H]1CCCCN1CC#N]

In certain embodiments, A is

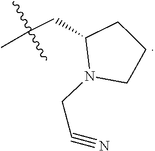 [SMILES: *C[C@@H]1CCCCN1CC#N]

In certain embodiments, A is

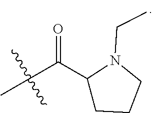 [SMILES: *C(=O)C1CCCN1CC]

In certain embodiments, A is

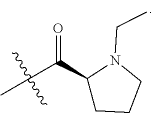 [SMILES: *C(=O)[C@@H]1CCCN1CC]

In certain embodiments, A is

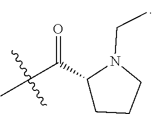 [SMILES: *C(=O)[C@H]1CCCN1CC]

In certain embodiments, A is

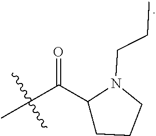 [SMILES: *C(=O)C1CCCN1CCC]

In certain embodiments, A is

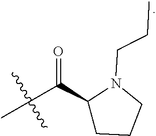 [SMILES: *C(=O)[C@@H]1CCCN1CCC]

In certain embodiments, A is

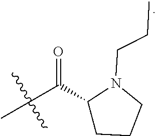 [SMILES: *C(=O)[C@H]1CCCN1CCC]

In certain embodiments, A is

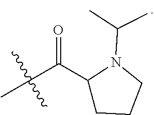 [SMILES: *C(=O)C1CCCN1C(C)C]

In certain embodiments, A is

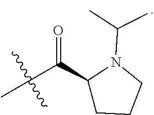 [SMILES: *C(=O)[C@@H]1CCCN1C(C)C]

In certain embodiments, A is

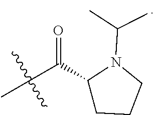 [SMILES: *C(=O)[C@H]1CCCN1C(C)C]

In certain embodiments, A is

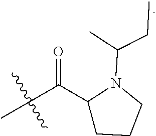 [SMILES: *C(=O)C1CCCN1C(C)CC]

In certain embodiments, A is

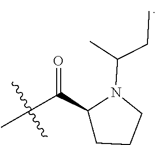 [SMILES: *C(=O)[C@@H]1CCCN1C(C)CC]

In certain embodiments, A is

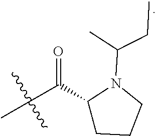 [SMILES: *C(=O)[C@H]1CCCN1C(C)CC]

In certain embodiments, A is

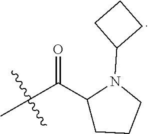 [SMILES: *C(=O)C1CCCN1C1CCC1]

In certain embodiments, A is

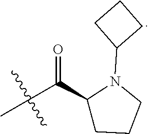 [SMILES: *C(=O)[C@@H]1CCCN1C1CCC1]

In certain embodiments, A is

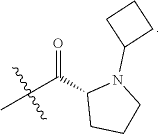 [SMILES: *C(=O)[C@H]1CCCN1C1CCC1]

In certain embodiments, A is

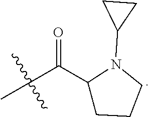 [SMILES: *C(=O)C1CCCN1C1CC1]

In certain embodiments, A is

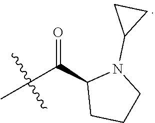 [SMILES: *C(=O)[C@@H]1CCCN1C1CC1]

In certain embodiments, A is

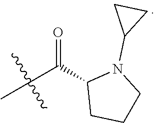 [SMILES: *C(=O)[C@H]1CCCN1C1CC1]

In certain embodiments, A is

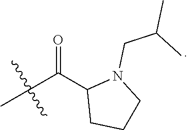 [SMILES: *C(=O)C1CCCN1CC(C)C]

In certain embodiments, A is

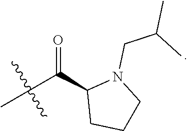 [SMILES: *C(=O)[C@@H]1CCCN1CC(C)C]

In certain embodiments, A is

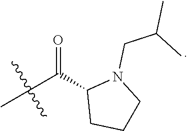 [SMILES: *C(=O)[C@H]1CCCN1CC(C)C]

In certain embodiments, A is

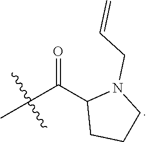 [SMILES: *C(=O)C1CCCN1CC=C]

In certain embodiments, A is

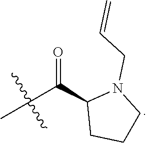 [SMILES: *C(=O)[C@@H]1CCCN1CC=C]

In certain embodiments A is

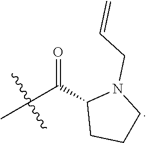 [SMILES: *C(=O)[C@H]1CCCN1CC=C]

In certain embodiments, A is

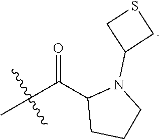 [SMILES: *C(=O)C1CCCN1C1CSC1]

In certain embodiments, A is

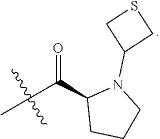 [SMILES: *C(=O)[C@@H]1CCCN1C1CSC1]

In certain embodiments, A is

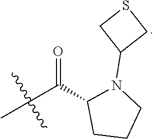 [SMILES: *C(=O)[C@H]1CCCN1C1CSC1]

In certain embodiments, A is

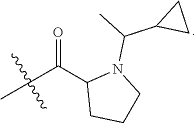 [SMILES: *C(=O)C1CCCN1C(C)C1CC1]

In certain embodiments, A is

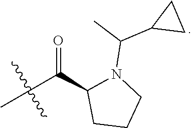 [SMILES: *C(=O)[C@@H]1CCCN1C(C)C1CC1]

In certain embodiments, A is

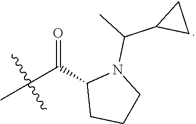 [SMILES: *C(=O)[C@H]1CCCN1C(C)C1CC1]

In certain embodiments, A is

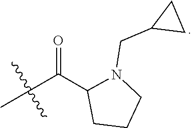 [SMILES: *C(=O)C1CCCN1CC1CC1]

In certain embodiments, A is

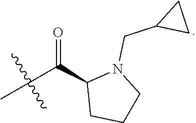 [SMILES: *C(=O)[C@@H]1CCCN1CC1CC1]

In certain embodiments, A is

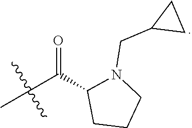 [SMILES: *C(=O)[C@H]1CCCN1CC1CC1]

In certain embodiments, A is

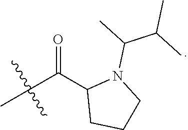 [SMILES: *C(=O)C1CCCN1C(C)C(C)C]

In certain embodiments, A is

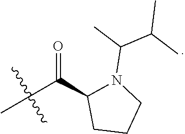 [SMILES: *C(=O)[C@@H]1CCCN1C(C)C(C)C]

In certain embodiments, A is

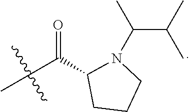 [SMILES: *C(=O)[C@H]1CCCN1C(C)C(C)C]

In certain embodiments, A is

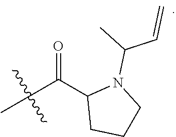 [SMILES: *C(=O)C1CCCN1C(C)C=C]

In certain embodiments, A is

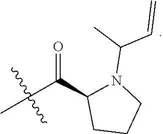 [SMILES: *C(=O)[C@@H]1CCCN1C(C)C=C]

In certain embodiments, A is

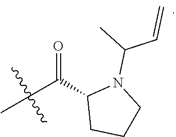 [SMILES: *C(=O)[C@H]1CCCN1C(C)C=C]

In certain embodiments, A is

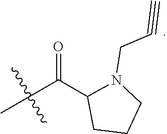 [SMILES: *C(=O)C1CCCN1CC#C]

In certain embodiments, A is

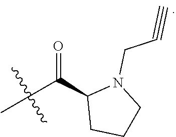 [SMILES: *C(=O)[C@@H]1CCCN1CC#C]

In certain embodiments A is

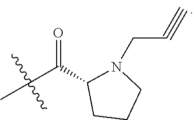 [SMILES: *C(=O)[C@H]1CCCN1CC#C]

In certain embodiments, A is

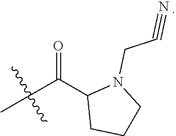 [SMILES: *C(=O)C1CCCN1CC#N]

In certain embodiments, A is

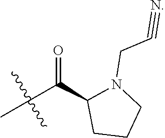 [SMILES: *C(=O)[C@@H]1CCCN1CC#N]

In certain embodiments, A is

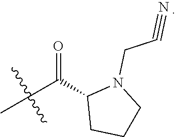 [SMILES: *C(=O)[C@H]1CCCN1CC#N]

In certain embodiments, A is

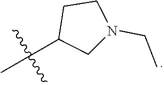 [SMILES: *C1CCN(CC)C1]

In certain embodiments, A is

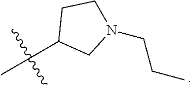 [SMILES: *C1CCN(CCC)C1]

In certain embodiments, A is

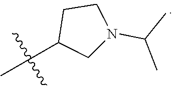 [SMILES: *C1CCN(C(C)C)C1]

In certain embodiments, A is

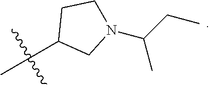 [SMILES: *C1CCN(C(C)CC)C1]

In certain embodiments, A is

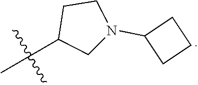 [SMILES: *C1CCN(C2CCC2)C1]

In certain embodiments, A is

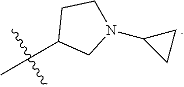 [SMILES: *C1CCN(C2CC2)C1]

In certain embodiments, A is

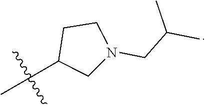 [SMILES: *C1CCN(CC(C)C)C1]

In certain embodiments, A is

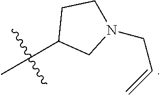 [SMILES: *C1CCN(CC=C)C1]

In certain embodiments, A is

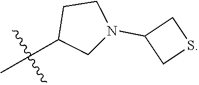 [SMILES: *C1CCN(C2CSC2)C1]

In certain embodiments, A is

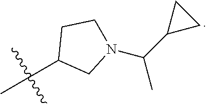 [SMILES: *C1CCN(C(C)C2CC2)C1]

In certain embodiments, A is

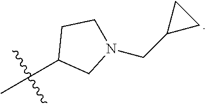 [SMILES: *C1CCN(CC2CC2)C1]

In certain embodiments, A is

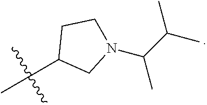 [SMILES: *C1CCN(C(C)C(C)C)C1]

In certain embodiments, A is

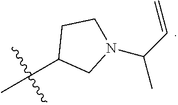 [SMILES: *C1CCN(C(C)C=C)C1]

In certain embodiments, A is

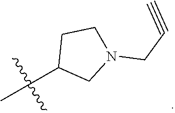 [SMILES: *C1CCN(CC#C)C1]

In certain embodiments, A is

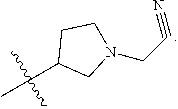 [SMILES: *C1CCN(CC#N)C1]

In certain embodiments, A is

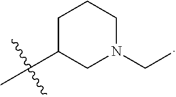

In certain embodiments, A is

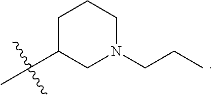 [SMILES: *C1CCCN(CCC)C1]

In certain embodiments, A is

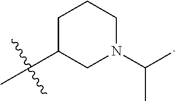

In certain embodiments A is

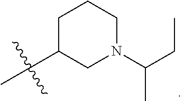 [SMILES: *C1CCCN(C(C)CC)C1]

In certain embodiments, A is

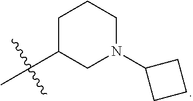 [SMILES: *C1CCCN(C2CCC2)C1]

In certain embodiments, A is

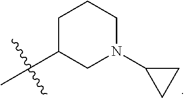 [SMILES: *C1CCCN(C2CC2)C1]

In certain embodiments, A is

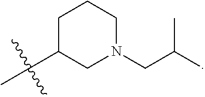

In certain embodiments, A is

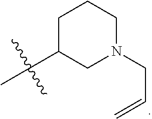 [SMILES: *C1CCCN(CC=C)C1]

In certain embodiments, A is

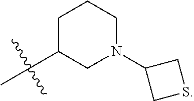 [SMILES: *C1CCCN(C2CSC2)C1]

In certain embodiments, A is

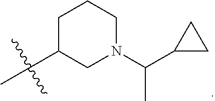 [SMILES: *C1CCCN(C(C)C2CC2)C1]

In certain embodiments, A is

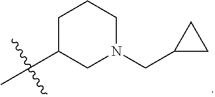 [SMILES: *C1CCCN(CC2CC2)C1]

In certain embodiments, A is

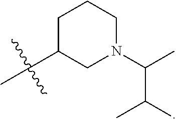

In certain embodiments, A is

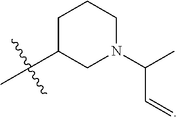

In certain embodiments, A is

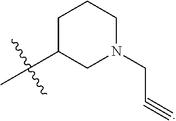

In certain embodiments, A is

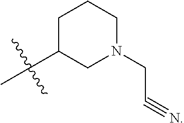 [SMILES: *C1CCCN(CC#N)C1]

In certain embodiments, the compound is selected from the group consisting of:
N-ethyl-N-(2-(5-fluoro-1H-indol-3-yl)ethyl)propan-2-amine;
N-(2-(5-fluoro-1H-indol-3-yl)ethyl)-N-methylpropan-2-amine;
N-(2-(5-fluoro-1H-indol-3-yl)ethyl)-N-methylbutan-2-amine;
N-(2-(5-fluoro-1H-indol-3-yl)ethyl)-N-isopropylpropan-1-amine;
N-(2-(5,6-difluoro-1H-indol-3-yl)ethyl)-N-ethylpropan-1-amine;
N-(2-(5-fluoro-1H-indol-3-yl)ethyl)-N-methylprop-2-en-1-amine;
N-(2-(5-fluoro-1H-indol-3-yl)ethyl)-N-isopropylprop-2-en-1-amine;
N-(2-(5-fluoro-1H-indol-3-yl)ethyl)-N-methylpropan-1-amine;
N-allyl-N-(2-(5-fluoro-1H-indol-3-yl)ethyl)cyclobutanamine;
N-allyl-N-(2-(5-fluoro-1H-indol-3-yl)ethyl)butan-2-amine;
2-((2-(5-fluoro-1H-indol-3-yl)ethyl)(isopropyl)amino)acetonitrile;
5,6-difluoro-3-(1-methylpyrrolidin-3-yl)-1H-indole;
N-(2-(5-fluoro-1H-indol-3-yl)ethyl)-N-methylthietan-3-amine;
N-(2-(4,5-difluoro-1H-indol-3-yl)ethyl)-N-ethylpropan-1-amine;
N-(2-(5-fluoro-1H-indol-3-yl)ethyl)-N-propylbutan-2-amine;
7-fluoro-3-(1-methylpyrrolidin-3-yl)-1H-indole;
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
(S)-5-fluoro-3-((1-methylpyrrolidin-2-yl)methyl)-1H-indole;
(R)-5-fluoro-3-((1-methylpyrrolidin-2-yl)methyl)-1H-indole;
5-fluoro-3-(1-methylazetidin-3-yl)-1H-indole;
N-ethyl-N-(2-(5-fluoro-1H-indol-3-yl)ethyl)butan-2-amine;
N-ethyl-N-(2-(5-fluoro-1H-indol-3-yl)ethyl)cyclobutanamine;
N-(2-(5-fluoro-1H-indol-3-yl)ethyl)-N-isopropylbutan-2-amine;
N-(2-(5-fluoro-1H-indol-3-yl)ethyl)-N-propylcyclobutanamine;
N-(2-(5-fluoro-1H-indol-3-yl)ethyl)-N-isopropylprop-2-yn-1-amine;
N-(2-(5-fluoro-1H-indol-3-yl)ethyl)-N-propylprop-2-en-1-amine;
N-ethyl-N-(2-(5-fluoro-1H-indol-3-yl)ethyl)prop-2-en-1-amine;
N-(2-(6-chloro-5-fluoro-1H-indol-3-yl)ethyl)-N-ethylpropan-1-amine;
1-cyclopropyl-N-(2-(5-fluoro-1H-indol-3-yl)ethyl)-N-methylethan-1-amine;
N-(2-(5-fluoro-1H-indol-3-yl)ethyl)-N,2-dimethylpropan-1-amine;
N-(2-(5-fluoro-1H-indol-3-yl)ethyl)-N-isopropylcyclobutanamine;
N-ethyl-N-(2-(7-fluoro-5-methoxy-1H-indol-3-yl)ethyl)propan-1-amine;
N-(2-(4-fluoro-1H-indol-3-yl)ethyl)-N-methylpropan-1-amine;
N-(2-(4-fluoro-1H-indol-3-yl)ethyl)-N-methylpropan-2-amine;
N-ethyl-N-(2-(4-fluoro-1H-indol-3-yl)ethyl)propan-2-amine;
N-(2-(4-fluoro-1H-indol-3-yl)ethyl)-N,2-dimethylpropan-1-amine;
N-(2-(4-fluoro-1H-indol-3-yl)ethyl)-N-methylcyclobutanamine;
N-(2-(4-fluoro-1H-indol-3-yl)ethyl)-N-methylprop-2-en-1-amine;
N-(2-(4-fluoro-1H-indol-3-yl)ethyl)-N-isopropyl-2-methylpropan-1-amine;
N-(2-(5-fluoro-1H-indol-3-yl)ethyl)-N-methylcyclobutanamine;
N-(2-(5-fluoro-1H-indol-3-yl)ethyl)-N-isopropyl-2-methylpropan-1-amine;
N-(2-(7-fluoro-1H-indol-3-yl)ethyl)-N-methylpropan-1-amine;
N-(2-(7-fluoro-1H-indol-3-yl)ethyl)-N-methylpropan-2-amine;
N-ethyl-N-(2-(7-fluoro-1H-indol-3-yl)ethyl)propan-2-amine;
N-(2-(7-fluoro-1H-indol-3-yl)ethyl)-N,2-dimethylpropan-1-amine;
N-(2-(7-fluoro-1H-indol-3-yl)ethyl)-N-methylcyclobutanamine;
N-(2-(7-fluoro-1H-indol-3-yl)ethyl)-N-methylprop-2-en-1-amine;
N-(2-(7-fluoro-1H-indol-3-yl)ethyl)-N-isopropyl-2-methylpropan-1-amine;
2-(5,6-difluoro-1H-indol-3-yl)-N-ethyl-N-methylethan-1-amine;
N-(2-(5,6-difluoro-1H-indol-3-yl)ethyl)-N-methylpropan-1-amine;
N-(2-(5,6-difluoro-1H-indol-3-yl)ethyl)-N-methylpropan-2-amine;
N-(2-(5,6-difluoro-1H-indol-3-yl)ethyl)-N-ethylpropan-2-amine;
N-(2-(5,6-difluoro-1H-indol-3-yl)ethyl)-N,2-dimethylpropan-1-amine;
N-(2-(5,6-difluoro-1H-indol-3-yl)ethyl)-N-methylcyclobutanamine;
N-(2-(5,6-difluoro-1H-indol-3-yl)ethyl)-N-methylprop-2-en-1-amine;
N-(2-(5,6-difluoro-1H-indol-3-yl)ethyl)-N-isopropyl-2-methylpropan-1-amine;
2-(5,7-difluoro-1H-indol-3-yl)-N-ethyl-N-methylethan-1-amine;
N-(2-(5,7-difluoro-1H-indol-3-yl)ethyl)-N-methylpropan-1-amine;
N-(2-(5,7-difluoro-1H-indol-3-yl)ethyl)-N-methylpropan-2-amine;
N-(2-(5,7-difluoro-1H-indol-3-yl)ethyl)-N-ethylpropan-1-amine;
N-(2-(5,7-difluoro-1H-indol-3-yl)ethyl)-N-ethylpropan-2-amine;
N-(2-(5,7-difluoro-1H-indol-3-yl)ethyl)-N,2-dimethylpropan-1-amine;
N-(2-(5,7-difluoro-1H-indol-3-yl)ethyl)-N-methylcyclobutanamine;
N-(2-(5,7-difluoro-1H-indol-3-yl)ethyl)-N-methylprop-2-en-1-amine;
N-(2-(5,7-difluoro-1H-indol-3-yl)ethyl)-N-isopropyl-2-methylpropan-1-amine;
2-(6,7-difluoro-1H-indol-3-yl)-N-ethyl-N-methylethan-1-amine;
N-(2-(6,7-difluoro-1H-indol-3-yl)ethyl)-N-methylpropan-1-amine;
N-(2-(6,7-difluoro-1H-indol-3-yl)ethyl)-N-methylpropan-2-amine;
N-(2-(6,7-difluoro-1H-indol-3-yl)ethyl)-N-ethylpropan-1-amine;
N-(2-(6,7-difluoro-1H-indol-3-yl)ethyl)-N-ethylpropan-2-amine;
N-(2-(6,7-difluoro-1H-indol-3-yl)ethyl)-N,2-dimethylpropan-1-amine;
N-(2-(6,7-difluoro-1H-indol-3-yl)ethyl)-N-methylcyclobutanamine;
N-(2-(6,7-difluoro-1H-indol-3-yl)ethyl)-N-methylprop-2-en-1-amine;
N-(2-(6,7-difluoro-1H-indol-3-yl)ethyl)-N-isopropyl-2-methylpropan-1-amine;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
4-fluoro-3-(1-methylpyrrolidin-3-yl)-1H-indole;
6-fluoro-3-(1-methylpyrrolidin-3-yl)-1H-indole;
4,5-difluoro-3-(1-methylpyrrolidin-3-yl)-1H-indole;
4,6-difluoro-3-(1-methylpyrrolidin-3-yl)-1H-indole;
5,7-difluoro-3-(1-methylpyrrolidin-3-yl)-1H-indole;
6,7-difluoro-3-(1-methylpyrrolidin-3-yl)-1H-indole;
4-fluoro-3-(1-methylpiperidin-3-yl)-1H-indole;
5-fluoro-3-(1-methylpiperidin-3-yl)-1H-indole;
6-fluoro-3-(1-methylpiperidin-3-yl)-1H-indole;
7-fluoro-3-(1-methylpiperidin-3-yl)-1H-indole;
4,5-difluoro-3-(1-methylpiperidin-3-yl)-1H-indole;
4,6-difluoro-3-(1-methylpiperidin-3-yl)-1H-indole;
5,6-difluoro-3-(1-methylpiperidin-3-yl)-1H-indole;
5,7-difluoro-3-(1-methylpiperidin-3-yl)-1H-indole;
6,7-difluoro-3-(1-methylpiperidin-3-yl)-1H-indole;
4-fluoro-3-(methylprolyl)-1H-indole;
6-fluoro-3-(methylprolyl)-1H-indole;
7-fluoro-3-(methylprolyl)-1H-indole;
4,5-difluoro-3-(methylprolyl)-1H-indole;
4,6-difluoro-3-(methylprolyl)-1H-indole;
5,6-difluoro-3-(methylprolyl)-1H-indole;
5,7-difluoro-3-(methylprolyl)-1H-indole;
6,7-difluoro-3-(methylprolyl)-1H-indole;
4-fluoro-3-((1-methylpyrrolidin-2-yl)methyl)-1H-indole;
6-fluoro-3-((1-methylpyrrolidin-2-yl)methyl)-1H-indole;
7-fluoro-3-((1-methylpyrrolidin-2-yl)methyl)-1H-indole;
4,5-difluoro-3-((1-methylpyrrolidin-2-yl)methyl)-1H-indole;
4,6-difluoro-3-((1-methylpyrrolidin-2-yl)methyl)-1H-indole;
5,6-difluoro-3-((1-methylpyrrolidin-2-yl)methyl)-1H-indole;
5,7-difluoro-3-((1-methylpyrrolidin-2-yl)methyl)-1H-indole;
6,7-difluoro-3-((1-methylpyrrolidin-2-yl)methyl)-1H-indole;
N-(sec-butyl)-N-(2-(5-fluoro-1H-indol-3-yl)ethyl)cyclobutanamine;
N-(cyclopropylmethyl)-N-(2-(5-fluoro-1H-indol-3-yl)ethyl)propan-2-amine;
N-(2-(5-fluoro-1H-indol-3-yl)ethyl)-N-methylcyclopropanamine;
N-(2-(5-fluoro-1H-indol-3-yl)ethyl)-N-propylcyclopropanamine;
N-(2-(7-fluoro-1H-indol-3-yl)ethyl)-N-propylcyclobutanamine;
N-(2-(7-fluoro-1H-indol-3-yl)ethyl)-N-propylbutan-2-amine;
N-ethyl-N-methyl-2-(5,6,7-trifluoro-1H-indol-3-yl)ethan-1-amine;
N-ethyl-N-(2-(5,6,7-trifluoro-1H-indol-3-yl)ethyl)propan-1-amine;
N-ethyl-N-(2-(7-fluoro-5-methyl-1H-indol-3-yl)ethyl)propan-1-amine;
N-ethyl-N-(2-(5-fluoro-1H-indol-3-yl)ethyl)cyclopropanamine;
N-(2-(5-fluoro-1H-indol-3-yl)ethyl)-N-isopropylcyclopropanamine;
N-ethyl-2-(5-fluoro-7-methyl-1H-indol-3-yl)-N-methylethan-1-amine;
N-(2-(5-fluoro-1H-indol-3-yl)ethyl)-N,3-dimethylbutan-2-amine;
N-(sec-butyl)-N-(2-(5-fluoro-1H-indol-3-yl)ethyl)cyclopropanamine;
N-cyclopropyl-N-(2-(5-fluoro-1H-indol-3-yl)ethyl)cyclobutanamine;
2-(7-bromo-5-fluoro-1H-indol-3-yl)-N-ethyl-N-methylethan-1-amine;
N-ethyl-2-(5-fluoro-6-methyl-1H-indol-3-yl)-N-methylethan-1-amine;
N-(2-(5-fluoro-1H-indol-3-yl)ethyl)-N-(prop-2-yn-1-yl)butan-2-amine;
2-(sec-butyl(2-(5-fluoro-1H-indol-3-yl)ethyl)amino)acetonitrile;
N-(2-(5-fluoro-1H-indol-3-yl)ethyl)-N-methylbut-3-en-2-amine;
2-(5-chloro-6-fluoro-1H-indol-3-yl)-N-ethyl-N-methylethan-1-amine;
N-(2-(7-fluoro-1H-indol-3-yl)ethyl)-N-propylprop-2-yn-1-amine;
2-((2-(7-fluoro-1H-indol-3-yl)ethyl)(propyl)amino)acetonitrile;
2-(7-chloro-5-fluoro-1H-indol-3-yl)-N-ethyl-N-methylethan-1-amine;
(R)-3-((1-ethylpyrrolidin-2-yl)methyl)-5-fluoro-1H-indole;
(R)-5-fluoro-3-(pyrrolidin-2-ylmethyl)-1H-indole;
(R)-5-fluoro-3-((1-propylpyrrolidin-2-yl)methyl)-1H-indole;
(R)-5,6-difluoro-3-((1-methylpyrrolidin-2-yl)methyl)-1H-indole;
(R)-7-fluoro-3-((1-methylpyrrolidin-2-yl)methyl)-1H-indole;
N-isopropyl-N-(2-(5,6,7-trifluoro-1H-indol-3-yl)ethyl)propan-1-amine;
N-methyl-N-(2-(5,6,7-trifluoro-1H-indol-3-yl)ethyl)butan-2-amine;
N-methyl-N-(2-(5,6,7-trifluoro-1H-indol-3-yl)ethyl)cyclobutanamine;
N-(2-(7-fluoro-5-methoxy-1H-indol-3-yl)ethyl)-N-isopropylpropan-1-amine;
N-(2-(7-fluoro-5-methoxy-1H-indol-3-yl)ethyl)-N-methylbutan-2-amine;
N-(2-(7-fluoro-5-methoxy-1H-indol-3-yl)ethyl)-N-methylbut-3-en-2-amine;
N-(2-(5,6-difluoro-1H-indol-3-yl)ethyl)-N-methylbut-3-en-2-amine;
N-(2-(6,7-difluoro-1H-indol-3-yl)ethyl)-N-methylbut-3-en-2-amine;
N-(2-(5,7-difluoro-1H-indol-3-yl)ethyl)-N-methylbut-3-en-2-amine;
N-methyl-N-(2-(5,6,7-trifluoro-1H-indol-3-yl)ethyl)but-3-en-2-amine;
3-(((2R)-1-(sec-butyl)pyrrolidin-2-yl)methyl)-5-fluoro-1H-indole;
(R)-5-fluoro-3-((1-isopropylpyrrolidin-2-yl)methyl)-1H-indole;
(R)-3-((1-cyclobutylpyrrolidin-2-yl)methyl)-5-fluoro-1H-indole;
(R)-3-((1-cyclopropylpyrrolidin-2-yl)methyl)-5-fluoro-1H-indole;
(R)-3-((1-allylpyrrolidin-2-yl)methyl)-5-fluoro-1H-indole;
3-(((2R)-1-(but-3-en-2-yl)pyrrolidin-2-yl)methyl)-5-fluoro-1H-indole;
(R)-5-fluoro-3-((1-(prop-2-yn-1-yl)pyrrolidin-2-yl)methyl)-1H-indole;
3-(((2R)-1-(sec-butyl)pyrrolidin-2-yl)methyl)-5,6,7-trifluoro-1H-indole;
(R)-5,6,7-trifluoro-3-((1-isopropylpyrrolidin-2-yl)methyl)-1H-indole;
(R)-3-((1-cyclobutylpyrrolidin-2-yl)methyl)-5,6,7-trifluoro-1H-indole;
(R)-3-((1-cyclopropylpyrrolidin-2-yl)methyl)-5,6,7-trifluoro-1H-indole;
(R)-3-((1-allylpyrrolidin-2-yl)methyl)-5,6,7-trifluoro-1H-indole;
3-(((2R)-1-(but-3-en-2-yl)pyrrolidin-2-yl)methyl)-5,6,7-trifluoro-1H-indole;
(R)-5,6,7-trifluoro-3-((1-(prop-2-yn-1-yl)pyrrolidin-2-yl)methyl)-1H-indole;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

In another aspect, the present disclosure provides a compound of formula (II), or a salt, prodrug, solvate, isotopologue, or stereoisomer thereof:

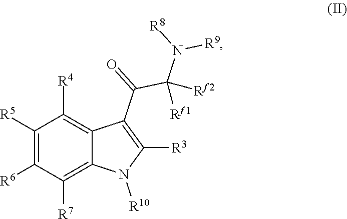

wherein:
   R³ is selected from the group consisting of H, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
   R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted C₁-C₈ heteroalkyl, optionally substituted benzyl, optionally substituted phenyl, and optionally substituted C₂-C₉ heteroaryl,
      wherein at least one of R⁴, R⁵, R⁶, and R⁷ is F;
   R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
   R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R^{f1} and R^{f2} are each independently selected from the group consisting of H and C₁-C₆ alkyl;
   each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
   each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

In certain embodiments, R³ is H. In certain embodiments, R³ is methyl. In certain embodiments, R³ is phenyl. In certain embodiments, R³ is benzyl.

In certain embodiments, R⁴ is optionally substituted C₁-C₈ haloalkyl. In certain embodiments, R⁴ is H. In certain embodiments, R⁴ is F. In certain embodiments, R⁴ is OH. In certain embodiments, R⁴ is OMe.

In certain embodiments, R⁵ is optionally substituted C₁-C₈ haloalkyl. In certain embodiments, R⁵ is H. In certain embodiments, R⁵ is F. In certain embodiments, R⁵ is OH. In certain embodiments, R⁵ is OMe.

In certain embodiments, R⁶ is optionally substituted C₁-C₈ haloalkyl. In certain embodiments, R⁶ is H. In certain embodiments, R⁶ is F. In certain embodiments, R⁶ is OH. In certain embodiments, R⁶ is OMe.

In certain embodiments, R⁷ is optionally substituted C₁-C₈ haloalkyl. In certain embodiments, R⁷ is H. In certain embodiments, R⁷ is F. In certain embodiments, R⁷ is OH. In certain embodiments, R⁷ is OMe.

In certain embodiments, R⁸ is methyl. In certain embodiments, R⁸ is ethyl. In certain embodiments, R⁸ is n-propyl. In certain embodiments, R⁸ is iso-propyl. In certain embodiments, R⁸ is sec-butyl. In certain embodiments, R⁸ is iso-butyl. In certain embodiments, R⁸ is n-butyl. In certain embodiments, R⁸ is cyclopropyl. In certain embodiments, R⁸ is cyclopropylmethyl. In certain embodiments, R⁸ is methylcyclopropyl. In certain embodiments, R⁸ is cyclopropylethyl.

In certain embodiments, R⁸ is cyclobutyl. In certain embodiments, R⁸ is allyl. In certain embodiments, R⁸ is methylallyl. In certain embodiments, R⁸ is 2-methylallyl. In certain embodiments, R⁸ is 3-methylallyl. In certain embodiments, R⁸ is allylmethyl. In certain embodiments, R⁸ is propargyl. In certain embodiments, R⁸ is cyanomethyl. In certain embodiments, R⁸ is 2-hydroxyethyl. In certain embodiments, R⁸ is and 2-methoxyethyl.

In certain embodiments, R⁹ is methyl. In certain embodiments, R⁹ is ethyl. In certain embodiments, R⁹ is n-propyl. In certain embodiments, R⁹ is iso-propyl. In certain embodiments, R⁹ is sec-butyl. In certain embodiments, R⁹ is iso-butyl. In certain embodiments, R⁹ is n-butyl. In certain embodiments, R⁹ is cyclopropyl. In certain embodiments, R⁹ is cyclopropylmethyl. In certain embodiments, R⁹ is methylcyclopropyl. In certain embodiments, R⁹ is cyclopropylethyl.

In certain embodiments, R⁹ is cyclobutyl. In certain embodiments, R⁹ is allyl. In certain embodiments, R⁹ is methylallyl. In certain embodiments, R⁹ is 2-methylallyl. In certain embodiments, R⁹ is 3-methylallyl. In certain embodiments, R⁹ is allylmethyl. In certain embodiments, R⁹ is propargyl. In certain embodiments, R⁹ is cyanomethyl. In certain embodiments, R⁹ is 2-hydroxyethyl. In certain embodiments, R⁹ is and 2-methoxyethyl.

In certain embodiments, R⁸ and R⁹ are different. In certain embodiments, R⁸ and R⁹ are identical. In certain embodiments, R⁸ and R⁹ are methyl. In certain embodiments, R⁸ is methyl and R⁹ is ethyl. In certain embodiments, R⁸ is ethyl and R⁹ is n-propyl.

In certain embodiments, R¹⁰ is H. In certain embodiments, R¹⁰ is methyl. In certain embodiments, R¹⁰ is phenyl. In certain embodiments, R¹⁰ is benzyl.

In certain embodiments, R¹ is H. In certain embodiments, R is H.

In certain embodiments, R⁴ is F, and each of R⁵, R⁶, and R⁷ is H. In certain embodiments, R⁵ is F, and each of R⁴, R⁶, and R⁷ is H. In certain embodiments, R⁶ is F, and each of R⁴, R⁵, and R⁷ is H. In certain embodiments, R⁷ is F, and each of R⁴, R⁵, and R⁶ is H. In certain embodiments, each of R⁴ and R⁵ is F, and each of R⁶ and R⁷ is H. In certain embodiments, each of R⁴ and R⁶ is F, and each of R⁵ and R⁷ is H. In certain embodiments, each of R⁴ and R⁷ is F, and each of R⁵ and R⁶ is H. In certain embodiments, each of R⁵ and R⁶ is F, and each of R⁴ and R⁷ is H. In certain embodiments, each of R⁵ and R⁷ is F, and each of R⁴ and R⁶ is H. In certain embodiment, each of R⁶ and R⁷ is F, and each of R⁴ and R⁵ is H.

In certain embodiments, the compound is selected from the group consisting of:
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
2-(dimethylamino)-1-(4-fluoro-1H-indol-3-yl)ethan-1-one;
2-(dimethylamino)-1-(5-fluoro-1H-indol-3-yl)ethan-1-one;
2-(dimethylamino)-1-(6-fluoro-1H-indol-3-yl)ethan-1-one;
2-(dimethylamino)-1-(7-fluoro-1H-indol-3-yl)ethan-1-one;
1-(4,5-difluoro-1H-indol-3-yl)-2-(dimethylamino)ethan-1-one;
1-(4,6-difluoro-1H-indol-3-yl)-2-(dimethylamino)ethan-1-one;
1-(5,6-difluoro-1H-indol-3-yl)-2-(dimethylamino)ethan-1-one;
1-(5,7-difluoro-1H-indol-3-yl)-2-(dimethylamino)ethan-1-one; and
1-(6,7-difluoro-1H-indol-3-yl)-2-(dimethylamino)ethan-1-one.

In another aspect, the present disclosure provides a compound of formula (III), or a salt, prodrug, solvate, isotopologue, or stereoisomer thereof:

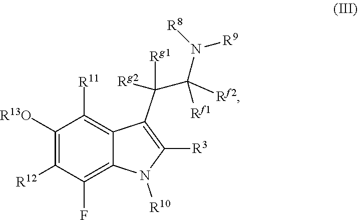

wherein:
   R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
   R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R¹¹ and R¹² are each independently selected from the group consisting of H, F, Cl, Br, I, OR^{A}, N(R^{A})(R^{B}), optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R¹³ is selected from the group consisting of optionally substituted C₁-C₆ alkyl, optionally substituted C₁-C₆ alkenyl, optionally substituted C₁-C₆ alkynyl, optionally substituted C₁-C₃ haloalkyl, optionally substituted benzyl, optionally substituted phenyl, and optionally substituted C₂-C₉ heteroaryl;
   R^{f1} and R^{f2} are each independently selected from the group consisting of H and C₁-C₆ alkyl;
   R^{g1} and R^{g2} are each independently selected from the group consisting of H and C₁-C₆ alkyl;
   each occurrence of R^{A} is independently selected from the group consisting of H, C₁-C₆ alkyl, C₂-C₆ alkenyl, C₁-C₆ haloalkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂₀(C₆-C₁₀ aryl), —S(═O)₂₀(C₁-C₆ alkyl), —S(═O)₂₀(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
   each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

In certain embodiments, R³ is H. In certain embodiments, R³ is methyl. In certain embodiments, R³ is phenyl. In certain embodiments, R³ is benzyl.

In certain embodiments, R⁸ is methyl. In certain embodiments, R⁸ is ethyl. In certain embodiments, R⁸ is n-propyl. In certain embodiments, R⁸ is iso-propyl. In certain embodiments, R⁸ is sec-butyl. In certain embodiments, R⁸ is iso-butyl. In certain embodiments, R⁸ is n-butyl. In certain embodiments, R⁸ is cyclopropyl. In certain embodiments, R⁸ is cyclopropylmethyl. In certain embodiments, R⁸ is methylcyclopropyl. In certain embodiments, R⁸ is cyclopropylethyl. In certain embodiments, R⁸ is cyclobutyl. In certain embodiments, R⁸ is allyl. In certain embodiments, R⁸ is methylallyl. In certain embodiments, R⁸ is 2-methylallyl. In certain embodiments, R⁸ is 3-methylallyl. In certain embodiments, R⁸ is allylmethyl. In certain embodiments, R⁸ is propargyl. In certain embodiments, R⁸ is cyanomethyl. In certain embodiments, R⁸ is 2-hydroxyethyl. In certain embodiments, R⁸ is and 2-methoxyethyl.

In certain embodiments, R⁹ is methyl. In certain embodiments, R⁹ is ethyl. In certain embodiments, R⁹ is n-propyl. In certain embodiments, R⁹ is iso-propyl. In certain embodiments, R⁹ is sec-butyl. In certain embodiments, R⁹ is iso-butyl. In certain embodiments, R⁹ is n-butyl. In certain embodiments, R⁹ is cyclopropyl. In certain embodiments, R⁹ is cyclopropylmethyl. In certain embodiments, R⁹ is methylcyclopropyl. In certain embodiments, R⁹ is cyclopropylethyl.

In certain embodiments, R⁹ is cyclobutyl. In certain embodiments, R⁹ is allyl. In certain embodiments, R⁹ is methylallyl. In certain embodiments, R⁹ is 2-methylallyl. In certain embodiments, R⁹ is 3-methylallyl. In certain embodiments, R⁹ is allylmethyl. In certain embodiments, R⁹ is propargyl. In certain embodiments, R⁹ is cyanomethyl. In certain embodiments, R⁹ is 2-hydroxyethyl. In certain embodiments, R⁹ is and 2-methoxyethyl.

In certain embodiments, R¹⁰ is H. In certain embodiments, R¹⁰ is methyl. In certain embodiments, R¹⁰ is phenyl. In certain embodiments, R¹⁰ is benzyl.

In certain embodiments, R¹¹ is optionally substituted C₁-C₆ haloalkyl. In certain embodiments, R¹¹ is H. In certain embodiments, R¹¹ is F. In certain embodiments, R¹¹ is OH. In certain embodiments, R¹¹ is OMe.

In certain embodiments, R¹² is optionally substituted C₁-C₆ haloalkyl. In certain embodiments, R¹² is H. In certain embodiments, R¹² is F. In certain embodiments, R¹² is OH. In certain embodiments, R¹² is OMe.

In certain embodiments, R¹³ is H. In certain embodiments, R¹³ is methyl. In certain embodiments, R¹³ is phenyl, optionally substituted with at least one halogen. In certain embodiments, R¹³ is benzyl. In certain embodiments, R¹³ is CF₃. In certain embodiments, R¹³ is CHF₂.

In certain embodiments, R¹ is H. In certain embodiments, R is H. In certain embodiments, R^{g1} is H. In certain embodiments, R^{g2} is H.

In certain embodiments, the compound is selected from the group consisting of.
N-ethyl-N-(2-(7-fluoro-5-methoxy-1H-indol-3-yl)ethyl)propan-1-amine;
2-(7-fluoro-5-methoxy-1H-indol-3-yl)-N,N-dimethylethan-1-amine;
N,N-diethyl-2-(7-fluoro-5-methoxy-1H-indol-3-yl)ethan-1-amine;
N-(2-(7-fluoro-5-methoxy-1H-indol-3-yl)ethyl)-N-propylpropan-1-amine;
3-(2-(dimethylamino)ethyl)-7-fluoro-1H-indol-5-ol;
3-(2-(diethylamino)ethyl)-7-fluoro-1H-indol-5-ol;
3-(2-(dipropylamino)ethyl)-7-fluoro-1H-indol-5-ol;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

In certain embodiments, each occurrence of alkyl, heteroalkyl, alkenyl, alkynyl, cycloalkyl, and heterocyclyl is independently optionally substituted with at least one substituent selected from the group consisting of C₁-C₆ alkyl, C₃-C₈ cycloalkyl, halo, cyano, OR^{A}, optionally substituted benzyl, optionally substituted phenyl, optionally substituted C₂-C₉ heteroaryl, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}).

In certain embodiments, each occurrence of optionally substituted alkyl, optionally substituted heteroalkyl, optionally substituted alkenyl, optionally substituted alkynyl, optionally substituted cycloalkyl, and optionally substituted heterocyclyl is independently optionally substituted with at least one substituent selected from the group consisting of C₁-C₆ alkyl, C₃-C₈ cycloalkyl, halo, cyano, OR^{A}, optionally substituted benzyl, optionally substituted phenyl, optionally substituted C₂-C₉ heteroaryl, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}) In certain embodiments, each occurrence of benzyl, phenyl, and heteroaryl is independently optionally substituted with at least one substituent selected from the group consisting of C₁-C₆ alkyl, C₃-C₈ cycloalkyl, halo, cyano, OR^{A}, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A} C(═O)N(R^{A})(R^{B}), and N(R^{A})(R^{B}).

In certain embodiments, each occurrence of optionally substituted benzyl, optionally substituted phenyl, and optionally substituted heteroaryl is independently optionally substituted with at least one substituent selected from the group consisting of C₁-C₆ alkyl, C₃-C₈ cycloalkyl, halo, cyano, OR^{A}, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}).

In another aspect, the present disclosure provides a compound selected from the group consisting of:
N-(2-(5-fluoro-1H-indol-3-yl)ethyl)prop-2-en-1-amine;
2-(5-fluoro-1H-indol-3-yl)-N,N-dimethylpropan-1-amine;
N-(2-(5-fluoro-1H-indol-3-yl)ethyl)-N-propylpropan-1-amine;
N,N-diethyl-2-(4-fluoro-1H-indol-3-yl)ethan-1-amine;
N-(2-(4-fluoro-1H-indol-3-yl)ethyl)-N-propylpropan-1-amine;
N-(2-(7-fluoro-1H-indol-3-yl)ethyl)-N-propylpropan-1-amine;
N-(2-(5,6-difluoro-1H-indol-3-yl)ethyl)-N-propylpropan-1-amine;
2-(5,7-difluoro-1H-indol-3-yl)-N,N-diethylethan-1-amine;
N-(2-(5,7-difluoro-1H-indol-3-yl)ethyl)-N-propylpropan-1-amine;
2-(6,7-difluoro-1H-indol-3-yl)-N,N-diethylethan-1-amine;
N-(2-(6,7-difluoro-1H-indol-3-yl)ethyl)-N-propylpropan-1-amine; and
2-(5-fluoro-1H-indol-3-yl)propan-1-amine.

The compounds of the invention may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Synthesis

The present invention further provides methods of preparing compounds of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula (I), formula (II), or formula (III) can be prepared from commercially available or previously documented starting materials, for example, according to the synthetic methods outlined herein, or according to methods known in the art. Fluorinated tryptamines (IV), can either be commercially acquired or synthesized according to procedures known to one of ordinary skill in the art.

In the following schemes (Schemes 1-8), R^{I} and R^{II} are each independently H or optionally substituted C₁-C₈ hydrocarbyl, R^{III} is optionally substituted C₁-C₈ hydrocarbyl, X is Cl or Br, and each of R¹, R², R³, R⁴, R⁵, R⁶, R⁷, L, R^{a1}, R^{b1}, R^{b2}, R^{c1}, R^{c2}, and R are defined within the scope of the present disclosure.

Reductive alkylation of IV using a suitable hydride source, including but not limited to NaBH₃CN, in the presence of carbonyl compound (V) provides VI (Scheme 1). In certain embodiments, carbonyl compound (V) is an aldehyde or ketone. Subsequent reductive alkylation of VI, in non-limiting examples, using NaBH₃CN in the presence of aldehyde (VII) provides the compound of formula (Ia). In certain embodiments, compound (VI) is isolated as an intermediate. In other embodiments, compound (VI) is further reductively alkylated in situ to provide Ia without isolation of compound (VI). Additionally, a compound of formula (Ia) may be prepared in one step by reductive amination of IV, in non-limiting examples, using NaBH₃CN in the presence of excess aldehyde (VII).

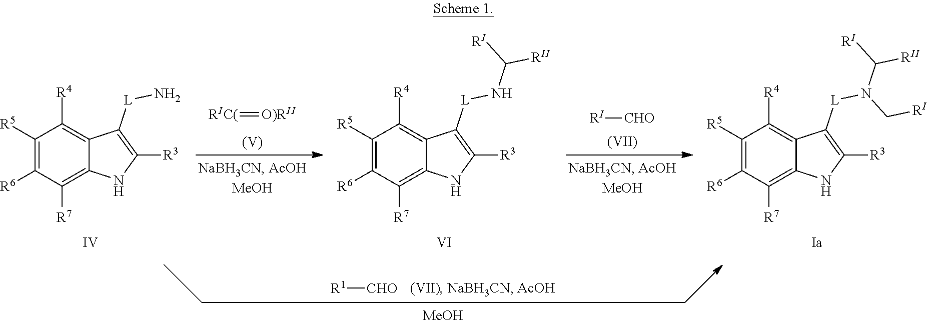

Formylation of IV to provide IX is achieved using a suitable formylating reagent, non-limiting examples including compound (VIII) (e.g., ethyl formate), under conditions including but not limited to heating and/or microwave irradiation (Scheme 2). Reduction of IX with a suitable reducing agent, including but not limited to Red-Al (i.e., sodium bis(2-methoxyethoxy)aluminium hydride), provides VIa. Reductive alkylation of VIa, in non-limiting examples using NaBH₃CN in the presence of aldehyde (VII) provides a compound of formula (Ib).

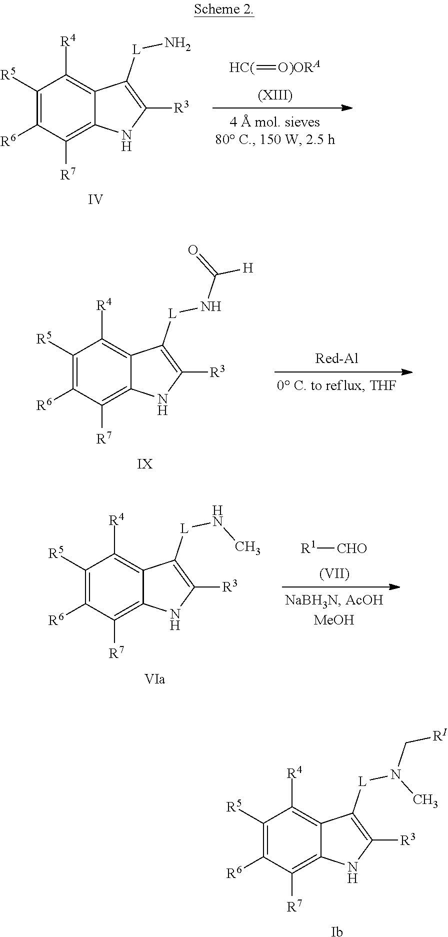

Alternatively, alkylation of VI can be achieved in the presence of a suitable base, non-limiting examples including Et₃N, and a suitable alkylating agent, non-limiting examples including alkyl, allyl, propargyl, and/or benzyl halides (X), to provide the compound of formula (Ic) (Scheme 3).

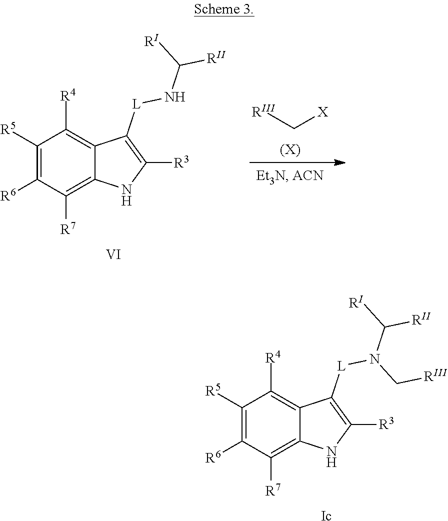

In certain embodiments, the compound of formula (I), which is the compound of formula (Id), may be prepared from fluorinated indole (XI). Acylation of XI with a suitable electrophilic oxalic acid derivative (XII), non-limiting examples including oxalyl chloride, followed by treatment with a suitable nucleophilic amine (XIII), provides XIV (Scheme 4). Subsequent reduction of XIV using a suitable reducing agent, non-limiting examples including LiAlH₄, provides the compound of formula (Id).

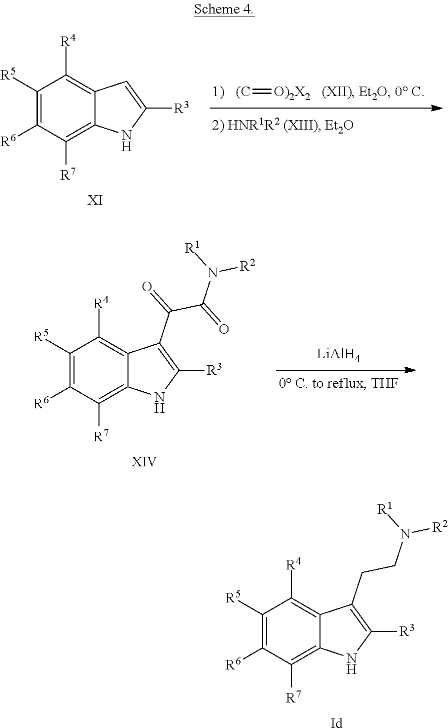

In certain embodiments, the compound of formula (I), which is the compound of formula (Ie), is prepared from XI utilizing a Michael addition/reduction sequence (Scheme 5). Treatment of XI with α,β-unsaturated amide XV in the presence of a suitable Lewis acid, including but not limited to ZnCl₂, under conditions suitable for reactivity, including but not limited to heat and/or microwave irradiation, provides XIV. Reduction of XIV with a suitable reducing agent, including but not limited to LiAlH₄, provides compound (Ie).

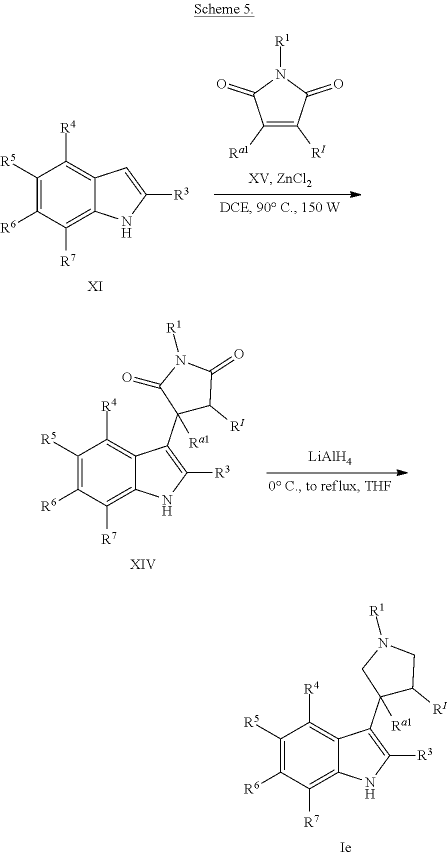

In certain embodiments, the compound of formula (I), which is the compound of formula (If), is prepared from XI utilizing a Friedel-Crafts acylation/S_{N}2 reaction sequence (Scheme 6). Treatment of XI with α-halo-acyl halide (XV) in the presence of a base, including but not limited to pyridine, provides XVI. In certain embodiments, the acyl halide is an acyl chloride or acyl bromide. In certain embodiments, the α-halo-acyl halide is an α-chloro-acyl halide or α-bromo-acyl halide. Nucleophilic displacement of the α-halide (XVI) with amine (XIII) in the presence of a suitable catalyst, including but not limited to potassium iodide, provides compound (If).

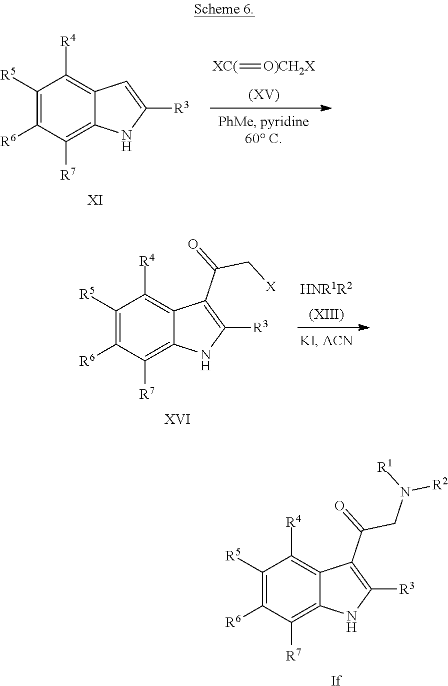

In certain embodiments, the compound of formula (I), which is the compound of formula (Ig), is prepared from XVII in a sequence comprising installation of an α,β-unsaturated nitro group, with subsequent Michael addition, nitro-reduction, and reductive alkylation (Scheme 7). Treatment of aldehyde XVII with nitromethane in the presence of a suitable base, including but not limited to NH₄OAc, under conditions suitable for reactivity, including but not limited to reflux conditions, permits [1,2]-carbonyl addition (i.e., Henry addition) with subsequent dehydration, to afford α,β-unsaturated nitro compound XVIII. Treatment of XVIII with a Grignard reagent, under conditions suitable for reactivity affords Michael adduct XIX. Reduction of XIX, in the presence of a suitable reducing agent, including but not limited to Pd/C, in the presence of H₂, provides amine XX. Reductive alkylation of amine XX using a suitable hydride source, including but not limited to NaBH₃CN, in the presence of carbonyl compound (XXI) provides the compound of formula (Ig).

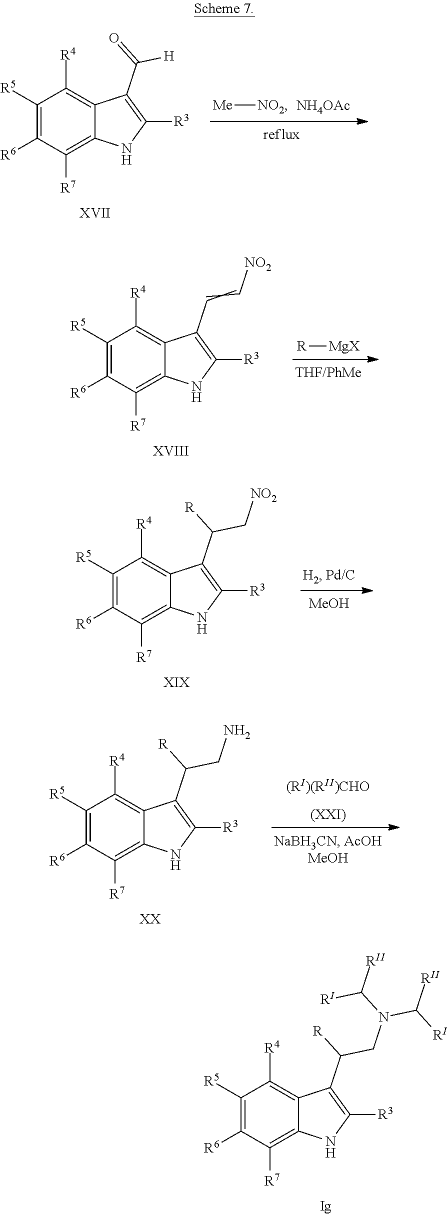

In certain embodiments, the compound of formula (I), which is the compound of formula (Ih), is prepared from XI utilizing a [1,2]-carbonyl addition/reduction sequence (Scheme 8). Treatment of XI with a suitable base, non-limiting examples including KOH, in a suitable solvent, non-limiting examples including methanol, in the presence of a N-protected heterocyclyl ketone (XXII) provides XXIII. In certain embodiments, the XXIII may be prepared by alternative methods. In certain embodiments, R is tert-butyl or benzyl. Reduction of XXIII with a suitable reducing agent, non-limiting examples including LiAlH₄ provides the compound of formula (Ih).

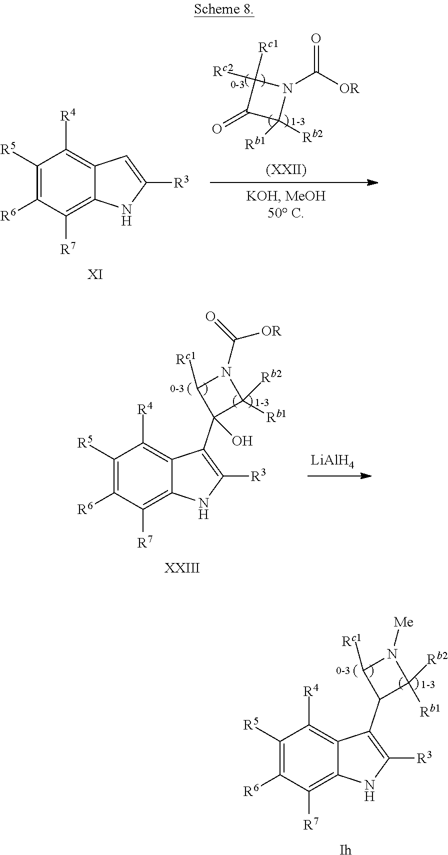

### Methods

In one aspect, the present disclosure provides a pharmaceutical composition comprising at least one compound of the present disclosure and a pharmaceutically acceptable carrier.

In one aspect, the present disclosure provides a method of treating, preventing, and/or ameliorating a psychiatric disease or disorder in a subject in need thereof, the method comprising administering to the subject a therapeutically effective amount of at least one compound of the present disclosure or a pharmaceutical composition of the present disclosure.

In certain embodiments, the psychiatric disease or disorder is selected from the group consisting of a depressive disorder, anxiety disorder, and eating disorder.

In certain embodiments, the psychiatric disease or disorder is selected from the group consisting of attention deficit disorder (ADD), attention deficit hyperactivity disorder (ADHD), treatment resistant depression, major depressive disorder (MDD), bipolar I disorder, bipolar II disorder, cyclothymic disorder, anti-social personality disorder, pain, sleep-wake disorders, disruptive mood dysregulation disorder, persistent depressive disorder (dysthymia) premenstrual dysphoric disorder, substance/medication-induced depressive disorder, post-partum depression, depressive disorder due to another medical condition, separation anxiety disorder, specific phobia, social anxiety disorder, panic disorder, panic attack, agoraphobia, generalized anxiety disorder, substance-medication induced anxiety disorder, anxiety disorder due to another medical condition, somatic symptom disorder, illness anxiety disorder, obsessive-compulsive disorder (OCD), obsessive-compulsive and related disorder (OCRD), OCRD due to another medical condition, substance-related disorders, alcohol-related disorders, cannabis-related disorders, hallucinogen-related disorders, inhalant-related disorders, cocaine-related disorders, opioid-related disorders, sedative-, hypnotic-, and/or anxiolytic-related disorders, stimulant-related disorders, tobacco-related disorders, non-substance-related disorders (gambling and/or gaming disorder), anorexia nervosa, bulimia nervosa, and binge-eating disorder.

In certain embodiments, the subject is further administered at least one additional agent useful for treating, preventing, and/or ameliorating the psychiatric disease or disorder. In certain embodiments, the at least one additional agent is selected from the group consisting of a selective serotonin reuptake inhibitor, triple reuptake inhibitor, serotonin and norepinephrine reuptake inhibitor, tricyclic antidepressant, tetracyclic antidepressant, dopamine reuptake inhibitor, mood stabilizer, anticonvulsant, antipsychotic, anxiolytics, benzodiazepines, monoamine releasers, dopamine receptor agonist, cannabinoids, triptans, anti-migraine medications, analgesics, anti-inflammatory, immune modulator, 5-HT_{1A} receptor antagonist, 5-HT₂ receptor antagonist, 5-HT₃ receptor antagonist, monoamine oxidase inhibitor, and noradrenergic antagonist. In certain embodiments, the subject is co-administered the at least one compound and the at least one additional agent. In certain embodiments, the at least one compound and the at least one additional agent are co-formulated.

In certain embodiments, the subject is a mammal. In certain embodiments, the mammal is a human.

### Pharmaceutical Compositions and Formulations

The invention provides pharmaceutical compositions comprising at least one compound of the invention or a salt, prodrug, solvate, isotopologue, or stereoisomer thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or any combinations of these. At least one compound of the invention may be present in the pharmaceutical composition in the form of a physiologically acceptable salt, such as in combination with a physiologically acceptable cation or anion, as is well known in the art.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered.

### Combination Therapies

In one aspect, the compounds of the invention are useful within the methods of the invention in combination with one or more additional agents useful for treating a psychiatric disorder. These additional agents may comprise compounds or compositions identified herein, or compounds (e.g., commercially available compounds) known to treat, prevent, or reduce the symptoms of one or more psychiatric disorders described herein.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Materials

All starting materials, reagents and solvents used in the syntheses were obtained from the following chemical vendors: AK Scientific (Union City, CA), Matrix Scientific (Columbia, SC), 1PlusChem (San Diego, CA), Enamine (Ukraine), ChemScene LLC (Monmouth Junction, NJ), Oakwood Chemical (Columbia, SC), and Sigma Aldrich (St Louis, MO).

### Mass and pH Measurements

pH readings were obtained by an Orion 3 star (Thermo Scientific, USA) pH meter equipped with either a Thermo pH electrode (9142BN) filled with 3M KCl ROSS Orion filling solution (Thermos Scientific, USA). An Ohaus ADVENTURER AX124 analytical balance (Ohaus, New Jersey, USA) was used. Samples were weighed on 3×3 inch low nitrogen weighing Fisherbrand paper (Fisherbrand, Pittsburgh, USA). For analytical samples, the analytical balance and pH meter were calibrated prior to use. Calibration of the analytical balance was confirmed with a 5 mg standard weight (Troemner, Thorofare, NJ) with 5±0.1 mg cutoff. The pH meter was calibration by utilizing pH 4.01 and pH 7 buffers from Orion application solutions (Thermo Scientific, USA) for a two point calibration.

### High Performance Liquid Chromatography (HPLC)

An Agilent 1260 Infinity system was used that includes a 1260 quaternary pump VL, a 1260 ALS autosampler, a 1260 Thermostatted Column Compartment, and a 1200 DAD Multiple Wavelength Detector (Agilent Technologies, Santa Clara, CA, USA). Detection wavelength was set at 220, and 254 nm. Separation for tryptamines was achieved using a Zorbax Eclipse Plus-C18 analytical column (5 μm, 4.6×150 mm) from Agilent (Agilent Technologies, Santa Clara, CA, USA). Mobile phase A was 10 mM aqueous ammonium formate buffer titrated to pH 4.5 using 10 mM formic acid solution. Mobile phase B was acetonitrile. The injection volume of samples was 40 μL, flow rate was 1.0 mL/min, and the column temperature was set at 25° C. All samples were injected with a wash (30:70 A:B) between runs. Run times were 10 minutes using an isocratic mobile phase ratio (isocratic) of 70% A and 30% B. Chromatograms and peak areas were analyzed using the Agilent ChemStation Software (Agilent Technologies, Santa Clara, CA, USA).

### High Resolution Mass Spectrometry (HRMS)

HRMS experiments were performed on a Thermo Orbitrap Exactive Mass Spectrometer with an Orbitrap mass analyzer, which was calibrated using electrospray ionization with Pierce™ LTQ ESI Positive Ion Calibration Solution (ThermoFisher Scientific, USA). Samples were ionized via an Atmospheric Solids Analysis Probe (ASAP). Thermo Xcalibur Qual Browser software was used for analysis of results and identity was confirmed if <5 ppm error. Measurement parameters: Aux gas flow rate-8, Spray Voltage-3.50 kV, Capillary temperature-275° C., Capillary Voltage-25.00 V, Tube Lens Voltage-65.00 V, Skimmer Voltage-14.00 V, Heater Temperature−100° C.

### Gas Chromatography-Mass Spectrometry (GC-MS)

A Thermo Scientific Trace 1300 Gas Chromatograph coupled to Thermo Scientific ISQ QD Single Quadrupole Mass Spectrometer was used for GC/MS experiments. A Thermo Scientific TraceGold TG-5MS GC Column (30 m×0.25 mm×0.25 μm) was used for separation of any components. All of the samples were made up at a concentration of 1 mg/mL in ethyl acetate. Ionization was achieved by electron impact (EI) at 70 eV. Data was analyzed using Thermo Xcalibur TM Software (version 3.1.66.10). Transfer line and ion source were set to 210° C. and 200° C., respectively. The oven starting temperature was set to 100° C. and held for 1 minute. Temperature was increased at a rate of 8° C./min until to 220° C. at which point it was held for the remainder of the run time. The total run time ranged from 20 to 40 minutes depending on target compound.

### Nuclear Magnetic Resonance Spectroscopy (NMR)

1D and 2D ¹H, ¹³C, ¹⁹F NMR spectra data were obtained on a Bruker Avance III with PA BBO 400S1 BBF-H-D-05 Z plus probe (Bruker Corporation, Billerica, MA, USA). Samples were prepared at a concentration of ˜20 mg/mL in DMSO (Sigma-Aldrich, St. Louis, MO) as the salt form unless otherwise described. Chemical shifts are reported in parts per million (ppm) against DMSO standard (δ=2.50 ppm), for ¹³C (δ=39.52 ppm) and trichlorofluoromethane for ¹⁹F (δ=0 ppm). ¹H, ¹³C, ¹⁹F APT, ¹H-¹³C HSQC, ¹H-¹³C HMBC and ¹H-¹H COSY experiments were performed.

### Melting Point

Melting point data were obtained on a Digimelt A160 SRS digital melting point apparatus (Stanford Research Systems, Sunnyvale, CA, USA) at a ramp rate of 2° C./min.

### Atmospheric Solids Analysis Probe Mass Spectroscopy (ASAP-MS)

ASAP MS experiments were performed on an Advion Expressions CMS Spectrometer with a quadrupole mass analyzer. Samples were ionized utilizing via Atmospheric Solids Analysis source using an Atmospheric Pressure Chemical Ionization (APCI) attachment. Data was analyzed via Advion Data Express software. Measurement parameters were set to. Capillary Temperature=150° C., Capillary Voltage=120 V, Source Gas Temperature=200° C., and APCI corona discharge=5 μA.

### Column Chromatography

Flash column chromatography was performed using a Biotage Isolera One Flash Chromatograph with Spektra UV detection. UV absorption was measured at 254 and 280 nm wavelengths. KP-Sil 50 g and 100 g cartridges were utilized for the separation. Cartridges were manually packed using 230-400 mesh, 60 Å silica gel (Sigma Aldrich, St Louis, MO). Unless otherwise indicated, a gradient of 5-20% EtOH in EtOAc with 1% Et₃N was utilized as the mobile phase. The flowrate was 100 mL/min.

### Short-Path Distillation

Short path distillation was performed utilizing a Kugelrohr short-path distillation apparatus (Sigma Aldrich, St Louis, MO). This was done under a vacuum (˜0.1 mmHg) utilizing a Welch (Skokie, IL) Gem 8890 Vacuum pump.

### Microwave Reactions

CEM Discover—SP w/activent was used as a microwave reactor. Reaction vessels were obtained from CEM in both 35 mL and 10 mL sizes. N₂ gas was used for cooling.

### Docking Studies

Rigid docking using UCSD Autodock 4.2.6 was utilized. The receptor used was 6WGT (Kim et al. 2020). Receptor and ligand structures were prepared using Discovery Studio Visualizer. Docking was performed using AutodockTools 1.5.6. Receptor preparation included removing ligand, adding polar hydrogen atoms, merging non-polar hydrogen atoms, and adding Gasteiger charges. The grid was centered around the orthosteric LSD binding site. Genetic algorithm was used for search parameters and output was Lamarckian genetic algorithm. Docking results were ranked by energy and exported, then individually viewed in PyMol.

### Example 1: N-ethyl-N-(2-(5-fluoro-1H-indol-3-yl)ethyl)propan-2-amine (1)

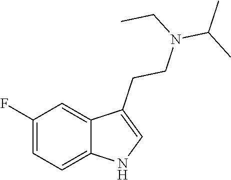 [SMILES: CCN(CCc1c[nH]c2ccc(F)cc12)C(C)C]

5-fluorotryptamine hydrochloride (0.8 g, 3.73 mmol) was dissolved in MeOH (20 mL, dried over 3 Å molecular sieves) under argon, and glacial acetic acid (21.33 μL) was added. NaCNBH₃ (0.26 g, 4.10 mmol) was added and the mixture was allowed to stir for 3 min to dissolve particulates. Acetone (0.82 mL, 11.2 mmol) was then added, and the reaction was allowed to stir at room temperature for 4 h. Upon completion (as confirmed by GC-MS, TLC, and ASAP-MS), acetaldehyde (0.63 mL, 11.2 mmol) was added, and the reaction was allowed to stir at room temperature for 1 h. Next, the reaction was poured into 300 mL of 0.2 M HCl and extracted with EtOAc (3×50 mL). These organic phases were pooled, extracted with 0.2 M aqueous HCl (3×75 mL), and the aqueous phases were combined with the original aqueous phase. This combined aqueous phases were basified by the addition of KOH flakes, and extracted with EtOAc (3×100 mL). The organic phases were combined, the pooled organic phases were washed with brine (20 mL), dried over anhydrous Na₂SO₄, and the solvent removed in vacuo to provide the crude product (0.98 g) as an amber oil. The crude product was purified by silica gel chromatography using a 50% EtOH/EtOAc (1% Et₃N v/v) mobile phase to provide the title compound as an amber solid (0.83 g, 3.34 mmol, 89.54% yield). The solid was dissolved in boiling cyclohexane and EtOAc (1 mL) and allowed to cool to room temperature. The solution was further cooled to 4° C. until thermodynamic equilibrium was attained, and subsequently placed at −20° C. for 72 h. The solution was decanted, and the resulting crystals were washed with hexanes. Crystallization was repeated once to provide the title compound as a white crystalline solid (m.p. 77.1-78.1° C.). HR-ASAP-MS: m/z 249.1752 (theoretical [M+H]⁺, C₁₅H₂₂FN₂⁺), m/z 249.1762 (observed, Δ=−4 ppm). (Free base)¹H-NMR (400 MHz, d₆-DMSO) δ 10.84 (s, 1H), 7.29 (dd, J=8.8, 4.6 Hz, 1H), 7.21 (d, J=2.3 Hz, 1H), 7.18 (dd, J=10.1, 2.5 Hz, 1H), 6.86 (td, J=9.2, 2.5 Hz, 1H), 2.94 (septet, J=6.6 Hz, 1H), 2.74-2.66 (m, 2H), 2.61-2.54 (m, 2H), 2.52-2.48 (m, 2H), 0.98 (t, J=7.1 Hz, 3H), 0.92 (d, J=6.6 Hz, 6H). ¹³C-NMR (101 MHz, d₆-DMSO) δ 156.55 (d, J=230.8 Hz, 1C), 132.79 (s, 1C), 127.51 (d, J=9.5 Hz, 1C), 124.65 (s, 1C), 113.36 (d, J=4.4 Hz, 1C), 112.16 (d, J=9.7 Hz, 1C), 108.77 (d, J=26.2 Hz, 1C), 102.80 (d, J=22.8 Hz, 1C), 50.17 (s, 1C), 49.74 (s, 1C), 43.46 (s, 1C), 25.14 (s, 1C), 18.32 (s, 2C), 14.45 (s, 1C). ¹⁹F-NMR (377 MHz, d₆-DMSO) δ−125.38 (s, 1F).

### Example 2: N-(2-(5-fluoro-1H-indol-3-yl)ethyl)-N-methylpropan-2-amine (2)

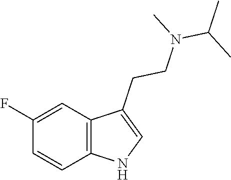 [SMILES: CC(C)N(C)CCc1c[nH]c2ccc(F)cc12]

N-(2-(5-fluoro-1H-indol-3-yl)ethyl)-N-methylpropan-2-amine (2) was synthesized in a similar manner as described above for N-ethyl-N-(2-(5-fluoro-1H-indol-3-yl)ethyl)propan-2-amine (1), starting from 5-fluorotryptamine hydrochloride (0.8 g, 3.73 mmol), acetone (0.65 g, 11.2 mmol), and formaldehyde (0.93 mL, 11.2 mmol, 36% in H₂O v/v). The crude product was purified by Kugelrohr distillation at 170-195° C. to provide the title compound as a yellow crystalline solid (0.81 g, 3.46 mmol, 92.8% yield). The solid was subsequently acidified to provide the HCl salt of the title compound as a white crystalline powder. HR-ASAP-MS: m/z 235.1600 (theoretical [M+H]⁺, C₁₄H₂OFN₂⁺), m/z 235.1605 (observed, Δ=−2.1 ppm). ¹H-NMR (400 MHz, d₆-DMSO) δ 11.14 (s, 1H), 10.69 (s, 1H), 7.46 (dd, J=10.1, 2.5 Hz, 1H), 7.36 (dd, J=8.8, 4.5 Hz, 2H), 7.34 (d, J=2.3 Hz, 1H), 6.93 (td, J=9.2, 2.5 Hz, 1H), 3.60 (septet of doublets, J=6.6, 2.3 Hz, 1H), 3.30-3.17 (m, 2H), 3.17-3.09 (m, 2H), 2.71 (d, J=5.1 Hz, 3H), 1.30 (d, J=6.6 Hz, 3H), 1.23 (d, J=6.6 Hz, 3H). ¹³C-NMR (101 MHz, d₆-DMSO) δ 156.74 (d, J=231.1 Hz, 1C), 132.86 (s, 1C), 126.99 (d, J=10.0 Hz, 1C), 125.46 (s, 1C), 112.45 (d, J=9.7 Hz, 1C), 109.63 (d, J=4.9 Hz, 1C), 109.30 (d, J=26.1 Hz, 1C), 103.15 (d, J=23.0 Hz, 1C), 55.69 (s, 1C), 52.27 (s, 1C), 34.07 (s, 1C), 20.05 (s, 1C), 16.82 (s, 1C), 14.90 (s, 1C). ¹⁹F-NMR (377 MHz, d₆-DMSO) δ−124.76 (s, 1F).

### Example 3: N-(2-(5-fluoro-1H-indol-3-yl)ethyl)-N-methylbutan-2-amine (3)

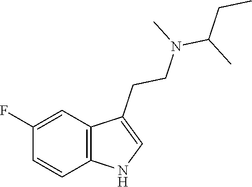 [SMILES: CCC(C)N(C)CCc1c[nH]c2ccc(F)cc12]

N-(2-(5-fluoro-1H-indol-3-yl)ethyl)-N-methylbutan-2-amine (3) was synthesized in a similar manner as described above for N-ethyl-N-(2-(5-fluoro-1H-indol-3-yl)ethyl)propan-2-amine (1), starting from 5-fluorotryptamine hydrochloride (0.8 g, 3.73 mmol), methyl ethyl ketone (0.81 g, 11.2 mmol), and formaldehyde (0.93 mL, 11.2 mmol, 36% in H₂O v/v), to provide the title compound as a yellow crystalline solid (0.82 g, 3.30 mmol, 88.5% yield), and subsequently the HCl salt as a white crystalline solid (m.p. 136.5-142° C.). HR-ASAP-MS: m/z 249.1756 (theoretical [M+H]⁺, C₁₅H₂₂FN₂⁺), m/z 249.1762 (observed, Δ=−2.4 ppm). ¹H NMR (400 MHz, DMSO) δ 10.89 (s, 1H), 7.31 (dd, J=8.8, 4.6 Hz, 1H), 7.22 (d, J=2.2 Hz, 1H), 7.21 (dd, J=9.8, 2.5 Hz, 1H), 6.88 (td, J=9.2, 2.5, Hz, 1H), 2.82-2.66 (m, 2H), 2.66-2.58 (m, 1H), 2.58-2.51 (m, 2H), 2.20 (s, 3H), 1.50-1.37 (m, 1H), 1.27-1.16 (m, 1H), 0.87 (d, J=6.5 Hz, 1H), 0.82 (t, J=7.4 Hz, 1H). ¹³C NMR (101 MHz, DMSO) δ 156.56 (d, J=230.5 Hz, 1C), 132.81 (s, 1C), 127.52 (d, J=9.5 Hz, 1C), 124.67 (s, 1C), 113.26 (d, J=4.9 Hz, 1C), 112.16 (d, J=9.9 Hz, 1C), 108.77 (d, J=26.0 Hz, 1C), 102.83 (d, J=22.8 Hz, 1C), 59.21 (s, 1C), 53.87 (s, 1C), 36.34 (s, 1C), 26.10 (s, 1C), 23.90 (s, 1C), 13.03 (s, 1C), 11.38 (s, 1C). ¹⁹F-NMR (377 MHz, d₆-DMSO) δ−124.78 (d, J=10.1 Hz, 1F).

### Example 4: N-(2-(5-fluoro-1H-indol-3-yl)ethyl)-N-isopropylpropan-1-amine (4)

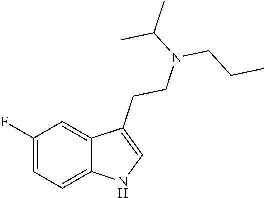 [SMILES: CCCN(CCc1c[nH]c2ccc(F)cc12)C(C)C]

N-(2-(5-fluoro-1H-indol-3-yl)ethyl)-N-isopropylpropan-1-amine (4) was synthesized in a similar manner as described above for N-ethyl-N-(2-(5-fluoro-1H-indol-3-yl)ethyl)propan-2-amine (1), starting from 5-fluorotryptamine hydrochloride (0.8 g, 3.73 mmol), acetone (0.65 g, 11.2 mmol), and propionaldehyde (0.65 g, 11.2 mmol) to provide the title compound as a yellow oil (0.68 g, 2.59 mmol, 69.4% yield), and subsequently the HCl salt as a white crystalline solid (m.p. 155.8-158° C.). HR-ASAP-MS: m/z 263.1912 (theoretical [M+H]⁺, C₁₄H₂OFN₂⁺), m/z 263.1918 (observed, Δ=−2.3 ppm). ¹H-NMR (400 MHz, d₆-DMSO) δ 11.14 (s, 1H), 10.39 (s, 1H), 7.44 (dd, J=10.0, 2.3 Hz, 1H), 7.39-7.33 (m, 2H), 6.93 (td, J=9.2, 2.4 Hz, 1H), 3.72-3.62 (m, 1H), 3.26-3.14 (m, 4H), 3.11-2.96 (m, 2H), 1.79 (sx, J=7.7 Hz, 2H), 1.30 (d, J=6.6 Hz, 6H), 0.94 (t, J=7.3 Hz, 3H). ¹³C-NMR (101 MHz, d₆-DMSO) δ 156.77 (d, J=231.2 Hz, 1C), 132.86 (s, 1C), 126.99 (d, J=9.8 Hz, 1C), 125.54 (s, 1C), 112.50 (d, J=9.8 Hz, 1C), 109.79 (d, J=4.4 Hz, 1C), 109.32 (d, J=26.2 Hz, 1C), 103.07 (d, J=23.2 Hz, 1C), 53.92 (s, 1C), 50.63 (s, 1C), 49.94 (s, 1C), 20.36 (s, 1C), 17.99 (s, 1C), 16.20 (s, 1C), 15.88 (s, 1C), 11.16 (s, 1C). ¹⁹F-NMR (377 MHz, d₆-DMSO) δ−124.69 (s, 1F).

### Example 5: N-(2-(5-fluoro-1H-indol-3-yl)ethyl)-N-propylpropan-1-amine (5)

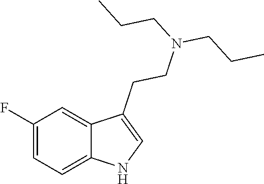 [SMILES: CCCN(CCC)CCc1c[nH]c2ccc(F)cc12]

5-fluorotryptamine hydrochloride (0.8 g, 3.73 mmol) was dissolved in MeOH (20 mL, dried over 3 Å molecular sieves) under argon and glacial acetic acid (21.33 μL) was added to the reaction. NaBH₃CN (0.26 g, 0.00410 mol) was then added and the mixture was allowed to stir for 3 min to dissolve particulates. Propionaldehyde (1.6 mL, 22.3 mmol) was then added, and the reaction was allowed to stir at room temperature for 2 h. Upon completion (confirmed by GC-MS, TLC, and ASAP-MS) the reaction was poured into 300 mL of 0.2 M aqueous hydrochloric acid (HCl) and washed with EtOAc (3×50 mL). The organic phases were pooled and extracted with 0.2 M HCl (3×75 mL). The aqueous extracts were then combined with the original aqueous phase. The combined aqueous phases were basified by the addition of KOH flakes, and subsequently extracted with EtOAc (3×100 mL). The pooled organic phases were then washed with brine (20 mL), dried over anhydrous Na₂SO₄, and the solvent removed in vacuo to provide the crude product as a yellow oil (0.92 g). The crude product was purified by short path vacuum distillation (i.e., Kugelrohr distillation) at 170-195° C. under vacuum to provide the title compound as a yellow solid (0.91 g, 3.46 mmol, 92.9% yield).

The free base was converted to the HCl salt by titrating the free base with concentrated HCl solution in ethanol until the pH<2. The solvent was then evaporated under a stream of warm air several times to yield crystalline material with the absence of excess acid or moisture. The resulting solids were washed with Et₂O (2×10 mL). Solids were dissolved in boiling EtOH (8 mL), then Et₂O (2 mL) was slowly added to provide a milky white opaque solution, which was subsequently allowed to cool to room temperature. The solution was further cooled to 4° C. until thermodynamic equilibrium was attained, then stored at −20° C. overnight. The solution was decanted, and the resulting crystals were washed with Et₂O (3×10 mL), and dried with gentle heat. Crystallization was repeated twice, to provide the HCl salt of the title compound as a white crystalline solid (m.p. 187.5-188.0° C.). HR-ASAP-MS: m/z 263.1911 (theoretical [M+H]⁺, C₁₆H₂₄FN₂⁺), 263.1918 (observed, =Δ−2.7 ppm). ¹H-NMR (400 MHz, d₆-DMSO) δ 11.14 (s, 1H), 10.64 (s, 1H), 7.44 (dd, J=10.1, 2.5 Hz, 1H), 7.38-7.33 (m, 2H), 6.93 (td, J=9.2, 2.5 Hz, 1H), 3.27-3.21 (m, 2H), 3.18-3.11 (m, 2H), 3.11-2.98 (m, 4H), 1.72 (sx, J=7.7 Hz, 4H), 0.92 (t, J=7.4 Hz, 6H). ¹³C-NMR (101 MHz, d₆-DMSO) δ 157.23 (d, J=231.1 Hz, 1C), 133.34 (s, 1C), 127.45 (d, J=10.1 Hz, 1C), 125.97 (s, 1C), 112.97 (d, J=9.9 Hz, 1C), 110.07 (d, J=4.6 Hz, 1C), 109.80 (d, J=26.2 Hz, 1C), 103.57 (d, J=23.1 Hz, 1C), 53.52 (s, 1C), 52.71 (s, 1C), 19.84 (s, 2C), 16.92 (s, 2C), 11.41 (s, 2C). ¹⁹F-NMR (377 MHz, d₆-DMSO) δ−124.75 (s, 1F).

### Example 6: N-ethyl-N-(2-(5-fluoro-1H-indol-3-yl)ethyl)propan-1-amine (6)

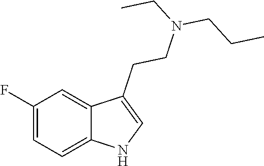 [SMILES: CCCN(CC)CCc1c[nH]c2ccc(F)cc12]

### Synthesis of N-(2-(5-fluoro-1H-indol-3-yl)ethyl)propan-1-amine

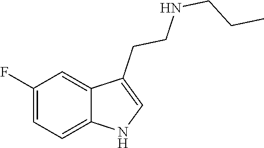 [SMILES: CCCNCCc1c[nH]c2ccc(F)cc12]

N-(2-(5-fluoro-1H-indol-3-yl)ethyl)propan-1-amine was synthesized in a similar manner as described above for N-ethyl-N-(2-(5-fluoro-1H-indol-3-yl)ethyl)propan-2-amine (1), starting from 5-fluorotryptamine hydrochloride (0.8 g, 3.73 mmol) and propionaldehyde (0.22 g, 3.73 mmol), wherein only one molar equivalent of propionaldehyde was used, to provide the title compound as a yellow oil (0.3 g, 1.36 mmol, 36.5% yield). HR-ASAP-MS: m/z 221.1444 (theoretical [M+H]⁺, C₁₃H₁₈FN₂⁺), m/z 221.1449 (observed, Δ=−2.3 ppm).

### Synthesis of N-ethyl-N-(2-(5-fluoro-1H-indol-3-yl)ethyl)propan-1-amine (6)

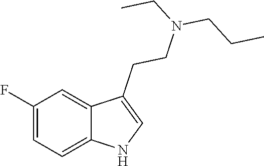 [SMILES: CCCN(CC)CCc1c[nH]c2ccc(F)cc12]

N-ethyl-N-(2-(5-fluoro-1H-indol-3-yl)ethyl)propan-1-amine (6) was synthesized in a manner as described above for N-(2-(5-fluoro-1H-indol-3-yl)ethyl)-N-propylpropan-1-amine (5), starting from N-(2-(5-fluoro-1H-indol-3-yl)ethyl)propan-1-amine (0.26 g, 1.18 mmol) and acetaldehyde (0.31 g, 7.08 mmol). The crude product was purified by silica gel chromatography using 20% EtOH/EtOAc (1% Et₃N v/v) as the mobile phase, as opposed to Kugelrohr distillation, to provide the title compound as a yellow oil (0.21 g, 0.846 mmol), and subsequently the corresponding HCl salt as a white crystalline solid (m.p. 154.5-155.4° C.). GC-MS (tᵣ)=17.02 min; HPLC: 99.4615% purity; HR-ASAP-MS: m/z 249.1757 (theoretical [M+H]⁺, C₁₅H₂₂FN₂⁺), m/z 249.1762 (observed, =Δ−2 ppm). ¹H-NMR (400 MHz, d₆-DMSO) δ 11.14 (s, 1H), 10.65 (s, 1H), 7.44 (dd, J=10.1, 2.5 Hz, 1H), 7.38-7.33 (m, 2H), 6.93 (td, J=9.2, 2.5 Hz, 1H), 3.27-3.17 (m 4H), 3.16-3.10 (m, 2H), 3.10-2.99 (m, 2H), 1.71 (sx, J=7.7 Hz, 2H), 1.26 (t, J=7.2 Hz, 3H), 0.92 (t, J=7.4 Hz, 3H). ¹³C-NMR (101 MHz, d₆-DMSO) δ 156.76 (d, J=231.2 Hz, 1C), 132.87 (s, 1C), 126.97 (d, J=9.8 Hz, 1C), 125.49 (s, 1C), 112.50 (d, J=9.9 Hz, 1C), 109.59 (d, J=4.7 Hz, 1C), 109.33 (d, J=26.3 Hz, 1C), 103.08 (d, J=23.3 Hz, 1C), 52.39 (s, 1C), 51.59 (s, 1C), 46.45 (s, 1C), 19.37 (s, 1C), 16.50 (s, 1C), 10.95 (s, 1C), 8.36 (s, 1C). ¹⁹F-NMR (377 MHz, d₆-DMSO) δ−124.74.

### Example 7: N-ethyl-2-(7-fluoro-1H-indol-3-yl)-N-methylethan-1-amine (7)

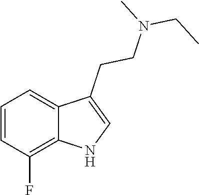 [SMILES: CCN(C)CCc1c[nH]c2c(F)cccc12]

### Synthesis of intermediate N-ethyl-2-(7-fluoro-1H-indol-3-yl)-N-methyl-2-oxoacetamide

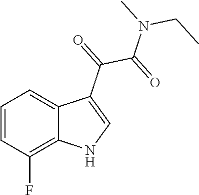 [SMILES: CCN(C)C(=O)C(=O)c1c[nH]c2c(F)cccc12]

A two-neck round bottom flask (RBF), addition funnel, rubber septa, and magnetic stir bar were dried in an oven overnight. The reaction apparatus was assembled and subsequently purged with argon. Under a positive pressure of argon, Et₂O (20 mL) was added to the flask through the addition funnel and the solvent was cooled to 0° C. in an ice-water bath. Oxalyl chloride (3.81 mL, 44.4 mmol) was added to the round bottom flask via a syringe. Next, a solution of 7-fluoroindole (5 g, 37.0 mmol) in Et₂O (40 mL) was added dropwise over 30 mins. Once the addition was complete, the reaction was allowed to stir for 1 h at 0° C., during which period a yellow precipitate formed. The reaction was warmed to room temperature and stirred for an additional 3 h. Next, a solution of N-methylethanamine (9.85 mL, 0.115 mol) in Et₂O (20 mL) was added dropwise with vigorous stirring and mixing over the period of approximately 10 min while under a positive pressure of argon (exothermic). Following the addition, volatiles (e.g., Et₂O and amines) were removed in vacuo to yield a whitish-brown solid. The solids were washed with water and collected by vacuum filtration. The resulting brownish-tan solids were allowed to dry in a fume hood overnight, then placed in a desiccator for >24 h to yield the title compound (6.7 g, 27.0 mmol, 73% yield). The compound was used in the subsequent step without further purification.

### Synthesis of N-ethyl-2-(7-fluoro-1H-indol-3-yl)-N-methylethan-1-amine (7)

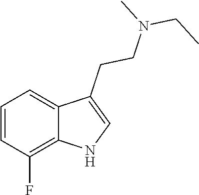 [SMILES: CCN(C)CCc1c[nH]c2c(F)cccc12]

A three neck 500 mL round bottom flask, 300 mL addition funnel, stir bar, and a condenser dried were dried overnight in an oven. The reaction apparatus was assembled and subsequently purged with argon. Next, THE (100 mL, dried over 3 Å molecular sieves) was added to flask and cooled to 0° C. Lithium aluminum hydride (LAH) (3.07 g, 0.081 mol) was added slowly to the round bottom flask with stirring and under argon atmosphere. Once the addition was complete, a solution of N-ethyl-2-(7-fluoro-1H-indol-3-yl)-N-methyl-2-oxoacetamide (6.7 g, 27.0 mmol mmol) in 100 mL of THF was added dropwise over 1 h at 0° C. The additional funnel was subsequently rinsed with THE (20 mL) and added to the reaction over a period of 10 min.

The reaction was then heated at reflux for 1.5 h, then cooled to 0° C., and quenched with a THF/Et₂O (˜1:1) mixture and ice. Once quenched, brine and aqueous KOH were added to ensure basicity, followed by EtOAc (100 mL). The inorganic solids were removed by gravity filtration over Whatman paper (24 cm diameter). The removed solids were then washed extensively with EtOAc. The filtrate was extracted with 0.2 M HCl (aq.) (3×166 mL). The aqueous phase was then basified by the addition of KOH pellets and extracted with EtOAc (3×100 mL). The pooled organic phases were washed with brine (20 mL), dried over anhydrous Na₂SO₄, and concentrated in vacuo to provide the crude product as a brownish-red solid (3.98 g). The crude product (free base) was recrystallized from boiling hexanes with a small amount of EtOAc and stored at −20° C. The recrystallization was repeated three times to provide the title compound as light yellow transparent crystals (1.8 g, 7.25 mmol, 34.03% yield). The purified material was converted to the HCl salt as a white crystalline solid (m.p. 133.8-135° C.). High-resolution atmospheric solid analysis probe mass spectrometry (HR-ASAP-MS): m/z 221.1445 (theory [M+H]⁺ C₁₃H₁₈FN₂⁺, m/z 221.1449, Δ=−1.8 ppm). ¹H-NMR (400 MHz, d₆-DMSO) δ 11.50 (s, 1H), 10.86 (s, 1H), 7.48 (d, J=7.7 Hz, 1H), 7.33 (d, J=2.4 Hz, 1H), 7.02-6.96 (m, 1H), 6.96-6.89 (m, 1H), 3.39-3.18 (m, 4H), 3.18-3.02 (m, 2H), 2.78 (d, J=3.7 Hz, 3H), 1.26 (t, J=7.3 Hz, 3H). ¹³C-NMR (101 MHz, d₆-DMSO) δ 149.24 (d, J=242.8 Hz, 1C), 130.78 (d, J=6.0 Hz, 1C), 124.46 (s, 1C), 123.94 (d, J=13.3 Hz, 1C), 118.83 (d, J=6.3 Hz, 1C), 114.60 (d, J=3.0 Hz, 1C), 110.45 (d, J=2.1 Hz, 1C), 106.04 (d, J=16.1 Hz, 1C), 54.26 (s, 1C), 49.80 (s, 1C), 38.17 (s, 1C), 19.63 (s, 1C), 8.75 (s, 1C). ¹⁹F-NMR (377 MHz, d₆-DMSO) δ−133.11 (s, 1F).

### Example 8: N,N-diethyl-2-(4-fluoro-1H-indol-3-yl)ethan-1-amine (8)

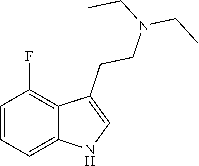 [SMILES: CCN(CC)CCc1c[nH]c2cccc(F)c12]

N,N-diethyl-2-(4-fluoro-1H-indol-3-yl)ethan-1-amine (8) was synthesized in a similar manner as described above for N-ethyl-2-(7-fluoro-1H-indol-3-yl)-N-methylene-1-amine (7), starting from 4-fluoroindole (5 g, 37 mmol) to provide intermediate N,N-diethyl-2-(4-fluoro-1H-indol-3-yl)-2-oxoacetamide 1.6 g, 6.10 mmol, 16.5% yield), which was further reacted to provide the title compound as an orange oil after column chromatography using an alumina stationary phase with an EtOAc eluent (0.81 g, 3.46 mmol, 56.7%). The free base was subsequently converted to the corresponding fumarate salt and washed with acetone to provide a white crystalline solid. High-resolution atmospheric solid analysis probe mass spectrometry (HR-ASAP-MS): m/z 235.1601 (theory [M+H]⁺ C₁₄H₂OFN₂⁺, m/z 235.1605, Δ=−1.7 ppm). ¹H-NMR (400 MHz, d₆-DMSO) δ 11.25 (s, 1H), 7.24 (d, J=2.1 Hz, 1H), 7.18 (d, J=8.1 Hz, 1H), 7.05-7.00 (m, 1H), 6.72 (dd, J=11.6, 7.8 Hz, 1H), 6.52 (s, 2H), 3.03 (s, 4H), 2.94 (q, J=7.2 Hz, 4H), 1.14 (t, J=7.2 Hz, 6H). ¹³C-NMR (101 MHz, d₆-DMSO) δ 167.42 (s, 1C), 156.26 (d, J=242.8 Hz, 1C), 139.27 (d, J=12.0 Hz, 1C), 134.81 (s, 1C), 123.85 (s, 1C), 121.53 (d, J=7.9 Hz, 1C), 115.19 (d, J=19.8 Hz, 1C), 108.89 (s, 1C), 108.14 (d, J=3.3 Hz, 1C), 103.33 (d, J=19.2 Hz, 1C), 52.28 (s, 1C), 46.01 (s, 1C), 21.55 (s, 2C), 9.66 (s, 2C). ¹⁹F-NMR (377 MHz, d₆-DMSO) δ−124.58 (s, 1F).

### Example 9: N-(2-(5,6-difluoro-1H-indol-3-yl)ethyl)-N-ethylpropan-1-amine (9)

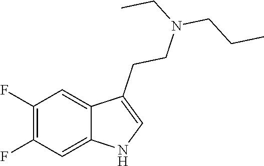 [SMILES: CCCN(CC)CCc1c[nH]c2cc(F)c(F)cc12]

N-(2-(5,6-difluoro-1H-indol-3-yl)ethyl)-N-ethylpropan-1-amine (9) was synthesized in a similar manner as described above for N-ethyl-2-(7-fluoro-1H-indol-3-yl)-N-methylene-1-amine (7), starting from 5,6-difluoroindole (2.5 g, 16.3 mmol) to provide intermediate 2-(5,6-difluoro-1H-indol-3-yl)-N-ethyl-2-oxo-N-propylacetamide (3.5 g, 13.9 mmol, 85.3% yield), a portion of which was further reacted under modified conditions (i.e., NH₄OH and AcOH used in place of KOH and HCl, respectively) to provide the crude product (2.14 g, 8.04 mmol, m.p. 83-86° C., 76.6% yield), which was purified by column chromatography using an alumina stationary phase and an EtOAc eluent. High-resolution atmospheric solid analysis probe mass spectrometry (HR-ASAP-MS): m/z 267.1665 (theory [M+H]⁺ C₁₅H₂₁F₂N₂⁺, m/z 267.1667, Δ=−0.7 ppm). (Free base) ¹H-NMR (400 MHz, d₆-DMSO) δ 10.93 (s, 1H), 7.44 (dd, J=11.4, 8.0 Hz, 1H), 7.30 (dd, J=11.3, 7.0 Hz, 1H), 7.21 (d, J=2.1 Hz, 1H), 2.78-2.69 (m, 2H), 2.68-2.61 (m, 2H), 2.58-2.51 (m, 2H), 2.41 (t, J=7.3 Hz, 2H), 1.40 (sx, J=7.4 Hz, 2H), 0.97 (t, J=7.1 Hz, 3H), 0.84 (d, J=14.7 Hz, 3H). ¹⁹F-NMR (377 MHz, d₆-DMSO) δ−145.11 (d, J=21.9 Hz, 1F), −148.53 (d, J=22.0 Hz, 1F).

### Example 10: N-(2-(5-fluoro-1H-indol-3-yl)ethyl)-N-methylprop-2-en-1-amine (10)

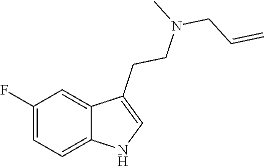 [SMILES: C=CCN(C)CCc1c[nH]c2ccc(F)cc12]

N-(2-(5-fluoro-1H-indol-3-yl)ethyl)-N-methylprop-2-en-1-amine (10) was synthesized in a similar manner as described above for N-ethyl-2-(7-fluoro-1H-indol-3-yl)-N-methylene-1-amine (7), starting from 5-fluoroindole (3 g, 22.2 mmol), to provide intermediate N-[2-(5-fluoro-1H-indol-3-yl)ethyl]-N-methylprop-2-en-1-amine (4.5 g, 17.3 mmol, 77.9% yield), a portion of which was further reacted to provide the title compound as a yellow oil after purification by column chromatography using a silica gel stationary phase, and subsequently using an alumina stationary phase and an EtOAc eluent (2.18 g, 9.38 mmol, 55.55% yield). High-resolution atmospheric solid analysis probe mass spectrometry (HR-ASAP-MS): m/z 233.1443 (theory [M+H]⁺ C₁₄H₁₈FN₂⁺, m/z 233.1449, Δ=−2.6 ppm). (Free base) ¹H-NMR (400 MHz, d₆-DMSO) δ 10.86 (s, 1H), 7.31 (dd, J=8.8, 4.6 Hz, 1H), 7.22 (dd, J=9.9, 2.4 Hz, 1H), 7.21 (d, J=2.3 Hz, 1H) 6.88 (dt, J=9.2, 2.5 Hz, 1H), 5.89-5.78 (m, 1H), 5.19 (d, J=20 Hz, 1H), 5.11 (td, J=10.2, 0.9 Hz, 1H), 3.03 (d, J=6.4 Hz, 1H), 2.79 (t, J=7.8 Hz, 1H), 2.61-2.54 (m, 1H), 2.23 (s, 1H). ¹⁹F-NMR (377 MHz, d₆-DMSO) δ−125.32 (s, 1F).

### Example 11: N-(2-(5-fluoro-1H-indol-3-yl)ethyl)-N-isopropylprop-2-en-1-amine (11)

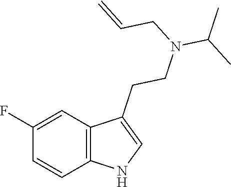 [SMILES: C=CCN(CCc1c[nH]c2ccc(F)cc12)C(C)C]

### Synthesis of N-[2-(5-fluoro-1H-indol-3-yl)ethyl]propan-2-amine

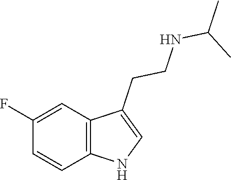 [SMILES: CC(C)NCCc1c[nH]c2ccc(F)cc12]

N-[2-(5-fluoro-1H-indol-3-yl)ethyl]propan-2-amine was synthesized in a similar manner as described above for N-(2-(5-fluoro-1H-indol-3-yl)ethyl)-N-propylpropan-1-amine (5), starting from 5-fluorotryptamine (1.3 g, 6.06 mmol), and acetone (2.11 g, 36.4 mmol), to provide the title compound as an amber solid (1.47 g, quantitative). The product was used in the subsequent reaction without further purification.

### Synthesis of N-(2-(5-fluoro-1H-indol-3-yl)ethyl)-N-isopropylprop-2-en-1-amine (11)

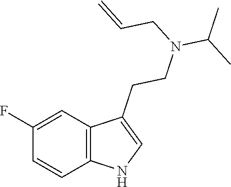 [SMILES: C=CCN(CCc1c[nH]c2ccc(F)cc12)C(C)C]

N-[2-(5-fluoro-1H-indol-3-yl)ethyl]propan-2-amine (0.7 g, 3.18 mmol) was dissolved in dry ACN (15 mL) in a heat dried 50 mL round bottom flask with stirring, under an argon atmosphere. Triethylamine (2.66 mL, 19.1 mmol), then allyl iodide (1.45 mL, 15.9 mmol) were added to the reaction vessel, resulting in an exothermic reaction (˜38° C.). The flask was wrapped with aluminum foil and allowed to stir at room temperature for 44 h. The reaction was poured into 200 mL of 0.2 M AcOH (aq.) and extracted with EtOAc (3×50 mL). The organic phases were combined and extracted with 0.2 M AcOH (aq.) (3×50 mL), then the aqueous phases were combined with the original aqueous phases. The combined aqueous phases were basified with NH₄OH (28-30% NH₃) and extracted with EtOAc (3×100 mL). The pooled organic phases were washed with brine, dried over anhydrous Na₂SO₄, and concentrated in vacuo to provide the crude product as a viscous amber/brown oil (699 mg). The crude product was purified by silica gel chromatography using 20% EtOH/EtOAc (1% Et₃N v/v) to provide the title compound as off-white solid (120 mg, m.p. 45.5-47° C., 14.49% yield). The purified material was subsequently converted to the HCl salt as described herein to provide a white crystalline solid (m.p. 132-134° C.). High-resolution atmospheric solid analysis probe mass spectrometry (HR-ASAP-MS): m/z 261.1756 (theory [M+H]⁺C₁₆H₂₂FN₂⁺, m/z 261.1762, Δ=−2.3 ppm). ¹H-NMR (400 MHz, d₆-DMSO) δ 11.14 (s, 1H), 10.87 (s, 1H), 7.40 (dd, J=10, 2.5 Hz, 1H), 7.38-7.33 (m, 2H), 6.93 (dt, J=9.2, 2.5 Hz, 1H), 6.24-6.11 (m, 1H), 5.57 (dd, J=17.1, 1.0 Hz, 1H), 5.47 (d, J=10.3 Hz, 1H), 3.88-3.79 (m, 2H), 3.71-3.61 (m, 1H), 3.29-3.21 (m, 1H), 3.21-3.15 (m, 2H), 3.15-3.06 (m, 1H), 1.32 (q, J=4.9 Hz, 6H). ¹³C-NMR (101 MHz, d₆-DMSO) δ 156.75 (d, J=231.2 Hz, 1C), 132.86 (s, 1C), 128.87 (s, 1C), 126.94 (d, J=9.9 Hz, 1C), 125.55 (s, 1C), 123.69 (s, 1C), 112.52 (d, J=9.7 Hz, 1C), 109.70 (d, J=4.7 Hz, 1C), 109.32 (d, J=26.0 Hz, 1C), 103.01 (d, J=23.0 Hz, 1C), 53.78 (s, 1C), 51.81 (s, 1C), 49.07 (s, 1C), 20.28 (s, 1C), 16.19 (s, 1C), 15.88 (s, 1C). ¹⁹F-NMR (377 MHz, d₆-DMSO) δ−124.66 (s, 1F).

### Example 12: N-ethyl-2-(5-fluoro-1H-indol-3-yl)-N-methylethan-1-amine (12)

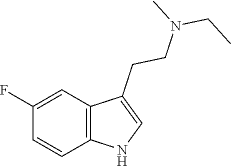 [SMILES: CCN(C)CCc1c[nH]c2ccc(F)cc12]

### Synthesis of N-[2-(5-fluoro-1H-indol-3-yl)ethyl]formamide

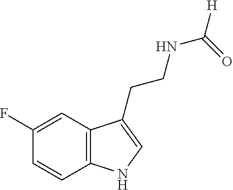 [SMILES: O=CNCCc1c[nH]c2ccc(F)cc12]

To a solution of 5-fluorotryptamine hydrochloride (3 g, 14.0 mmol) in H₂O (200 mL) with stirring was added KOH until a precipitate was obtained. The aqueous mixture was extracted with EtOAc (3×70 mL), the organic phases were pooled, washed with brine, dried over anhydrous Na₂SO₄, and concentrated in vacuo. Residual EtOAc was removed by azeotropic distillation with ethyl formate (3×20 mL). The resulting 5-fluorotryptamine free base was transferred to a 30 mL oven-dried microwave vessel containing 3 Å molecular sieves (3.3 g). Ethyl formate (20 mL, 248 mmol) was added to the microwave vessel and the mixture was reacted for 2.5 h at 80° C. with 150 W in a microwave reactor. Upon completion, ethyl formate was removed under reduced pressure to provide N-[2-(5-fluoro-1H-indol-3-yl)ethyl]formamide (1.7 g, 8.24 mmol, 58.9% yield). The product was used in the subsequent reaction without further purification.

### Synthesis of 2-(5-fluoro-1H-indol-3-yl)-N-methylethan-1-amine

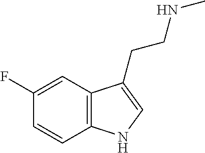

A three neck 500 mL round bottom flask, 300 mL addition funnel, stir bar, and condenser were dried overnight in an oven at 70° C., then further dried with external heat while flushing argon through the sealed system. Once drying was deemed completion the round bottom flask was placed into an ice bath (0° C.), and the apparatus was allowed to cool to room temperature. THF (100 mL) was added via the addition funnel and was allowed to reach 0° C., then sodium bis(2-methoxyethoxy)aluminium hydride (Red-Al) (18.41 mL, 59.6 mmol) was added to the reaction vessel. Next, N-[2-(5-fluoro-1H-indol-3-yl)ethyl]formamide (4.1 g, 19.8 mmol) in THE (20 mL) was added dropwise to the reaction vessel, with stirring, under argon over 30 mins at 0° C. Following the addition, the reaction was heated at reflux for 4 h. Upon completion, the reaction was cooled to 0° C. and cautiously quenched by the dropwise addition of THF/H₂O (1:1, v/v) with ice. Once quenched a small amount of KOH (aq.), then 100 mL of EtOAc were added.

The inorganic solids were then gravity filtered and washed with EtOAc. The filtrate was extracted with 0.2 M HCl (aq.) (3×166 mL). The pooled aqueous phases were then basified with KOH pellets and extracted with EtOAc (3×100 mL). The pooled organic extractions were washed with brine (20 mL), dried over anhydrous Na₂SO₄, and concentrated in vacuo to provide 2-(5-fluoro-1H-indol-3-yl)-N-methylethan-1-amine as a yellow oil (3.14 g, 0.0163 mol, 82.32% yield). The product was used in the subsequent reaction without further purification. ASAP-MS: m/z 193.2 (theoretical [M+H]⁺, C₁₁H₁₃FN₂⁺), m/z 193.2 (observed).

### Synthesis of N-ethyl-2-(5-fluoro-1H-indol-3-yl)-N-methylethan-1-amine (12)

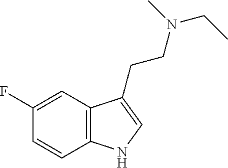 [SMILES: CCN(C)CCc1c[nH]c2ccc(F)cc12]

N-ethyl-2-(5-fluoro-1H-indol-3-yl)-N-methylethan-1-amine (12) was synthesized in a similar manner as described above for N-(2-(5-fluoro-1H-indol-3-yl)ethyl)-N-propylpropan-1-amine (5), starting from 2-(5-fluoro-1H-indol-3-yl)-N-methylethan-1-amine (0.7 g, 3.64 mmol), and acetaldehyde (0.96 g, 21.8 mmol), to provide the title compound as a colorless oil after purification by column chromatography using silica gel as a stationary phase and 20% EtOH/EtOAc (1% Et₃N v/v) as the mobile phase (0.62 g, 2.81 mmol, 77.2% yield), and subsequently the corresponding HCl salt as a white crystalline solid. HR-ASAP-MS: m/z 221.1442 (theoretical [M+H]⁺, C₁₃H₁₈FN₂⁺), m/z 221.1449 (observed, Δ=−3.2 ppm). ¹H-NMR (400 MHz, d₆-DMSO) δ 11.15 (s, 1H), 10.74 (s, 1H), 7.44 (dd, J=10.1, 2.5 Hz, 1H), 7.36 (dd, J=8.8, 4.6 Hz, 1H), 7.33 (d, J=2.3 Hz, 1H), 6.93 (dt, J=9.2, 2.5 Hz, 1H), 3.31-3.13 (m, 4H), 3.13-3.05 (m, 2H), 2.78 (d, J=3.1 Hz, 3H), 1.26 (t, J=7.3 Hz, 3H). ¹³C-NMR (101 MHz, d₆-DMSO) δ 156.74 (d, J=231.1 Hz, 1C), 132.88 (s, 1C), 126.96 (d, J=10.0 Hz, 1C), 125.42 (s, 1C), 112.48 (d, J=9.9 Hz, 1C), 109.51 (s, 1C), 109.32 (d, J=26.0 Hz, 1C), 103.13 (d, J=23.1 Hz, 1C), 54.37 (s, 1C), 49.82 (s, 1C), 38.13 (s, 1C), 19.68 (s, 1C), 8.79 (s, 1C). ¹⁹F-NMR (377 MHz, d₆-DMSO) δ−124.79 (s, 1F).

### Example 13: N-(2-(5-fluoro-1H-indol-3-yl)ethyl)-N-methylpropan-1-amine (13)

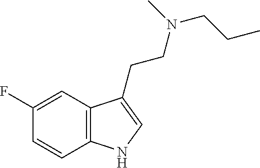 [SMILES: CCCN(C)CCc1c[nH]c2ccc(F)cc12]

N-(2-(5-fluoro-1H-indol-3-yl)ethyl)-N-methylpropan-1-amine (13) was synthesized in a similar manner as described above for N-(2-(5-fluoro-1H-indol-3-yl)ethyl)-N-propylpropan-1-amine (5), starting from 2-(5-fluoro-1H-indol-3-yl)-N-methylethan-1-amine (0.7 g, 3.64 mmol), and propionaldehyde (1.27 g, 21.8 mmol), to provide the title compound as a white crystalline solid (0.2 g, 0.854 mmol, m.p. 80-82° C., 23.46% yield). HR-ASAP-MS: m/z 235.1600 (theoretical [M+H]⁺, C₁₄H₂OFN₂⁺), m/z 235.1605 (observed, Δ=−2.1 ppm). (Free base) ¹H-NMR (400 MHz, d₆-DMSO) δ 10.86 (s, 1H), 7.31 (dd, J=8.8, 4.6 Hz, 1H), 7.25-7.20 (m, 2H), 6.88 (td, J=9.2, 2.5 Hz, 1H), 2.77 (t, J=7.8 Hz, 2H), 2.59-2.52 (m, 2H), 2.32 (t, J=7.3 Hz, 2H), 2.22 (s, 3H), 1.43 (sx, J=7.4 Hz, 2H), 0.85 (t, J=7.4 Hz, 3H). ¹³C NMR (101 MHz, d₆-DMSO) δ 156.56 (d, J=230.9 Hz, 1C), 132.80 (s, 1C), 127.47 (d, J=9.5 Hz, 1C), 124.64 (s, 1C), 113.11 (d, J=5.0 Hz, 1C), 112.16 (d, J=9.7 Hz, 1C), 108.82 (d, J=26.0 Hz, 1C), 102.88 (d, J=22.9 Hz, 1C), 59.04 (s, 1C), 58.02 (s, 1C), 41.76 (s, 1C), 22.58 (s, 1C), 20.04 (s, 1C), 11.82 (s, 1C). ¹⁹F-NMR (377 MHz, d₆-DMSO) δ−125.33 (s, 1F).

### Example 14: N-allyl-N-(2-(5-fluoro-1H-indol-3-yl)ethyl)cyclobutanamine (14)

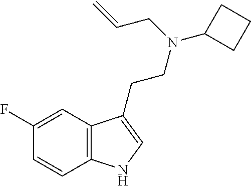 [SMILES: C=CCN(CCc1c[nH]c2ccc(F)cc12)C1CCC1]

### Synthesis of N-(2-(5-fluoro-1H-indol-3-yl)ethyl)cyclobutanamine

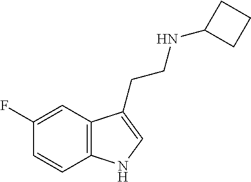 [SMILES: Fc1ccc2[nH]cc(CCNC3CCC3)c2c1]

N-(2-(5-fluoro-1H-indol-3-yl)ethyl)cyclobutanamine was synthesized in a similar manner as described above for N-(2-(5-fluoro-1H-indol-3-yl)ethyl)-N-propylpropan-1-amine (5), starting from 5-fluorotryptamine (1.0 g, 4.66 mmol), and cyclobutanone (1.96 g, 28.0 mmol), to provide the title compound after purification by column chromatography using silica gel as a stationary phase and 20% EtOH/EtOAc (1% Et₃N v/v) as the mobile phase (456 mg, 1.99 mmol, 42.7% yield).

### Synthesis of N-allyl-N-(2-(5-fluoro-1H-indol-3-yl)ethyl)cyclobutanamine (14)

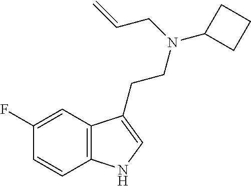 [SMILES: C=CCN(CCc1c[nH]c2ccc(F)cc12)C1CCC1]

N-allyl-N-(2-(5-fluoro-1H-indol-3-yl)ethyl)cyclobutanamine (14) was synthesized in a similar manner as described above for N-(2-(5-fluoro-1H-indol-3-yl)ethyl)-N-isopropylprop-2-en−1-amine (11), starting from N-(2-(5-fluoro-1H-indol-3-yl)ethyl)cyclobutanamine (0.21 g, 0.000904 mol) and allyl iodide (0.77 g, 0.00461 mol) as the starting materials to give N-allyl-N-(2-(5-fluoro-1H-indol-3-yl)ethyl)cyclobutanamine (0.11 g, 0.000404 mol, 44.7% yield) as a yellow-white solid. The HCl salt was collected as white crystalline solid, m.p. 196.3-199.0° C. High-resolution atmospheric solid analysis probe mass spectrometry (HR-ASAP-MS): m/z 273.1759 (theory [M+H]⁺ C₁₇H₂₂FN₂⁺, m/z 273.1762, Δ=−1.1 ppm). [HCl] ¹H NMR (400 MHz, d₆-DMSO) δ 11.38 (s, 1H), 11.13 (s, 1H), 7.39-7.32 (m, 3H), 6.93 (td, J=9.2, 2.4 Hz, 1H), 6.11-6.00 (m, 1H), 5.63 (dd, J=17.0, 0.9 Hz, 1H), 5.51 (dd, J=10.2, 1.1 Hz, 1H), 3.86-3.73 (m, 2H), 3.73-3.64 (m, 1H), 3.17-2.99 (m, 4H), 2.49-2.38 (m, 2H)*, 2.27-2.14 (m, 2H), 1.80-1.58 (m, 2H). *=Coalescing with water. [HCl] ¹³C NMR (101 MHz, d₆-DMSO) δ 156.76 (d, J=231.4 Hz, 1C), 132.85 (s, 1C), 127.04 (s, 1C), 126.88 (d, J=9.8 Hz, 1C), 125.51 (s, 1C), 124.81 (s, 1C), 112.56 (d, J=9.8 Hz, 1C), 109.56 (d, J=4.7 Hz, 1C), 109.35 (d, J=26.0 Hz, 1C), 102.88 (d, J=23.2 Hz, 1C), 56.44 (s, 1C), 50.80 (s, 1C), 48.95 (s, 1C), 25.85 (s, 1C), 25.78 (s, 1C), 19.03 (s, 1C), 13.38 (s, 1C). [HCl] ¹⁹F NMR (377 MHz, d₆-DMSO) δ−124.82 (s, 1F).

### Example 15: 5-fluoro-3-(1-methylpyrrolidin-3-yl)-1H-indole (15)

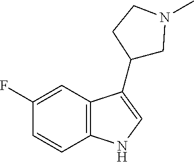 [SMILES: CN1CCC(c2c[nH]c3ccc(F)cc23)C1]

### Synthesis of 3-(5-fluoro-1H-indol-3-yl)-1-methylpyrrolidine-2,5-dione

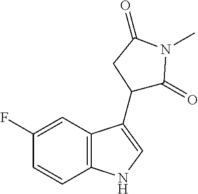 [SMILES: CN1C(=O)CC(c2c[nH]c3ccc(F)cc23)C1=O]

An oven-dried 35 mL microwave reaction vessel was charged with 5-fluoroindole (1.5 g, 11.1 mmol) and 1,2-dichloroethane (15 mL), then N-methyl maleimide (1.23 g, 11.1 mmol) and anhydrous ZnCl₂ (300 mg, 2.2 mmol) were added. The reaction vessel was heated to 90° C. at 150 W in a microwave reactor, with stirring, for 24 h. Upon completion, the solvent was removed in vacuo to provide a crude oil. The crude oil was dissolved in EtOAc, washed with water (3×100 mL) and brine (10 mL), dried over anhydrous Na₂SO₄, and concentrated in vacuo to provide the crude product. The crude product was recrystallized from boiling EtOAc to provide 3-(5-fluoro-1H-indol-3-yl)-1-methylpyrrolidine-2,5-dione as a yellow solid (2.4 g, 9.75 mmol, 87.8% yield).

### Synthesis of 5-fluoro-3-(1-methylpyrrolidin-3-yl)-1H-indole (15)

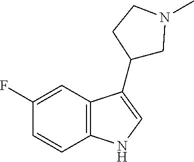 [SMILES: CN1CCC(c2c[nH]c3ccc(F)cc23)C1]

A three-neck 500 mL round bottom flask, 300 mL addition funnel, stir bar, and condenser was dried overnight at 70° C. The reaction apparatus was assembled and subsequently purged with argon, then placed in an ice-water bath (0° C.). Dry THF (100 mL, dried over 3 Å molecular sieves) was added to the round bottom flask and was allowed to reach 0° C. Next, LiAlH₄ (0.84 g, 0.022 mol) was added slowly to the reaction vessel. Once the addition was complete, a solution of 3-(5-fluoro-1H-indol-3-yl)-1-methylpyrrolidine-2,5-dione (1.82 g, 0.00740 mol) in THE (100 mL) was added dropwise over 1 h at 0° C. The addition funnel was rinsed with an additional THE (20 mL), which was added over a period of 10 min. The reaction was heated at reflux for 2 h. Upon completion, the reaction was cooled to 0° C. and quenched by the addition of a mixture of THF/H₂O (1:1 v/v) with ice added. Next, brine and KOH (aq.) were added, followed by EtOAc (100 mL). The inorganic solids were removed by gravity filtration over Whatman paper (24 cm diameter). The removed solids were then washed with EtOAc, and the filtrate was extracted with 0.2 M HCl (aq.) (3×166 mL) of a 0.2 M aqueous HCl solution. The aqueous phases were pooled and was basified by the addition of KOH flakes, then extracted with EtOAc (3×100 mL). The organic phases were pooled, washed with brine (20 mL), dried over anhydrous Na₂SO₄, and concentrated in vacuo to provide the crude product as a tannish-brown solid (1.53 g). The crude material was purified via flash column chromatography using silica as the stationary phase and 20% EtOH/EtOAc to provide the title compound as an off-white solid (1.13 g, 5.18 mmol, 70.0% yield, m.p. 122.8-125.1° C.). High-resolution atmospheric solid analysis probe mass spectrometry (HR-ASAP-MS): m/z 219.1287 (theory [M+H]⁺C₁₃H₁₆FN₂⁺, m/z 219.1292, Δ=−2.3 ppm). (Freebase)¹H-NMR (400 MHz, d₆-DMSO) δ 10.89 (s, 1H), 7.35 (dd, J=10.2, 2.6 Hz, 1H)*, 7.33 (dd, J=8.8, 4.4 Hz, 1H)*, 7.22 (d, J=2.4 Hz, 1H), 6.90 (dt, J=9.2, 2.5 Hz, 1H), 3.56-3.45 (m, 1H), 2.93 (t, J=8.4 Hz, 1H), 2.70-2.56 (m, 2H), 2.49-2.44 (m, 1H), 2.33 (s, 3H), 2.29-2.21 (m, 1H), 1.92-1.81 (m, 1H). *=coalescing, **=coalescing ¹³C NMR (101 MHz, DMSO) δ 156.42 (d, J=230.5 Hz, 1C), 133.30 (s, 1C), 126.44 (d, J=9.6 Hz, 1C), 123.28 (s, 1C), 118.43 (d, J=4.6 Hz, 1C), 112.27 (d, J=9.9 Hz, 1C), 108.95 (d, J=26.2 Hz, 1C), 103.53 (d, J=23.0 Hz, 1C), 62.44 (s, 1C), 55.90 (s, 1C), 42.01 (s, 1C), 34.52 (s, 1C), 31.96 (s, 1C). ¹⁹F-NMR (377 MHz, d₆-DMSO) δ−125.17 (s, 1F).

### Example 16: N-allyl-N-(2-(5-fluoro-1H-indol-3-yl)ethyl)butan-2-amine (16)

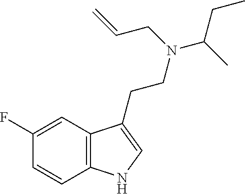 [SMILES: C=CCN(CCc1c[nH]c2ccc(F)cc12)C(C)CC]

### Synthesis of N-(2-(5-fluoro-1H-indol-3-yl)ethyl)butan-2-amine

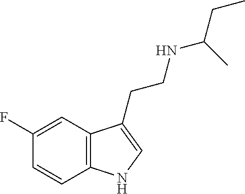 [SMILES: CCC(C)NCCc1c[nH]c2ccc(F)cc12]

N-(2-(5-fluoro-1H-indol-3-yl)ethyl)butan-2-amine was synthesized in a similar manner as described above for N-ethyl-N-(2-(5-fluoro-1H-indol-3-yl)ethyl)propan-2-amine (1), starting from 5-fluorotryptamine hydrochloride (1 g, 0.00466 mol), and methyl ethyl ketone (2.02 g, 0.0280 mol) as the starting materials to give N-(2-(5-fluoro-1H-indol-3-yl)ethyl)butan-2-amine as an off-yellow crystalline solid (0.69 g, 0.00294 mol, 63.1% yield). The material was purified by column chromatography utilizing silica gel as the stationary phase and a gradient from 5% to 20% EtOH in EtOAc with 1% TEA as the mobile phase.

### Synthesis of N-allyl-N-(2-(5-fluoro-1H-indol-3-yl)ethyl)butan-2-amine (16)

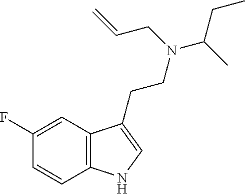 [SMILES: C=CCN(CCc1c[nH]c2ccc(F)cc12)C(C)CC]

N-allyl-N-(2-(5-fluoro-1H-indol-3-yl)ethyl)butan-2-amine was synthesized in a similar manner as described above for N-(2-(5-fluoro-1H-indol-3-yl)ethyl)-N-isopropylprop-2-en-1-amine (11), starting from N-(2-(5-fluoro-1H-indol-3-yl)ethyl)butan-2-amine (0.56 g, 0.00239 mol), and allyl iodide (2.02 g, 0.0120 mol) as the starting materials to give N-allyl-N-(2-(5-fluoro-1H-indol-3-yl)ethyl)butan-2-amine (0.18 g, 0.000656 mol, 27.5% yield) as a yellow oil. The HCl salt was collected as a white crystalline solid (m.p. 157.0-161.8° C.). High-resolution atmospheric solid analysis probe mass spectrometry (HR-ASAP-MS): m/z 275.1915 (theory [M+H]⁺ C₁₇H₂₄FN₂⁺, m/z 275.1918, Δ=−1.1 ppm). [Freebase] ¹H NMR (400 MHz, DMSO) δ 10.85 (s, 1H), 7.30 (dd, J=8.8 Hz, 4.6 Hz, 1H), 7.19 (d, J=2.3 Hz, 1H)*, 7.17 (dd, J=10.1, 2.5 Hz, 1H)*, 6.88 (td, J=9.2, 2.5 Hz, 1H), 5.82 (dddd, J=17.2, 8.6, 5.4, 3.4 Hz, 1H), 5.20 (dq, J=17.2, 1.7 Hz, 1H), 5.05 (d, J=10.0 Hz, 1H), 3.21 (ddt, J=14.5, 5.2, 1.6 Hz, 1H), 3.00 (dd, J=14.6, 6.9 Hz, 1H), 2.83-2.73 (m, 1H)**, 2.73-2.61 (m, 3H)**, 2.57-2.51 (m, 1H)***, 1.49-1.37 (m, 1H), 1.27-1.15 (m, 1H), 0.88 (d, J=6.5 Hz, 3H), 0.83 (t, J=7.4 Hz, 3H). *=coalescing, **=coalescing, **=coalescing with DMSO reference peak. [Freebase] ¹³C NMR (101 MHz, DMSO) δ 156.55 (d, J=230.8 Hz, 1C), 138.46 (s, 1C), 132.83 (s, 1C), 127.50 (d, J=9.6 Hz, 1C), 124.68 (s, 1C), 115.61 (s, 1C), 113.20 (d, J=4.9 Hz, 1C), 112.18 (d, J=9.7 Hz, 1C), 108.78 (d, J=26.0 Hz, 1C), 102.81 (d, J=22.8 Hz, 1C), 56.14 (s, 1C), 52.78 (s, 1C), 50.15 (s, 1C), 26.48 (s, 1C), 24.83 (s, 1C), 13.85 (s, 1C), 11.50 (s, 1C). [Freebase] ¹⁹F NMR (377 MHz, DMSO) δ−125.38 (s, 1F).

### Example 17: 2-((2-(5-fluoro-1H-indol-3-yl)ethyl)(isopropyl)amino)acetonitrile (17)

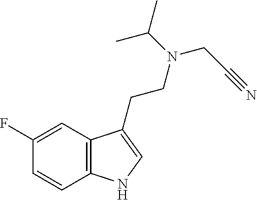 [SMILES: CC(C)N(CC#N)CCc1c[nH]c2ccc(F)cc12]

2-((2-(5-fluoro-1H-indol-3-yl)ethyl)(isopropyl)amino)acetonitrile was synthesized in a similar manner as described above for N-(2-(5-fluoro-1H-indol-3-yl)ethyl)-N-isopropylprop-2-en-1-amine (11), starting from N-(2-(5-fluoro-1H-indol-3-yl)ethyl)propan-2-amine (0.5 g, 0.00227 mol) and bromo acetonitrile (1.35 g, 0.0114 mol) as the starting materials to give 2-((2-(5-fluoro-1H-indol-3-yl)ethyl)(isopropyl)amino)acetonitrile as a yellow oil (0.11 g, 0.000424 mol, 18.67% yield). The HCl salt was collected as a slightly yellow solid, m.p. 147.0-150.0° C. with decomposition. High-resolution atmospheric solid analysis probe mass spectrometry (HR-ASAP-MS): m/z 260.1552 (theory [M+H]⁺ C₁₅H₁₉FN₃⁺, m/z 260.1558, Δ=−2.3 ppm). [HCl]¹H NMR (400 MHz, d₆-DMSO) δ 11.14 (s, 1H), 7.43 (dd, J=10.0, 2.4 Hz, 1H), 7.39-7.32 (m, 2H), 6.93 (td, J=9.2, 2.5 Hz, 1H), 4.63 (s, 2H), 3.72 (bs, 1H), 3.29 (s, 2H), 3.24-3.14 (m, 2H), 1.33 (d, J=6.5 Hz, 6H). [HCl] ¹³C NMR (101 MHz, d₆-DMSO) δ 156.79 (d, J=231.2 Hz, 1C), 132.88 (s, 1C), 126.95 (d, J=10.0 Hz, 1C), 125.67 (s, 1C), 114.22 (s, 1C), 112.57 (d, J=9.8 Hz, 1C), 109.38 (d, J=26.2 Hz, 1C), 109.33 (s, 1C), 103.00 (d, J=23.1 Hz, 1C), 56.41 (s, 1C), 50.60 (s, 1C), 36.70 (s, 1C), 20.38 (s, 1C), 16.51 (s, 1C). [HCl] ¹⁹F NMR (377 MHz, d₆-DMSO) δ−124.66 (s, 1F).

### Example 18: N-(2-(5-fluoro-1H-indol-3-yl)ethyl)prop-2-en-1-amine (18)

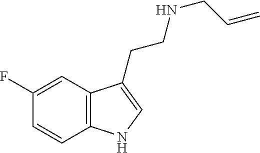 [SMILES: C=CCNCCc1c[nH]c2ccc(F)cc12]

### Synthesis of 2-(5-fluoro-1H-indol-3-yl)-2-oxo-N-(prop-2-en-1-yl)acetamide

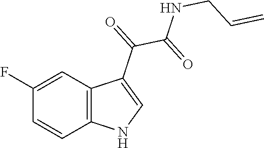 [SMILES: C=CCNC(=O)C(=O)c1c[nH]c2ccc(F)cc12]

2-(5-fluoro-1H-indol-3-yl)-2-oxo-N-(prop-2-en-1-yl)acetamide was synthesized in a similar manner as described above for N-ethyl-2-(7-fluoro-1H-indol-3-yl)-N-methylene-1-amine (7), starting from 5-fluoro-indole (5 g, 0.0370 mol), oxalyl chloride (5.64 g, 0.0444 mol), and allyl amine (7.59 g, 0.133 mol) as the starting material to give 2-(5-fluoro-1H-indol-3-yl)-2-oxo-N-(prop-2-en-1-yl)acetamide as an orange powder (9.5 g, 0.0386 mol, quantitative yield). Compound was used without further purification.

### Synthesis of N-(2-(5-fluoro-1H-indol-3-yl)ethyl)prop-2-en-1-amine (18)

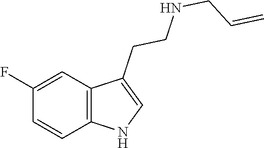 [SMILES: C=CCNCCc1c[nH]c2ccc(F)cc12]

N-(2-(5-fluoro-1H-indol-3-yl)ethyl)prop-2-en-1-amine was synthesized in a similar manner as described above for N-ethyl-2-(7-fluoro-1H-indol-3-yl)-N-methylene-1-amine (7), starting from 2-(5-fluoro-1H-indol-3-yl)-2-oxo-N-(prop-2-en-1-yl)acetamide (9.11 g, 0.0370 mol) as starting material to give N-(2-(5-fluoro-1H-indol-3-yl)ethyl)prop-2-en-1-amine as an orange oil (1.5 g, 0.00687 mol, 18.57% yield). High-resolution atmospheric solid analysis probe mass spectrometry (HR-ASAP-MS): m/z 219.1287 (theory [M+H]⁺ C₁₃H₁₆FN₂⁺, m/z 219.1292, Δ=−2.3 ppm). [Freebase] ¹H NMR (400 MHz, d₆-DMSO) δ 10.89 (s, 1H), 7.32 (dd, J=8.8, 4.6 Hz, 1H), 7.26 (dd, J=10.1, 2.5 Hz, 1H), 7.22 (d, J=2.2 Hz, 1H), 6.89 (td, J=9.2, 2.5 Hz, 1H), 5.85 (ddt, 17.3, 10.3, 5.74 Hz, 1H), 5.15 (dq, J=17.2, 1.7 Hz, 1H), 5.03 (dq, J=10.2, 1.4 Hz, 1H), 3.20 (dt, J=5.7, 1.3 Hz, 2H). 2.83-2.72 (m, 4H). [Freebase] ¹³C NMR (101 MHz, d₆-DMSO) δ 156.59 (d, J=230.6 Hz, 1C), 137.72 (s, 1C), 132.87 (s, 1C), 127.50 (d, J=9.6 Hz, 1C), 124.74 (s, 1C), 115.09 (s, 1C), 112.98 (d, J=5.0 Hz, 1C), 112.20 (d, J=9.9 Hz, 1C), 108.88 (d, J=26.2 Hz, 1C), 102.97 (d, J=23.0 Hz, 1C), 51.57 (s, 1C), 49.43 (s, 1C), 25.30 (s, 1C). [Freebase] ¹⁹F NMR (377 MHz, DMSO) δ−125.34 (s, 1F).

### Example 19: 5,6-difluoro-3-(1-methylpyrrolidin-3-yl)-1H-indole (19)

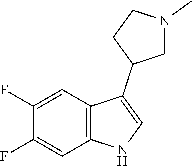 [SMILES: CN1CCC(c2c[nH]c3cc(F)c(F)cc23)C1]

### Synthesis of 3-(5,6-difluoro-H-indol-3-yl)-1-methylpyrrolidine-2,5-dione

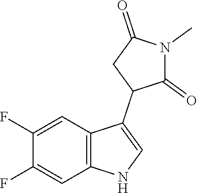 [SMILES: CN1C(=O)CC(c2c[nH]c3cc(F)c(F)cc23)C1=O]

3-(5,6-difluoro-1H-indol-3-yl)-1-methylpyrrolidine-2,5-dione was synthesized in a similar manner as described for 5-fluoro-3-(1-methylpyrrolidin-3-yl)-1H-indole (15), starting from 5,6-difluoroindole (1 g, 0.00653 mol) and N-methyl-maleimide (0.73 g, 0.00653 mol) as starting materials to give 3-(5,6-difluoro-1H-indol-3-yl)-1-methylpyrrolidine-2,5-dione as an orange solid (1.72 g, 0.00651 mol, 99.7% yield). Compound was used without further purification.

### Synthesis of 5,6-difluoro-3-(I-methylpyrrolidin-3-yl)-1H-indole (19)

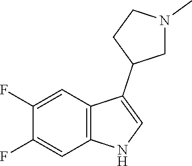 [SMILES: CN1CCC(c2c[nH]c3cc(F)c(F)cc23)C1]

5,6-difluoro-3-(1-methylpyrrolidin-3-yl)-1H-indole was synthesized in a similar manner as described for 5-fluoro-3-(1-methylpyrrolidin-3-yl)-1H-indole (15), starting from 3-(5,6-difluoro-1H-indol-3-yl)-1-methylpyrrolidine-2,5-dione (1.7 g, 0.00643 mol) as starting materials to provide the title compound (0.93 g, 0.00394 mol, 61.3% yield) (m.p. 119.5-120.3° C.). High-resolution atmospheric solid analysis probe mass spectrometry (HR-ASAP-MS): m/z 237.1196 (theory [M+H]⁺ C₁₃H₁₅F₂N₂⁺, m/z 237.1198, Δ=−0.8 ppm). [Freebase] ¹H NMR (400 MHz, d₆-DMSO) δ 10.93 (s, 1H), 7.57 (dd, J=11.6, 8.1 Hz, 1H), 7.31 (dd, J=11.3, 7.0 Hz, 1H), 7.20 (d, J=2.3 Hz, 1H), 3.48 (dq, J=14.1, 7.3 Hz, 1H), 2.88 (t, J=8.3 Hz, 1H), 2.65-2.54 (m, 2H), 2.44 (dd, J=8.8, 7.1 Hz, 1H), 2.30 (s, 3H), 2.27-2.18 (m, 1H), 1.87-1.76 (m, 1H). [Freebase] ¹³C NMR (101 MHz, d₆-DMSO) δ 146.45 (dd, J=236.4, 16.1 Hz, 1C), 144.65 (dd, J=233.6, 14.9 Hz, 1C), 131.55 (d, J=10.7 Hz, 1C), 123.07 (d, J=3.7 Hz, 1C), 121.64 (d, J=7.7 Hz, 1C), 118.94 (dd, J=4.4, 1.5 Hz, 1C), 105.45 (d, J=18.8 Hz, 1C), 99.14 (d, J=20.9 Hz, 1C), 62.51 (s, 1C), 55.91 (s, 1C), 42.01 (s, 1C), 34.38 (s, 1C), 32.06 (s, 1C). [Freebase] ¹⁹F NMR (377 MHz, d₆-DMSO) δ−145.54 (d, J=22.8 Hz, 1F), −148.96 (d, J=22.0 Hz, 1F).

### Example 20: N-(2-(5-fluoro-1H-indol-3-yl)ethyl)-N-methylthietan-3-amine (20)

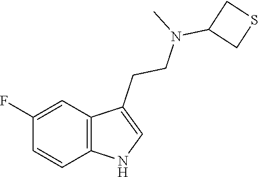 [SMILES: CN(CCc1c[nH]c2ccc(F)cc12)C1CSC1]

N-(2-(5-fluoro-1H-indol-3-yl)ethyl)-N-methylthietan-3-amine was synthesized in a similar manner as described above for N-ethyl-N-(2-(5-fluoro-1H-indol-3-yl)ethyl)propan-2-amine (1), starting from 5-fluorotryptamine hydrochloride 5-fluoro-tryptamine hydrochloride (0.5 g, 0.00233 mol), thietan-3-one (0.62 g, 0.00699 mol) and 36% formaldehyde in water (0.58 mL, 0.00699 mol) as starting materials to give the title compound as a cloudy white oil (0.19 g, 0.000719 mol, 30.9% yield). The HCl was collected as a white crystalline solid (m.p. 221.0-223.3° C.). High-resolution atmospheric solid analysis probe mass spectrometry (HR-ASAP-MS): m/z 265.1166 (theory [M+H]⁺ C₁₄H₁₈FSN₂⁺, m/z 265.1169, Δ=−1.1 ppm). [HCl] ¹H NMR (400 MHz, d₆-DMSO) δ 11.68 (s, 1H), 11.13 (s, 1H), 7.44 (dd, J=10, 2.5, Hz, 1H), 7.36 (dd, J=9.0, 4.7 Hz, 1H)*, 7.34 (d, J=2.1 Hz, 1H), 6.93 (dt, J=9.2, 2.5, Hz, 1H), 4.61-4.47 (m, 1H), 3.92-3.77 (m, 2H), 3.25-2.99 (m, 6H), 2.71 (s, 3H). *=coalescing. [HCl] ¹³C NMR (101 MHz, d₆-DMSO) δ 156.78 (d, J=231.3 Hz, 1C), 132.86 (s, 1C), 126.92 (d, J=9.9 Hz, 1C), 125.55 (s, 1C), 112.52 (d, J=9.8 Hz, 1C), 109.37 (d, J=4.5 Hz, 1C)*, 109.37 (d, J=26.1 Hz, 1C)*, 103.09 (d, J=23.1 Hz, 1C), 58.80 (s, 1C), 50.92 (s, 1C), 34.39 (s, 1C), 29.12 (s, 1C), 28.76 (s, 1C), 19.29 (s, 1C). *=coalescing. ¹⁹F NMR (377 MHz, DMSO) δ−124.69 (s, 1F).

### Example 21: N-(2-(4,5-difluoro-1H-indol-3-yl)ethyl)-N-ethylpropan-1-amine (21)

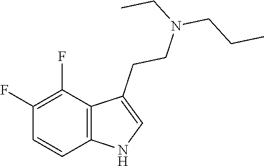 [SMILES: CCCN(CC)CCc1c[nH]c2ccc(F)c(F)c12]

### Synthesis of 2-(4,5-difluoro-H-indol-3-yl)-N-ethyl-2-oxo-N-propylacetamide

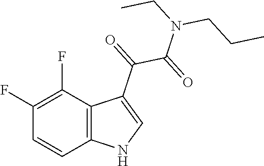 [SMILES: CCCN(CC)C(=O)C(=O)c1c[nH]c2ccc(F)c(F)c12]

2-(4,5-difluoro-1H-indol-3-yl)-N-ethyl-2-oxo-N-propylacetamide was synthesized in a similar manner as described above for N-ethyl-2-(7-fluoro-1H-indol-3-yl)-N-methylene-1-amine (7), starting from 4,5-difluoroindole (5 g, 0.0327 mol), oxalyl chloride (4.98 g, 0.0392 mol), and N-ethylpropyl amine (4.1 g, 0.047 mol) as the starting materials to give 2-(4,5-difluoro-1H-indol-3-yl)-N-ethyl-2-oxo-N-propylacetamide as an orange solid (6.9 g, 0.0234 mol, 71.6% yield). Column chromatography was performed utilizing silica gel as the stationary phase with 20% EtOH in EtOAc with 1% triethylamine as the mobile phase. Compound was used without further purification.

### Synthesis of N-[2-(4,5-difluoro-1H-indol-3-yl)ethyl]-N-ethylpropan-1-amine (21)

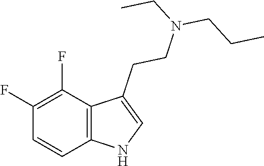 [SMILES: CCCN(CC)CCc1c[nH]c2ccc(F)c(F)c12]

N-(2-(4,5-difluoro-1H-indol-3-yl)ethyl)-N-ethylpropan-1-amine was synthesized in a similar manner as described above for N-ethyl-2-(7-fluoro-1H-indol-3-yl)-N-methylene-1-amine (7), starting from 2-(4,5-difluoro-1H-indol-3-yl)-N-ethyl-2-oxo-N-propylacetamide (6.9 g, 0.0234 mol) as the starting material to give provide the fumarate salt of the title compound as a white crystalline solid (2.1 g, 0.00549 mol, 23.5% yield) (m.p. 182.1-183.3° C.). The free base was immediately converted in the hemi fumarate salt upon evaporating. High-resolution atmospheric solid analysis probe mass spectrometry (HR-ASAP-MS): m/z 267.1660 (theory [M+H]⁺ C₁₅H₂₁F₂N₂⁺, m/z 267.1667, Δ=−2.6 ppm). [Hemi Fumarate] ¹H NMR (400 MHz, d₆-DMSO) δ 11.26 (s, 1H), 7.28 (d, J=2.1 Hz, 1H), 7.13 (dd, J=8.8, 3.6 Hz, 1H), 7.010-7.02 (m, 1H), 6.50 (s, 1H), 3.05-2.90 (m, 2H), 2.9-2.81 (m, 2H), 2.75 (q, J=7.1 Hz, 2H), 2.67-2.59 (m, 2H), 1.57-1.42 (m, 2H), 1.06 (t, J=7.1 Hz, 3H), 0.86 (t, J=7.3 Hz, 3H). [Hemi Fumarate] ¹³C NMR (101 MHz, DMSO) δ 167.53 (s, 1C), 142.94 (dd, J=11.3, 230.7 Hz, 1C), 142.69 (dd, J=14.3, 244.1 Hz, 1C), 134.92 (s, 1C)*, 134.91 (d, J=10.4 Hz, 1C)*, 125.67 (s, 1C), 116.24 (d, J=15.4 Hz, 1C), 110.57 (dd, J=2.2, 5.1 Hz, 1C)**, 110.38 (d, J=21.4 Hz, 1C)**, 107.58 (dd, J=3.8, 7.8 Hz, 1C), 54.12 (s, 1C), 53.73 (s, 1C), 46.77 (s, 1C), 22.36 (s, 1C), 18.83 (s, 1C), 11.57 (s, 1C), 10.71 (s, 1C). *=coalescing, **=coalescing. [Hemi Fumarate] ¹⁹F NMR (377 MHz, DMSO) δ−152.36 (d, J=21.8 Hz, 1F), −154.01 (d, J==21.8 Hz, 1F).

### Example 22: N-(2-(5-fluoro-1H-indol-3-yl)ethyl)-N-propylbutan-2-amine (22)

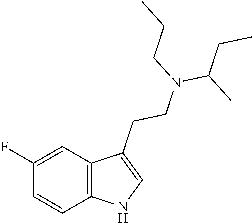 [SMILES: CCCN(CCc1c[nH]c2ccc(F)cc12)C(C)CC]

N-(2-(5-fluoro-1H-indol-3-yl)ethyl)-N-propylbutan-2-amine was synthesized in a similar manner as described above for N-ethyl-N-(2-(5-fluoro-1H-indol-3-yl)ethyl)propan-2-amine (1), starting from 5-fluorotryptamine hydrochloride 5-fluoro-tryptamine hydrochloride (0.5 g, 0.00233 mol), methyl ethyl ketone (0.51 g, 0.00699 mol), and propionaldehyde (0.41 g, 0.0699 mol) as the starting materials to provide the title compound as a yellow oil (0.34 g, 0.00110 mol, 47.2% yield). The hydrochloride salt was collected as a white solid (m.p. 153.2-155.4° C.). High-resolution atmospheric solid analysis probe mass spectrometry (HR-ASAP-MS): m/z 277.2069 (theory [M+H]⁺ C₁₃H₂₆FN₂⁺, m/z 277.2075, Δ=−2.2 ppm). [Freebase] ¹H NMR (400 MHz, DMSO) δ 10.85 (s, 1H), 7.31 (dd, J=8.8, 4.6 Hz, 1H), 7.21 (d, J=2.2 Hz, 1H), 7.17 (dd, J=10.0, 2.5 Hz, 1H), 6.88 (td, J=9.1, 2.5 Hz, 1H), 2.80-2.57 (m, 4H), 2.55-2.5 (m, 1H)*, 2.46-2.29 (m, 2H), 1.48-1.31 (m, 3H), 1.26-1.14 (m, 1H), 0.89-0.91 (m, 9H). *=coalescing with DMSO. [Freebase] ¹³C NMR (101 MHz, DMSO) δ 156.56 (d, J=230.7 Hz, 1C), 132.83 (s, 1C), 127.49 (d, J=9.6 Hz, 1C), 124.70 (s, 1C), 113.28 (d, J=4.6 Hz, 1C), 112.18 (d, J=9.9 Hz, 1C), 108.76 (d, J=26.0 Hz, 1C), 102.71 (d, J=22.8 Hz, 1C), 56.33 (s, 1C), 51.51 (s, 1C), 50.61 (s, 1C), 26.55 (s, 1C), 25.26 (s, 1C), 21.95 (s, 1C), 13.78 (s, 1C), 11.81 (s, 1C), 11.60 (s, 1C). [Freebase] ¹⁹F NMR (377 MHz, DMSO) δ−125.40 (s, 1F).

### Example 23: 7-fluoro-3-(1-methylpyrrolidin-3-yl)-1H-indole (23)

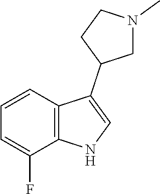 [SMILES: CN1CCC(c2c[nH]c3c(F)cccc23)C1]

### Synthesis of 3-(7-fluoro-1H-indol-3-yl)-1-methylpyrrolidine-2,5-dione

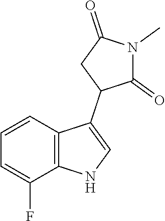 [SMILES: CN1C(=O)CC(c2c[nH]c3c(F)cccc23)C1=O]

3-(7-fluoro-1H-indol-3-yl)-1-methylpyrrolidine-2,5-dione was synthesized in a similar manner as described for 5-fluoro-3-(1-methylpyrrolidin-3-yl)-1H-indole (15), starting from 7-fluoroindole (01 g, 0.00740 mol) and N-methyl maleimide (0.82 g, 0.0074 mol) as the starting materials to give 3-(7-fluoro-1H-indol-3-yl)-1-methylpyrrolidine-2,5-dione as a yellow/orange crystalline solid (1.4 g, 0.00569 mol, 76.9% yield). The compound was used without further purification. 7-fluoro-3-(I-methylpyrrolidin-3-yl)-1H-indole (23)

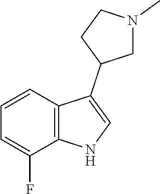 [SMILES: CN1CCC(c2c[nH]c3c(F)cccc23)C1]

7-Fluoro-3-(1-methylpyrrolidin-3-yl)-1H-indole was synthesized in a similar manner as described for 5-fluoro-3-(1-methylpyrrolidin-3-yl)-1H-indole (15), utilizing 3-(7-fluoro-1H-indol-3-yl)-1-methylpyrrolidine-2,5-dione (1.4 g, 0.00569 mol) to provide the title compound as a light orange crystalline solid, that was recrystallized from ethyl acetate and hexanes to give white crystalline solid clusters that discolored slightly discolored (beige-tan) on exposure to air (0.49 g, 0.00224 mol, 39.37% yield), m.p. 129.0-132.1° C. High-resolution atmospheric solid analysis probe mass spectrometry (HR-ASAP-MS): m/z 219.1291 (theory [M+H]⁺ C₁₃H₁₆FN₂⁺, m/z 219.1292, Δ=−0.5 ppm). [Freebase] ¹H NMR (400 MHz, d₆-DMSO) δ 11.26 (s, 1H), 7.41 (d, J=7.5 Hz, 1H), 7.20 (d, J=2.3 Hz, 1H), 6.96-6.85 (m, 2H), 3.59-3.50 (m, 1H), 2.96 (t, J=8.3 Hz, 1H), 2.70 (q, J=7.6 Hz, 1H), 2.61-2.54 (m, 1H), 2.50-2.44 (m, 1H)*, 2.32 (s, 3H), 2.30-2.22 (m, 1H), 1.94-1.83 (m, 1H). *=coalescing with DMSO [Freebase] ¹³C NMR (101 MHz, d₆-DMSO) δ 149.28 (d, J=242.4 Hz, 1C), 130.46 (d, J=6.1 Hz, 1C), 124.35 (d, J=13.1 Hz, 1C), 122.38 (s, 1C), 119.18 (d, J=1.8 Hz, 1C), 118.41 (d, J=6.1 Hz, 1C), 115.16 (d, J=3.1 Hz, 1C), 105.73 (d, J=16.0 Hz, 1C), 62.48 (s, 1C), 55.83 (s, 1C), 42.01 (s, 1C), 34.62 (s, 1C), 31.93 (s, 1C). [Freebase] ¹⁹F NMR (377 MHz, d₆-DMSO) δ−133.27 (s, 1F).

### Example 24: 2-(ethyl(propyl)amino)-1-(5-fluoro-1H-indol-3-yl)ethan-1-one (24)

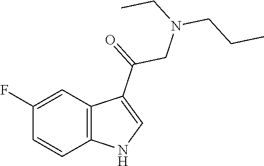 [SMILES: CCCN(CC)CC(=O)c1c[nH]c2ccc(F)cc12]

### Synthesis of 2-chloro-1-(5-fluoro-1H-indol-3-yl)ethan-1-one

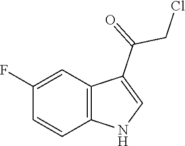 [SMILES: O=C(CCl)c1c[nH]c2ccc(F)cc12]

5-fluoroindole (2.5 g, 0.0185 mol) was dissolved in 40 mL of toluene along with pyridine (1.46 g, 0.0185 mol). This was then placed in a 60° C. water bath and an addition funnel was fitted with chloroacetyl chloride (2.09 g, 0.0185 mol) in 15 mL of toluene and added dropwise over 30 minutes. This was then allowed to react for 30 more minutes after the addition was complete. The reaction removed from the heat and a solution of 30 mL H₂O and 6 mL MeOH was added and allowed to stir for a 2 h period prior to vacuum filtering the solids from the reaction. After suction filtration was complete 2-chloro-1-(5-fluoro-1H-indol-3-yl)ethan-1-one was obtained as a yellow crystalline solid (1.3 g, 0.00614 mol, 33.3% yield). The product was used without further purification.

### Synthesis of 2-(ethyl(propyl)amino)-1-(5-fluoro-1H-indol-3-yl)ethan-1-one (24)

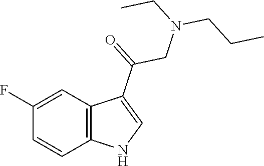 [SMILES: CCCN(CC)CC(=O)c1c[nH]c2ccc(F)cc12]

In a dried 35 mL microwave vessel, stir bar, and cap were dried in the oven overnight and heated additionally externally with hot air from a heat gun in order to drive off any moisture and then 25 mL of acetonitrile was added to the microwave vessel along with N-ethylpropylamine (0.75 g, 0.00860 mol). Then potassium iodide (1.43 g, 0.00860 mol) was added, and finally 2-chloro-1-(5-fluoro-1H-indol-3-yl)ethan-1-one (0.61 g, 0.00287 mol) was added to the reaction allowed to react for about 3 h. The reaction mixture was taken and dissolved in 250 mL of 0.174 M acetic acid solution. Two extractions were performed with EtOAc utilizing 50 mL each time. The extractions were pooled and were extracted 3×250 mL of 0.174 M acetic acid solution to extract any product that may have partitioned into the organic phase. The acetic acid washes were pooled with the initial acetic acid solution to form a total of 500 mL of 0.174 M acetic acid solution. This was basified with KOH until pH>12 and the precipitate was extracted three times with 100 mL of EtOAc each time (3×100 mL). Once the EtOAc extractions were complete, they were combined and washed with 60 mL of brine solution. Finally, the organic phase was dried utilizing sodium sulfate and was decanted, and removed under reduced pressure to yield 635 mg as a yellow solid.

The freebase was converted to the HCl salt by dissolving the freebase in 20 mL of EtOH and then adding 206 μL of 36.5% HCl directly to the solution. The solvent was then evaporated under a stream of warm air several times to yield crystalline material with the absence of excess acid or moisture. The resulting solids were washed with EtOAc (2×5 mL) and Et₂O (10 mL). Crystallization was performed by dissolving the solids into 7 mL of boiling EtOH and then slowly adding 25 mL Et₂O until the solution was milky white and opaque. The solution was allowed to recover to room temperature (RT) and allowed to sit for 1 h, before placing at 4° C. until thermodynamic equilibrium was attained and then stored at −20° C. overnight. The resulting crystals were collected by decanting the supernatant and the recrystallization was repeated a total of three time to yield 340 mg (39.7% yield) of a yellow crystalline solid, m.p. 203.4-205.6° C. High-resolution atmospheric solid analysis probe mass spectrometry (HR-ASAP-MS): m/z 263.1549 (theory [M+H]⁺C₁₅H₂₀FON₂⁺, m/z 263.1554, Δ=−1.9 ppm). [HCl] ¹H NMR (400 MHz, d₆-DMSO) δ 12.87 (s, 1H), 9.83 (s, 1H), 8.63 (d, J=3.2 Hz, 1H), 7.83 (dd, J=9.7, 2.5 Hz, 1H), 7.58 (dd, J=8.9, 4.6 Hz, 1H), 7.13 (td, J=9.1, 2.4 Hz, 1H), 4.80 (s, 2H), 3.26 (q, J=6.8 Hz, 2H), 3.11 (at, J=7.7 Hz, 2H), 1.72 (sex, J=7.7 Hz, 2H), 1.27 (t, J=7.2 Hz, 3H), 0.90 (t, J=7.3 Hz, 3H). [HCl] ¹³C NMR (101 MHz, d₆-DMSO) δ 185.74 (s, 1C), 158.93 (d, J=235.6 Hz, 1C), 137.17 (s, 1C), 133.23 (s, 1C), 125.71 (d, J=11.1 Hz, 1C), 114.05 (d, J=9.9 Hz, 1C), 113.29 (d, J=4.3 Hz, 1C), 111.62 (d, J=25.9 Hz, 1C), 105.87 (d, J=24.7 Hz, 1C), 56.75 (s, 1C), 55.03 (s, 1C), 49.36 (s, 1C), 16.83 (s, 1C), 10.80 (s, 1C), 8.89 (s, 1C). [HCl] ¹⁹F NMR (377 MHz, DMSO) δ−120.14 (s, 1F).

### Example 25: 2-(ethyl(methyl)amino)-1-(5-fluoro-1H-indol-3-yl)ethan-1-one (25)

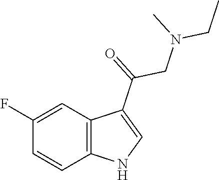 [SMILES: CCN(C)CC(=O)c1c[nH]c2ccc(F)cc12]

2-(ethyl(methyl)amino)-1-(5-fluoro-1H-indol-3-yl)ethan-1-one was synthesized in a similar manner as described above for 2-(ethyl(propyl)amino)-1-(5-fluoro-1H-indol-3-yl)ethan-1-one, starting from 2-chloro-1-(5-fluoro-1H-indol-3-yl)ethan-1-one (0.6 g, 0.00284 mol) and N-methylethylamine (0.5 g, 0.00852 mol) as the starting materials to provide the title compound as a white/yellow crystalline solid (0.18 g, 0.000768 mol, 27.0% yield), m.p. 160.1-164.8° C. The product was purified by column chromatography utilizing silica gel as the stationary phase and the mobile phase gradient of 2% EtOH to 20% EtOH in EtOAc with 1% triethylamine. High-resolution atmospheric solid analysis probe mass spectrometry (HR-ASAP-MS): m/z 235.1233 (theory [M+H]⁺C₁₃H₁₆FON₂⁺, m/z 235.1241, Δ=−3.4 ppm). [Freebase] ¹H NMR (400 MHz, d₆-DMSO) δ 11.99 (s, 1H), 8.51 (s, 1H), 7.85 (dd, J=10.0, 2.6 Hz, 1H), 7.48 (dd, J=8.8, 4.6 Hz, 1H), 7.06 (td, J=9.1, 2.6 Hz, 1H), 3.58 (s, 2H), 2.54-2.51 (m, J=2.5 Hz, 2H)*, 2.25 (s, 3H), 1.03 (t, J=7.1 Hz, 3H). [Freebase] ¹³C NMR (101 MHz, DMSO) δ 193.44 (s, 1C), 158.59 (d, J=234.2 Hz, 1C), 135.64 (s, 1C), 132.81 (s, 1C), 126.25 (d, J=11.0 Hz, 1C), 115.10 (d, J=4.4 Hz, 1C), 113.31 (d, J=10.0 Hz, 1C), 110.83 (d, J=25.9 Hz, 1C), 106.09 (d, J=24.4 Hz, 1C), 64.43 (s, 1C), 51.09 (s, 1C), 41.85 (s, 1C), 12.30 (s, 1C). [Freebase] ¹⁹F NMR (377 MHz, DMSO) δ−121.35 (s, 1F).

### Example 26: 2-(5-fluoro-1H-indol-3-yl)-N,N-dimethylpropan-1-amine (26)

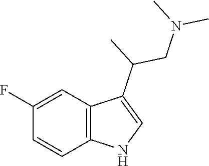 [SMILES: CC(CN(C)C)c1c[nH]c2ccc(F)cc12]

### Synthesis of 5-fluoro-3-[2-nitroethenyl]-1H-indole

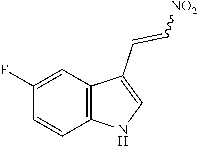 [SMILES: O=[N+]([O-])C=Cc1c[nH]c2ccc(F)cc12]

A dry 100 mL one neck round bottom flask was dried in an oven overnight and then, with internal flow of argon, heated externally with hot air from a heat gun, and was allowed to cool to room temperature under argon flow. After the round bottom flask has cooled to room temperature, 5-fluoroindole-3-carboxaldehyde (5 g, 0.0306 mol) was added to the flask. Then nitromethane (40 mL) was added to the round bottom flask, along with ammonium acetate (1.34 g, 0.0174 mol) and reflux was initiated under a heat mantle. This reflux was maintained for 3 h. After 3 h, the reaction was terminated by removing the nitromethane under reduced pressure to yield a red-orange crystalline material. This material was recrystallized in the same round bottom flask by dissolving the crystalline material in 60 mL of boiling Isopropanol (IPA) and allowed to cool to room temperature. After cooling down to RT, the round bottom flask with a septum was placed at −20° C. and allowed to recrystallize overnight. Following overnight recrystallization, the IPA was decanted and the crystalline material was dried under reduced pressure to provide 5-fluoro-3-(2-nitroethenyl)-1H-indole as an orange crystalline solid (5.14 g, 0.0249 mol, 81.4% yield). The product was used without further purification.

### Synthesis of 5-fluoro-3-(I-nitropropan-2-yl)-1H-indole

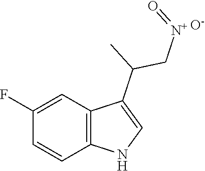

In an oven dried two-neck 250 mL round bottom flask was fitted with an addition funnel and 70 mL of THE was added to the round bottom flask. Then 1.4 M methylmagnesium bromide (43.47 mL, 0.0608 mol) in 1:3 THF/Toluene solution was added to round bottom flask through a syringe under argon flow over 30 minutes. Once the addition was complete, 5-fluoro-3-[2-nitroethenyl]-1H-indole (5 g, 0.0243 mol) was added to the round bottom flask and dissolved in 30 mL of THF. Once dissolved in THF, the 5-fluoro-3-[2-nitroethenyl]-1H-indole was added dropwise over 30 minutes keeping the temperature below 35° C. Once addition was complete, the reaction was allowed to stir for 1.5 h. After 1.5 h, a small portion of the reaction was taken and examined by TLC in 20% EtOAc in hexanes and no starting material was detected. The reaction was then worked up by mixing the reaction solution in 700 mL of saturated ammonium chloride solution and was extracted five times with 110 mL of EtOAc (5×110 mL). Once extracted the EtOAc extractions were pooled and washed with 60 mL of brine (1×60 mL). Then the EtOAc was dried with sodium sulfate and decanted to remove the solvent under pressure. The dark brown oil was then purified by column chromatography utilizing silica gel as the stationary phase and 20% EtOAc in Hexanes (isomers) as the mobile phase to yield pure 5-fluoro-3-(1-nitropropan-2-yl)-1H-indole as an orange-red oil (3.0 g, 0.0137 mol, 56.4% yield).

The product was used without further purification.

### Synthesis of 2-(5-fluoro-1H-indol-3-yl)propan-1-amine

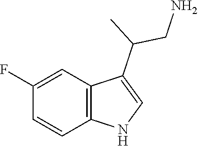

A dry 100 mL one neck round bottom flask was dried in an oven overnight and then, with internal flow of argon, heated externally with hot air from a heat gun, and was allowed to cool back down to RT under argon flow. After cooling down to RT, methanol was added (70 mL) to the round bottom flask along with 10% Palladium on carbon (1.46 g, 0.0137 mol). Then 5-fluoro-3-(1-nitropropan-2-yl)-1H-indole (3.04 g, 0.0137 mol) was dissolve in methanol (30 mL) and added to the round bottom while under constant argon flow. Then a balloon that was filled with hydrogen was attached to a modified syringe and pierced through the septum and allowed to react for 4 h. After 4 h the reaction was terminated by filtering the Pd-C through a celite plug and collecting the filtrate. The methanol was removed under reduced pressure and the material was dissolved in 100 mL of EtOAc. This was then extracted five times with 100 mL of 0.285 M HCl each time (5×100 mL). The extractions were pooled and basified with KOH to obtain a pH>12. Then following basified the product was extracted from the aqueous phase three times with 100 mL of EtOAc each time (3×100 mL). Once the final extraction was performed, they were pooled and washed with 60 mL of brine once (1×60 mL). The organic phase was dried with sodium sulfate, decanted, and removed under reduced pressure to obtain 2.24 g of crude oil.

This was purified by column chromatography utilizing silica gel as the stationary phase and a gradient of 20% to 50% EtOH in EtOAc with 1% triethylamine to provide the title compound as a yellow oil (1.27 g, 0.00661 mol, 48.3% yield). The portion of the freebase was converted to the HCl salt by dissolving the freebase in 20 mL of EtOH and then titrating the pH<2. The solvent was then evaporated under a stream of warm air several times to yield crystalline material with the absence of excess acid or moisture. The resulting solids were washed with 2×7 mL of EtOAc and with 1×10 mL of Et₂O. Crystallization was performed by dissolving the solids into 7 mL of boiling EtOH and then slowly adding 25 mL Et₂O until the solution was milky white and opaque. The solution was allowed to recover to room temperature and allowed to sit for 1 h, before placing at 4° C. until thermodynamic equilibrium was attained and then stored at −20° C. overnight. The resulting crystals were collected by decanting the supernatant and the recrystallization was repeated a total of three time to yield a white crystalline solid (m.p. 193.3-194.0° C.). High-resolution atmospheric solid analysis probe mass spectrometry (HR-ASAP-MS): m/z 193.1135 (theory [M+H]⁺ C₁₁H₁₄FN₂⁺, m/z 193.1136, Δ=−0.5 ppm). [HCl]¹H NMR (400 MHz, d₆-DMSO) δ 11.17 (s, 1H), 8.08 (s, 3H), 7.42 (dd, J=10.2, 2.5 Hz, 1H), 7.36 (dd, J=8.8, 4.6 Hz, 1H), 7.31 (d, J=2.5 Hz, 1H), 6.92 (td, J=9.2, 2.5 Hz, 1H), 3.36-3.27 (m, 1H)*, 3.14-2.99 (m, 1H), 2.99-2.86 (m, 1H), 1.36 (d, J=7.0 Hz, 3H). *=coalescing with water. [HCl] ¹³C NMR (101 MHz, d₆-DMSO) δ 156.64 (d, J=231.0 Hz, 1C), 133.14 (s, 1C), 126.22 (d, J=9.7 Hz, 1C), 124.34 (s, 1C), 115.88 (d, J=4.7 Hz, 1C), 112.52 (d, J=9.8 Hz, 1C), 109.21 (d, J=26.1 Hz, 1C), 103.26 (d, J=23.3 Hz, 1C), 44.71 (s, 1C), 28.95 (s, 1C), 18.40 (s, 1C). [HCl] ¹⁹F NMR (377 MHz, DMSO) δ−124.82 (s, 1F).

### Synthesis of 2-(5-fluoro-1H-indol-3-yl)-N,N-dimethylpropan-1-amine (26)

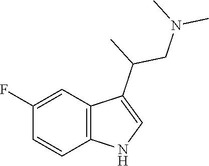 [SMILES: CC(CN(C)C)c1c[nH]c2ccc(F)cc12]

2-(5-fluoro-1H-indol-3-yl)-N,N-dimethylpropan-1-amine was synthesized in a similar manner as described above for N-(2-(5-fluoro-1H-indol-3-yl)ethyl)-N-propylpropan-1-amine (5), starting from 2-(5-fluoro-1H-indol-3-yl)propan-1-amine (0.5 g, 0.00260 mol), and 36% formaldehyde solution in H₂O (1.3 mL, 0.0156 mol) as the starting materials to provide the title compound as a white crystalline solid (0.36 g, 0.00164 mol, 63.2% yield). Column chromatography was performed utilizing silica gel as the stationary phase, and a gradient from 5% to 20% EtOH in EtOAc with 1% triethylamine. High-resolution atmospheric solid analysis probe mass spectrometry (HR-ASAP-MS): m/z 221.1445 (theory [M+H]⁺ C₁₃H₁₈FN₂⁺, m/z 221.1449, Δ=−1.8 ppm). [HCl] ¹H NMR (400 MHz, d₆-DMSO) δ 11.24 (s, 1H), 9.92 (s, 1H), 7.45 (dd, J=10.2, 2.5 Hz, 1H), 7.39 (d, J=2.9 Hz, 1H)*, 7.37 (dd, J=8.9, 4.7 Hz, 1H)*6.93 (dt, J=2.5, 9.2 Hz, 1H), 3.43-3.37 (m, 1H)*3.57-3.45 (m, 1H), 3.32-3.25 (m, 1H), 2.75 (dd, J=7.1, 4.8 Hz, 6H), 1.38 (d, J=6.8 Hz, 3H). *=coalescing with water. [HCl] ¹³C NMR (101 MHz, d₆-DMSO) δ 156.67 (d, J=231.0 Hz, 1C), 133.09 (s, 1C), 125.86 (d, J=10.0 Hz, 1C), 124.57 (s, 1C), 115.65 (d, J=4.7 Hz, 1C), 112.58 (d, J=9.8 Hz, 1C), 109.31 (d, J=26.1 Hz, 1C), 103.38 (d, J=23.3 Hz, 1C), 62.03 (s, 1C), 43.46 (s, 1C), 41.97 (s, 1C), 26.49 (s, 1C), 19.61 (s, 1C). [HCl] ¹⁹F NMR (377 MHz, DMSO) δ−124.67 (s, 1F).

### Example 27: (S)-5-fluoro-3-((1-methylpyrrolidin-2-yl)methyl)-1H-indole (27)

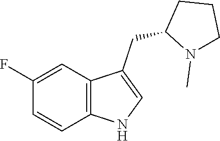 [SMILES: CN1CCC[C@H]1Cc1c[nH]c2ccc(F)cc12]

### Synthesis of benzyl (2S)-2-(5-fluoro-1H-indole-3-carbonyl)pyrrolidine-1-carboxylate

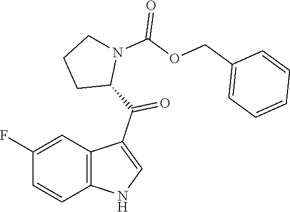 [SMILES: O=C(c1c[nH]c2ccc(F)cc12)[C@@H]1CCCN1C(=O)OCc1ccccc1]

All glassware was dried in the oven overnight and dried externally with hot air from a heat gun and allowed to cool to RT prior to setup up the reactions. 5-fluoroindole (5.41 g, 0.0400 mol) was dissolved in toluene (50 mL) and then 1.4 M methylmagnesium bromide (28.6 mL, 0.0400 mol) in 1:3 THF/Toluene was added through a syringe over 30 minutes, and then allowed to react for 3 h total. While the Grignard is forming, N-benzyloxylcarbonyl-L-proline (5 g, 0.0200 mol) was dissolved in dichloromethane (50 mL) in a separate round bottom flask along with oxalyl chloride (3.8 g, 0.0300 mol) and allowed to react for 2 h. After 2 h, the dichloromethane and excess oxalyl chloride was removed under reduced pressure. To ensure all oxalyl chloride was removed, 4 Å dried hexanes was added after all the dichloromethane was removed and the hexanes and residual oxalyl chloride was removed under reduced pressure.

Once the hexanes were removed the acid chloride was dissolved in diethyl ether (75 mL) and added to the Grignard reagent. This was allowed to react overnight. A sample of the reaction was taken and was examined by TLC to confirm the reaction was complete. The reaction was then poured in a saturated bicarbonate solution (400 mL) and 100 mL of EtOAc was added and allowed to stir for 1 h. After 1 h, the organic phase was separated and the aqueous phase was extracted from twice more with 100 mL of EtOAc (2×100 mL). Organic phases were combined and washed with saline, dried with sodium sulfate and solvent removed under reduced pressure.

Once the solvent was removed, 30 mL of EtOAc and 100 mL of diethyl ether was added to the round bottom flask containing the product and allowed to stir for 1 h prior to vacuum filtering the crystalline material to provide the title compound (mp: 194.0-196.0° C.) as a white crystalline solid (3.54 g, 0.00966 mol, 48.3% yield). The product was used without further purification.

### Synthesis of 5-fluoro-3-{[(2S)-1-methylpyrrolidin-2-yl]methyl}-1H-indole (27)

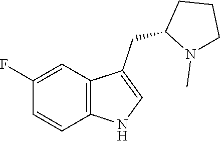 [SMILES: CN1CCC[C@H]1Cc1c[nH]c2ccc(F)cc12]

In an oven dried three neck round bottom flask was dried externally with heat gun while argon was flowing through the system and allowed to cool to RT prior to starting the reaction. The three neck was equipped with a condenser, and an addition funnel, which was utilized to add 80 mL of THE to the round bottom flask. Then LiAlH₄ (1.04 g, 0.0273 mol) was added to the reaction. LiAlH₄ that was stuck to the walls of the round bottom flask was rinsed with ˜ 20 mL THF. Following LiAlH₄ addition, benzyl (2S)-2-(5-fluoro-1H-indole-3-carbonyl)pyrrolidine-1-carboxylate (2 g, 0.00546 mol) was added to the addition funnel and dissolved in 80 mL of THF, which was added dropwise over 30 minutes. Once the addition was complete, a heating mantle was utilized to heat the reaction to a moderate reflux. Once the reflux was obtained the reaction was allowed to continue for 2 h. Following the 2 h period, the reaction was placed in an ice bath to cool to <10° C. The reaction was then quenched with −10° C. 50% THE in H₂O with 60 mL total volume. Upon quenching a small amount of KOH was added to the quenched reaction and was then filled with ˜ 200 mL of EtOAc and allowed to stir for a couple minutes. After stirring the solution, the solids in the reaction mixture were gravity filtered, and wash excessive with EtOAc. Washing was done until no more oil was eluting out of the washes. The organic phase was then extracted from with 0.3 M HCl three times (3×166 mL). These extractions were pooled and basified to a pH>12 with KOH and were extracted three times with 100 mL of EtOAc (3×100 mL). These organic extractions were pooled, and washed with ˜ 60 mL of brine (1×60 mL), dried with sodium sulfate, decanted and removed under reduced pressure to yield 1.25 g of a white/yellow solid. The material was purified by column chromatography utilizing silica gel as the stationary phase with 20% EtOH in EtOAc with 1% triethylamine as the mobile phase to provide the title compound as a white solid with a slightly yellow tint (1.17 g, 0.00504 mol, 92.3% yield) (m.p. 135-136.4° C.) High-resolution atmospheric solid analysis probe mass spectrometry (HR-ASAP-MS): m/z 233.1445 (theory [M+H]⁺ C₁₄H₁₈F₁N₂⁺, m/z 233.1449, Δ=−1.7 ppm). [Freebase] ¹H NMR (400 MHz, d₆-DMSO) δ 10.88 (s, 1H), 7.31 (dd, J=8.8, 4.6 Hz, 1H), 7.22 (dd, J=10.3, 2.4 Hz, 1H)*, 7.21 (d, J=2.3 Hz, 1H)*, 6.88 (td, J=9.2, 2.5 Hz, 1H), 3.02-2.90 (m, 2H), 2.49-2.42 (m, 1H)**, 2.33 (s, 3H)***, 2.36-2.25 (m, 1H)***, 2.09 (q, J=8.7 Hz, 1H), 1.73-1.63 (m, 1H), 1.63-1.48 (m, 2H), 1.48-1.38 (m, 1H). *=coalescing, **=DMSO Coalescing, ***=coalescing. [Freebase] ¹³C NMR (101 MHz, d₆-DMSO) δ 156.58 (d, J=230.6 Hz, 1C), 132.75 (s, 1C), 127.76 (d, J=9.7 Hz, 1C), 125.06 (s, 1C), 112.43 (d, J=4.9 Hz, 1C), 112.15 (d, J=9.7 Hz, 1C), 108.77 (d, J=26.1 Hz, 1C), 102.96 (d, J=22.8 Hz, 1C), 66.12 (s, 1C), 56.93 (s, 1C), 40.49 (s, 1C), 30.81 (s, 1C), 29.14 (s, 1C), 21.57 (s, 1C). [Freebase] ¹⁹F NMR (377 MHz, d₆-DMSO) δ−125.34 (s, 1F).

### Example 28: (R)-5-fluoro-3-((1-methylpyrrolidin-2-yl)methyl)-1H-indole (28)

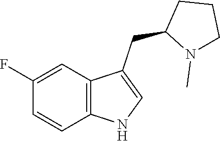 [SMILES: CN1CCC[C@@H]1Cc1c[nH]c2ccc(F)cc12]

### Synthesis of benzyl (2R)-2-(5-fluoro-1H-indole-3-carbonyl)pyrrolidine-1-carboxylate

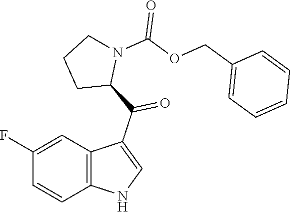 [SMILES: O=C(c1c[nH]c2ccc(F)cc12)[C@H]1CCCN1C(=O)OCc1ccccc1]

benzyl (2R)-2-(5-fluoro-1H-indole-3-carbonyl)pyrrolidine-1-carboxylate was synthesized in a similar manner as described above for (S)-5-fluoro-3-((1-methylpyrrolidin-2-yl)methyl)-1H-indole, starting from 5-fluoroindole (5.41 g, 0.0400 mol), N-benzyloxylcarbonyl-D-proline (5 g, 0.0200 mol), oxalyl chloride (3.8 g, 0.0300 mol), and 1.4 M methylmagnesium bromide in 1:3 THF/Toluene (28.6 mL, 0.0400 mol) as the starting materials to provide the title compound as a white solid (3.05 g, 0.00832 mol, 41.6% yield). The product was used without further purification.

### Synthesis of (R)-5-fluoro-3-((I-methylpyrrolidin-2-yl)methyl)-1H-indole (28)

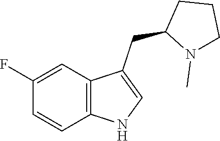 [SMILES: CN1CCC[C@@H]1Cc1c[nH]c2ccc(F)cc12]

5-fluoro-3-(((2R)-1-methylpyrrolidin-2-yl)methyl)-1H-indole was synthesized in a similar manner as described above for (S)-5-fluoro-3-((1-methylpyrrolidin-2-yl)methyl)-1H-indole, starting from benzyl (2R)-2-(5-fluoro-1H-indole-3-carbonyl)pyrrolidine-1-carboxylate (3 g, 0.00819 mol) as the starting material to provide the title compound as white yellow solid (1.42 g, 0.00611 mol, 74.6% yield), m.p. 127.6-131.7° C. High-resolution atmospheric solid analysis probe mass spectrometry (HR-ASAP-MS): m/z 233.1446 (theory [M+H]⁺ C₁₄H₁₈F₁N₂⁺, m/z 233.1449, Δ=−1.3 ppm). [Freebase] ¹H NMR (400 MHz, d₆-DMSO) δ 10.88 (s, 1H), 7.31 (dd, J=8.8, 4.6 Hz, 1H), 7.22 (dd, J=10.4, 2.4 Hz, 1H)*, 7.21 (d, J=2.4 Hz)*, 6.88 (td, J=9.2, 2.5 Hz, 1H), 3.02-2.92 (m, 2H), 2.49-2.42 (m, 1H)*, 2.33 (s, 3H), 2.37-2.26 (m, 1H), 2.09 (q, J=8.7 Hz, 1H), 1.73-1.63 (m, 1H), 1.63-1.48 (m, 2H), 1.48-1.38 (m, 1H)=Coalescing. [Freebase] ¹³C NMR (101 MHz, d₆-DMSO) δ 156.59 (d, J=230.6 Hz, 1C), 132.75 (s, 1C), 127.76 (d, J=9.7 Hz, 1C), 125.07 (s, 1C), 112.41 (d, J=4.6 Hz, 1C), 112.16 (d, J=9.7 Hz, 1C), 108.77 (d, J=26.0 Hz, 1C), 102.96 (d, J=22.8 Hz, 1C), 66.13 (s, 1C), 56.93 (s, 1C), 40.48 (s, 1C), 30.81 (s, 1C), 29.13 (s, 1C), 21.56 (s, 1C). [Freebase] ¹⁹F NMR (377 MHz, d₆-DMSO) δ−125.35 (s, 1F).

### Example 29: 5-fluoro-3-(1-methylazetidin-3-yl)-1H-indole (29)

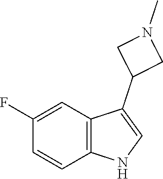

### Synthesis of tert-butyl 3-(5-fluoro-1H-indol-3-yl)-3-hydroxyazetidine-1-carboxylate

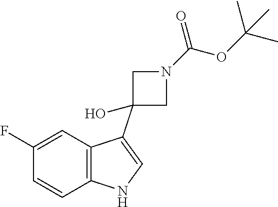 [SMILES: CC(C)(C)OC(=O)N1CC(O)(c2c[nH]c3ccc(F)cc23)C1]

In an oven dried 100 mL round bottom flask that was heated externally with hot air from a heat gun and cooled to RT under the flow of under was utilized for this reaction. 5-fluoroindole (1 g, 0.00740 mol) was dissolved in MeOH (40 mL) and then KOH (0.46 g, 0.00814 mol) was added and dissolved in the solution. Then N-Boc-3-azetidinone (1.39 g, 0.00814 mol) was added all at once and heated via a water bath at 50° C. for 15 h over two days. After two days, the reaction was worked up by removing the MeOH under reduced pressure and then re-dissolved in EtOAc (150 mL). The organic phase was washed with 50 mL of H₂O (3×50 mL), brine (1×60 mL), and the organic phase separated, dried with anhydrous sodium sulfate, decanted and removed under reduced pressure to provide the title compound as an orange oil (1.78 g, 0.00581 mol, 78.5% yield). The product was used without further purification.

### Synthesis of 5-fluoro-3-(I-methylazetidin-3-yl)-1H-indole (29)

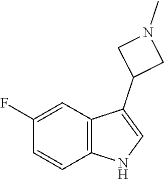

In an oven dried three neck round bottom flask was dried externally with hot air from a heat gun while argon was flowing through the system and allowed to cool to RT prior to starting the reaction. The three neck was equipped with a condenser, and an addition funnel, which was utilized to add 80 mL of THF to the round bottom flask. Then LiAlH₄ (1.06 g, 0.0278 mol) was added to the reaction. Any LiAlH₄ that was stuck to the walls of the round bottom flask was washed with ˜ 20 mL THF. Following LiAlH₄ addition, tert-butyl 3-(5-fluoro-1H-indol-3-yl)-3-hydroxyazetidine-1-carboxylate (1.7 g, 0.00555 mol) was added to the addition funnel and dissolved in 80 mL of THF. This was added dropwise over 30 minutes. Once the addition was complete, a heating mantle was utilized to heat the reaction to a moderate reflux. Once the reflux was obtained the reaction was allowed to continue for 2 h. Following the 2 h period, the reaction was then placed in an ice bath to cool to <10° C. The reaction was then quenched with −10° C. 50% THE in H₂O with 60 mL total volume. Upon quenching a small amount of KOH was added to the quenched reaction and was then filled with ˜ 200 mL of EtOAc and allowed to stir for a couple minutes. After stirring the solution, the solids in the reaction mixture were gravity filtered, and wash excessive with EtOAc. Washing was done until no more oil was eluting out of the washes. The organic phase was then extracted from with 0.3 M HCl three times (3×166 mL). These extractions were pooled and basified to a pH>12 with KOH and were extracted three times with 100 mL of EtOAc (3×100 mL). These organic extractions were pooled and washed with ˜ 60 mL of brine (1×60 mL), dried with sodium sulfate, decanted and removed under reduced pressure to yield 1.02 g of a yellow oil. This was purified by column chromatography utilizing silica gel as the stationary phase with a gradient of 5% to 20% EtOH in EtOAc with 1% triethylamine as the mobile phase to provide the title compound as an orange-white solid, which was crystallized from EtOAc and hexanes to give white solids (0.60 g, 0.00294 mol, 53.0%), m.p. 119.0-122.8° C. High-resolution atmospheric solid analysis probe mass spectrometry (HR-ASAP-MS): m/z 205.1133 (theory [M+H]⁺C₁₂H₁₄FN₂⁺, m/z 205.1136, Δ=−1.5 ppm). [Freebase] ¹H NMR (400 MHz, d₆-DMSO) δ 10.99 (s, 1H), 7.37 (dd, J=10.2, 2.5 Hz, 1H), 7.32 (dd, J=9.0, 4.9 Hz, 1H)*, 7.30 (d, J=2.8 Hz, 1H)*, 6.90 (td, J=9.2, 2.5 Hz, 1H), 3.76-3.65 (m, 3H), 3.17-3.09 (m, 2H), 2.30 (s, 3H). *=Coalescing peaks [Freebase] ¹³C NMR (101 MHz, d₆-DMSO) δ 156.58 (d, J=230.8 Hz, 1C), 133.06 (s, 1C), 126.42 (d, J=9.7 Hz, 1C), 124.23 (s, 1C), 116.34 (d, J=4.8 Hz, 1C), 112.33 (d, J=9.8 Hz, 1C), 109.12 (d, J=26.2 Hz, 1C), 103.27 (d, J=23.1 Hz, 1C), 62.91 (s, 2C), 45.86 (s, 1C), 26.55 (s, 1C). [Freebase]¹⁹F NMR (377 MHz, d₆-DMSO) δ−125.05 (s, 1F).

### Example 30: N-ethyl-N-(2-(5-fluoro-1H-indol-3-yl)ethyl)butan-2-amine (30)

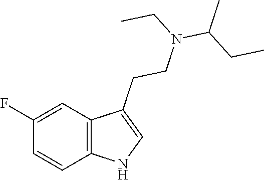 [SMILES: CCC(C)N(CC)CCc1c[nH]c2ccc(F)cc12]

N-ethyl-N-(2-(5-fluoro-1H-indol-3-yl)ethyl)butan-2-amine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), methyl ethyl ketone (0.5 g, 0.00699 mol), and acetaldehyde (0.62 g, 0.0140 mol) as the starting materials to provide the title compound as a transparent oil (0.35 g, 0.00132 mol, 56.7% yield). The conditions employed differ from that which is described for compound (1) in that 6 mol equivalents of acetaldehyde were utilized as opposed to 3 mol equivalents. High-resolution atmospheric solid analysis probe mass spectrometry (HR-ASAP-MS): m/z 263.1914 (theory [M+H]⁺ C₁₆H₂₄FN₂⁺, m/z 263.1918, Δ=−1.5 ppm). [Freebase] ¹H NMR (400 MHz, d₆-DMSO) δ 10.85 (s, 1H), 7.31 (dd, J=8.8, 4.6 Hz, 1H), 7.21 (d, J=2.1 Hz, 1H), 7.18 (dd, J=10.1, 2.4 Hz, 1H), 6.88 (td, J=9.2, 2.5 Hz, 1H), 2.81-2.71 (m, 1H), 2.71-2.61 (m, 3H), 2.60-2.51 (m, 2H)*, 2.44-2.36 (m, 1H), 1.54-1.33 (m, 1H), 1.27-1.13 (m, 1H), 0.99 (t, J=7.1 Hz, 3H), 0.87 (d, J=6.5 Hz, 3H), 0.83 (t, J=7.4 Hz, 3H). *=Coalescing peaks [Freebase] ¹³C NMR (101 MHz, d₆-DMSO) δ 156.58 (d, J=230.6 Hz, 1C), 132.84 (s, 1C), 127.53 (d, J=9.5 Hz, 1C), 124.71 (s, 1C), 113.36 (d, J=5.0 Hz, 1C), 112.20 (d, J=9.8 Hz, 1C), 108.79 (d, J=26.2 Hz, 1C), 102.77 (d, J=22.8 Hz, 1C), 56.09 (s, 1C), 50.22 (s, 1C), 43.32 (s, 1C), 26.54 (s, 1C), 25.27 (s, 1C), 14.66 (s, 1C), 13.92 (s, 1C), 11.57 (s, 1C). [Freebase]¹⁹F NMR (377 MHz, d₆-DMSO) δ−125.41 (s, 1F).

### Example 31: N-ethyl-N-(2-(5-fluoro-1H-indol-3-yl)ethyl)cyclobutanamine (31)

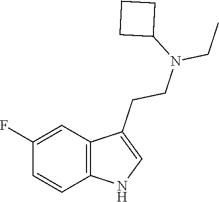 [SMILES: CCN(CCc1c[nH]c2ccc(F)cc12)C1CCC1]

N-ethyl-N-(2-(5-fluoro-1H-indol-3-yl)ethyl)cyclobutanamine was synthesized in a similar manner as described above for N-(2-(5-fluoro-1H-indol-3-yl)ethyl)-N-propylpropan-1-amine (5), starting from N-(2-(5-fluoro-1H-indol-3-yl)ethyl)cyclobutanamine (0.33 g, 0.00142 mol), and acetaldehyde (0.38 g, 0.00853 mol) as the starting materials to provide the title compound as a yellow/white solid (0.27 g, 0.00102 mol, 71.8% yield), m.p. 89.4-90.3° C. Only variation is that material was purified by column chromatography utilizing silica gel as the stationary phase and 5% to 20% EtOH in EtOAc with 1% TEA as the mobile phase. High-resolution atmospheric solid analysis probe mass spectrometry (HR-ASAP-MS): m/z 261.1757 (theory [M+H]⁺C₁₅H₂₁F₂N₂⁺, m/z 261.1762, Δ=−1.9 ppm). [Freebase] ¹H NMR (400 MHz, d₆-DMSO) δ 10.87 (s, 1H), 7.31 (dd, J=8.8, 4.6 Hz, H), 7.21 (d, J=2.2 Hz, 1H), 7.18 (dd, J=10.0, 2.5 Hz, 1H), 6.88 (td, J=9.2, 2.5 Hz, 1H), 3.19-3.09 (m, 1H), 2.74-2.65 (m, 2H), 2.65-2.56 (m, 2H), 2.56-2.50 (m, 2H)*, 2.03-1.94 (m, 2H), 1.85-1.71 (m, 2H), 1.65-1.50 (m, 2H), 0.96 (t, J=7.1 Hz, 3H). *=coalescing. [Freebase] ¹³C NMR (101 MHz, d₆-DMSO) δ 156.57 (d, J=231.0 Hz, 1C), 132.85 (s, 1C), 127.43 (d, J=9.6 Hz, 1C), 124.61 (s, 1C), 113.22 (d, J=4.9 Hz, 1C), 112.24 (d, J=9.7 Hz, 1C), 108.84 (d, J=26.0 Hz, 1C), 102.72 (d, J=22.8 Hz, 1C), 57.23 (s, 1C), 49.46 (s, 1C), 42.81 (s, 1C), 28.10 (s, 2C), 21.99 (s, 1C), 14.09 (s, 1C), 11.47 (s, 1C). [Freebase] ¹⁹F NMR (377 MHz, d₆-DMSO) δ−125.28 (s, 1F).

### Example 32: N-(2-(5-fluoro-1H-indol-3-yl)ethyl)-N-isopropylbutan-2-amine (32)

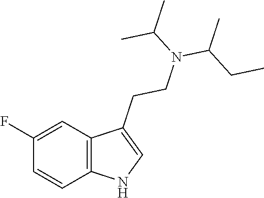 [SMILES: CCC(C)N(CCc1c[nH]c2ccc(F)cc12)C(C)C]

N-(2-(5-fluoro-1H-indol-3-yl)ethyl)-N-isopropylbutan-2-amine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), methyl ethyl ketone (0.5 g, 0.00699 mol), and acetone (23.52 g, 0.405 mol) as the starting materials to provide the title compound as a light brown oil (0.36 g, 0.00130 mol, 55.8% yield). The HCl salt was collected as a white fluffy solid (m.p. 193.7-195° C.). The only variation from the method described for compound (1) is that 30 mL of acetone and 20 mL MeOH were utilized as solvent as opposed to methanol. High-resolution atmospheric solid analysis probe mass spectrometry (HR-ASAP-MS): m/z 277.2069 (theory [M+H]⁺ C₁₇H₂₅FN₂⁺, m/z 277.2075, Δ=−2.2 ppm). [Freebase] ¹H NMR (400 MHz, d₆-DMSO) δ 10.86 (s, 1H), 7.31 (dd, J=8.3, 4.3 Hz, 1H), 7.21 (s, 1H), 7.16 (d, J=9.6 Hz, 1H), 6.88 (t, J=8.4 Hz, 1H), 3.08-2.97 (m, 1H), 2.78-2.54 (m, 5H), 1.44-1.28 (m, 1H), 1.25-1.16 (m, 1H), 1.06-0.89 (m, 9H), 0.83 (t, J=7.1 Hz, 3H). [Freebase] ¹³C NMR (101 MHz, d₆-DMSO) δ 156.56 (d, J=230.9 Hz, 1C), 132.83 (s, 1C), 127.51 (d, J=9.7 Hz, 1C), 124.70 (s, 1C), 113.38 (d, J=5.4 Hz, 1C), 112.19 (d, J=9.8 Hz, 1C), 108.74 (d, J=26.1 Hz, 1C), 102.68 (d, J=22.7 Hz, 1C), 53.71 (s, 1C), 47.67 (s, 1C), 45.64 (s, 1C), 28.11 (s, 1C), 26.82 (s, 1C), 22.11 (s, 1C), 19.89 (s, 1C), 17.31 (s, 1C), 11.68 (s, 1C). [Freebase] ¹⁹F NMR (377 MHz, DMSO) δ−125.40 (s, 1F).

### Example 33: N-(2-(5-fluoro-1H-indol-3-yl)ethyl)-N-propylcyclobutanamine (33)

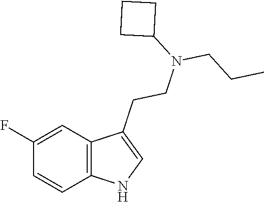 [SMILES: CCCN(CCc1c[nH]c2ccc(F)cc12)C1CCC1]

N-(2-(5-fluoro-1H-indol-3-yl)ethyl)-N-propylcyclobutanamine was synthesized in a similar manner as described above for N-(2-(5-fluoro-1H-indol-3-yl)ethyl)-N-propylpropan-1-amine (5), starting from N-(2-(5-fluoro-1H-indol-3-yl)ethyl)cyclobutanamine (0.33 g, 0.00142 mol), and propionaldehyde (0.38 g, 0.00853 mol) as the starting materials to provide the title compound as a yellow/white solid (0.28 g, 0.00101 mol, 71.1% yield), m.p. 110.0-112.5° C. The product was purified by column chromatography utilizing silica gel as the stationary phase and 5% to 20% EtOH in EtOAc with 1% TEA as the mobile phase. [Freebase] ¹H NMR (400 MHz, DMSO) δ 10.86 (s, 1H), 7.31 (dd, J=8.8, 4.6 Hz, 1H), 7.21 (d, J=2.3 Hz, 1H), 7.17 (dd, J=10.0, 2.5 Hz, 1H), 6.88 (td, J=9.1, 2.5 Hz, 1H), 3.21-3.10 (m, 1H), 2.75-2.66 (m, 2H), 2.66-2.58 (m, 2H), 2.43-2.35 (m, 2H), 2.03-1.94 (m, 2H), 1.84-1.71 (m, 2H), 1.63-1.51 (m, 2H), 1.47 (sex, J=7.4 Hz, 2H), 0.85 (t, J=7.3 Hz, 3H). [Freebase] ¹³C NMR (101 MHz, DMSO) δ 156.57 (d, J=230.9 Hz, 1C), 132.85 (s, 1C), 127.42 (d, J=9.8 Hz, 1C), 124.61 (s, 1C), 113.22 (d, J=4.9 Hz, 1C), 112.24 (d, J=9.7 Hz, 1C), 108.84 (d, J=25.9 Hz, 1C), 102.70 (d, J=22.8 Hz, 1C), 57.72 (s, 1C), 51.56 (s, 1C), 50.48 (s, 1C), 28.09 (s, 2C), 21.95 (s, 1C), 19.88 (s, 1C), 14.06 (s, 1C), 12.00 (s, 1C). [Freebase] ¹⁹F NMR (377 MHz, DMSO) δ−125.29 (s, 1F).

### Example 34: N-(2-(5-fluoro-1H-indol-3-yl)ethyl)-N-isopropylprop-2-yn-1-amine (34)

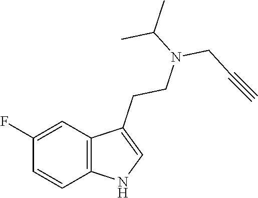 [SMILES: C#CCN(CCc1c[nH]c2ccc(F)cc12)C(C)C]

N-(2-(5-fluoro-1H-indol-3-yl)ethyl)-N-isopropylprop-2-yn-1-amine was synthesized in a similar manner as described above for N-(2-(5-fluoro-1H-indol-3-yl)ethyl)-N-isopropylprop-2-en-1-amine (11), starting from N-(2-(5-fluoro-1H-indol-3-yl)ethyl)propan-2-amine (0.5 g, 0.00227 mol) as the starting materials to provide the title compound as brown oil (0.21 g, 0.00809 mol, 35.6% yield). The product was converted to the fumarate salt. High-resolution atmospheric solid analysis probe mass spectrometry (HR-ASAP-MS): m/z 259.1601 (theory [M+H]⁺ C₁₆H₂OFN₂⁺, m/z 259.1605, Δ=−1.5 ppm).

### Example 35: N-(2-(5-fluoro-1H-indol-3-yl)ethyl)-N-propylprop-2-en-1-amine (35)

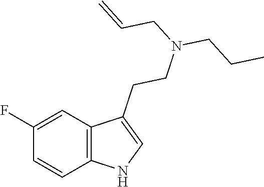 [SMILES: C=CCN(CCC)CCc1c[nH]c2ccc(F)cc12]

N-(2-(5-fluoro-1H-indol-3-yl)ethyl)-N-propylprop-2-en-1-amine was synthesized in a similar manner as described above for N-(2-(5-fluoro-1H-indol-3-yl)ethyl)-N-propylpropan-1-amine (5), starting from N-(2-(5-fluoro-1H-indol-3-yl)ethyl)prop-2-en-1-amine (0.5 g, 0.00229 mol) and propionaldehyde (495 μL, 0.00687 mol) as starting materials to provide the title compound as a pale-yellow odorless oil (0.48 g, 0.00198 mol, 80.5% yield). The material was converted to the HCl salt as described to provide white crystalline solids (m.p. 175.1-177.2° C.). Variations from the method used to prepare compound (5) include the use of 0.65 mol. eq. of NaBH₃CN, direct work-up with base, and purification by flash column chromatography using silica gel as the stationary phase with gradient elution by EtOAc (0.5% Et₃N) to 5% EtOH in EtOAc (0.5% Et₃N). High-resolution atmospheric solid analysis probe mass spectrometry (HR-ASAP-MS): m/z 261.1755 (theory [M+H]⁺ C₁₆H₂₂FN₂⁺, m/z 261.1762, Δ=−2.7 ppm). [HCl]¹H NMR (400 MHz, d₆-DMSO) δ 11.14 (s, 1H), 11.04 (s, 1H), 7.41 (dd, J=10.1, 2.5 Hz, 1H), 7.36 (dd, J=8.9, 4.6 Hz, 1H)*, 7.33 (d, J=2.4 Hz, 1H)*, 6.93 (td, J=9.2, 2.5 Hz, 1H), 6.15-6.03 (m, 1H), 5.60 (dd, J=17.1, 1.0 Hz, 1H), 5.51 (dd, J=10.3, 1.0 Hz, 1H), 3.89-3.79 (m 2H), 3.26-3.14 (m, 4H), 3.12-2.99 (m, 2H), 1.75 (sex, J=7.7 Hz, 2H), 0.91 (t, J=7.4 Hz, 3H). [HCl]¹³C NMR (101 MHz, d₆-DMSO) δ 156.75 (d, J=231.4 Hz, 1C), 132.87 (s, 1C), 127.54 (s, 1C), 126.94 (d, J=9.8 Hz, 1C), 125.49 (s, 1C), 124.66 (s, 1C), 112.52 (d, J=9.8 Hz, 1C), 109.53 (s, 1C), 109.34 (d, J=26.0 Hz, 1C), 103.05 (d, J=23.1 Hz, 1C), 53.91 (s, 1C), 52.95 (s, 1C), 51.89 (s, 1C), 19.31 (s, 1C), 16.51 (s, 1C), 10.94 (s, 1C). [HCl] ¹⁹F NMR (377 MHz, d₆-DMSO) δ−124.77 (s, 1F).

### Example 36: N-ethyl-N-(2-(5-fluoro-1H-indol-3-yl)ethyl)prop-2-en-1-amine (36)

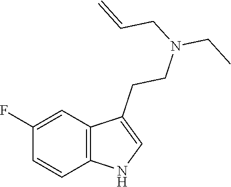 [SMILES: C=CCN(CC)CCc1c[nH]c2ccc(F)cc12]

N-ethyl-N-(2-(5-fluoro-1H-indol-3-yl)ethyl)prop-2-en-1-amine was synthesized in a similar manner as described above for N-(2-(5-fluoro-1H-indol-3-yl)ethyl)-N-propylpropan-1-amine (5), starting from N-(2-(5-fluoro-1H-indol-3-yl)ethyl)prop-2-en-1-amine (0.5 g, 0.00229 mol) and acetaldehyde (0.30 g, 0.00687 mol,) as starting materials to provide the title compound as a yellowish oil (0.42 g, 0.00161 mol, 74.4% yield). The hydrochloride salt was collected as a white crystalline material (m.p. 123.7-128.5° C.). Variations from the method used to prepare compound (5) include the use of 1.1 molar eq. of acetic acid, 0.65 mol. eq. of NaBH₃CN and direct work-up with base. The product was purified by flash column chromatography using silica gel as the stationary phase with gradient elution by EtOAc (0.5% Et₃N) to 10% EtOH in EtOAc (0.5% Et₃N). High-resolution atmospheric solid analysis probe mass spectrometry (HR-ASAP-MS): m/z 247.1600 (theory [M+H]⁺ C₁₅H₂OFN₂⁺, m/z 247.1605, Δ=−2.0 ppm). [HCl] ¹H NMR (400 MHz, d₆-DMSO) δ 11.14 (s, 1H), 11.08 (s, 1H), 7.41 (dd, J=10.0, 2.4 Hz, 1H), 7.36 (dd, J=9.0, 4.7 Hz, 1H), 7.34 (d, J=2.7 Hz, 1H), 6.93 (td, J=9.2, 2.5 Hz, 1H), 6.14-5.99 (m, 1H), 5.60 (dd, J=6.1, 1.3 Hz, 1H), 5.51 (d, J=10.5 Hz, 1H), 3.89-3.77 (m, 2H), 3.24-3.11 (m, 6H)*, 1.28 (t, J=7.2 Hz, 3H). *coalescing. [HCl] ¹³C NMR (101 MHz, d₆-DMSO) δ 156.75 (d, J=231.2 Hz, 1C), 132.87 (s, 1C), 127.55 (s, 1C), 126.94 (d, J=10.1 Hz, 1C), 125.49 (s, 1C), 124.58 (s, 1C), 112.52 (d, J=9.9 Hz, 1C), 109.52 (s, 1C), 109.33 (d, J=26.0 Hz, 1C), 103.03 (d, J=23.2 Hz, 1C), 53.24 (s, 1C), 51.27 (s, 1C), 46.38 (s, 1C), 19.34 (s, 1C), 8.45 (s, 1C). [HCl]19F NMR (377 MHz, d₆-DMSO) δ−124.77 (s, 1F).

### Example 37: N-(2-(6-chloro-5-fluoro-1H-indol-3-yl)ethyl)-N-ethylpropan-1-amine (37)

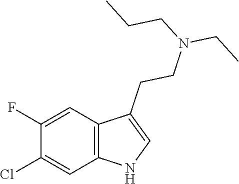 [SMILES: CCCN(CC)CCc1c[nH]c2cc(Cl)c(F)cc12]

### Synthesis of 2-(6-chloro-5-fluoro-1H-indol-3-yl)-N-ethyl-2-oxo-N-propylacetamide

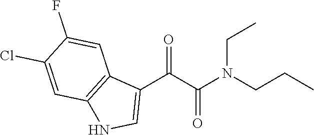 [SMILES: CCCN(CC)C(=O)C(=O)c1c[nH]c2cc(Cl)c(F)cc12]

2-(6-chloro-5-fluoro-1H-indol-3-yl)-N-ethyl-2-oxo-N-propylacetamide was synthesized in a similar manner as described above for N-ethyl-2-(7-fluoro-1H-indol-3-yl)-N-methylene-1-amine (7), starting from 6-chloro-5-fluoro-1H-indole (4.83 g, 0.02843 mol), oxalyl chloride (2.99 mL, 35.58 mmol), and N-ethylpropylamine (4.95 mL, 0.0424 mol), and TEA (17.7 mL, 0.127 mol) as starting material, yielding crude material as tanned-sand fine powder (8.16 g, 0.02626 mol, 89.0% yield). The product was used without further purification.

### Synthesis N-(2-(6-chloro-5-fluoro-1H-indol-3-yl)ethyl)-N-ethylpropan-1-amine (37)

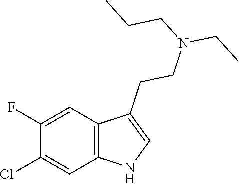 [SMILES: CCCN(CC)CCc1c[nH]c2cc(Cl)c(F)cc12]

A three neck 500 mL round bottom flask, 300 mL addition funnel, stir bar, and condenser were dried overnight in the oven and additionally dried externally with a stream of hot air while maintaining argon flow through the system, and assembled. Once deemed dry, the round bottom flask was placed into an ice-water bath (0° C.) and allowed to cool. Once cool, 70 mL of dry (3 Å molecular sieves) tetrahydrofuran (THF) was added to the round bottom flask and was allowed to reach −0° C. Lithium aluminum hydride (LiAlH₄) (2.95 g, 0.0777 mol) was added slowly to the round bottom flask while stirring and under argon atmosphere. Meanwhile, 100 mL of dry THE was added to an oven-dried Erlenmeyer flask, placed into an ice-water bath (−0° C.) and allowed to cool. Once cool, AlCl₃ (3.55 g, 0.02663 mol) was added portion-wise with swirling. The solution was transferred into the addition funnel and added dropwise to the stirred, cooled LiAlH₄ suspension over 30 min at ˜12-17° C. An additional 3×25 mL of THF was added to the addition funnel to wash any remaining material. Once the addition was complete, a solution of 2-(6-chloro-5-fluoro-1H-indol-3-yl)-N-ethyl-2-oxo-N-propylacetamide (8.05 g, 0.0259 mol) in 100 mL of THE was added dropwise over 1 h at 0° C. An additional 20 mL of THE was added to the addition funnel to wash any remaining material and this was added over 10 mins. The reaction was then heated to reflux and maintained at reflux, with additional dry THE added during the reaction as it evaporated, for 3 h and 15 min while stirring (Teflon coated magnetic stir-bar) under argon. At this point the reaction was confirmed to be complete (as confirmed by TLC and ASAP-MS). The reaction was then placed back on an ice-water bath and quenched by the cautious addition of ˜1:1 THF/H₂O with ice added. Once quenched, a few mL of brine, and aqueous KOH solution were added to ensure basicity, followed by 100 mL of EtOAc. The inorganic solids were removed by gravity filtration over Whatman paper (24 cm diameter). The removed solids were then washed extensively with EtOAc. After washing the solids, the mother liquor was taken and extracted 4×125 mL of a 0.2 M aqueous HCl solution. The aqueous phase was then basified with KOH pellets and extracted with 3×100 mL EtOAc. The pooled organic phases were washed with brine (20 mL), dried over anhydrous Na₂SO₄ and the solvent removed under reduced pressure to yield 4.56 g of an orange oil, which crystalized upon cooling into white crystalline solid. The free base was purified by column chromatography using silica gel as the stationary phase employing gradient elution from EtOAc (1% TEA) to 10% EtOH in EtOAc (1% TEA), to provide the title compound as a pale white solid (2.52 g, 0.00891 mol, 34.4% yield), m.p. 92.4-93.6° C. The hydrochloride salt was collected as a white powder (m.p. 231.0-232.6° C.). High-resolution atmospheric solid analysis probe mass spectrometry (HR-ASAP-MS): m/z 283.1367 (theory [M+H]⁺ C₁₅H₂₁FClN₂⁺, m/z 283.1372, Δ=−1.8 ppm). [HCl] ¹H NMR (400 MHz, d₆-DMSO) δ 11.29 (s, 1H), 10.68 (s, 1H), 7.69 (d, J=10.3 Hz, 1H), 7.54 (d, J=6.4 Hz, 1H), 7.41 (d, J=2.3 Hz, 1H), 3.27-3.11 (m, 6H)*, 3.11-2.98 (m, 2H), 1.71 (sex, J=9.7 Hz, 2H), 1.25 (t, J=7.2 Hz, 3H), 0.92 (t, J=7.4 Hz, 3H). *coalescing. [HCl] ¹³C NMR (101 MHz, d₆-DMSO) δ 151.51 (d, J=234.6 Hz, 1C), 132.55 (s, 1C), 126.32 (s, 1C), 125.76 (d, J=8.6 Hz, 1C), 113.27 (d, J=21.4 Hz, 1C), 112.51 (s, 1C), 109.99 (d, J=4.5 Hz, 1C), 104.73 (d, J=23.0 Hz, 1C), 52.35 (s, 1C), 51.51 (s, 1C), 46.41 (s, 1C), 19.24 (s, 1C), 16.47 (s, 1C), 10.94 (s, 1C), 8.33 (s, 1C). [HCl] ¹⁹F NMR (377 MHz, d₆-DMSO) δ−128.02 (s, 1F).

### Example 38: 1-cyclopropyl-N-(2-(5-fluoro-1H-indol-3-yl)ethyl)-N-methylethan-1-amine (38)

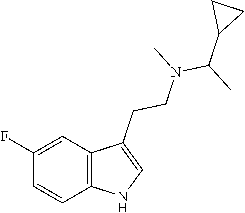 [SMILES: CC(C1CC1)N(C)CCc1c[nH]c2ccc(F)cc12]

1-Cyclopropyl-N-(2-(5-fluoro-1H-indol-3-yl)ethyl)-N-methylethan-1-amine was synthesized in a similar manner as described above for N-ethyl-N-(2-(5-fluoro-1H-indol-3-yl)ethyl)propan-2-amine (1), starting from 5-fluorotryptamine hydrochloride (0.6 g, 0.00279 mol) and cyclopropyl methyl ketone (0.704 g, 0.00837 mol) and 37% aqueous solution of formaldehyde (0.62 mL, 0.00837 mol) as starting materials to provide the title compound as a white solid (0.44 g, 0.00169 mol, 60.4% yield). The hydrochloride salt of the title compound was collected as white round pearl-shaped solids. Variations from the method used for the synthesis of compound (1) are as follows: the reaction mixture was heated to reflux to form the secondary amine intermediate, cooled to RT, then fresh NaBH₃CN (0.114 g, 0.00181 mol) and formaldehyde were added; and the reaction was worked up directly with base. The product was purified by flash column chromatography using silica gel as the stationary phase with gradient elution by EtOAc (0.5% TEA) to 35% EtOH in EtOAc (0.5% TEA). High-resolution atmospheric solid analysis probe mass spectrometry (HR-ASAP-MS): m/z 261.1757 (theory [M+H]⁺ C₁₅H₂₁FN₂⁺, m/z 261.1762, Δ=−1.9 ppm). HCl m.p. 100.7-101.5° C. [Freebase] ¹H NMR (400 MHz, d₆-DMSO) δ 10.86 (s, 1H), 7.31 (dd, J=8.8, 4.6 Hz, 1H), 7.22 (d, J=2.3 Hz, 1H)*, 7.22 (dd, J=9.8, 2.5 Hz, 1H)*, 6.88 (td, J=9.1, 2.4 Hz, 1H), 2.79-2.68 (m, 4H), 2.32 (s, 3H), 2.02-1.89 (m, 1H), 1.00 (d, J=6.6 Hz, 3H), 0.81-0.70 (m, 1H), 0.49-0.41 (m, 1H), 0.41-0.32 (m, 1H), 0.27-0.17 (m, 1H), 0.06-(−0.04) (m, 1H). [Freebase] ¹³C NMR (101 MHz, d₆-DMSO) δ 156.56 (d, J=230.6 Hz, 1C), 132.81 (s, 1C), 127.53 (d, J=9.6 Hz, 1C), 124.62 (s, 1C), 113.30 (d, J=4.9 Hz, 1C), 112.15 (d, J=9.5 Hz, 1C), 108.79 (d, J=26.1 Hz, 1C), 102.88 (d, J=22.8 Hz, 1C), 62.62 (s, 1C), 54.49 (s, 1C), 37.41 (s, 1C), 23.50 (s, 1C), 15.43 (s, 1C), 13.88 (s, 1C), 4.93 (s, 1C), 2.04 (s, 1C). ¹⁹F NMR (377 MHz, d₆-DMSO) δ−125.37 (s, 1F).

### Example 39: N-(2-(5-fluoro-1H-indol-3-yl)ethyl)-N,2-dimethylpropan-1-amine (39)

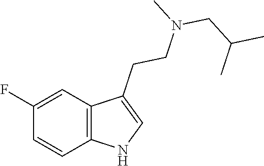 [SMILES: CC(C)CN(C)CCc1c[nH]c2ccc(F)cc12]

N-(2-(5-fluoro-1H-indol-3-yl)ethyl)-N,2-dimethylpropan-1-amine was synthesized in a similar manner as described above for N-ethyl-N-(2-(5-fluoro-1H-indol-3-yl)ethyl)propan-2-amine (1), starting from 5-fluorotryptamine hydrochloride (0.6 g, 0.00279 mol) and isobutyraldehyde (0.604 g, 0.00837 mol) and 37% aqueous solution of formaldehyde (623 μL, 0.00837 mmol) as starting materials to provide the title compound as a transparent amber oil (0.23 g, 0.00926 mol, 33.0% yield). The crude product was purified by flash column chromatography using silica gel as the stationary phase and eluted with 50% hexane and 50% EtOAc (0.5% TEA). The hydrochloride salt of the title compound was collected as white opaque shining crystalline solids (m.p. 150.0-151.5° C.). High-resolution atmospheric solid analysis probe mass spectrometry (HR-ASAP-MS): m/z 249.1757 (theory [M+H]⁺ C₁₅H₂₂FN₂⁺, m/z 249.1762, Δ=−2.0 ppm). [HCl] ¹H NMR (400 MHz, d₆-DMSO) δ 11.21 (s, 1H), 10.46 (s, 1H), 7.50 (dd, J=10.1, 2.5 Hz, 1H), 7.36 (dd, J=8.8, 4.6 Hz, 1H), 7.31 (d, J=2.4 Hz, 1H), 6.92 (td, J=9.3, 2.5 Hz, 1H), 3.33-3.12 (m, 4H), 3.11-3.02 (m, 1H), 2.98-2.85 (m, 1H), 2.81 (d, J=4.8 Hz, 3H), 2.16-2.01 (m, 1H), 1.03 (d, J=6.6 Hz, 3H), 0.98 (d, J=6.6 Hz, 3H). [HCl] ¹³C NMR (101 MHz, d₆-DMSO) δ 156.51 (d, J=231.1 Hz, 1C), 132.67 (s, 1C), 126.76 (d, J=10.1 Hz, 1C), 125.18 (s, 1C), 112.26 (d, J=9.8 Hz, 1C), 109.27 (d, J=4.9 Hz, 1C), 109.07 (d, J=26.1 Hz, 1C), 103.00 (d, J=23.2 Hz, 1C), 61.53 (s, 1C), 55.92 (s, 1C), 39.52 (s, 1C), 23.47 (s, 1C), 20.42 (s, 1C), 20.19 (s, 1C), 19.25 (s, 1C). [HCl] ¹⁹F NMR (377 MHz, d₆-DMSO) δ−124.90 (s, 1F).

### Example 40: N-(2-(5-fluoro-1H-indol-3-yl)ethyl)-N-isopropylcyclobutanamine (40)

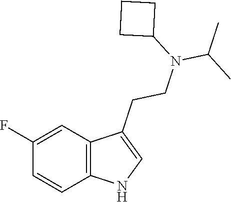 [SMILES: CC(C)N(CCc1c[nH]c2ccc(F)cc12)C1CCC1]

N-(2-(5-fluoro-1H-indol-3-yl)ethyl)-N-isopropylcyclobutanamine was synthesized in a similar manner as described above for N-ethyl-N-(2-(5-fluoro-1H-indol-3-yl)ethyl)propan-2-amine (1), starting from 5-fluoro-tryptamine hydrochloride (0.6 g, 0.00279 mol) and acetone (0.205 mL, 0.00558 mol) as starting materials to provide the title compound as a colorless oil that solidified into white solid (110 mg, 0.0004 mol, 14.4% yield). The hydrochloride salt was collected as a white crystalline powder, m.p. 191.6-193.0° C. Variations from the method B include: removal of the solvent was performed under reduced pressure with continued additions of MeOH once full conversion was observed; the residue was re-dissolved in dry MeOH, followed by the addition of acetic acid (16 μL), NaBH₃CN (0.114 g, 0.00181 mol) and cyclobutanone (0.83 mL, 0.01116 mol) and reaction was set to reflux. After 24 h, new NaBH₃CN (0.114 g, 0.00181 mol) and cyclobutanone (0.42 mL, 0.00558 mmol) were added and refluxed continued until full completion (GC-MS) after 44 h (total). The reaction was then cooled to RT and worked up by diluting with H₂O, basifying and extracting as described elsewhere herein. The purification was done by flash column chromatography using silica gel as the stationary phase with elution by EtOAc (0.5% TEA). High-resolution atmospheric solid analysis probe mass spectrometry (HR-ASAP-MS): m/z 275.1912 (theory [M+H]⁺C₁₇H₂₄FN₂⁺, m/z 275.1918, Δ=−2.2 ppm). [HCl] ¹H NMR (400 MHz, d₆-DMSO) δ 11.14 (s, 1H), 10.83 (s, 1H), 7.41-7.33 (m, 3H), 6.93 (td, J=9.2, 2.5 Hz, 1H), 3.97-3.83 (m, 1H), 3.67-3.54 (m, 1H), 3.21-3.09 (m, 3H), 3.09-2.98 (m, 1H), 2.66 (p, J=10.0 Hz, 1H), 2.59-2.51 (m, 1H)*, 2.30-2.14 (m, 1H), 1.80-1.70 (m, 1H), 1.70-1.60 (m, 1H), 1.35 (d, J=6.6 Hz, 3H), 1.24 (d, J=6.7 Hz, 3H).*coalescing with d₆-DMSO. [HCl] ¹³C NMR (101 MHz, d₆-DMSO) δ 156.77 (d, J=231.2 Hz, 1C), 132.86 (s, 1C), 126.87 (d, J=9.7 Hz, 1C), 125.56 (s, 1C), 112.53 (d, J=9.7 Hz, 1C), 109.96 (d, J=4.9 Hz, 1C), 109.32 (d, J=26.2 Hz, 1C), 102.90 (d, J=23.1 Hz, 1C), 55.75 (s, 1C), 52.66 (s, 1C), 47.65 (s, 1C), 27.09 (s, 1C), 27.04 (s, 1C), 21.07 (s, 1C), 16.89 (s, 1C), 15.85 (s, 1C), 14.19 (s, 1C). [HCl] ¹⁹F NMR (377 MHz, d₆-DMSO) δ−124.65 (s, 1F).

### Example 41: N-ethyl-N-(2-(7-fluoro-5-methoxy-1H-indol-3-yl)ethyl)propan-1-amine (41)

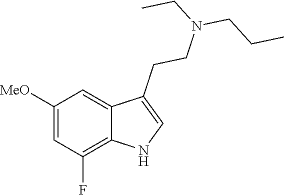 [SMILES: CCCN(CC)CCc1c[nH]c2c(F)cc(OC)cc12]

### Synthesis of N-ethyl-2-(7-fluoro-5-methoxy-1H-indol-3-yl)-2-oxo-N-propylacetamide

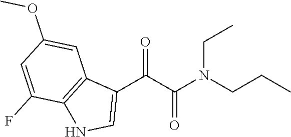 [SMILES: CCCN(CC)C(=O)C(=O)c1c[nH]c2c(F)cc(OC)cc12]

N-ethyl-2-(7-fluoro-5-methoxy-1H-indol-3-yl)-2-oxo-N-propylacetamide was synthesized in a similar manner as described above for N-ethyl-2-(7-fluoro-1H-indol-3-yl)-N-methylene-1amine, utilizing 5-methoxy-7-fluoroindole (1 g, 0.00605 mol), oxalyl chloride (0.99 g, 0.00787 mol), and ethyl propyl amine (0.92 mL, 0.00787 mol) to give N-ethyl-2-(7-fluoro-5-methoxy-1H-indol-3-yl)-2-oxo-N-propylacetamide as an yellow oil that crystallized in upon cooling in the refrigerator (2.35 g, 0.00767 mol, quant.).

### Synthesis of N-ethyl-N-(2-(7-fluoro-5-methoxy-1H-indol-3-yl)ethyl)propan-1-amine

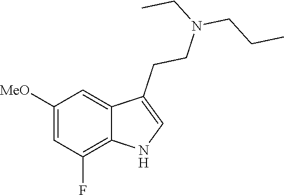 [SMILES: CCCN(CC)CCc1c[nH]c2c(F)cc(OC)cc12]

N-ethyl-N-(2-(7-fluoro-5-methoxy-1H-indol-3-yl)ethyl)propan-1-amine (41) was synthesized in a similar manner as described above for N-ethyl-2-(7-fluoro-1H-indol-3-yl)-N-methylene-1-amine (7), utilizing N-ethyl-2-(7-fluoro-5-methoxy-1H-indol-3-yl)-2-oxo-N-propylacetamide (1.7 g, 0.00555 mol) to give N-ethyl-N-(2-(7-fluoro-5-methoxy-1H-indol-3-yl)ethyl)propan-1-amine as a transparent light yellow oil (0.76 g, 0.02273 mol, 49.19% yield). The HCl salt was collected as a white crystalline powder. High-resolution atmosphere solid analysis probe mass spectrometry (HR-ASAP-MS): m/z 279.1871 (theory [M+H]+C₁₆H₂₄FN₂O*, m/z 279.1867, Δ=1.4 ppm). ¹H NMR (400 MHz, DMSO) δ 11.30 (s, 1H), 10.60 (s, 1H), 7.30 (d, J=2.4 Hz, 1H), 7.03 (d, J=2.0 Hz, 1H), 6.63 (dd, J=12.7, 2.0 Hz, 1H), 3.79 (s, 3H), 3.27-3.22 (m, 4H), 3.15-3.10 (m, 2H), 3.09-3.02 (m, 2H), 1.71 (sex, J=7.7 Hz, 2H), 1.26 (t, J=7.2 Hz, 3H), 0.92 (t, J=7.3 Hz, 3H). [HCl] ¹³C NMR (101 MHz, DMSO) δ 153.24 (d, J=9.3 Hz, 1C), 148.90 (d, J=243.2 Hz, 1C), 130.19 (d, J=7.3 Hz, 1C), 125.10 (s, 1C), 119.13 (d, J=13.5 Hz, 1C), 110.30 (d, J=2.3 Hz, 1C), 97.15 (d, J=19.4 Hz, 1C), 96.48 (d, J=3.1 Hz, 1C), 55.91 (s, 1C), 52.46 (s, 1C), 51.40 (s, 1C), 46.52 (s, 1C), 19.44 (s, 1C), 16.53 (s, 1C), 10.98 (s, 1C), 8.38 (s, 1C). [HCl] ¹⁹F NMR (377 MHz, d₆-DMSO) δ−131.29 (s, 1F).

### Example 42: N-(2-(5-fluoro-1H-indol-3-yl)ethyl)-N-methylcyclobutanamine (51)

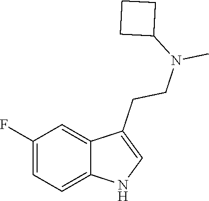 [SMILES: CN(CCc1c[nH]c2ccc(F)cc12)C1CCC1]

N-[2-(5-fluoro-1H-indol-3-yl)ethyl]-N-methylcyclobutanamine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), cyclobutanone (0.98 g, 0.014 mol), and 37% formaldehyde in water (0.52 mL, 0.00699 mol) as the starting materials to provide the title compound as transparent amber oil. The hydrochloride salt was collected as a white crystalline solid (0.26 g, 0.000920 mol, 39.5% yield) (m.p. 219.4-219.7° C.). High-resolution atmospheric solid analysis probe mass spectrometry (HR-ASAP-MS). m/z 247.1616 (theory [M+H]⁺ C₁₅H₂OFN₂⁺, m/z 247.1605, Δ=4.5 ppm). [HCl] ¹H NMR (400 MHz, d₆-DMSO) δ 11.18 (s, 1H), 11.13 (s, 1H), 7.42 (dd, J=10.0, 2.5 Hz, 1H), 7.36 (dd, J=8.8, 4.6 Hz, 1H)*, 7.33 (d, J=2.4 Hz, 1H)*, 6.93 (td, J=9.2, 2.5 Hz, 1H), 3.71 (sex, J=8.3 Hz, 1H), 3.23-2.99 (m, 4H), 2.69 (d, J=4.7 Hz, 3H), 2.48-2.30 (m, 2H), 2.25-2.11 (m, 2H), 1.79-1.58 (m, 2H).*=Coalescing. [HCl] ¹³C NMR (101 MHz, d₆-DMSO) δ 156.75 (d, J=231.2 Hz, 1C), 132.87 (s, 1C), 126.91 (d, J=10.1 Hz, 1C), 125.42 (s, 1C), 112.50 (d, J=9.7 Hz, 1C), 109.55 (d, J=4.8 Hz, 1C), 109.33 (d, J=26.2 Hz, 1C), 103.01 (d, J=23.1 Hz, 1C), 58.43 (s, 1C), 52.08 (s, 1C), 35.53 (s, 1C), 25.41 (s, 1C), 25.08 (s, 1C), 19.45 (s, 1C), 12.93 (s, 1C). [HCl] ¹⁹F NMR (377 MHz, d₆-DMSO) δ−124.73 (s, 1F).

### Example 43: N-(sec-butyl)-N-(2-(5-fluoro-1H-indol-3-yl)ethyl)cyclobutanamine (158)

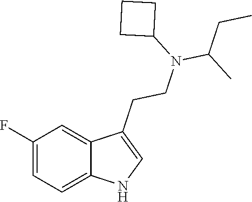 [SMILES: CCC(C)N(CCc1c[nH]c2ccc(F)cc12)C1CCC1]

N-(butan-2-yl)-N-[2-(5-fluoro-1H-indol-3-yl)ethyl]cyclobutanamine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), methyl ethyl ketone (0.63 mL, 0.00699 mol), and cyclobutanone (0.65 g, 0.00932 mol) as the starting materials to provide the title compound as transparent colorless crystals (0.66 g, 0.00229 mol, 98% yield). The hydrochloride salt was collected as a white crystalline solid. The conditions employed differ from that which is described for compound (1) the material was purified in 80% EtOAc in hexanes with 0.5% triethylamine. Also, after addition of the cyclobutanone the reaction was held at reflux for 7.5 h. High-resolution atmospheric solid analysis probe mass spectrometry (HR-ASAP-MS): m/z 289.2061 (theory [M+H]⁺C₁₈H₂₆FN₂⁺, m/z 289.2075, Δ=−4.84 ppm). [Free base] ¹H NMR (400 MHz, d₆-DMSO) δ 10.90 (s, 1H), 7.31 (dd, J=8.8, 4.6 Hz, 1H), 7.22 (d, J=1.7 Hz, 1H), 7.16 (dd, J=9.9, 1.6 Hz, 1H), 6.89 (td, J=13.8, 2.5 Hz, 1H), 3.31-3.22 (m, 1H)*2.82-2.59 (m, 4H)**, 2.59-2.51 (m, 1H)*/**, 2.04-1.75 (m, 4H), 1.63-1.51 (m, 2H)***, 1.51-1.36 (m, 1H)***, 1.31-1.15 (m, 1H), 0.89 (d, J=6.1 Hz, 3H), 0.86 (t, J=7.4 Hz, 3H).*=coalescing with H₂O, **=Coalescing with DMSO solvent peak, ***=coalescing. [Free base] ¹³C NMR (101 MHz, d₆-DMSO) δ 156.58 (d, J=230.9 Hz, 1C), 141.29 (s, 1C), 132.86 (s, 1C), 127.41 (d, J=9.2 Hz, 1C), 124.74 (s, 1C), 112.27 (d, J=9.8 Hz, 1C), 108.83 (d, J=26.1 Hz, 1C), 102.71 (d, J=22.8 Hz, 1C), 55.35 (s, 2C), 47.24 (s, 1C), 29.57 (s, 1C), 29.01 (s, 1C), 26.95 (s, 1C), 14.98 (s, 1C), 14.67 (s, 2C), 11.71 (s, 1C). [Free base] ¹⁹F NMR (377 MHz, d₆-DMSO) δ−125.28 (s, 1F).

### Example 44: N-(cyclopropylmethyl)-N-(2-(5-fluoro-1H-indol-3-yl)ethyl)propan-2-amine (159)

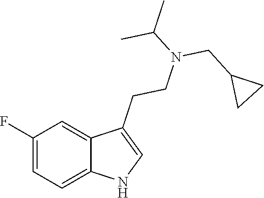 [SMILES: CC(C)N(CCc1c[nH]c2ccc(F)cc12)CC1CC1]

N-(cyclopropylmethyl)-N-(2-(5-fluoro-1H-indol-3-yl)ethyl)propan-2-amine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), acetone (0.41 g, 0.00699 mol), and cyclopropanecarboxaldehyde (0.49 g, 0.00699 mol) as the starting materials to provide the title compound as a transparent amber oil (0.58 g, 0.00211 mol, 91% yield). The hydrochloride salt was collected as a white crystalline solid (m.p. 191.0-192.5° C.). The conditions employed differ from that which is described for compound (1) the material was purified with a gradient starting with pure ethyl acetate and 0.5% TEA to 5% EtOH in EtOAc with 0.5% TEA. High-resolution atmospheric solid analysis probe mass spectrometry (HR-ASAP-MS): m/z 275.1908 (theory [M+H]⁺C₁₅H₂₁F₂N₂⁺, m/z 275.1918, Δ=−3.6 ppm) [HCl] ¹H NMR (400 MHz, d₆-DMSO) δ 11.15 (s, 1H), 10.32 (s, 1H), 7.43 (dd, J=10.1, 2.5 Hz, 1H), 7.36 (dd, J=8.5, 4.8 Hz, 1H)*, 7.36 (d, J=3.0 Hz, 1H)*, 6.93 (td, J=9.2, 2.5 Hz, 1H), 3.91-3.76 (m, 1H), 3.31-3.24 (m, 2H)*, 3.23-3.17 (m, 2H)**, 3.15-2.98 (m, 2H), 1.31 (d, J=6.7 Hz, 3H)***, 1.29 (d, J=6.7 Hz, 3H)**, 1.26-1.18 (m, J=4.0 Hz, 1H)**, 0.69-0.61 (m, 2H), 0.53-0.37 (m, 2H).*=Coalescing, **=Coalescing, ***=Coalescing. [HCl] ¹³C NMR (101 MHz, d₆-DMSO) δ 156.78 (d, J=231.3 Hz, 1C), 132.89 (s, 1C), 127.00 (d, J=10.1 Hz, 1C), 125.51 (s, 1C), 112.54 (d, J=9.8 Hz, 1C), 109.85 (d, J=4.7 Hz, 1C), 109.35 (d, J=26.0 Hz, 1C), 103.05 (d, J=23.1 Hz, 1C), 53.57 (s, 1C), 53.46 (s, 1C), 49.13 (s, 1C), 20.39 (s, 1C), 16.02 (s, 2C), 6.26 (s, 1C), 4.72 (s, 1C), 4.37 (s, 1C). [HCl] ¹⁹F NMR (377 MHz, d₆-DMSO) δ−124.70 (s, 1F).

### Example 45: N-[2-(5,7-difluoro-1H-indol-3-yl)ethyl]-N-ethylpropan-1-amine (72)

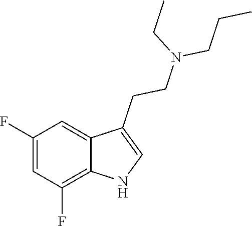 [SMILES: CCCN(CC)CCc1c[nH]c2c(F)cc(F)cc12]

### Synthesis of 2-(5,7-difluoro-1H-indol-3-yl)-N-ethyl-2-oxo-N-propylacetamide

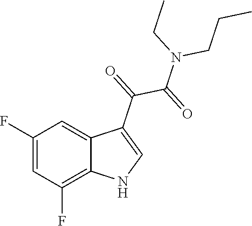 [SMILES: CCCN(CC)C(=O)C(=O)c1c[nH]c2c(F)cc(F)cc12]

2-(5,7-difluoro-1H-indol-3-yl)-N-ethyl-2-oxo-N-propylacetamide was synthesized in a similar manner as described above for N-ethyl-2-(7-fluoro-1H-indol-3-yl)-N-methylene-1-amine (7), starting from 5,7-DiF-1H-indole (2.0 g, 0.0131 mol), oxalyl chloride (1.4 mL, 0.017 mol), and N-ethylpropylamine (1.4 g, 0.017 mol), and TEA (5.46 mL, 0.0392 mol) as starting material, yielding crude material as an orange solid (4.37 g, 0.0263 mol, quantitative yield). The product was used without further purification.

### Synthesis of N-(2-(5,7-difluoro-H-indol-3-yl)ethyl)-N-ethylpropan-1-amine (72)

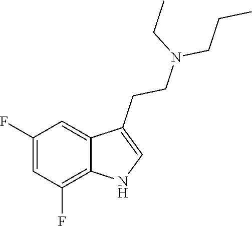 [SMILES: CCCN(CC)CCc1c[nH]c2c(F)cc(F)cc12]

N-(2-(5,7-difluoro-1H-indol-3-yl)ethyl)-N-ethylpropan-1-amine was synthesized in a similar manner as described above for N-ethyl-2-(7-fluoro-1H-indol-3-yl)-N-methylene-1-amine (7), starting from 2-(5,7-difluoro-1H-indol-3-yl)-N-ethyl-2-oxo-N-propylacetamide (2.0 g, 0.00676 mol) as the starting materials to provide the title compound as transparent yellow oil. The hydrochloride salt was collected as white crystalline solids (0.77 g, 0.00254 mol, 37.6% yield). High-resolution atmospheric solid analysis probe mass spectrometry (HR-ASAP-MS). m/z 267.1677 (theory [M+H]⁺ C₁₅H₂₁F₂N₂⁺, m/z 267.1667, Δ=3.7 ppm). [HCl] ¹H NMR (400 MHz, d₆-DMSO) δ 11.63 (s, 1H), 10.80 (s, 1H), 7.43 (d, J=2.2 Hz, 1H), 7.36 (dd, J=9.5, 2.0 Hz, 1H), 6.97 (atd, J=15.8, 1.6 Hz, 1H), 3.27-3.12 (m, 6H), 3.11-3.00 (m, 2H), 1.71 (sex, J=7.8 Hz, 2H), 1.26 (t, J=7.2 Hz, 3H), 0.92 (t, J=7.3 Hz, 3H). [HCl] ¹³C NMR (101 MHz, d₆-DMSO) δ 155.61 (dd, J=233.7, 10.0 Hz, 1C), 148.23 (dd, J=246.2, 14.6 Hz, 1C), 129.51 (dd, J=11.2, 7.3 Hz, 1C), 126.45 (s, 1C), 120.75 (d, J=13.3 Hz, 1C), 110.99 (s, 1C), 99.54 (dd, J=3.7, 23.2 Hz, 1C), 96.32 (dd, J=30.5, 20.8 Hz, 1C), 52.35 (s, 1C), 51.42 (s, 1C), 46.40 (s, 1C), 19.24 (s, 1C), 16.45 (s, 1C), 10.94 (s, 1C), 8.31 (s, 1C). [HCl] ¹⁹F NMR (377 MHz, d₆-DMSO) δ −122.07 (d, J=2.7 Hz, 1F), −129.43 (d, J=1.4 Hz, 1F).

### Example 46: N-(2-(5-fluoro-1H-indol-3-yl)ethyl)-N-methylcyclopropanamine (160)

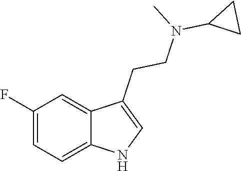 [SMILES: CN(CCc1c[nH]c2ccc(F)cc12)C1CC1]

### Synthesis of N-[2-(5-fluoro-1H-indol-3-yl)ethyl]cyclopropanamine

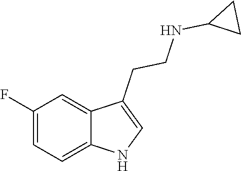 [SMILES: Fc1ccc2[nH]cc(CCNC3CC3)c2c1]

N-(2-(5-fluoro-1H-indol-3-yl)ethyl)cyclopropanamine was synthesized in a similar manner as described above for N-(2-(5-fluoro-1H-indol-3-yl)ethyl)-N-isopropylprop-2-en-1-amine (11), starting from 5-F-bromoethylindole (3.18 g, 13.14 mmol), and cyclopropylamine (3.64 mL, 52.54 mmol), to provide the title compound as a transparent amber oil (2.32 g, 0.0106 mol, 81% yield). The product was used in the subsequent reaction without further purification. High-resolution atmospheric solid analysis probe mass spectrometry (HR-ASAP-MS): m/z 219.1296 (theory [M+H]⁺ C₁₃H₁₆FN₂⁺, m/z 219.1292, Δ=1.8 ppm)

### Synthesis of N-(2-(5-fluoro-1H-indol-3-yl)ethyl)-N-methylcyclopropanamine (160)

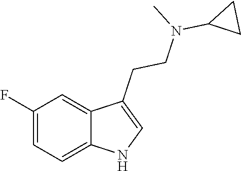 [SMILES: CN(CCc1c[nH]c2ccc(F)cc12)C1CC1]

N-(2-(5-fluoro-1H-indol-3-yl)ethyl)-N-methylcyclopropanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.4 g, 0.00183 mol) and 37% formaldehyde in water (0.38 mL, 0.00549 mol) to provide the title compound as a white powder (0.38 g, 0.00163 mol, 89% yield). The hydrochloride salt was collected as a white crystalline solid (m.p. 218.8-219.3° C.). High-resolution atmospheric solid analysis probe mass spectrometry (HR-ASAP-MS): m/z 233.1455 (theory [M+H]⁺ C₁₄H₁₈FN₂⁺, m/z 233.1449, Δ=2.6 ppm). [HCl] ¹H NMR (400 MHz, d₆-DMSO) δ 11.12 (s, 1H), 10.88 (s, 1H), 7.43 (dd, J=10.0, 2.4 Hz, 1H), 7.36 (dd, J=8.8, 4.6 Hz, 1H), 7.33 (d, J=2.3 Hz, 1H), 6.93 (td, J=9.2, 2.5 Hz, 1H), 3.47-3.36 (m, 2H)*, 3.28-3.10 (m, 2H), 2.98-2.81 (m, 1H)**, 2.87 (s, 3H)**, 1.37-1.19 (m, 1H), 1.13-0.98 (m, 1H), 0.96-0.75 (m, 2H).*=coalescing with water, **=coalescing. [HCl]¹³C NMR (101 MHz, d₆-DMSO) δ 156.74 (d, J=231.3 Hz, 1C), 132.88 (s, 1C), 126.92 (d, J=9.7 Hz, 1C), 125.33 (s, 1C), 112.51 (d, J=9.8 Hz, 1C), 109.58 (d, J=4.5 Hz, 1C), 109.33 (d, J=26.2 Hz, 1C), 103.03 (d, J=23.1 Hz, 1C), 56.38 (s, 1C), 40.79 (s, 1C), 38.56 (s, 1C), 19.87 (s, 1C), 5.01 (s, 1C), 3.13 (s, 1C). [HCl] ¹⁹F NMR (377 MHz, d₆-DMSO) δ−124.77 (s, 1F).

### Example 47: N-[2-(5-fluoro-1H-indol-3-yl)ethyl]-N-propylcyclopropanamine (161)

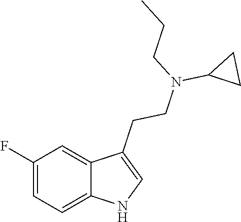 [SMILES: CCCN(CCc1c[nH]c2ccc(F)cc12)C1CC1]

N-[2-(5-fluoro-1H-indol-3-yl)ethyl]-N-propylcyclopropanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.4 g, 0.00183 mol) and propionaldehyde (0.32 g, 0.00549 mol) to provide the title compound as a colorless oil (0.30 g, 0.00115 mol, 63% yield). The hydrochloride salt was collected as a white crystalline solid. High-resolution atmospheric solid analysis probe mass spectrometry (HR-ASAP-MS): m/z 261.1764 (theory [M+H]⁺C₁₆H₂₂FN₂⁺, m/z 261.1762, Δ=0.8 ppm). [HCl] ¹H NMR (400 MHz, d₆-DMSO) δ 11.11 (s, 1H), 10.60 (s, 1H), 7.42 (dd, J=10.0, 2.4 Hz, 1H), 7.36 (dd, J=8.8, 4.5 Hz, 1H)*, 7.35 (d, J=3.5 Hz, 1H)*, 6.93 (td, J=9.2, 2.4 Hz, 1H), 3.41-3.34 (m, 2H)**, 3.26-3.14 (m, 4H), 2.94-2.85 (m, 1H), 1.91-1.1.71 (m, 2H), 1.26-1.05 (m, 2H), 0.93 (t, J=7.4 Hz, 3H), 0.89 (d, J=7.2 Hz, 2H). *=coalescing, **=coalescing with H₂O. [HCl] ¹³C NMR (101 MHz, d₆-DMSO) δ 156.76 (d, J=231.2 Hz, 1C), 132.87 (s, 1C), 126.94 (d, J=10.0 Hz, 1C), 125.40 (s, 1C), 112.52 (d, J=9.7 Hz, 1C), 109.66 (d, J=4.9 Hz, 1C), 109.34 (d, J=26.2 Hz, 1C), 102.97 (d, J=23.3 Hz, 1C), 55.81 (s, 1C), 54.49 (s, 1C), 36.50 (s, 1C), 19.50 (s, 1C), 16.75 (s, 1C), 11.02 (s, 1C), 4.20 (s, 1C), 4.19 (s, 1C). [HCl] ¹⁹F NMR (377 MHz, d₆-DMSO) δ−124.72 (s, 1F).

### Example 48: N-(2-(7-fluoro-1H-indol-3-yl)ethyl)-N-propylcyclobutanamine (162)

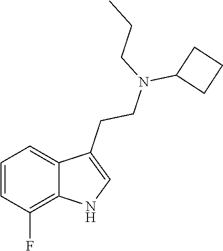 [SMILES: CCCN(CCc1c[nH]c2c(F)cccc12)C1CCC1]

### Synthesis of 2-(7-fluoro-1H-indol-3-yl)-2-oxo-N-propylacetamide

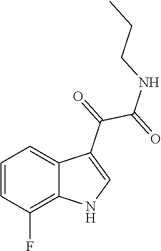 [SMILES: CCCNC(=O)C(=O)c1c[nH]c2c(F)cccc12]

2-(7-fluoro-1H-indol-3-yl)-2-oxo-N-propylacetamide was synthesized in a similar manner as described above for N-ethyl-2-(7-fluoro-1H-indol-3-yl)-N-methylene-1-amine (7), starting from 7-F-Indole (3.0 g, 0.0222 mol), oxalyl chloride (2.44 mL, 0.0289 mol), N-propylamine (2.63 mL, 0.032 mol), and TEA (13.3 mL, 0.0955 mol) as starting material, yielding crude material as a beige color (5.87 g, 0.0236 mol, quantitative yield). The product was used without further purification.

### Synthesis of N-(2-(7-fluoro-1H-indol-3-yl)ethyl)propan-1-amine

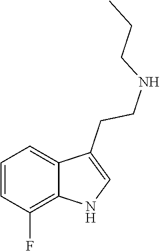 [SMILES: CCCNCCc1c[nH]c2c(F)cccc12]

N-(2-(7-fluoro-1H-indol-3-yl)ethyl)propan-1-amine was synthesized in a similar manner as described above for N-ethyl-2-(7-fluoro-1H-indol-3-yl)-N-methylene-1-amine (7), starting from 2-(7-fluoro-1H-indol-3-yl)-2-oxo-N-propylacetamide (5.85 g, 0.0236 mol) as the starting materials to provide the title compound (1.7 g, 0.00772 mol, 32.7% yield). High-resolution atmospheric solid analysis probe mass spectrometry (HR-ASAP-MS): m/z 221.1457 (theory [M+H]⁺ C₁₃H₁₈FN₂⁺, m/z 221.1449, Δ=3.6 ppm).

### Synthesis of N-(2-(7-fluoro-1H-indol-3-yl)ethyl)-N-propylcyclobutanamine (162)

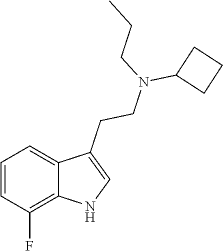 [SMILES: CCCN(CCc1c[nH]c2c(F)cccc12)C1CCC1]

N-[2-(7-fluoro-1H-indol-3-yl)ethyl]-N-propylcyclobutanamine was synthesized in a similar manner as described above for N-ethyl-N-(2-(5-fluoro-1H-indol-3-yl)ethyl)propan-2-amine (1), starting from N-[2-(7-fluoro-1H-indol-3-yl)ethyl]propan-1-amine (0.4 g, 0.00182 mol) and cyclobutanone (0.38 g, 0.00545 mol) to provide the title compound as a white-yellow tinted solid (0.38 g, 0.000902 mol, 76% yield). The hydrochloride salt was collected as a white crystalline solid (m.p. 231.7-237.2° C.). High-resolution atmospheric solid analysis probe mass spectrometry (HR-ASAP-MS): m/z 275.1931 (theory [M+H]⁺ C₁₇H₂₄FN₂⁺, m/z 275.1918, Δ=4.7 ppm). [HCl] ¹H NMR (400 MHz, d₆-DMSO) δ 11.48 (s, 1H), 10.96 (s, 1H), 7.43 (d, J=7.8 Hz, 1H), 7.36 (s, 1H), 7.04-6.96 (m, 1H)*, 6.96-6.88 (m, 1H)*, 3.92-3.77 (m, 1H), 3.22-3.07 (m, 4H), 3.07-2.90 (m, 2H), 2.48-2.34 (m, 2H)**, 2.26-2.13 (m, 2H), 1.81-1.58 (m, 4H), 0.92 (t, J=7.2 Hz, 3H). *=coalescing, **=coalescing with DMSO solvent peak. [HCl] ¹³C NMR (101 MHz, d₆-DMSO) δ 149.27 (d, J=243.1 Hz, 1C), 130.73 (d, J=5.9 Hz, 1C), 124.64 (s, 1C), 123.95 (d, J=13.2 Hz, 1C), 118.96 (d, J=6.0 Hz, 1C), 114.38 (d, J=3.1 Hz, 1C), 110.53 (s, 1C), 106.06 (d, J=16.2 Hz, 1C), 56.64 (s, 1C), 50.21 (s, 1C), 48.86 (s, 1C), 25.80 (s, 1C), 25.73 (s, 1C), 18.92 (s, 1C), 16.22 (s, 1C), 13.30 (s, 1C), 11.04 (s, 1C). [HCl] ¹⁹F NMR (377 MHz, d₆-DMSO) δ−133.06 (s, 1F).

### Example 49: 2-(5,6-difluoro-1H-indol-3-yl)-N-ethyl-N-methylethan-1-amine (61)

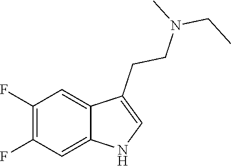 [SMILES: CCN(C)CCc1c[nH]c2cc(F)c(F)cc12]

### Synthesis of 2-(5,6-difluoro-H-indol-3-yl)-N-ethyl-N-methyl-2-oxoacetamide

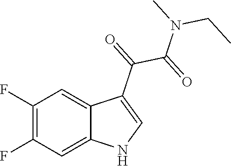 [SMILES: CCN(C)C(=O)C(=O)c1c[nH]c2cc(F)c(F)cc12]

2-(5,6-difluoro-1H-indol-3-yl)-N-ethyl-N-methyl-2-oxoacetamide was synthesized in a similar manner as described above for N-ethyl-2-(7-fluoro-1H-indol-3-yl)-N-methylene-1-amine (7), starting from 5,6-DiF-1H-indole (2.0 g, 0.0131 mol), oxalyl chloride (1.34 mL, 0.0157 mol), and N-methylethylamine (1.11 g, 0.0188 mol), and TEA (8.75 mL, 0.0628 mol) as starting material, yielding crude material as a pink powder (2.92 g, 0.0110 mol, 83.7% yield). The product was used without further purification.

### Synthesis of 2-(5,6-difluoro-H-indol-3-yl)-N-ethyl-N-methylethan-1-amine (61)

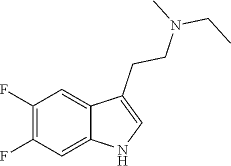 [SMILES: CCN(C)CCc1c[nH]c2cc(F)c(F)cc12]

2-(5,6-difluoro-1H-indol-3-yl)-N-ethyl-N-methylethan-1-amine was synthesized in a similar manner as described above for N-ethyl-2-(7-fluoro-1H-indol-3-yl)-N-methylene-1-amine (7), starting from 2-(5,6-difluoro-1H-indol-3-yl)-N-ethyl-N-methyl-2-oxoacetamide (2.9 g, 0.0109 mol) as the starting materials to provide the title compound as a white crystalline solid (0.82 g, 0.00344 mol, 31.6% yield). The hydrochloride salt was collected as a white crystalline solid (m.p. 166.3-168.7° C.). [HCl] ¹H NMR (400 MHz, d₆-DMSO) δ 11.21 (d, J=6.9 Hz, 1H), 10.71 (bs, 1H), 7.70 (atd, J=14.6, 1.3 Hz, 1H), 7.37 (dd, J=11.3, 7.0 Hz, 1H), 7.32 (d, J=1.8 Hz, 1H), 3.31-3.13 (m, 4H)*, 3.13-3.02 (m, 2H)*, 2.78 (s, 3H), 1.26 (t, J=7.2 Hz, 3H). *=Coalescing. [HCl] ¹³C NMR (101 MHz, d₆-DMSO) δ 146.61 (dd, J=236.9, 15.8 Hz, 1C), 145.00 (dd, J=234.1, 14.9 Hz, 1C), 131.18 (d, J=10.8 Hz, 1C), 125.30 (s, 1C), 122.13 (d, J=8.0 Hz, 1C), 109.76 (s, 1C), 105.12 (d, J=18.9 Hz, 1C), 99.36 (d, J=21.2 Hz, 1C), 54.33 (s, 1C), 49.82 (s, 1C), 38.10 (s, 1C), 19.63 (s, 1C), 8.78 (s, 1C). [HCl] ¹⁹F NMR (377 MHz, d₆-DMSO) δ−145.14 (d, J=22.5 Hz, 1F), −148.60 (d, J=21.7 Hz, 1F).

### Example 50: N-[2-(7-fluoro-1H-indol-3-yl)ethyl]-N-propylbutan-2-amine (163)

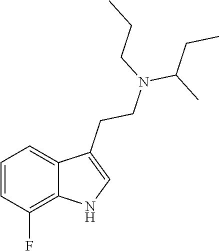 [SMILES: CCCN(CCc1c[nH]c2c(F)cccc12)C(C)CC]

N-[2-(7-fluoro-1H-indol-3-yl)ethyl]-N-propylbutan-2-amine was synthesized in a similar manner as described above for N-ethyl-N-(2-(5-fluoro-1H-indol-3-yl)ethyl)propan-2-amine (1), starting from N-[2-(7-fluoro-1H-indol-3-yl)ethyl]propan-1-amine (0.4 g, 0.00182 mol) and methyl ethyl ketone (0.79 g, 0.0109 mol) to provide the title compound (0.43 g, 0.00156 mol, 86% yield). The hydrochloride salt was collected as an off-white solid (m.p. 136.5-137.1° C.). [Free base] ¹H NMR (400 MHz, d₆-DMSO) δ 11.28 (s, 1H), 7.28 (d, J=7.8 Hz, 1H), 7.19 (s, 1H), 6.96-6.90 (m, 1H)*, 6.90-6.83 (m, 1H)*, 2.83-2.57 (m, 4H)**, 2.57-2.51 (m, 1H)**/***, 2.46-2.29 (m, 2H), 1.47-1.31 (m, 3H), 1.26-1.14 (m, 1H), 0.90-0.78 (m, 9H).*=coalescing, **=coalescing, ***=coalescing with DMSO solvent peak. [Free base] ¹³C NMR (101 MHz, d₆-DMSO) δ 149.30 (d, J=242.4 Hz, 1C), 131.44 (d, J=6.1 Hz, 1C), 123.88 (d, J=12.8 Hz, 1C), 123.78 (s, 1C), 118.40 (d, J=6.3 Hz, 1C), 114.37 (d, J=3.0 Hz, 1C), 114.12 (d, J=1.6 Hz, 1C), 105.56 (d, J=16.2 Hz, 1C), 56.37 (s, 1C), 51.55 (s, 1C), 50.65 (s, 1C), 26.58 (s, 1C), 25.39 (s, 1C), 21.97 (s, 1C), 13.82 (s, 1C), 11.83 (s, 1C), 11.62 (s, 1C). [Free base] ¹⁹F NMR (377 MHz, d₆-DMSO) δ−133.38 (s, 1F).

### Example 51: 2-(5,7-difluoro-1H-indol-3-yl)-N-ethyl-N-methylethan-1-amine (69)

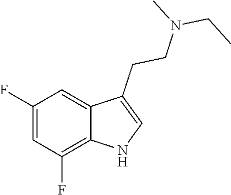 [SMILES: CCN(C)CCc1c[nH]c2c(F)cc(F)cc12]

### Synthesis of 2-(5,7-difluoro-1H-indol-3-yl)-N-ethyl-N-methyl-2-oxoacetamide

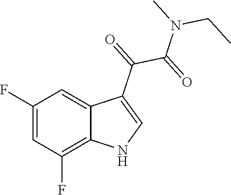 [SMILES: CCN(C)C(=O)C(=O)c1c[nH]c2c(F)cc(F)cc12]

2-(5,7-difluoro-1H-indol-3-yl)-N-ethyl-N-methyl-2-oxoacetamide was synthesized in a similar manner as described above for N-ethyl-2-(7-fluoro-1H-indol-3-yl)-N-methylene-1-amine (7), starting from 5,7-DiF-1H-indole (1.0 g, 0.00653 mol), oxalyl chloride (1.08 g, 0.00849 mol), and N-methylethylamine (0.56 g, 0.0094 mol), and TEA (3.81 mL, 0.0281 mol) as starting material, yielding crude material as a pink powder (1.61 g, 0.00605 mol, 93% yield). The product was used without further purification.

### Synthesis of 2-(5,7-difluoro-1H-indol-3-yl)-N-ethyl-N-methylethan-1-amine (69)

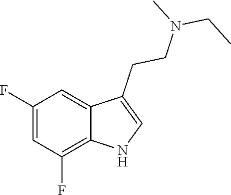 [SMILES: CCN(C)CCc1c[nH]c2c(F)cc(F)cc12]

2-(5,7-difluoro-1H-indol-3-yl)-N-ethyl-N-methylethan-1-amine was synthesized in a similar manner as described above for N-ethyl-2-(7-fluoro-1H-indol-3-yl)-N-methylene-1-amine (7), starting from 2-(5,7-difluoro-1H-indol-3-yl)-N-ethyl-N-methyl-2-oxoacetamide (1.6 g, 0.006 mol) as the starting materials to provide the title compound as a transparent amber oil (1.32 g, 0.00546 mol, 92.3% yield). The hydrochloride salt was collected as a white crystalline solid (m.p. 152.7-153.5° C.). High-resolution atmospheric solid analysis probe mass spectrometry (HR-ASAP-MS): m/z 239.1355 (theory [M+H]⁺ C₁₃H₁₇F₂N₂⁺, m/z 239.1354, Δ=2.8 ppm). [HCl]¹H NMR (400 MHz, d₆-DMSO) δ 11.63 (s, 1H), 10.84 (s, 1H), 7.41 (d, J=2.2 Hz, 1H), 7.37 (dd, J=9.5, 1.9 Hz, 1H), 7.01-6.91 (m, 1H), 3.32-3.22 (m, 2H)*, 3.22-3.15 (m, 2H)*, 3.15-3.03 (m, 2H)*, (2.78 (s, 3H), 1.26 (t, J=7.2 Hz, 3H). *=coalescing. [HCl] ¹³C NMR (101 MHz, d₆-DMSO) δ 155.61 (dd, J=233.8, 10.0 Hz, 1C), 148.23 (dd, J=246.2, 14.7 Hz, 1C), 129.50 (dd, J=11.3, 7.3 Hz, 1C), 126.41 (s, 1C), 120.78 (d, J=13.1 Hz, 1C), 110.94 (s, 1C), 99.58 (dd, J=23.2, 3.8 Hz, 1C), 96.33 (dd, J=30.5, 20.9 Hz, 1C), 54.19 (s, 1C), 49.82 (s, 1C), 38.12 (s, 1C), 19.56 (s, 1C), 8.76 (s, 1C). [HCl] ¹⁹F NMR (377 MHz, d₆-DMSO) δ−122.11 (d, J=2.7 Hz, 1F), −129.44 (d, J=2.7 Hz, 1F).

### Example 52: N-ethyl-2-(7-fluoro-5-methoxy-1H-indol-3-yl)-N-methylethan-1-amine (88)

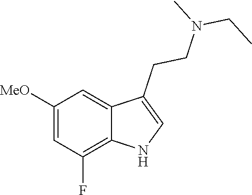 [SMILES: CCN(C)CCc1c[nH]c2c(F)cc(OC)cc12]

### Synthesis of N-ethyl-2-(7-fluoro-5-methoxy-1H-indol-3-yl)-N-methyl-2-oxoacetamide

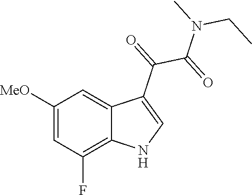 [SMILES: CCN(C)C(=O)C(=O)c1c[nH]c2c(F)cc(OC)cc12]

N-ethyl-2-(7-fluoro-5-methoxy-1H-indol-3-yl)-N-methyl-2-oxoacetamide was synthesized in a similar manner as described above for N-ethyl-2-(7-fluoro-1H-indol-3-yl)-N-methylene-1-amine (7), starting from 5-MeO-7-F-indole (1.0 g, 0.00620 mol), oxalyl chloride (0.94 g, 0.00744 mol), and N-methylethylamine (0.53 g, 0.00893 mol), and TEA (3.02 g, 0.0298 mol) as starting material, yielding crude material as a tan brown powder (1.40 g, 0.00503 mol, 80.9% yield). The product was used without further purification.

### Synthesis of N-ethyl-2-(7-fluoro-5-methoxy-1H-indol-3-yl)-N-methylethan-1-amine (88)

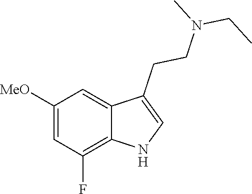 [SMILES: CCN(C)CCc1c[nH]c2c(F)cc(OC)cc12]

N-ethyl-2-(7-fluoro-5-methoxy-1H-indol-3-yl)-N-methylethan-1-amine was synthesized in a similar manner as described above for N-ethyl-2-(7-fluoro-1H-indol-3-yl)-N-methylene-1-amine (7), starting from N-ethyl-2-(7-fluoro-5-methoxy-1H-indol-3-yl)-N-methyl-2-oxoacetamide (1.4 g, 0.00503 mol) as the starting materials to provide the title compound as a transparent purple tinted oil (1.18 g, 0.00471 mol, 93.7% yield). The hydrochloride salt was collected as a white crystalline solid (m.p. 142.7-143.2° C.). High-resolution atmospheric solid analysis probe mass spectrometry (HR-ASAP-MS): m/z 251.1561 (theory [M+H]+C₁₄H₂OFN₂O, m/z 251.1554, Δ=2.8 ppm) [HCl] ¹H NMR (400 MHz, d₆-DMSO) δ 11.29 (s, 1H), 10.88 (s, 1H), 7.27 (d, J=2.0 Hz, 1H), 7.05 (m, 1H), 6.63 (dd, J=12.7, 1.5 Hz, 1H), 3.79 (s, 3H), 3.30-3.21 (m, 2H)*, 3.21-3.13 (m, 2H)*, 3.13-3.02 (m, 2H)*, 2.78 (s, 3H), 1.27 (t, J=7.2 Hz, 3H). *=Coalescing. [HCl] ¹³C NMR (101 MHz, d₆-DMSO) δ 153.20 (d, J=9.2 Hz, 1C), 148.85 (d, J=243.3 Hz, 1C), 130.16 (d, J=7.1 Hz, 1C), 124.97 (s, 1C), 119.11 (d, J=13.4 Hz, 1C), 110.21 (s, 1C), 97.12 (d, J=19.4 Hz, 1C), 96.55 (d, J=3.0 Hz, 1C), 55.90 (s, 1C), 54.19 (s, 1C), 49.78 (s, 1C), 38.14 (s, 1C), 19.70 (s, 1C). [HCl] ¹⁹F NMR (377 MHz, d₆-DMSO) δ−131.36 (s, 1F).

### Example 53: N-ethyl-N-methyl-2-(5,6,7-trifluoro-1H-indol-3-yl)ethan-1-amine (164)

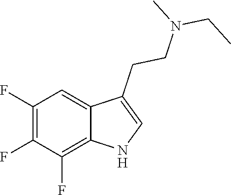 [SMILES: CCN(C)CCc1c[nH]c2c(F)c(F)c(F)cc12]

### Synthesis of N-ethyl-N-methyl-2-oxo-2-(5,6,7-trifluoro-1H-indol-3-yl)acetamide

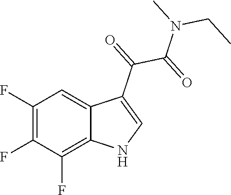 [SMILES: CCN(C)C(=O)C(=O)c1c[nH]c2c(F)c(F)c(F)cc12]

N-ethyl-N-methyl-2-oxo-2-(5,6,7-trifluoro-1H-indol-3-yl)acetamide was synthesized in a similar manner as described above for N-ethyl-2-(7-fluoro-1H-indol-3-yl)-N-methylene-1-amine (7), starting from 5,6,7-TriF-Indole (1.0 g, 0.00584 mol), oxalyl chloride (0.96 g, 0.00760 mol), and N-methylethylamine (0.50 g, 0.00842 mol), and TEA (2.54 g, 0.0251 mol) as starting material, yielding crude material as a tan solids (1.22 g, 0.00504 mol, 86.2% yield). The product was used without further purification.

### Synthesis of N-ethyl-N-methyl-2-(5,6,7-trifluoro-1H-indol-3-yl)ethan-1-amine (164)

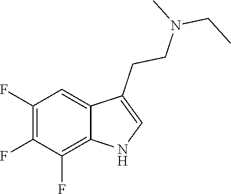 [SMILES: CCN(C)CCc1c[nH]c2c(F)c(F)c(F)cc12]

N-ethyl-N-methyl-2-(5,6,7-trifluoro-1H-indol-3-yl)ethan-1-amine was synthesized in a similar manner as described above for N-ethyl-2-(7-fluoro-1H-indol-3-yl)-N-methylene-1-amine (7), starting from N-ethyl-N-methyl-2-oxo-2-(5,6,7-trifluoro-1H-indol-3-yl)acetamide (1.4 g, 0.00503 mol) as the starting materials to provide the title compound. The hydrochloride salt was collected as a white crystalline solid (0.35 g, 0.00162 mol, 32.2% yield) (m.p. 212.1-212.5° C.). High-resolution atmospheric solid analysis probe mass spectrometry (HR-ASAP-MS): m/z 257.1269 (theory [M+H]⁺ C₁₃H₁₆F₃N₂⁺, m/z 257.1260, Δ=3.5 ppm). [HCl] ¹H NMR (400 MHz, d₆-DMSO) δ 11.83 (s, 1H), 10.76 (s, 1H), 7.62 (ddd, J=10.7, 6.8, 1.1 Hz, 1H), 7.44 (d, J=2.2 Hz, 1H), 3.32-3.22 (m, 2H), 3.22-3.15 (m, 2H), 3.15-3.03 (m, 2H), 2.78 (s, 3H), 1.26 (t, J=7.3 Hz, 3H). [HCl] ¹³C NMR (101 MHz, d₆-DMSO) δ 145.16 (dd, J=235.7, 12.1 Hz, 1C), 137.31 (ddd, J=247.9, 13.2, 4.7, Hz, 1C), 135.17 (ddd, J=238.2, 18.8, 12.6 Hz, 1C), 126.44 (d, J=3.0 Hz, 1C), 122.94 (dd, J=9.1, 6.3 Hz, 1C), 120.45 (dd, J=9.5, 1.5 Hz, 1C), 110.95 (d, J=4.9 Hz, 1C), 100.46 (dd, J=19.1, 3.6 Hz, 1C), 54.14 (s, 1C), 49.86 (s, 1C), 38.13 (s, 1C), 19.38 (s, 1C), 8.78 (s, 1C). [HCl] ¹⁹F NMR (377 MHz, d₆-DMSO) δ−146.19 (dd, J=21.9, 10.9 Hz, 1F), −155.18 (dd, J=20.4, 2.7 Hz, 1F), −170.45 (td, J=21.8, 6.8 Hz, 1F).

### Example 54: N-ethyl-N-[2-(5,6,7-trifluoro-1H-indol-3-yl)ethyl]propan-1-amine (165)

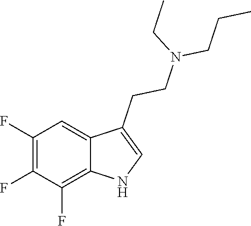 [SMILES: CCCN(CC)CCc1c[nH]c2c(F)c(F)c(F)cc12]

### Synthesis of N-ethyl-2-oxo-N-propyl-2-(5,6,7-trifluoro-1H-indol-3-yl)acetamide

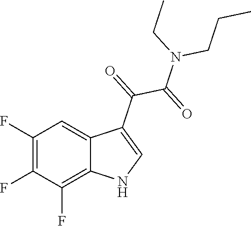 [SMILES: CCCN(CC)C(=O)C(=O)c1c[nH]c2c(F)c(F)c(F)cc12]

N-ethyl-2-oxo-N-propyl-2-(5,6,7-trifluoro-1H-indol-3-yl)acetamide was synthesized in a similar manner as described above for N-ethyl-2-(7-fluoro-1H-indol-3-yl)-N-methylene-1-amine (7), starting from 5,6,7-TriF-indole (1.0 g, 0.00584 mol), oxalyl chloride (0.96 g, 0.00760 mol), and N-ethylpropylamine (0.89 mL, 0.00760 mol), and TEA (2.44 mL, 0.0175 mol) as starting material, yielding crude material as brown solids (2.96 g, 0.00948 mol, quant yield). The product was used without further purification.

### Synthesis of N-ethyl-N-[2-(5,6,7-trifluoro-1H-indol-3-yl)ethyl]propan-1-amine (165)

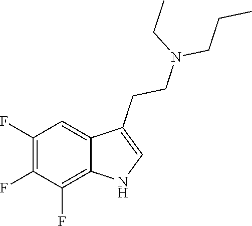 [SMILES: CCCN(CC)CCc1c[nH]c2c(F)c(F)c(F)cc12]

N-ethyl-N-[2-(5,6,7-trifluoro-1H-indol-3-yl)ethyl]propan-1-amine was synthesized in a similar manner as described above for N-ethyl-2-(7-fluoro-1H-indol-3-yl)-N-methylene-1-amine (7), starting from N-ethyl-2-oxo-N-propyl-2-(5,6,7-trifluoro-1H-indol-3-yl)acetamide (2.96 g, 0.00948 mol) as the starting materials to provide the title compound as an amber oil (1.25 g, 0.00440, 46.4% yield). The hydrochloride salt was collected as a white crystalline solid (m.p. 188.0-188.8° C.). High-resolution atmospheric solid analysis probe mass spectrometry (HR-ASAP-MS): m/z 285.1586 (theory [M+H]⁺ C₁₅H₂₁F₃N₂⁺, m/z 285.1573, Δ=4.6 ppm). [HCl]¹H NMR (400 MHz, d₆-DMSO) δ 11.84 (s, 1H), 10.87 (s, 1H), 7.62 (ddd, J=10.8, 6.7, 1.2 Hz, 1H), 7.46 (d, J=2.1 Hz, 1H), 3.28-3.3.12 (m, 6H), 3.11-3.00 (m, 2H), 1.71 (sex, J=7.6 Hz, 2H), 1.25 (t, J=7.2 Hz, 3H), 0.92 (t, J=7.3 Hz, 3H). [HCl] ¹³C NMR (101 MHz, d₆-DMSO) δ 145.14 (dd, J=235.8, 11.9 Hz, 1C), 137.28 (ddd, J=247.9, 13.0, 4.5 Hz, 1C), 135.14 (ddd, J=238.4, 18.8, 12.2 Hz, 1C), 126.44 (d, J=2.7 Hz, 1C), 122.94 (dd, J=9.4, 5.9 Hz, 1C), 120.39 (dd, J=9.9, 2.0 Hz, 1C), 111.07 (d, J=2.5 Hz, 1C), 100.42 (dd, J=19.2, 3.5 Hz, 1C), 52.33 (s, 1C), 51.39 (s, 1C), 46.37 (s, 1C), 19.06 (s, 1C), 16.44 (s, 1C), 10.93 (s, 1C), 8.30 (s, 1C). [HCl]¹⁹F NMR (377 MHz, d₆-DMSO) δ−146.17 (d, J=21.7 Hz, 1F), −155.19 (d, J=20.4 Hz, 1F), −170.47 (t, J=21.1 Hz, 1F).

### Example 55: N-ethyl-N-(2-(7-fluoro-5-methyl-1H-indol-3-yl)ethyl)propan-1-amine (166)

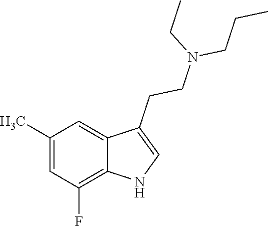 [SMILES: CCCN(CC)CCc1c[nH]c2c(F)cc(C)cc12]

### Synthesis of N-ethyl-2-(7-fluoro-5-methyl-1H-indol-3-yl)-2-oxo-N-propylacetamide

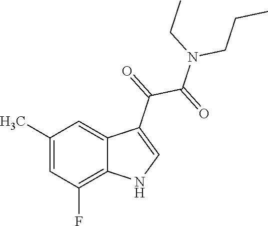 [SMILES: CCCN(CC)C(=O)C(=O)c1c[nH]c2c(F)cc(C)cc12]

N-ethyl-2-(7-fluoro-5-methyl-1H-indol-3-yl)-2-oxo-N-propylacetamide was synthesized in a similar manner as described above for N-ethyl-2-(7-fluoro-1H-indol-3-yl)-N-methylene-1-amine (7), starting from 5-Me-7-F-indole (1.0 g, 0.00670 mol), oxalyl chloride (0.74 g, 0.00872 mol), and N-ethylpropylamine (2.0 mL, 0.0172 mol), and TEA (4.00 mL, 0.0287 mol) as starting material, yielding crude material as beige solids (2.32 g, 0.00799 mol, quant yield). The product was used without further purification.

### Synthesis of N-ethyl-N-(2-(7-fluoro-5-methyl-1H-indol-3-yl)ethyl)propan-1-amine (166)

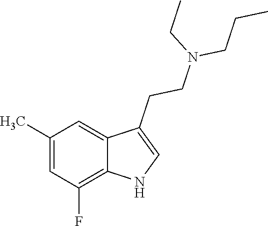 [SMILES: CCCN(CC)CCc1c[nH]c2c(F)cc(C)cc12]

N-ethyl-N-[2-(7-fluoro-5-methyl-1H-indol-3-yl)ethyl]propan-1-amine was synthesized in a similar manner as described above for N-ethyl-2-(7-fluoro-1H-indol-3-yl)-N-methylene-1-amine (7), starting from N-ethyl-2-(7-fluoro-5-methyl-1H-indol-3-yl)-2-oxo-N-propylacetamide (1.83 g, 0.00629 mol) as the starting materials to provide the title compound. The hydrochloride salt was collected as off-white crystalline solids (0.85 g, 0.00284, 45.2% yield). High-resolution atmospheric solid analysis probe mass spectrometry (HR-ASAP-MS): m/z 263.1919 (theory [M+H]⁺ C₁₆H₂₄FN₂⁺, m/z 263.1918, Δ=0.4 ppm). [HCl] ¹H NMR (400 MHz, d₆-DMSO) δ 11.34 (s, 1H), 10.81 (s, 1H), 7.29 (d, J=2.2 Hz, 1H), 7.24 (s, 1H), 6.76 (d, J=12.4 Hz, 1H), 3.28-3.16 (m, 4H)*, 3.16-3.10 (m, 2H)*, 3.10-3.01 (m, 2H)*, 2.38 (s, 3H), 1.72 (sex, J=7. Hz, 2H), 1.26 (t, J=7.2 Hz, 3H), 0.92 (t, J=7.4 Hz, 3H). *=coalescing. [HCl] ¹³C NMR (101 MHz, d₆-DMSO) δ 148.82 (d, J=242.8 Hz, 1C), 130.78 (d, J=6.1 Hz, 1C), 128.19 (d, J=5.7 Hz, 1C), 124.52 (s, 1C), 122.18 (d, J=13.6 Hz, 1C), 113.94 (d, J=2.8 Hz, 1C), 110.00 (d, J=2.1 Hz, 1C), 107.53 (d, J=15.8 Hz, 1C), 52.44 (s, 1C), 51.45 (s, 1C), 46.48 (s, 1C), 21.06 (s, 1C), 19.30 (s, 1C), 16.48 (s, 1C), 10.95 (s, 1C), 8.35 (s, 1C). [HCl] ¹⁹F NMR (377 MHz, d₆-DMSO) δ−133.64 (s, 1F).

### Example 56: N-(2-(6,7-difluoro-1H-indol-3-yl)ethyl)-N-ethylpropan-1-amine (82)

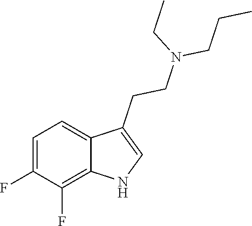 [SMILES: CCCN(CC)CCc1c[nH]c2c(F)c(F)ccc12]

### Synthesis of 2-(6,7-difluoro-1H-indol-3-yl)-N-ethyl-2-oxo-N-propylacetamide

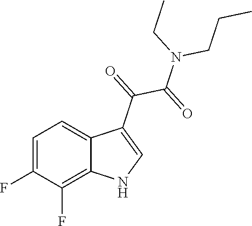 [SMILES: CCCN(CC)C(=O)C(=O)c1c[nH]c2c(F)c(F)ccc12]

2-(6,7-difluoro-1H-indol-3-yl)-N-ethyl-2-oxo-N-propylacetamide was synthesized in a similar manner as described above for N-ethyl-2-(7-fluoro-1H-indol-3-yl)-N-methylene-1-amine (7), starting from 6,7-DiF-indole (1.0 g, 0.00653 mol), oxalyl chloride (0.72 mL, 0.00850 mol), and N-ethylpropylamine (1.14 g, 0.0131 mol), and TEA (1.32 g, 0.01306 mol) as starting material, yielding crude material as a beige solid (1.22 g, 0.00415 mol, 63.55% yield). The product was used without further purification.

### Synthesis of N-(2-(6,7-difluoro-1H-indol-3-yl)ethyl)-N-ethylpropan-1-amine (82)

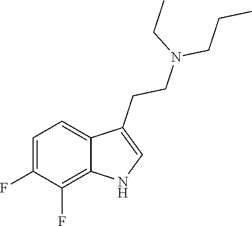 [SMILES: CCCN(CC)CCc1c[nH]c2c(F)c(F)ccc12]

N-[2-(6,7-difluoro-1H-indol-3-yl)ethyl]-N-ethylpropan-1-amine was synthesized in a similar manner as described above for N-ethyl-2-(7-fluoro-1H-indol-3-yl)-N-methylene-1-amine (7), starting from 2-(6,7-difluoro-1H-indol-3-yl)-N-ethyl-2-oxo-N-propylacetamide (1.22 g, 0.00415 mol) as the starting materials to provide the title compound. The hydrochloride salt was collected as an off-white crystalline solid (0.17 g, 0.000638, 15.4% yield). High-resolution atmospheric solid analysis probe mass spectrometry (HR-ASAP-MS): m/z 267.1668 (theory [M+H]⁺ C₁₅H₂₁F₂N₂⁺, m/z 267.1667, Δ=0.4 ppm). [HCl] ¹H NMR (400 MHz, d₆-DMSO) δ 11.69 (s, 1H), 10.85 (s, 1H), 7.46 (dd, J=8.7, 4.1 Hz, 1H), 7.38 (d, J=2.2 Hz, 1H), 7.10-7.00 (m, 1H), 3.30-3.12 (m, 6H)*, 3.12-2.99 (m, 2H)*, 1.72 (sex, J=7.7 Hz, 2H), 1.26 (t, J=7.2 Hz, 3H), 0.92 (t, J=7.4 Hz, 3H). *=coalescing. [HCl] ¹³C NMR (101 MHz, d₆-DMSO) δ 145.17 (dd, J=234.3, 9.2 Hz, 1C), 136.84 (dd, J=245.2, 16.1 Hz, 1C), 126.71 (d, J=4.6 Hz, 1C), 125.22 (d, J=3.0 Hz, 1C), 124.59 (d, J=9.8 Hz, 1C), 114.15 (dd, J=8.4, 3.7 Hz, 1C), 110.69 (s, 1C), 108.29 (d, J=20.1 Hz, 1C), 52.39 (s, 1C), 51.37 (s, 1C), 46.44 (s, 1C), 19.11 (s, 1C), 16.44 (s, 1C), 10.94 (s, 1C), 8.31 (s, 1C). [HCl] ¹⁹F NMR (377 MHz, d₆-DMSO) δ−150.00 (d, J=21.7 Hz, 1F), −159.49 (d, J=21.8 Hz, 1F).

### Example 57: N-ethyl-N-(2-(5-fluoro-1H-indol-3-yl)ethyl)cyclopropanamine (167)

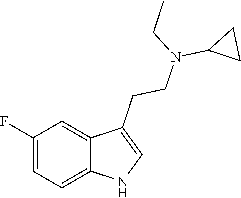 [SMILES: CCN(CCc1c[nH]c2ccc(F)cc12)C1CC1]

N-ethyl-N-[2-(5-fluoro-1H-indol-3-yl)ethyl]cyclopropanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.4 g, 0.00183 mol) and acetaldehyde (0.48 g, 0.0110 mol) to provide the title compound as a colorless oil (0.41 g, 0.00166 mol, 91% yield). The hydrochloride salt was collected as a white crystalline powder (m.p. 219.7-220.1° C.). High-resolution atmospheric solid analysis probe mass spectrometry (HR-ASAP-MS): m/z 247.1607 (theory [M+H]⁺ C₁₆H₂₂FN₂⁺, m/z 247.1605, Δ=0.8 ppm). [HCl] ¹H NMR (400 MHz, d₆-DMSO) δ 11.12 (s, 1H), 10.80 (s, 1H), 7.42 (dd, J=10.0, 2.4 Hz, 1H), 7.36 (dd, J=8.6, 4.6 Hz, 1H)*, 7.35 (d, J=3.1 Hz, 1H)*, 6.93 (td, J=9.2, 2.4 Hz, 1H), 3.36-3.27 (m, 4H)**, 3.26-3.11 (m, 2H)**, 2.94-2.83 (m, 1H), 1.32 (t, J=7.3 Hz, 3H), 1.29-1.20 (m, 1H), 1.20-1.10 (m, 1H), 0.96-0.84 (m, 2H). *=coalescing, **=coalescing with H₂O peak and other peak. [HCl] ¹³C NMR (101 MHz, d₆-DMSO) δ 156.75 (d, J=231.2 Hz, 1C), 132.87 (s, 1C), 126.94 (d, J=9.8 Hz, 1C), 125.38 (s, 1C), 112.51 (d, J=9.8 Hz, 1C), 109.69 (d, J=4.8 Hz, 1C), 109.33 (d, J=26.0 Hz, 1C), 102.96 (d, J=23.2 Hz, 1C), 53.74 (s, 1C), 49.32 (s, 1C), 35.92 (s, 1C), 19.51 (s, 1C), 8.77 (s, 1C), 4.12 (s, 1C), 3.99 (s, 1C). [HCl] ¹⁹F NMR (377 MHz, d₆-DMSO) δ−124.71 (s, 1F).

### Example 58: N-(2-(5-fluoro-1H-indol-3-yl)ethyl)-N-isopropylcyclopropanamine (168)

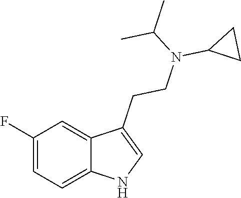 [SMILES: CC(C)N(CCc1c[nH]c2ccc(F)cc12)C1CC1]

N-[2-(5-fluoro-1H-indol-3-yl)ethyl]-N-(propan-2-yl)cyclopropanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.4 g, 0.00183 mol) and acetone (8.0 mL, 0.108 mol) to provide the title compound as transparent white crystals. The hydrochloride salt was collected as a white crystalline powder (0.42 g, 0.00142 mol, 77.6% yield) (m.p. 196.4-196.8° C.). Variation to (1) was that acetone was utilized as the solvent system as opposed to using MeOH. High-resolution atmospheric solid analysis probe mass spectrometry (HR-ASAP-MS): m/z 261.1774 (theory [M+H]⁺ C₁₆H₂₂FN₂⁺, m/z 261.1762, Δ=4.6 ppm). [HCl] ¹H NMR (400 MHz, d₆-DMSO) δ 11.12 (s, 1H), 10.61 (s, 1H), 7.42 (dd, J=10.0, 2.4 Hz, 1H), 7.36 (d, J=2.8 Hz, 1H)*, 7.36 (dd, J=8.7, 4.7 Hz, 1H)*, 6.93 (td, J=9.2, 2.4 Hz, 1H), 3.79-3.66 (m, 1H), 3.32-3.17 (m, 4H)**, 2.94-2.83 (m, 1H), 1.43-1.29 (m, 7H), 1.21-1.11 (m, 1H), 0.97-0.84 (m, 2H).*=coalescing, **=coalescing with H₂O peak. [HCl] ¹³C NMR (101 MHz, d₆-DMSO) δ 156.76 (d, J=231.2 Hz, 1C), 132.86 (s, 1C), 126.98 (d, J=9.8 Hz, 1C), 125.43 (s, 1C), 112.50 (d, J=9.7 Hz, 1C), 109.83 (d, J=4.7 Hz, 1C), 109.32 (d, J=26.2 Hz, 1C), 102.94 (d, J=23.2 Hz, 1C), 56.49 (s, 1C), 50.86 (s, 1C), 33.88 (s, 1C), 20.02 (s, 1C), 16.57 (s, 1C), 16.50 (s, 1C), 4.25 (s, 1C), 4.10 (s, 1C). [HCl] ¹⁹F NMR (377 MHz, d₆-DMSO) δ−124.69 (s, 1F).

### Example 59: N-ethyl-2-(5-fluoro-7-methyl-1H-indol-3-yl)-N-methylethan-1-amine (169)

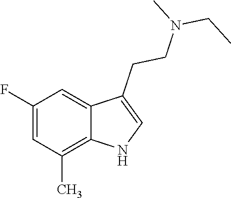 [SMILES: CCN(C)CCc1c[nH]c2c(C)cc(F)cc12]

### Synthesis of N-ethyl-2-(5-fluoro-7-methyl-1H-indol-3-yl)-N-methyl-2-oxoacetamide

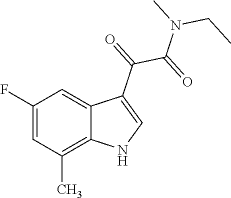 [SMILES: CCN(C)C(=O)C(=O)c1c[nH]c2c(C)cc(F)cc12]

N-ethyl-2-(5-fluoro-7-methyl-1H-indol-3-yl)-N-methyl-2-oxoacetamide was synthesized in a similar manner as described above for N-ethyl-2-(7-fluoro-1H-indol-3-yl)-N-methylene-1-amine (7), starting from 5-F-7-Me-Indole (0.95 g, 0.00637 mol), oxalyl chloride (0.97 g, 0.00764 mol), and N-methylethylamine (0.54 g, 0.00917 mol), and TEA (3.1 g, 0.0306 mol) as starting material, yielding crude material as orange solids (1.20 g, 0.00457 mol, 59.89% yield). The product was used without further purification.

### Synthesis of N-ethyl-2-(5-fluoro-7-methyl-1H-indol-3-yl)-N-methylethan-1-amine (169)

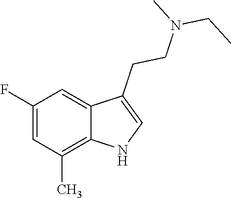 [SMILES: CCN(C)CCc1c[nH]c2c(C)cc(F)cc12]

N-ethyl-2-(5-fluoro-7-methyl-1H-indol-3-yl)-N-methylethan-1-amine was synthesized in a similar manner as described above for N-ethyl-2-(7-fluoro-1H-indol-3-yl)-N-methylene-1-amine (7), starting from N-ethyl-2-(5-fluoro-7-methyl-1H-indol-3-yl)-N-methyl-2-oxoacetamide (1.15 g, 0.00438 mol) as the starting materials to provide the title compound as a transparent amber oil (0.77 g, 0.00329 mol, 75.1% yield). The material formed an amorphous solid upon converting into HCl salt so that material was converted back and kept as the free base. High-resolution atmospheric solid analysis probe mass spectrometry (HR-ASAP-MS): m/z 235.1612 (theory [M+H]⁺ C₁₄H₂OFN₂⁺, m/z 235.1605, Δ=3.0 ppm). [Free base] ¹H NMR (400 MHz, d₆-DMSO) δ 10.84 (s, 1H), 7.20 (d, J=2.1 Hz, 1H), 7.04 (dd, J=9.9, 2.2 Hz, 1H), 6.72 (dd, J=10.2, 1.7 Hz, 1H), 2.79-2.72 (m, 2H), 2.60-2.52 (m, 2H), 2.43 (s, 3H)*, 2.42 (q, J=7.1 Hz, 1H)*, 2.21 (s, 3H), 0.99 (t, J=7.1 Hz, 3H). *=coalescing. [Free base] ¹³C NMR (101 MHz, d₆-DMSO) δ 156.60 (d, J=230.6 Hz, 1C), 132.29 (s, 1C), 126.87 (d, J=10.2 Hz, 1C), 124.10 (s, 1C), 122.09 (d, J=9.5 Hz, 1C), 113.55 (d, J=5.0 Hz, 1C), 109.30 (d, J=25.8 Hz, 1C), 100.24 (d, J=22.7 Hz, 1C), 57.58 (s, 1C), 50.73 (s, 1C), 41.20 (s, 1C), 22.73 (s, 1C), 16.58 (d, J=1.1 Hz, 1C), 12.23 (s, 1C). [Free base] ¹⁹F NMR (377 MHz, d₆-DMSO) δ−125.57 (s, 1F).

### Example 60: (R)-3-((1-ethylpyrrolidin-2-yl)methyl)-5-fluoro-1H-indole (170)

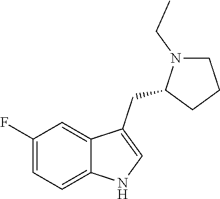 [SMILES: CCN1CCC[C@@H]1Cc1c[nH]c2ccc(F)cc12]

### Synthesis of benzyl (2R)-2-(5-fluoro-H-indole-3-carbonyl)pyrrolidine-1-carboxylate

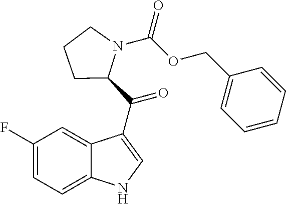 [SMILES: O=C(c1c[nH]c2ccc(F)cc12)[C@H]1CCCN1C(=O)OCc1ccccc1]

Benzyl (2R)-2-(5-fluoro-1H-indole-3-carbonyl)pyrrolidine-1-carboxylate was synthesized in a similar manner as described above for (S)-5-fluoro-3-((1-methylpyrrolidin-2-yl)methyl)-1H-indole, starting from 5-fluoroindole (5.41 g, 0.0400 mol), N-benzyloxylcarbonyl-D-proline (5 g, 0.0200 mol), oxalyl chloride (3.8 g, 0.0300 mol), and 1.4 M methylmagnesium bromide in 1:3 THF/Toluene (28.6 mL, 0.0400 mol) as the starting materials to provide the title compound as a white solid (2.8 g, 0.00764 mol, 38.2% yield). The product was used without further purification.

### Synthesis of (5-fluoro-1H-indol-3-yl)[(2R)-pyrrolidin-2-yl]methanone

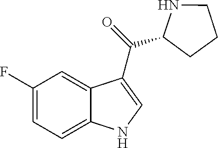 [SMILES: O=C(c1c[nH]c2ccc(F)cc12)[C@H]1CCCN1]

In an oven dried 300 mL one neck round bottom flask was added benzyl (2R)-2-(5-fluoro-1H-indole-3-carbonyl)pyrrolidine-1-carboxylate (1 g, 0.00273 mol), and was dissolved in 60 mL of MeOH by gentling heating the solution. Once the material was dissolved 10% Pd-C (0.29 g, 0.00273 mol) was added to the reaction and then a balloon of H₂ was flushed through the reaction vessel. Once the balloon was empty a new balloon of H₂ was added to the septum of the reaction and allowed to react for 4 h. After 4 h, the reaction was confirmed to be complete by TLC and MS. The Pd-C was filtered and the mother liquor was filtered through celite directly into a 500 mL RBF. This filtrate was removed under reduced pressure to yield the titled compound as an orange solid (0.60 g, 0.00258 mol, 94.5% yield). The product was used without further purification.

### Synthesis of 1-[(2R)-2-(5-fluoro-1H-indole-3-carbonyl)pyrrolidin-1-yl]ethan-1-one

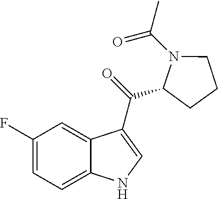 [SMILES: CC(=O)N1CCC[C@@H]1C(=O)c1c[nH]c2ccc(F)cc12]

In an 100 mL one neck round bottom flask 1-[(2R)-2-(5-fluoro-1H-indole-3-carbonyl)pyrrolidin-1-yl]ethan-1-one (0.4 g, 0.00172 mol) was added to 10 mL of dichloromethane but did not dissolve. TEA (0.52 g, 0.00516 mol) was added to the reaction and then the round bottom flask was put on an ice bath before adding acetyl chloride (0.16 g, 0.00206 mol). Upon adding acetyl chloride the precipitate went into solution and was allowed to react for 1 h. Upon 1 h, the reaction was shown to be complete by TLC and MS. The reaction was worked up by quenching with water and extracting three times with EtOAc (3*50 mL). Upon completing the extractions, the organic phase was washed with brine and dried with Na₂SO₄. The solvent was removed under reduced pressure to yield the title compound as an orange solid (0.45 g, 0.00164 mol, 95.3% yield).

### Synthesis of (R)-3-((I-ethylpyrrolidin-2-yl)methyl)-5-fluoro-1H-indole (170)

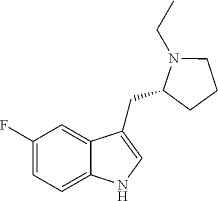 [SMILES: CCN1CCC[C@@H]1Cc1c[nH]c2ccc(F)cc12]

(5-fluoro-3-{[(2R)-pyrrolidin-2-yl]methyl}-1H-indole) was synthesized in a similar manner as described above for N-ethyl-2-(7-fluoro-1H-indol-3-yl)-N-methylene-1-amine (7), starting from 1-[(2R)-2-(5-fluoro-1H-indole-3-carbonyl)pyrrolidin-1-yl]ethan-1-one (0.45 g, 0.00164 mol) as the starting material to provide the title compound (0.28 g, 0.00114 mol, 69.51% yield). The freebase was collected as an amber oil. [Free base] ¹H NMR (400 MHz, d₆-DMSO) δ 10.88 (s, 1H), 7.30 (dd, J=8.7, 4.6 Hz, 1H), 7.22 (dd, J=9.3, 2.3 Hz, 1H)*, 7.21 (d, J=2.3 Hz, 1H)*, 6.89 (td, J=13.7, 2.3 Hz, 1H), 3.13-3.03 (m, 1H)**, 3.03-2.90 (m, 2H)**, 2.61-2.51 (m, 1H)***, 2.47 (d, J=9.2 Hz, 1H), 2.26-2.12 (m, 1H)****, 2.12-2.01 (m, 1H)****, 1.73-1.58 (m, 2H), 1.58-1.42 (m, 2H), 1.07 (t, J=7.2 Hz, 3H). *=coalescing, **=coalescing, ***=coalescing with DMSO solvent, ****=coalescing. [Free base] ¹³C NMR (101 MHz, d₆-DMSO) δ 156.59 (d, J=230.6 Hz, 1C), 132.74 (s, 1C), 127.79 (d, J=9.5 Hz, 1C), 125.13 (s, 1C), 112.42 (s, 1C), 112.15 (d, J=9.9 Hz, 1C), 108.77 (d, J=26.1 Hz, 1C), 102.96 (d, J=22.8 Hz, 1C), 64.47 (s, 1C), 53.10 (s, 1C), 47.75 (s, 1C), 30.42 (s, 1C), 29.53 (s, 1C), 21.72 (s, 1C), 13.79 (s, 1C). [Free base] ¹⁹F NMR (377 MHz, d₆-DMSO) δ−125.40 (s, 1F).

### Example 61: N-(2-(5-fluoro-1H-indol-3-yl)ethyl)-N,3-dimethylbutan-2-amine (171)

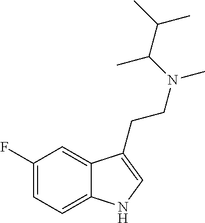 [SMILES: CC(C)C(C)N(C)CCc1c[nH]c2ccc(F)cc12]

N-[2-(5-fluoro-1H-indol-3-yl)ethyl]-N,3-dimethylbutan-2-amine was synthesized in a similar manner as described above for N-ethyl-N-(2-(5-fluoro-1H-indol-3-yl)ethyl)propan-2-amine (1), starting from 5-fluorotryptamine hydrochloride (0.5 g, 0.00233 mol), 3-methyl butan-2-one (1.2 g, 0.0140 mol) to provide the title compound white solid (0.35 g, 0.0133 mol, 57% Yield). The hydrochloride salt was collected as an off-white powder (m.p. 157.5-158.1° C.). High-resolution atmospheric solid analysis probe mass spectrometry (HR-ASAP-MS): m/z 263.1923 (theory [M+H]⁺ C₁₆H₂₄FN₂⁺, m/z 263.1918, Δ=1.9 ppm). [Free base] ¹H NMR (400 MHz, d₆-DMSO) δ 11.06 (s, 1H), 7.31 (dd, J=8.8, 4.6 Hz, 1H), 7.20 (d, J=2.5 Hz, 1H)*, 7.18 (dd, J=10.1, 2.4 Hz, 1H)*, 6.87 (td, J=9.2, 2.4 Hz, 1H), 2.83-2.58 (m, 3H), 2.50-2.44 (m, 1H), 2.23-2.13 (m, 1H)*, 2.19 (s, 3H)*, 1.62-1.48 (m, 1H), 0.88 (d, J=6.6 Hz, 3H), 0.83 (dd, J=9.7, 6.6 Hz, 6H). *=coalescing [Free base] ¹³C NMR (101 MHz, d₆-DMSO) δ 156.57 (d, J=230.9 Hz, 1C), 132.89 (s, 1C), 127.53 (d, J=9.7 Hz, 1C), 124.74 (s, 1C), 113.16 (d, J=4.8 Hz, 1C), 112.23 (d, J=9.7 Hz, 1C), 108.72 (d, J=26.1 Hz, 1C), 102.74 (d, J=22.8 Hz, 1C), 63.97 (s, 1C), 54.17 (s, 1C), 36.85 (s, 1C), 31.22 (s, 1C), 23.85 (s, 1C), 20.82 (s, 1C), 20.00 (s, 1C), 9.37 (s, 1C). [Free base] ¹⁹F NMR (377 MHz, d₆-DMSO) δ−125.51 (s, 1F).

### Example 62: N-(sec-butyl)-N-(2-(5-fluoro-1H-indol-3-yl)ethyl)cyclopropanamine (172)

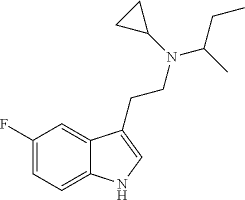 [SMILES: CCC(C)N(CCc1c[nH]c2ccc(F)cc12)C1CC1]

N-(butan-2-yl)-N-[2-(5-fluoro-1H-indol-3-yl)ethyl]cyclopropanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.35 g, 0.00160 mol), methyl ethyl ketone (10 mL, 0.112 mol) to provide the title compound colorless oil (0.35 g, 0.0128 mol, 79.6% Yield). The hydrochloride salt was collected as a white solid. [Free base]¹H NMR (400 MHz, d₆-DMSO) δ 10.85 (s, 1H), 7.31 (dd, J=8.8, 4.6 Hz, 1H), 7.20 (d, J=2.2 Hz, 1H), 7.17 (dd, J=10.0, 2.5 Hz, 1H), 6.88 (td, J=9.2, 2.5 Hz, 1H), 2.92-2.68 (m, 5H), 2.00-1.91 (m, 1H), 1.56-1.43 (m, 1H), 1.32-1.21 (m, 1H), 0.98 (d, J=6.6 Hz, 3H), 0.81 (t, J=7.4 Hz, 3H), 0.56-0.47 (m, 1H), 0.47-0.38 (m, 2H), 0.28-0.20 (m, 1H). [Free base] ¹³C NMR (101 MHz, d₆-DMSO) δ 156.56 (d, J=230.7 Hz, 1C), 132.85 (s, 1C), 127.45 (d, J=9.6 Hz, 1C), 124.59 (s, 1C), 113.44 (d, J=4.6 Hz, 1C), 112.23 (d, J=9.9 Hz, 1C), 108.81 (d, J=26.0 Hz, 1C), 102.66 (d, J=22.8 Hz, 1C), 58.41 (s, 1C), 51.08 (s, 1C), 33.39 (s, 1C), 26.63 (s, 1C), 24.77 (s, 1C), 14.77 (s, 1C), 11.53 (s, 1C), 7.86 (s, 1C), 5.71 (s, 1C). [Free base] ¹⁹F NMR (377 MHz, d₆-DMSO) δ−125.36 (s, 1F).

### Example 63: N-cyclopropyl-N-(2-(5-fluoro-1H-indol-3-yl)ethyl)cyclobutanamine (173)

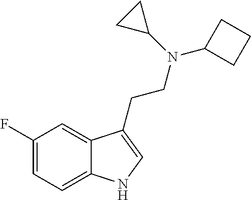 [SMILES: Fc1ccc2[nH]cc(CCN(C3CCC3)C3CC3)c2c1]

N-cyclopropyl-N-[2-(5-fluoro-1H-indol-3-yl)ethyl]cyclobutanamine was synthesized in a similar manner as described above for N-ethyl-N-(2-(5-fluoro-1H-indol-3-yl)ethyl)propan-2-amine (1), starting from N-[2-(5-fluoro-1H-indol-3-yl)ethyl]cyclopropanamine (0.35 g, 0.00165 mol), cyclobutanone (0.49 mL, 0.00660 mol) to provide the title compound semi-crystalline white solid (0.39 g, 0.00143 mol, 86.7% yield). The hydrochloride salt was collected as a beige crystalline solid (m.p. 211.7-212.3° C.). [HCl] ¹H NMR (400 MHz, d₆-DMSO) δ 11.15 (s, 1H)*, 11.12 (s, 1H)*, 7.39 (dd, J=10.0, 2.3 Hz, 1H)**, 7.36 (dd, J=7.9, 3.3 Hz, 1H)**, 7.35 (d, J=2.5 Hz, 1H)**, 6.95 (td, J=13.8, 2.5 Hz, 1H), 4.05-3.91 (m, 1H), 3.25-3.17 (m, 4H), 2.84-2.72 (m, 1H), 2.61-2.51 (m, 1H)***, 2.48-2.39 (m, 1H)*** 2.28-2.12 (m, 2H), 1.78-1.62 (m, 2H), 1.27-1.08 (m, 2H), 0.90-0.78 (m, 2H).*=coalescing, **=coalescing,***=coalescing with DMSO solvent peak. [HCl] ¹³C NMR (101 MHz, d₆-DMSO) δ 156.76 (d, J=231.2 Hz, 1C), 132.87 (s, 1C), 126.89 (d, J=9.7 Hz, 1C), 125.39 (s, 1C), 112.56 (d, J=9.8 Hz, 1C), 109.77 (d, J=4.7 Hz, 1C), 109.35 (d, J=26.2 Hz, 1C), 102.84 (d, J=23.2 Hz, 1C), 58.74 (s, 1C), 52.36 (s, 1C), 34.22 (s, 1C), 26.32 (s, 1C), 26.08 (s, 1C), 19.19 (s, 1C), 13.92 (s, 1C), 3.55 (s, 1C), 2.24 (s, 1C). [HCl] ¹⁹F NMR (377 MHz, d₆-DMSO) δ−124.65 (s, 1F).

### Example 64: 2-(7-bromo-5-fluoro-1H-indol-3-yl)-N-ethyl-N-methylethan-1-amine (174)

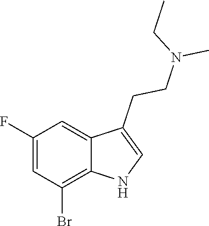 [SMILES: CCN(C)CCc1c[nH]c2c(Br)cc(F)cc12]

2-(7-bromo-5-fluoro-1H-indol-3-yl)-N-ethyl-N-methylethan-1-amine was prepared as described above for (37) with the appropriately substituted indole and appropriately substituted amine to produce the substituted glyoxylamide as a yellow solid (1.02 g, 0.00311, 69.9% yield). The title compound was prepared from reduction of the glyoxylamide to yield the title compound as a slightly yellow solid (0.72, 0.00241 mol, 77.5% Yield). The hydrochloride salt was collected a yellow tinted crystalline solid (m.p 228.3-230.5° C.). [HCl] ¹H NMR (400 MHz, d₆-DMSO) δ 11.35 (s, 1H), 10.79 (s, 1H), 7.56 (dd, J=9.6, 1.6 Hz, 1H), 7.42 (d, J=2.4 Hz, 1H), 7.29 (dd, J=9.0, 2.1 Hz, 1H), 3.31-3.21 (m, 2H)*, 3.21-3.01 (m, 4H)*, 2.78 (s, 3H), 1.26 (t, J=7.2 Hz, 3H). *=coalescing. [HCl] ¹³C NMR (101 MHz, d₆-DMSO) δ 156.11 (d, J=235.4 Hz, 1C), 131.61 (s, 1C), 127.82 (d, J=10.3 Hz, 1C), 126.85 (s, 1C), 112.23 (d, J=29.3 Hz, 1C), 111.24 (d, J=5.1 Hz, 1C), 103.88 (d, J=12.5 Hz, 1C), 103.17 (d, J=23.3 Hz, 1C), 54.16 (s, 1C), 49.83 (s, 1C), 38.13 (s, 1C), 19.66 (s, 1C), 8.77 (s, 1C). [HCl] ¹⁹F NMR (377 MHz, d₆-DMSO) δ−122.95 (s, 1F).

### Example 65: N-ethyl-2-(5-fluoro-6-methyl-1H-indol-3-yl)-N-methylethan-1-amine (175)

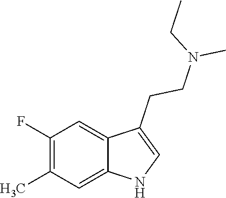 [SMILES: CCN(C)CCc1c[nH]c2cc(C)c(F)cc12]

N-ethyl-2-(5-fluoro-6-methyl-1H-indol-3-yl)-N-methylethan-1-amine was prepared as described above for (7) with the appropriately substituted indole and appropriately substituted amine to produce the glyoxylamide as a yellow solid (1.42 g, 0.00541, 84.7% yield). The title compound was prepared from reduction of the glyoxylamide to yield the title compound as a slightly yellow solid (0.97, 0.00414 mol, 76.5% yield). The hydrochloride salt was collected an off-yellow solid. [HCl] ¹H NMR (400 MHz, d₆-DMSO) δ 10.97 (s, 1H), 10.60 (s, 1H), 7.38 (d, J=10.8 Hz, 1H), 7.22 (s, 1H), 7.22 (d, J=8.4 Hz, 1H), 3.31-3.11 (m, 4H)*, 3.11-3.03 (m, 2H), 2.78 (s, 3H), 2.30 (d, J=1.3 Hz, 3H), 1.25 (t, J=7.3 Hz, 3H). *=coalescing with water. [HCl]¹³C NMR (101 MHz, d₆-DMSO) δ 155.76 (d, J=231.7 Hz, 1C), 132.89 (s, 1C), 125.13 (d, J=10.0 Hz, 1C), 124.30 (s, 1C), 118.12 (d, J=21.4 Hz, 1C), 112.98 (d, J=5.7 Hz, 1C), 109.23 (d, J=4.7 Hz, 1C), 102.87 (d, J=24.2 Hz, 1C), 54.41 (s, 1C), 49.87 (s, 1C), 38.20 (s, 1C), 19.79 (s, 1C), 15.02 (d, J=4.1 Hz, 1C), 8.82 (s, 1C). [HCl] ¹⁹F NMR (377 MHz, d₆-DMSO) δ−128.49 (s, 1F).

### Example 66: N-(2-(5-fluoro-1H-indol-3-yl)ethyl)-N-(prop-2-yn-1-yl)butan-2-amine (176)

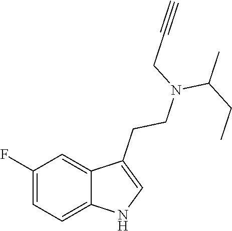 [SMILES: C#CCN(CCc1c[nH]c2ccc(F)cc12)C(C)CC]

N-[2-(5-fluoro-1H-indol-3-yl)ethyl]-N-(prop-2-yn-1-yl)butan-2-amine was prepared as described above for (11) with N-(2-(5-fluoro-1H-indol-3-yl)ethyl)butan-2-amine (0.5 g, 0.00213 mol), 9.2 M propargyl bromide solution (1.16 mL, 0.0107 mol), and TEA (1.29 g, 0.0128 mol) as the starting materials to yield the title compound as an orange oil (0.22 g, 0.000805 mol, 37.8% yield). This was kept as the free base. [Free base] ¹H NMR (400 MHz, d₆-DMSO) δ 10.86 (s, 1H), 7.31 (dd, J=8.8, 4.6 Hz, 1H), 7.24 (dd, J=7.1, 2.5 Hz, 1H)*, 7.22 (d, J=2.9 Hz, 1H)*, 6.88 (td, J=9.2, 2.5 Hz, 1H), 3.44 (dd, J=5.7, 2.3 Hz, 2H), 3.08-3.05 (m, 1H), 2.83-2.65 (m, 5H), 1.53-1.42 (m, 1H), 1.31-1.20 (m, 1H), 0.96 (d, J=6.6 Hz, 3H), 0.81 (t, J=7.4 Hz, 3H). *=coalescing. [Free base] ¹³C NMR (101 MHz, d₆-DMSO) δ 156.56 (d, J=230.9 Hz, 1C), 132.82 (s, 1C), 127.48 (d, J=9.6 Hz, 1C), 124.70 (s, 1C), 112.98 (d, J=4.7 Hz, 1C), 112.16 (d, J=9.7 Hz, 1C), 108.80 (d, J=26.0 Hz, 1C), 102.85 (d, J=23.0 Hz, 1C), 81.96 (s, 1C), 74.36 (s, 1C), 57.70 (s, 1C), 49.44 (s, 1C), 38.64 (s, 1C), 26.65 (s, 1C), 24.06 (s, 1C), 15.02 (s, 1C), 11.00 (s, 1C). [Freebase] ¹⁹F NMR (377 MHz, d₆-DMSO) δ−125.31 (s, 1F).

### Example 67: 2-(sec-butyl(2-(5-fluoro-1H-indol-3-yl)ethyl)amino)acetonitrile (177)

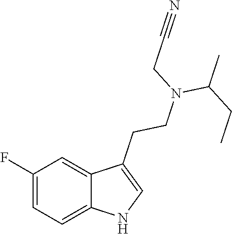 [SMILES: CCC(C)N(CC#N)CCc1c[nH]c2ccc(F)cc12]

2-(Sec-butyl(2-(5-fluoro-1H-indol-3-yl)ethyl)amino)acetonitrile was prepared as described above for (11) with N-(2-(5-fluoro-1H-indol-3-yl)ethyl)butan-2-amine (0.76 g, 0.00324 mol) bromoacetonitrile (1.94 g, 0.0162 mol), and TEA (1.97 g, 0.0195 mol) as the starting materials to yield the title compound as an amber oil (0.37 g, 0.00135 mol, 41.7% Yield). This was isolated as the free base. [Free base]¹H NMR (400 MHz, d₆-DMSO) δ 10.90 (s, 1H), 7.32 (dd, J=8.8, 4.6 Hz, 1H), 7.28 (dd, J=10.2, 2.6 Hz, 1H), 7.25 (d, J=2.3 Hz, 1H), 6.89 (td, J=9.2, 2.5 Hz, 1H), 3.80 (s, 2H), 2.86-2.78 (m, 2H)*, 2.78-2.70 (m, 2H)*, 2.70-2.62 (m, 1H), 1.54-1.41 (m, 1H), 1.33-1.20 (m, 1H), 0.98 (d, J=6.6 Hz, 3H), 0.78 (t, J=7.4 Hz, 3H). *=coalescing. [Free base] ¹³C NMR (101 MHz, d₆-DMSO) δ 156.61 (d, J=230.9 Hz, 1C), 132.81 (s, 1C), 127.42 (d, J=9.7 Hz, 1C), 124.89 (s, 1C), 118.62 (s, 1C), 112.43 (d, J=4.8 Hz, 1C), 112.21 (d, J=9.8 Hz, 1C), 108.88 (d, J=26.0 Hz, 1C), 102.90 (d, J=22.9 Hz, 1C), 58.78 (s, 1C), 50.16 (s, 1C), 37.89 (s, 1C), 26.45 (s, 1C), 23.59 (s, 1C), 14.80 (s, 1C), 14.10 (s, 1C), 10.90 (s, 1C). [Free base] ¹⁹F NMR (377 MHz, d₆-DMSO) δ−125.29 (s, 1F).

### Example 68: N-(2-(5-fluoro-1H-indol-3-yl)ethyl)-N-methylbut-3-en-2-amine (178)

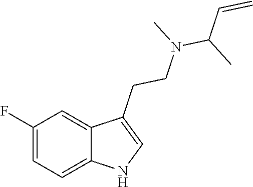 [SMILES: C=CC(C)N(C)CCc1c[nH]c2ccc(F)cc12]

N-[2-(5-fluoro-1H-indol-3-yl)ethyl]-N-methylbut-3-en-2-amine was prepared as described above from (1) with N-[2-(5-fluoro-1H-indol-3-yl)ethyl]but-3-en-2-amine (0.4 g, 0.00172 mol), and 37% formaldehyde solution in water (w/v) (0.43 mL, 0.00516 mol) as the starting materials to yield the title compound as an off yellow-solid (0.19 g, 0.000771 mol, 44.8% yield) (m.p. 80.1-81.1° C.). This material was kept as the free base. [Free base] ¹H NMR (400 MHz, d₆-DMSO) δ 10.86 (s, 1H), 7.31 (dd, J=8.8, 4.6 Hz, 1H), 7.21 (d, J=2.4 Hz, 1H)*, 7.21 (dd, J=10.1, 2.4 Hz, 1H)*, 6.88 (td, J=9.2, 2.5 Hz, 1H), 5.81 (ddd, J=17.3, 10.5, 6.8 Hz, 1H), 5.14-5.05 (m, 2H), 3.23-3.13 (m, 1H), 2.83-2.71 (m, 2H), 2.68-2.54 (m, 2H), 2.24 (s, 3H), 1.06 (d, J=6.6 Hz, 3H).*=coalescing. [Free base] ¹³C NMR (101 MHz, d₆-DMSO) δ 156.55 (d, J=230.9 Hz, 1C), 140.57 (s, 1C), 132.80 (s, 1C), 127.48 (d, J=9.7 Hz, 1C), 124.64 (s, 1C), 115.02 (s, 1C), 113.13 (d, J=4.6 Hz, 1C), 112.17 (d, J=9.7 Hz, 1C), 108.81 (d, J=26.0 Hz, 1C), 102.87 (d, J=22.9 Hz, 1C), 60.39 (s, 1C), 54.21 (s, 1C), 37.39 (s, 1C), 23.30 (s, 1C), 15.83 (s, 1C). [Free base]¹⁹F NMR (377 MHz, d₆-DMSO) δ−125.36 (s, 1F).

### Example 69: 2-(5-chloro-6-fluoro-1H-indol-3-yl)-N-ethyl-N-methylethan-1-amine (179)

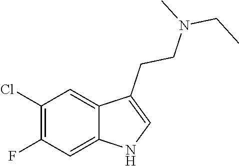 [SMILES: CCN(C)CCc1c[nH]c2cc(F)c(Cl)cc12]

2-(5-chloro-6-fluoro-1H-indol-3-yl)-N-ethyl-N-methylethan-1-amine was prepared as described above from (37) with the appropriately substituted indole and appropriately substituted amine to produce the appropriately substituted glyoxylamide as a beige solid (1 g, 0.00393 mol, 70.1% Yield). The title compound was prepared from reduction of the glyoxylamide as an oil. The hydrochloride salt was collected a beige solid. (0.54, 0.00191 mol, 48.6% yield). [HCl]¹H NMR (400 MHz, d₆-DMSO) δ 11.32 (s, 1H), 10.70 (s, 1H), 7.87 (d, J=7.4 Hz, 1H), 7.37 (d, J=10.2 Hz, 1H), 7.33 (d, J=2.2 Hz, 1H), 3.32-3.3.15 (m, 4H)*, 3.15-3.3.06 (m, 2H)*, 2.78 (s, 3H), 1.26 (t, J=7.3 Hz, 3H).*=coalescing. [HCl] ¹³C NMR (101 MHz, d₆-DMSO) δ 153.22 (d, J=237.1 Hz, 1C), 134.40 (d, J=11.4 Hz, 1C), 125.39 (d, J=3.4 Hz, 1C), 124.01 (s, 1C), 119.26 (s, 1C), 111.02 (d, J=19.9 Hz, 1C), 109.37 (s, 1C), 99.12 (d, J=25.2 Hz, 1C), 54.36 (s, 1C), 49.84 (s, 1C), 38.13 (s, 1C), 19.51 (s, 1C), 8.80 (s, 1C). [HCl] ¹⁹F NMR (377 MHz, d₆-DMSO) δ−124.29 (s, 1F).

### Example 70: N-(2-(7-fluoro-1H-indol-3-yl)ethyl)-N-propylprop-2-yn-1-amine (180)

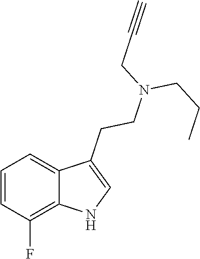 [SMILES: C#CCN(CCC)CCc1c[nH]c2c(F)cccc12]

N-[2-(7-fluoro-1H-indol-3-yl)ethyl]-N-propylprop-2-yn-1-amine was prepared as described above from (11) utilizing N-[2-(7-fluoro-1H-indol-3-yl)ethyl]propan-1-amine (0.33 g, 0.00150 mol), 9.2 M Propargyl bromide solution (0.81 mL, 0.00749 mol) and TEA (1.25 mL, 0.00899 mol) as the starting materials to yield the titled compound as an amber oil (0.46 g, 0.00176 mol, quant yield). The hydrochloride salt was collected as a beige solid. [HCl] ¹H NMR (400 MHz, d₆-DMSO) δ 11.64 (s, 1H)*, 11.51 (s, 1H)*, 7.48 (d, J=7.6 Hz, 1H), 7.33 (s, 1H), 7.07-6.81 (m, 2H), 4.36-4.09 (m, 2H), 3.86 (s, 1H), 3.55-3.36 (m, 2H)**, 3.28-2.99 (m, 4H), 1.77 (sextet, J=7.5 Hz, 2H), 0.92 (t, J=7.2 Hz, 3H). *=coalescing, *=coalescing with H₂O. [HCl] ¹³C NMR (101 MHz, d₆-DMSO) δ 149.26 (d, J=243.0 Hz, 1C), 130.75 (d, J=6.0 Hz, 1C), 124.54 (s, 1C), 123.97 (d, J=13.4 Hz, 1C), 118.91 (d, J=6.2 Hz, 1C), 114.52 (d, J=2.9 Hz, 1C), 110.22 (s, 1C), 106.08 (d, J=16.0 Hz, 1C), 81.19 (s, 1C), 72.97 (s, 1C), 53.85 (s, 1C), 52.43 (s, 1C), 40.99 (s, 1C), 19.61 (s, 1C), 16.80 (s, 1C), 10.91 (s, 1C). [HCl]¹⁹F NMR (377 MHz, d₆-DMSO) δ−133.06 (s, 1F).

### Example 71: 2-((2-(7-fluoro-1H-indol-3-yl)ethyl)(propyl)amino)acetonitrile (181)

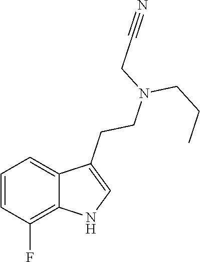 [SMILES: CCCN(CC#N)CCc1c[nH]c2c(F)cccc12]

2-((2-(7-fluoro-1H-indol-3-yl)ethyl)(propyl)amino)acetonitrile was prepared as described above from (11) utilizing N-[2-(7-fluoro-1H-indol-3-yl)ethyl]propan-1-amine (0.33 g, 0.00150 mol), bromoacetonitrile (0.52 mL, 0.00749 mol) and TEA (1.25 mL, 0.00899 mol) as the starting materials to yield the titled compound as an orange oil (0.45 g, 0.00173 mol, quant yield). The hydrochloride was collected as beige solid. [Free base] ¹H NMR (400 MHz, d₆-DMSO) δ 11.48 (s, 1H), 7.47 (d, J=7.6 Hz, 1H), 7.33 (s, 1H), 7.08-6.82 (m, 2H), 4.57 (s, 2H), 3.47-3.24 (m, 2H)*, 3.24-3.15 (m, 2H)*, 3.15-2.94 (m, 2H)*, 1.82-1.65 (m, 2H), 0.92 (t, J=7.2 Hz, 3H). *=coalescing. [Free base] ¹³C NMR (101 MHz, d₆-DMSO) δ 149.27 (d, J=242.9 Hz, 1C), 130.80 (d, J=6.0 Hz, 1C), 124.56 (s, 1C), 123.96 (d, J=13.3 Hz, 1C), 118.89 (d, J=6.1 Hz, 1C), 114.50 (d, J=2.8 Hz, 1C), 113.58 (s, 1C), 110.42 (s, 1C), 106.06 (d, J=16.0 Hz, 1C), 54.94 (s, 1C), 53.66 (s, 1C), 39.94 (s, 1C)*, 20.07 (s, 1C), 17.25 (s, 1C), 10.92 (s, 1C). *=coalescing with DMSO solvent peak. [Free base] ¹⁹F NMR (377 MHz, d₆-DMSO) δ−133.11 (s, 1F).

### Example 72: 2-(7-chloro-5-fluoro-1H-indol-3-yl)-N-ethyl-N-methylethan-1-amine (182)

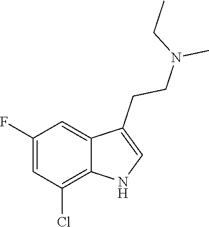 [SMILES: CCN(C)CCc1c[nH]c2c(Cl)cc(F)cc12]

2-(7-chloro-5-fluoro-1H-indol-3-yl)-N-ethyl-N-methylethan-1-amine was prepared as described above from (37) with the appropriately substituted indole and appropriately substituted amine to produce the desired glyoxylamide as a brown solid (1.13 g, 0.00397 mol, 70.9% Yield). The title compound was prepared from reduction of the glyoxylamide as an oil (0.33 g, 0.00130 mol, 32.7% yield). The hydrochloride salt was collected a beige solid. [HCl] ¹H NMR (400 MHz, d₆-DMSO) δ 11.50 (s, 1H), 10.82 (s, 1H), 7.53 (dd, J=9.6, 2.0 Hz, 1H), 7.43 (d, J=2.2 Hz, 1H), 7.17 (dd, J=9.2, 2.1 Hz, 1H), 3.33-3.15 (m, 4H)*, 3.15-3.02 (m, 2H)*, 2.78 (s, 3H), 1.26 (t, J=7.2 Hz, 3H). *=coalescing. [HCl] ¹³C NMR (101 MHz, d₆-DMSO) δ 156.02 (d, J=234.4 Hz, 1C), 130.02 (s, 1C), 128.07 (d, J=10.4 Hz, 1C), 126.76 (s, 1C), 115.96 (d, J=13.2 Hz, 1C), 111.17 (d, J=5.1 Hz, 1C), 109.44 (d, J=29.5 Hz, 1C), 102.78 (d, J=23.2 Hz, 1C), 54.17 (s, 1C), 49.82 (s, 1C), 38.12 (s, 1C), 19.60 (s, 1C), 8.77 (s, 1C). [HCl] ¹⁹F NMR (377 MHz, d₆-DMSO) δ−122.95 (s, 1F).

### Example 73: (R)-5-fluoro-3-(pyrrolidin-2-ylmethyl)-1H-indole (183)

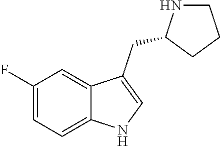 [SMILES: Fc1ccc2[nH]cc(C[C@H]3CCCN3)c2c1]

(R)-5-fluoro-3-(pyrrolidin-2-ylmethyl)-1H-indole was prepared as described above from (60) with the appropriately substituted indole. 5-Fluoro-1H-indol-3-yl[(2R)-pyrrolidin-2-yl]methanone (0.47 g, 0.00202 mol) was utilized as the starting material to yield the title compound as a transparent amber oil (0.17 g, 0.000779 mol, 38.6% yield). This was kept as the free base. [Free base] ¹H NMR (400 MHz, d₆-DMSO) δ 10.88 (s, 1H), 7.30 (dd, J=8.8, 4.6 Hz, 1H), 7.26 (dd, J=10.2, 2.5 Hz, 1H), 7.22 (d, J=2.2 Hz, 1H), 6.88 (td, J=9.1, 2.5 Hz, 1H), 3.27-3.15 (m, 2H)*, 2.95-2.83 (m, 1H), 2.76-2.62 (m, 2H), 1.80-1.54 (m, 3H), 1.35-1.23 (m, 1H). *=coalescing with H₂O. [Free base] ¹³C NMR (101 MHz, d₆-DMSO) δ 156.73 (d, J=230.8 Hz, 1C), 132.93 (s, 1C), 127.60 (d, J=9.8 Hz, 1C), 125.39 (s, 1C), 112.29 (d, J=9.4 Hz, 1C), 112.06 (d, J=4.2 Hz, 1C), 108.97 (d, J=26.0 Hz, 1C), 103.10 (d, J=23.1 Hz, 1C), 59.28 (s, 1C), 45.17 (s, 1C), 30.79 (s, 1C), 29.85 (s, 1C), 24.20 (s, 1C). [Free base] ¹⁹F NMR (377 MHz, d₆-DMSO) δ−125.39 (s, 1F).

### Example 74: (R)-5-fluoro-3-((1-propylpyrrolidin-2-yl)methyl)-1H-indole (184)

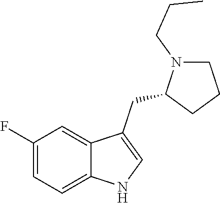 [SMILES: CCCN1CCC[C@@H]1Cc1c[nH]c2ccc(F)cc12]

(5-fluoro-3-{[(2R)-1-propylpyrrolidin-2-yl]methyl}-1H-indole was prepared as described above from (60) with the appropriately substituted indole. 1-[(2R)-2-(5-fluoro-1H-indole-3-carbonyl)pyrrolidin-1-yl]propan-1-one (0.55 g, 0.00191 mol) was utilized as the starting material to yield the title compound as a white solid (0.18 g, 0.000691 mol, 36.2% yield) This was kept as the free base. [Free base] ¹H NMR (400 MHz, d₆-DMSO) δ 10.87 (s, 1H), 7.30 (dd, J=8.7, 4.5 Hz, 1H), 7.22 (dd, J=7.7, 2.2 Hz, 1H)*, 7.21 (d, J=2.2 Hz, 1H)*, 6.89 (td, J=13.7, 2.3 Hz, 1H), 3.10-3.01 (m, 1H), 2.98-2.91 (m, 1H), 2.88-2.79 (m, 1H), 2.49-2.41 (m, 2H)**, 2.18-2.01 (m, 2H), 1.68-1.58 (m, 2H), 1.58-1.48 (m, 2H), 1.48-1.37 (m, 2H), 0.90 (t, J=7.3 Hz, 3H). *=coalescing, **=coalescing with DMSO solvent peak. [Free base] ¹³C NMR (101 MHz, d₆-DMSO) δ 156.62 (d, J=230.7 Hz, 1C), 132.84 (d, J=17.4 Hz, 1C), 127.84 (d, J=9.6 Hz, 1C), 125.12 (s, 1C), 112.52 (d, J=4.9 Hz, 1C), 112.16 (d, J=9.8 Hz, 1C), 108.76 (d, J=26.1 Hz, 1C), 102.95 (d, J=22.8 Hz, 1C), 64.77 (s, 1C), 56.19 (s, 1C), 53.63 (s, 1C), 30.38 (s, 1C), 29.65 (s, 1C), 21.88 (s, 1C), 21.72 (s, 1C), 12.04 (s, 1C). [Free base] ¹⁹F NMR (377 MHz, d₆-DMSO) δ −125.45 (s, 1F).

### Example 75: (R)-5,6-difluoro-3-((1-methylpyrrolidin-2-yl)methyl)-1H-indole (185)

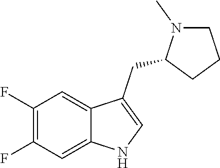 [SMILES: CN1CCC[C@@H]1Cc1c[nH]c2cc(F)c(F)cc12]

5,6-difluoro-3-{[(2R)-1-methylpyrrolidin-2-yl]methyl}-1H-indole was prepared as described above from (27) with the appropriately substituted indole. Benzyl (2R)-2-(5,6-difluoro-1H-indole-3-carbonyl)pyrrolidine-1-carboxylate (1.14 g, 0.00297 mol) was utilized as the starting material to yield the title compound as a white solid (0.61 g, 0.00244 mol, 82.1% yield). This material was kept as the free base. [Free base] ¹H NMR (400 MHz, d₆-DMSO) δ 10.96 (s, 1H), 7.44 (dd, J=11.5, 8.1 Hz, 1H), 7.30 (dd, J=11.3, 7.0 Hz, 1H), 7.20 (d, J=2.0 Hz, 1H), 3.01-2.90 (m, 2H), 2.49-2.42 (m, 1H)*, 2.37-2.26 (m, 1H), 2.33 (s, 3H), 2.09 (q, J=8.7 Hz, 1H), 1.74-1.63 (m, 1H)**, 1.63-1.49 (m, 2H)**, 1.47-1.36 (m, 1H). *=coalescing with DMSO solvent peak, **=coalescing. [Free base] ¹³C NMR (101 MHz, d₆-DMSO) δ 146.37 (dd, J=236.3, 15.7 Hz, 1C), 144.82 (dd, J=233.6, 14.9 Hz, 1C), 130.95 (d, J=10.5 Hz, 1C), 124.88 (d, J=3.6 Hz, 1C), 122.96 (d, J=7.6 Hz, 1C), 112.63 (d, J=4.3 Hz, 1C), 104.93 (d, J=18.5 Hz, 1C), 99.04 (d, J=21.1 Hz, 1C), 66.05 (s, 1C), 56.90 (s, 1C), 40.46 (s, 1C), 30.73 (s, 1C), 28.99 (s, 1C), 21.58 (s, 1C). [Free base] ¹⁹F NMR (377 MHz, d₆-DMSO) δ−145.88 (d, J=21.5 Hz, 1F), −149.15 (d, J=21.8 Hz, 1F).

### Example 76: (R)-7-fluoro-3-((1-methylpyrrolidin-2-yl)methyl)-1H-indole (186)

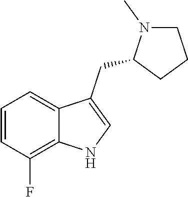 [SMILES: CN1CCC[C@@H]1Cc1c[nH]c2c(F)cccc12]

(R)-7-fluoro-3-((1-methylpyrrolidin-2-yl)methyl)-1H-indole was prepared as described above from (27) with the appropriately substituted indole. Benzyl (2R)-2-(7-fluoro-1H-indole-3-carbonyl)pyrrolidine-1-carboxylate (2.0 g, 0.00546 mol) was utilized as the starting material to yield the title compound as slightly orange solid (0.82 g, 0.00353 mol, 64.65% yield). This material was kept as the free base. [Free base] ¹H NMR (400 MHz, d₆-DMSO) δ 11.27 (s, 1H), 7.32 (d, J=7.7 Hz, 1H), 7.19 (d, J=2.0 Hz, 1H), 6.97-6.90 (m, 1H)*, 6.90-6.83 (m, 1H)*, 3.02 (dd, J=14.0, 3.4 Hz, 1H), 2.99-2.92 (m, 1H), 2.50-2.44 (m, 1H)**, 2.40-2.27 (m, 1H)***, 2.33 (s, 3H)***, 2.09 (q, J=8.7 Hz, 1H), 1.72-1.63 (m, 1H), 1.63-1.50 (m, 2H), 1.50-1.38 (m, 1H). *=coalescing, **=coalescing with DMSO solvent peak. ¹³C NMR (101 MHz, d₆-DMSO) δ 149.26 (d, J=242.3 Hz, 1C), 131.66 (d, J=5.9 Hz, 1C), 124.08 (s, 1C), 123.80 (d, J=12.9 Hz, 1C), 118.39 (d, J=6.3 Hz, 1C), 114.61 (d, J=3.1 Hz, 1C), 113.31 (d, J=2.0 Hz, 1C), 105.55 (d, J=16.1 Hz, 1C), 66.09 (s, 1C), 56.94 (s, 1C), 40.47 (s, 1C), 30.80 (s, 1C), 29.24 (s, 1C), 21.56 (s, 1C). [Free base] ¹⁹F NMR (377 MHz, d₆-DMSO) δ−133.57 (s, 1F).

### Example 77: Selected Compounds of the Present Invention

TABLE 1
Selected compounds of the present invention.

| Cmpd | Structure | Name |
|---|---|---|
| 1 | 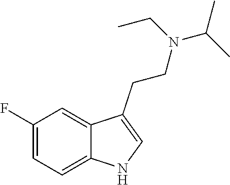 [SMILES: CCN(CCc1c[nH]c2ccc(F)cc12)C(C)C] | N-ethyl-N-(2-(5-fluoro-1H-indol-3- yl)ethyl)propan-2-amine |
| 2 | 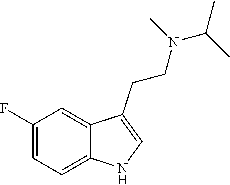 [SMILES: CC(C)N(C)CCc1c[nH]c2ccc(F)cc12] | N-(2-(5-fluoro-1H-indol-3-yl)ethyl)-N- mehtylpropan-2-amine |
| 3 | 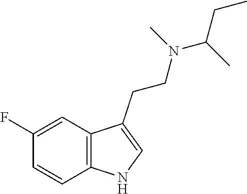 [SMILES: CCC(C)N(C)CCc1c[nH]c2ccc(F)cc12] | N-(2-(5-fluoro-1H-indol-3-yl)ethyl)-N- methylbutan-2-amine |
| 4 | 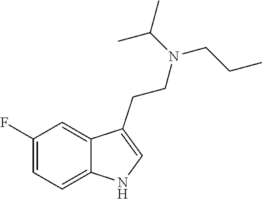 [SMILES: CCCN(CCc1c[nH]c2ccc(F)cc12)C(C)C] | N-(2-(5-fluoro-1H-indol-3-yl)ethyl)-N- isopropylpropan-1-amine |
| 5 | 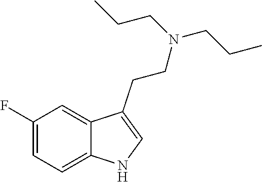 [SMILES: CCCN(CCC)CCc1c[nH]c2ccc(F)cc12] | N-(2-(5-fluoro-1H-indol-3-yl)ethyl)-N- propylpropan-1-amine |
| 6 | 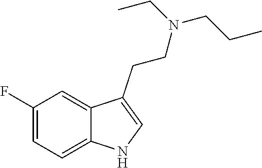 [SMILES: CCCN(CC)CCc1c[nH]c2ccc(F)cc12] | N-ethyl-N-(2-(5-fluoro-1H-indol-3- yl)ethyl)propan-1-amine |
| 7 | 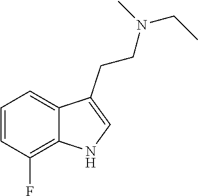 [SMILES: CCN(C)CCc1c[nH]c2c(F)cccc12] | N-ethyl-2-(7-fluoro-1H-indol-3-yl)-N- methylethan-1-amine |
| 8 | 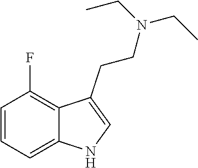 [SMILES: CCN(CC)CCc1c[nH]c2cccc(F)c12] | N,N-diethyl-2-(4-fluoro-1H-indol-3-yl)ethan- 1-amine |
| 9 | 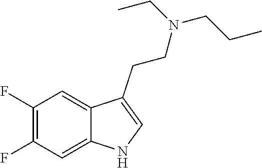 [SMILES: CCCN(CC)CCc1c[nH]c2cc(F)c(F)cc12] | N-(2-(5,6-difluoro-1H-indol-3-yl)ethyl)-N- ethylpropan-1-amine |
| 10 | 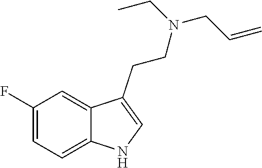 [SMILES: C=CCN(CC)CCc1c[nH]c2ccc(F)cc12] | N-(2-(5-fluoro-1H-indol-3-yl)ethyl)-N- methylprop-2-en-1-amine |
| 11 | 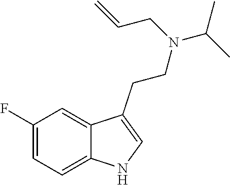 [SMILES: C=CCN(CCc1c[nH]c2ccc(F)cc12)C(C)C] | N-(2-(5-fluoro-1H-indol-3-yl)ethyl)-N- isopropylprop-2-en-1-amine |
| 12 | 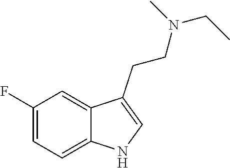 [SMILES: CCN(C)CCc1c[nH]c2ccc(F)cc12] | N-ethyl-2-(5-fluoro-1H-indol-3-yl)-N- methylethan-1-amine |
| 13 | 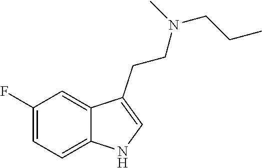 [SMILES: CCCN(C)CCc1c[nH]c2ccc(F)cc12] | N-(2-(5-fluoro-1H-indol-3-yl)ethyl)-N- methylpropan-1-amine |
| 14 | 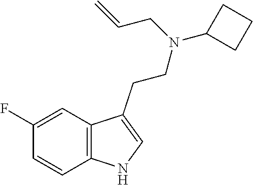 [SMILES: C=CCN(CCc1c[nH]c2ccc(F)cc12)C1CCC1] | N-allyl-N-(2-(5-fluoro-1H-indol-3- yl)ethyl)cyclobutanamine |
| 15 | 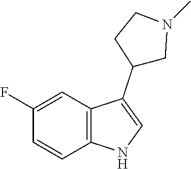 [SMILES: CN1CCC(c2c[nH]c3ccc(F)cc23)C1] | 5-fluoro-3-(1-methylpyrrolidin-3-yl)-1H- indole |
| 16 | 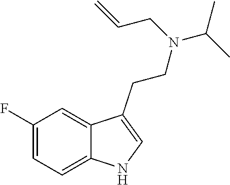 [SMILES: C=CCN(CCc1c[nH]c2ccc(F)cc12)C(C)C] | N-allyl-N-(2-(5-fluoro-1H-indol-3- yl)ethyl)butan-2-amine |
| 17 | 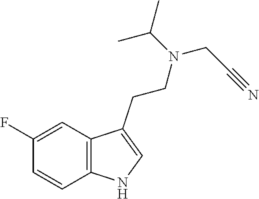 [SMILES: CC(C)N(CC#N)CCc1c[nH]c2ccc(F)cc12] | 2-((2-(5-fluoro-1H-indol-3- yl)ethyl)(isopropyl)amino)acetonitrile |
| 18 | 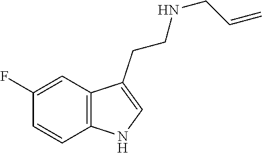 [SMILES: C=CCNCCc1c[nH]c2ccc(F)cc12] | N-(2-(5-fluoro-1H-indol-3-yl)ethyl)prop-2- en-1-amine |
| 19 | 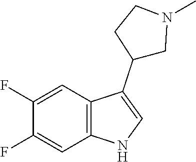 [SMILES: CN1CCC(c2c[nH]c3cc(F)c(F)cc23)C1] | 5,6-difluoro-3-(1-methylpyrrolidin-3-yl)-1H- indole |
| 20 | 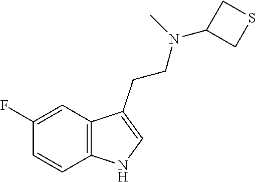 [SMILES: CN(CCc1c[nH]c2ccc(F)cc12)C1CSC1] | N-(2-(5-fluoro-1H-indol-3-yl)ethyl)-N- methylthietan-3-amine |
| 21 | 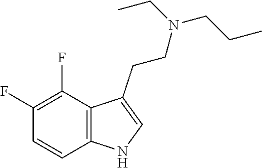 [SMILES: CCCN(CC)CCc1c[nH]c2ccc(F)c(F)c12] | N-(2-(4,5-difluoro-1H-indol-3-yl)ethyl)-N- ethylpropan-1-amine |
| 22 | 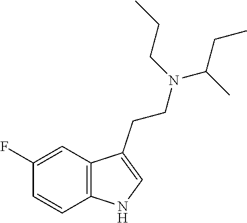 [SMILES: CCCN(CCc1c[nH]c2ccc(F)cc12)C(C)CC] | N-(2-(5-fluoro-1H-indol-3-yl)ethyl)-N- propylbutan-2-amine |
| 23 | 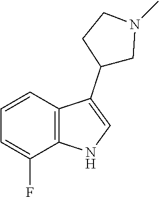 [SMILES: CN1CCC(c2c[nH]c3c(F)cccc23)C1] | 7-fluoro-3-(1-methylpyrrolidin-3-yl)-1H- indole |
| 24 | 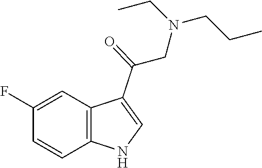 [SMILES: CCCN(CC)CC(=O)c1c[nH]c2ccc(F)cc12] | 2-(ethyl(propyl)amino)-1-(5-fluoro-1H- indol-3-yl)ethan-1-one |
| 25 | 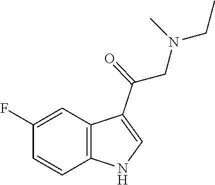 [SMILES: CCN(C)CC(=O)c1c[nH]c2ccc(F)cc12] | 2-(ethyl(methyl)amino)-1-(5-fluoro-1H- indol-3-yl)ethan-1-one |
| 26 | 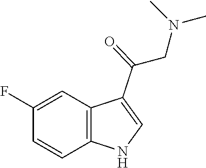 [SMILES: CN(C)CC(=O)c1c[nH]c2ccc(F)cc12] | 2-(5-fluoro-1H-indol-3-yl)-N,N- dimethylpropan-1-amine |
| 27 | 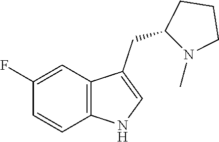 [SMILES: CN1CCC[C@H]1Cc1c[nH]c2ccc(F)cc12] | (S)-5-fluoro-3-((1-methylpyrrolidin-2- yl)methyl)-1H-indole |
| 28 | 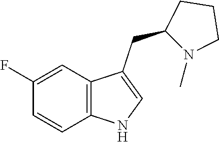 [SMILES: CN1CCC[C@@H]1Cc1c[nH]c2ccc(F)cc12] | (R)-5-fluoro-3-((1-methylpyrrolidin-2- yl)methyl)-1H-indole |
| 29 | 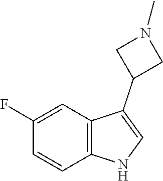 | 5-fluoro-3-(1-methylazetidin-3-yl)-1H-indole |
| 30 | 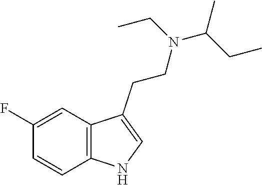 [SMILES: CCC(C)N(CC)CCc1c[nH]c2ccc(F)cc12] | N-ethyl-N-(2-(5-fluoro-1H-indol-3- yl)ethyl)butan-2-amine |
| 31 | 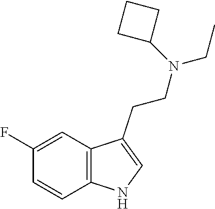 [SMILES: CCN(CCc1c[nH]c2ccc(F)cc12)C1CCC1] | N-ethyl-N-(2-(5-fluoro-1H-indol-3- yl)ethyl)cyclobutanamine |
| 32 | 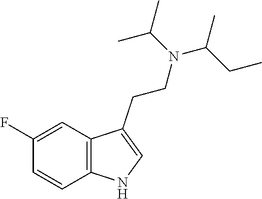 [SMILES: CCC(C)N(CCc1c[nH]c2ccc(F)cc12)C(C)C] | N-(2-(5-fluoro-1H-indol-3-yl)ethyl)-N- isopropylbutan-2-amine |
| 33 | 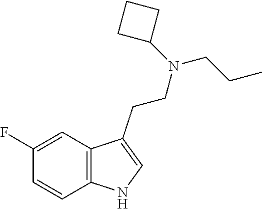 [SMILES: CCCN(CCc1c[nH]c2ccc(F)cc12)C1CCC1] | N-(2-(5-fluoro-1H-indol-3-yl)ethyl)-N- propylcyclobutanamine |
| 34 | 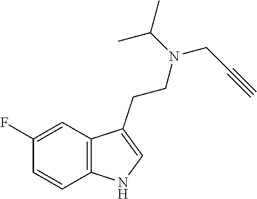 [SMILES: C#CCN(CCc1c[nH]c2ccc(F)cc12)C(C)C] | N-(2-(5-fluoro-1H-indol-3-yl)ethyl)-N- isopropylprop-2-yn-1-amine |
| 35 | 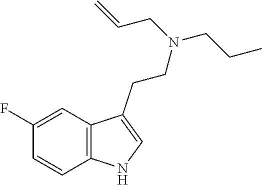 [SMILES: C=CCN(CCC)CCc1c[nH]c2ccc(F)cc12] | N-(2-(5-fluoro-1H-indol-3-yl)ethyl)-N- propylprop-2-en-1-amine |
| 36 | 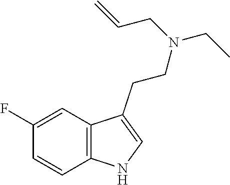 [SMILES: C=CCN(CC)CCc1c[nH]c2ccc(F)cc12] | N-ethyl-N-(2-(5-fluoro-1H-indol-3- yl)ethyl)prop-2-en-1-amine |
| 37 | 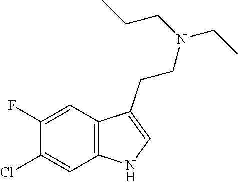 [SMILES: CCCN(CC)CCc1c[nH]c2cc(Cl)c(F)cc12] | N-(2-(6-chloro-5-fluoro-1H-indol-3- yl)ethyl)-N-ethylpropan-1-amine |
| 38 | 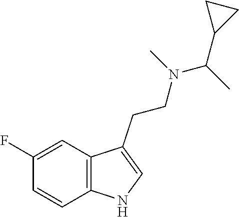 [SMILES: CC(C1CC1)N(C)CCc1c[nH]c2ccc(F)cc12] | 1-cyclopropyl-N-(2-(5-fluoro-1H-indol-3- yl)ethyl)-N-methylethan-1-amine |
| 39 | 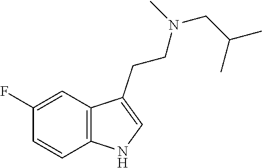 [SMILES: CC(C)CN(C)CCc1c[nH]c2ccc(F)cc12] | N-(2-(5-fluoro-1H-indol-3-yl)ethyl)-N,2- dimethylpropan-1-amine |
| 40 | 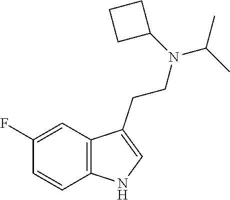 [SMILES: CC(C)N(CCc1c[nH]c2ccc(F)cc12)C1CCC1] | N-(2-(5-fluoro-1H-indol-3-yl)ethyl)-N- isopropylcyclobutanamine |
| 41 | 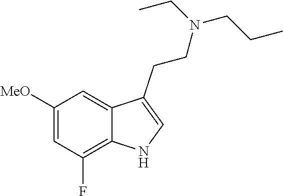 [SMILES: CCCN(CC)CCc1c[nH]c2c(F)cc(OC)cc12] | N-ethyl-N-(2-(7-fluoro-5-methoxy-1H-indol- 3-yl)ethyl)propan-1-amine |
| 42 | 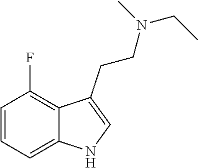 [SMILES: CCN(C)CCc1c[nH]c2cccc(F)c12] | N-ethyl-2-(4-fluoro-1H-indol-3-yl)-N- methylethan-1-amine |
| 43 | 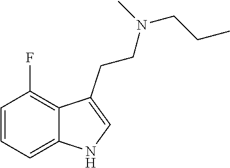 [SMILES: CCCN(C)CCc1c[nH]c2cccc(F)c12] | N-(2-(4-fluoro-1H-indol-3-yl)ethyl)-N- methylpropan-1-amine |
| 44 | 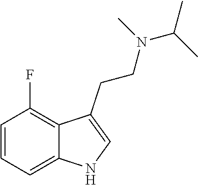 [SMILES: CC(C)N(C)CCc1c[nH]c2cccc(F)c12] | N-(2-(4-fluoro-1H-indol-3-yl)ethyl)-N- methylpropan-2-amine |
| 45 | 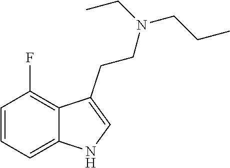 [SMILES: CCCN(CC)CCc1c[nH]c2cccc(F)c12] | N-ethyl-N-(2-(4-fluoro-1H-indol-3- yl)ethyl)propan-1-amine |
| 46 | 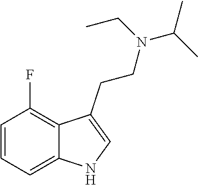 [SMILES: CCN(CCc1c[nH]c2cccc(F)c12)C(C)C] | N-ethyl-N-(2-(4-fluoro-1H-indol-3- yl)ethyl)propan-2-amine |
| 47 | 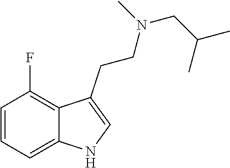 [SMILES: CC(C)CN(C)CCc1c[nH]c2cccc(F)c12] | N-(2-(4-fluoro-1H-indol-3-yl)ethyl)-N,2- dimethylpropan-1-amine |
| 48 | 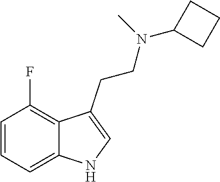 [SMILES: CN(CCc1c[nH]c2cccc(F)c12)C1CCC1] | N-(2-(4-fluoro-1H-indol-3-yl)ethyl)-N- methylcyclobutanamine |
| 49 | 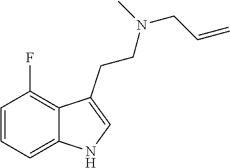 [SMILES: C=CCN(C)CCc1c[nH]c2cccc(F)c12] | N-(2-(4-fluoro-1H-indol-3-yl)ethyl)-N- methylprop-2-en-1-amine |
| 50 | 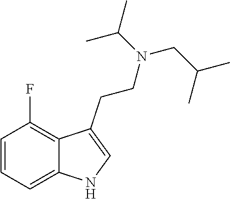 [SMILES: CC(C)CN(CCc1c[nH]c2cccc(F)c12)C(C)C] | N-(2-(4-fluoro-1H-indol-3-yl)ethyl)-N- isopropyl-2-methylpropan-1-amine |
| 51 | 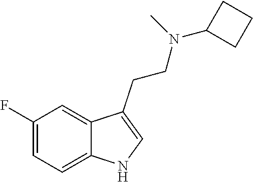 [SMILES: CN(CCc1c[nH]c2ccc(F)cc12)C1CCC1] | N-(2-(5-fluoro-1H-indol-3-yl)ethyl)-N- methylcyclobutanamine |
| 52 | 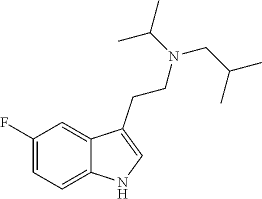 [SMILES: CC(C)CN(CCc1c[nH]c2ccc(F)cc12)C(C)C] | N-(2-(5-fluoro-1H-indol-3-yl)ethyl)-N isopropyl-2-methylpropan-1-amine |
| 53 | 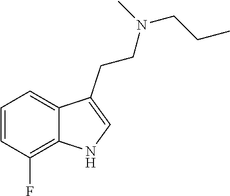 [SMILES: CCCN(C)CCc1c[nH]c2c(F)cccc12] | N-(2-(7-fluoro-1H-indol-3-yl)ethyl)-N- methylpropan-1-amine |
| 54 | 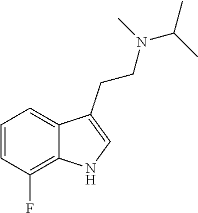 [SMILES: CC(C)N(C)CCc1c[nH]c2c(F)cccc12] | N-(2-(7-fluoro-1H-indol-3-yl)ethyl)-N- methylpropan-2-amine |
| 55 | 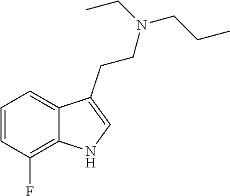 [SMILES: CCCN(CC)CCc1c[nH]c2c(F)cccc12] | N-ethyl-N-(2-(7-fluoro-1H-indol-3- yl)ethyl)propan-1-amine |
| 56 | 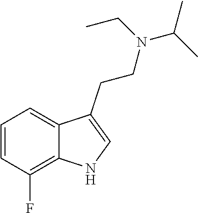 [SMILES: CCN(CCc1c[nH]c2c(F)cccc12)C(C)C] | N-ethyl-N-(2-(7-fluoro-1H-indol-3- yl)ethyl)propan-2-amine |
| 57 | 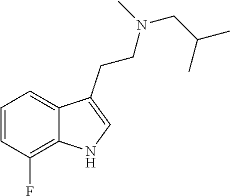 [SMILES: CC(C)CN(C)CCc1c[nH]c2c(F)cccc12] | N-(2-(7-fluoro-1H-indol-3-yl)ethyl)-N,2- dimethylpropan-1-amine |
| 58 | 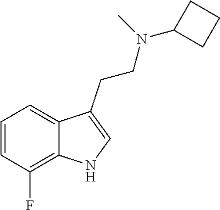 [SMILES: CN(CCc1c[nH]c2c(F)cccc12)C1CCC1] | N-(2-(7-fluoro-1H-indol-3-yl)ethyl)-N- methylcyclobutanamine |
| 59 | 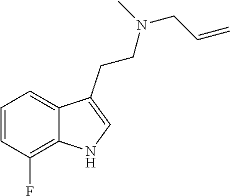 [SMILES: C=CCN(C)CCc1c[nH]c2c(F)cccc12] | N-(2-(7-fluoro-1H-indol-3-yl)ethyl)-N- methylprop-2-en-1-amine |
| 60 | 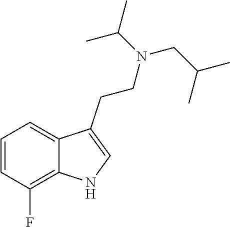 [SMILES: CC(C)CN(CCc1c[nH]c2c(F)cccc12)C(C)C] | N-(2-(7-fluoro-1H-indol-3-yl)ethyl)-N- isopropyl-2-methylpropan-1-amine |
| 61 | 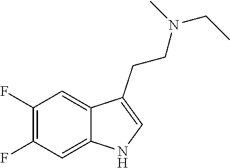 [SMILES: CCN(C)CCc1c[nH]c2cc(F)c(F)cc12] | 2-(5,6-difluoro-1H-indol-3-yl)-N-ethyl-N- methylethan-1-amine |
| 62 | 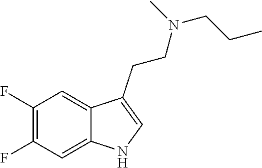 [SMILES: CCCN(C)CCc1c[nH]c2cc(F)c(F)cc12] | N-(2-(5,6-difluoro-1H-indol-3-yl)ethyl)-N- methylpropan-1-amine |
| 63 | 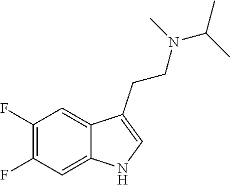 [SMILES: CC(C)N(C)CCc1c[nH]c2cc(F)c(F)cc12] | N-(2-(5,6-difluoro-1H-indol-3-yl)ethyl)-N- methylpropan-2-amine |
| 64 | 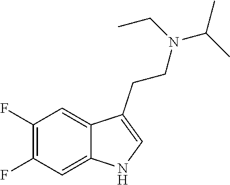 [SMILES: CCN(CCc1c[nH]c2cc(F)c(F)cc12)C(C)C] | N-(2-(5,6-difluoro-1H-indol-3-yl)ethyl)-N- ethylpropan-2-amine |
| 65 | 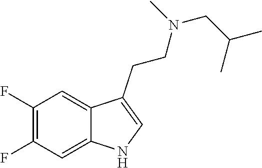 [SMILES: CC(C)CN(C)CCc1c[nH]c2cc(F)c(F)cc12] | N-(2-(5,6-difluoro-1H-indol-3-yl)ethyl)-N,2- dimethylpropan-1-amine |
| 66 | 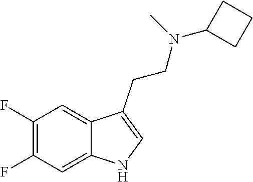 [SMILES: CN(CCc1c[nH]c2cc(F)c(F)cc12)C1CCC1] | N-(2-(5,6-difluoro-1H-indol-3-yl)ethyl)-N- methylcyclobutanamine |
| 67 | 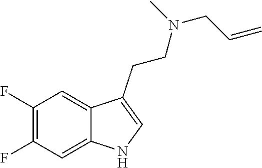 [SMILES: C=CCN(C)CCc1c[nH]c2cc(F)c(F)cc12] | N-(2-(5,6-difluoro-1H-indol-3-yl)ethyl)-N- methylprop-2-en-1-amine |
| 68 | 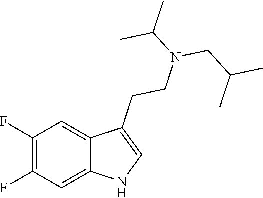 [SMILES: CC(C)CN(CCc1c[nH]c2cc(F)c(F)cc12)C(C)C] | N-(2-(5,6-difluoro-1H-indol-3-yl)ethyl)-N- isopropyl-2-methylpropan-1-amine |
| 69 | 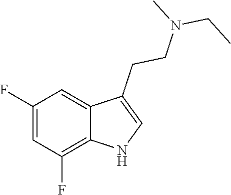 [SMILES: CCN(C)CCc1c[nH]c2c(F)cc(F)cc12] | 2-(5,7-difluoro-1H-indol-3-yl)-N-ethyl-N- methylethan-1-amine |
| 70 | 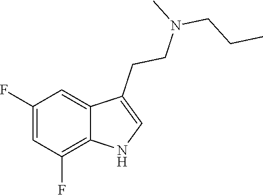 [SMILES: CCCN(C)CCc1c[nH]c2c(F)cc(F)cc12] | N-(2-(5,7-difluoro-1H-indol-3-yl)ethyl)-N- methylpropan-1-amine |
| 71 | 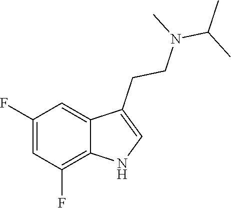 [SMILES: CC(C)N(C)CCc1c[nH]c2c(F)cc(F)cc12] | N-(2-(5,7-difluoro-1H-indol-3-yl)ethyl)-N- methylpropan-2-amine |
| 72 | 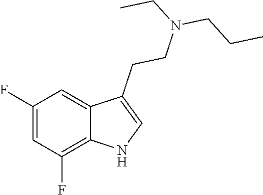 [SMILES: CCCN(CC)CCc1c[nH]c2c(F)cc(F)cc12] | N-(2-(5,7-difluoro-1H-indol-3-yl)ethyl)-N- ethylpropan-1-amine |
| 73 | 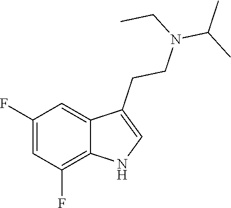 [SMILES: CCN(CCc1c[nH]c2c(F)cc(F)cc12)C(C)C] | N-(2-(5,7-difluoro-1H-indol-3-yl)ethyl)-N- ethylpropan-2-amine |
| 74 | 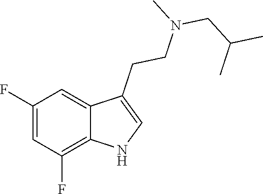 [SMILES: CC(C)CN(C)CCc1c[nH]c2c(F)cc(F)cc12] | N-(2-(5,7-difluoro-1H-indol-3-yl)ethyl)-N,2- dimethylpropan-1-amine |
| 75 | 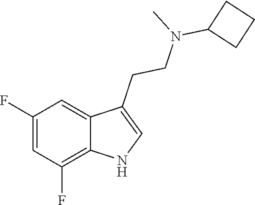 [SMILES: CN(CCc1c[nH]c2c(F)cc(F)cc12)C1CCC1] | N-(2-(5,7-difluoro-1H-indol-3-yl)ethyl)-N- methylcyclobutanamine |
| 76 | 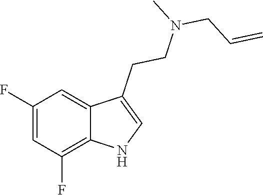 [SMILES: C=CCN(C)CCc1c[nH]c2c(F)cc(F)cc12] | N-(2-(5,7-difluoro-1H-indol-3-yl)ethyl)-N- methylprop-2-en-1-amine |
| 78 | 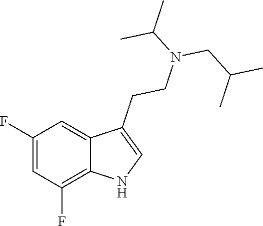 [SMILES: CC(C)CN(CCc1c[nH]c2c(F)cc(F)cc12)C(C)C] | N-(2-(5,7-difluoro-1H-indol-3-yl)ethyl)-N- isopropyl-2-methylpropan-1-amine |
| 79 | 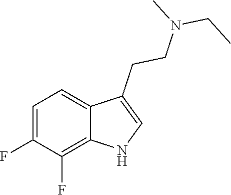 [SMILES: CCN(C)CCc1c[nH]c2c(F)c(F)ccc12] | 2-(6,7-difluoro-1H-indol-3-yl)-N-ethyl-N- methylethan-1-amine |
| 80 | 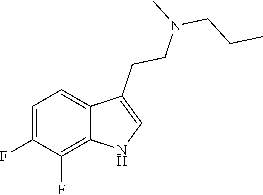 [SMILES: CCCN(C)CCc1c[nH]c2c(F)c(F)ccc12] | N-(2-(6,,7-difluoro-1H-indol-3-yl)ethyl)-N- methylpropan-1-amine |
| 81 | 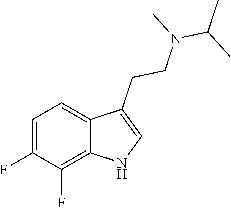 [SMILES: CC(C)N(C)CCc1c[nH]c2c(F)c(F)ccc12] | N-(2-(6,7-difluoro-1H-indol-3-yl)ethyl)-N- methylpropan-2-amine |
| 82 | 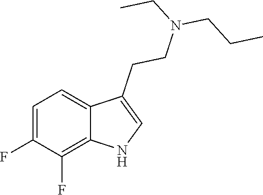 [SMILES: CCCN(CC)CCc1c[nH]c2c(F)c(F)ccc12] | N-(2-(6,7-difluoro-1H-indol-3-yl)ethyl)-N- ethylpropan-1-amine |
| 83 | 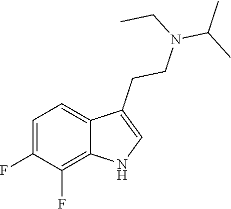 [SMILES: CCN(CCc1c[nH]c2c(F)c(F)ccc12)C(C)C] | N-(2-(6,7-difluoro-1H-indol-3-yl)ethyl)-N- ethylpropan-2-amine |
| 84 | 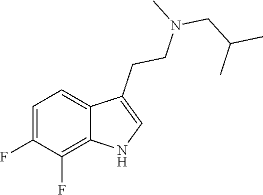 [SMILES: CC(C)CN(C)CCc1c[nH]c2c(F)c(F)ccc12] | N-(2-(6,7-difluoro-1H-indol-3-yl)ethyl)-N,2- dimethylpropan-1-amine |
| 85 | 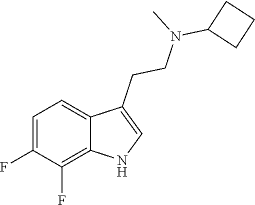 [SMILES: CN(CCc1c[nH]c2c(F)c(F)ccc12)C1CCC1] | N-(2-(6,7-difluoro-1H-indol-3-yl)ethyl)-N- methylcyclobutanamine |
| 86 | 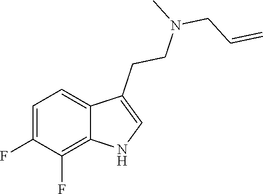 [SMILES: C=CCN(C)CCc1c[nH]c2c(F)c(F)ccc12] | N-(2-(6,7-difluoro-1H-indol-3-yl)ethyl)-N- methylprop-2-en-1-amine |
| 87 | 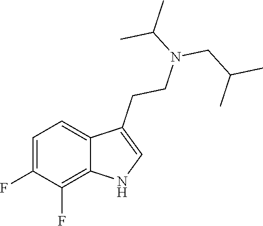 [SMILES: CC(C)CN(CCc1c[nH]c2c(F)c(F)ccc12)C(C)C] | N-(2-(6,7-difluoro-1H-indol-3-yl)ethyl)-N- isopropyl-2-methylpropan-1-amine |
| 88 | 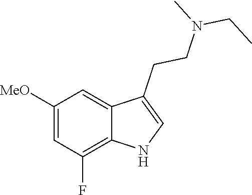 [SMILES: CCN(C)CCc1c[nH]c2c(F)cc(OC)cc12] | N-ethyl-2-(7-fluoro-5-methoxy-1H-indol-3- yl)-N-methylethan-1-amine |
| 89 | 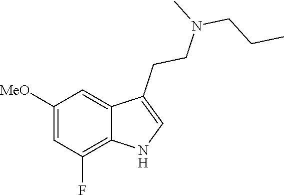 [SMILES: CCCN(C)CCc1c[nH]c2c(F)cc(OC)cc12] | N-(2-(7-fluoro-5-methoxy-1H-indol-3- yl)ethyl)-N-methylpropan-1-amine |
| 90 | 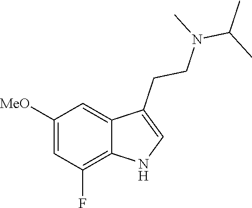 [SMILES: COc1cc(F)c2[nH]cc(CCN(C)C(C)C)c2c1] | N-(2-(7-fluoro-5-methoxy-1H-indol-3- yl)ethyl)-N-methylpropan-2-amine |
| 91 | 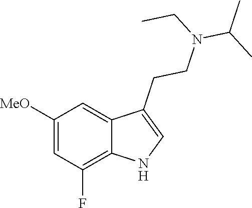 [SMILES: CCN(CCc1c[nH]c2c(F)cc(OC)cc12)C(C)C] | N-ethyl-N-(2-(7-fluoro-5-methoxy-1H-indol- 3-yl)ethyl)propan-2-amine |
| 92 | 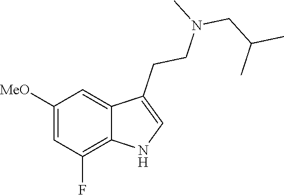 [SMILES: COc1cc(F)c2[nH]cc(CCN(C)CC(C)C)c2c1] | N-(2-(7-fluoro-5-methoxy-1H-indol-3- yl)ethyl)-N,2-dimethylpropan-1-amine |
| 93 | 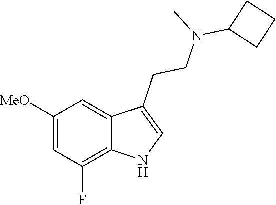 [SMILES: COc1cc(F)c2[nH]cc(CCN(C)C3CCC3)c2c1] | N-(2-(7-fluoro-5-methoxy-1H-indol-3- yl)ethyl)-N-methylcyclobutanamine |
| 94 | 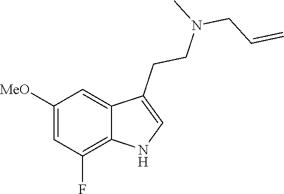 [SMILES: C=CCN(C)CCc1c[nH]c2c(F)cc(OC)cc12] | N-(2-(7-fluoro-5-methoxy-1H-indol-3- yl)ethyl)-N-methylprop-2-en-1-amine |
| 95 | 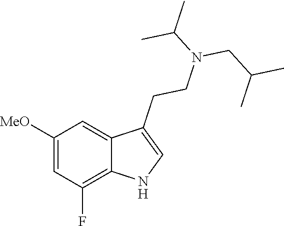 [SMILES: COc1cc(F)c2[nH]cc(CCN(CC(C)C)C(C)C)c2c1] | N-(2-(7-fluoro-5-methoxy-1H-indol-3- yl)ethyl)-N-isopropyl-2-methylpropan-1- amine |
| 96 | 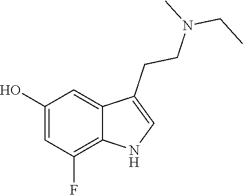 [SMILES: CCN(C)CCc1c[nH]c2c(F)cc(O)cc12] | 3-(2-(ethyl(methyl)amino)ethyl)-7-fluoro- 1H-indol-5-ol |
| 97 | 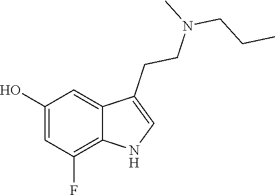 [SMILES: CCCN(C)CCc1c[nH]c2c(F)cc(O)cc12] | 7-fluoro-3-(2-(methyl(propyl)amino)ethyl)- 1H-indol-5-ol |
| 98 | 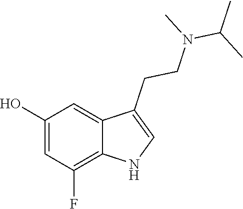 [SMILES: CC(C)N(C)CCc1c[nH]c2c(F)cc(O)cc12] | 7-fluoro-3-(2- (isopropyl(methyl)amino)ethyl)-1H-indol-5- ol |
| 99 | 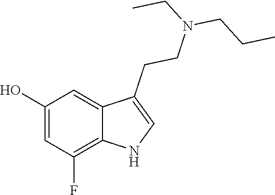 [SMILES: CCCN(CC)CCc1c[nH]c2c(F)cc(O)cc12] | 3-(2-(ethyl)propyl)amino)ethyl)-7-fluoro-1H- indol-5-ol |
| 100 | 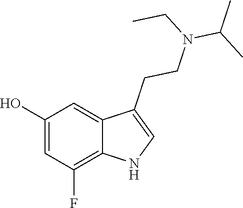 [SMILES: CCN(CCc1c[nH]c2c(F)cc(O)cc12)C(C)C] | 3-(2-(ethyl(isopropyl)amino)ethyl)-7-fluoro- 1H-indol-5-ol |
| 101 | 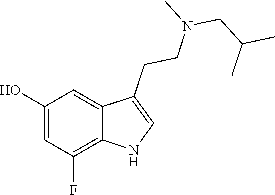 [SMILES: CC(C)CN(C)CCc1c[nH]c2c(F)cc(O)cc12] | 7-fluoro-3-(2-(isobutyl)methyl)amino)ethyl)- 1H-indol-5-ol |
| 102 | 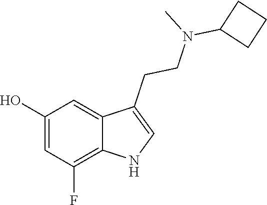 [SMILES: CN(CCc1c[nH]c2c(F)cc(O)cc12)C1CCC1] | 3-(2-(cyclobutyl(methyl)amino)ethyl)-7- fluoro-1H-indol-5-ol |
| 103 | 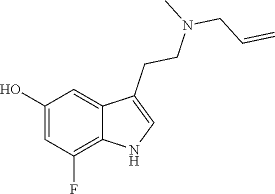 [SMILES: C=CCN(C)CCc1c[nH]c2c(F)cc(O)cc12] | 3-(2-(allyl(methyl)amino)ethyl)-7-fluoro-1H- indol-5-ol |
| 104 | 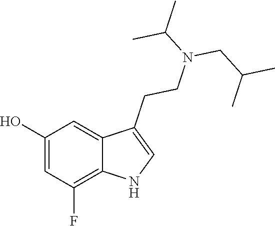 [SMILES: CC(C)CN(CCc1c[nH]c2c(F)cc(O)cc12)C(C)C] | 7-fluoro-3-(2- (isobutyl(isopropyl)amino)ethyl)-1H-indol-5- ol |
| 105 | 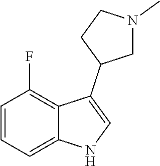 [SMILES: CN1CCC(c2c[nH]c3cccc(F)c23)C1] | 4-fluoro-3-(1-methylpyrrolidin-3-yl)-1H- indole |
| 106 | 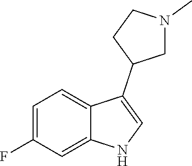 [SMILES: CN1CCC(c2c[nH]c3cc(F)ccc23)C1] | 6-fluoro-3-(1-methylpyrrolidin-3-yl)-1H- indole |
| 107 | 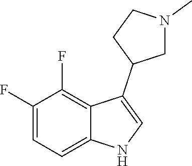 [SMILES: CN1CCC(c2c[nH]c3ccc(F)c(F)c23)C1] | 4,5-difluoro-3-(1-methylpyrrolidin-3-yl)-1H- indole |
| 108 | 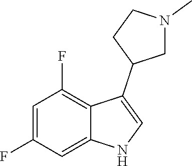 [SMILES: CN1CCC(c2c[nH]c3cc(F)cc(F)c23)C1] | 4,6-difluoro-3-(1-methylpyrrolidin-3-yl)-1H- indole |
| 109 | 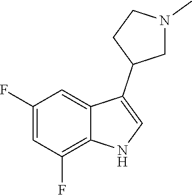 [SMILES: CN1CCC(c2c[nH]c3c(F)cc(F)cc23)C1] | 5,7-difluoro-3-(1-methylpyrrolidin-3-yl)-1H- indole |
| 110 | 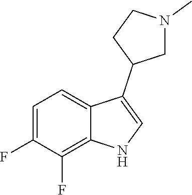 [SMILES: CN1CCC(c2c[nH]c3c(F)c(F)ccc23)C1] | 6,7-difluoro-3-(1-methylpyrrolidin-3-yl)-1H- indole |
| 111 | 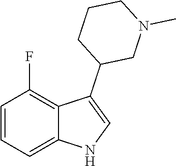 [SMILES: CN1CCCC(c2c[nH]c3cccc(F)c23)C1] | 4-fluoro-3-(1-methylpiperidin-3-yl)-1H- indole |
| 112 | 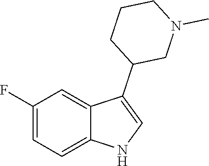 [SMILES: CN1CCCC(c2c[nH]c3ccc(F)cc23)C1] | 5-fluoro-3-(1-methylpiperidin-3-yl)-1H- indole |
| 113 | 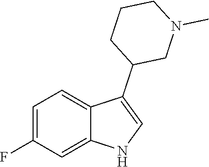 [SMILES: CN1CCCC(c2c[nH]c3cc(F)ccc23)C1] | 6-fluoro-3-(1-methylpiperidin-3-yl)-1H- indole |
| 114 | 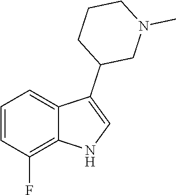 [SMILES: CN1CCCC(c2c[nH]c3c(F)cccc23)C1] | 7-fluoro-3-(1-methylpiperidin-3-yl)-1H- indole |
| 115 | 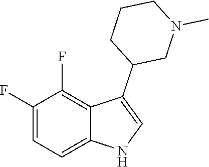 [SMILES: CN1CCCC(c2c[nH]c3ccc(F)c(F)c23)C1] | 4,5-difluoro-3-(1-methylpiperidin-3-yl)-1H- indole |
| 116 | 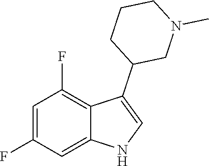 [SMILES: CN1CCCC(c2c[nH]c3cc(F)cc(F)c23)C1] | 4,6-difluoro-3-(1-methylpiperidin-3-yl)-1H- indole |
| 117 | 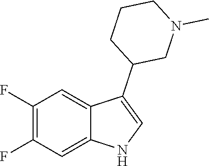 [SMILES: CN1CCCC(c2c[nH]c3cc(F)c(F)cc23)C1] | 5,6-difluoro-3-(1-methylpiperidin-3-yl)-1H- indole |
| 118 | 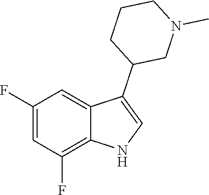 [SMILES: CN1CCCC(c2c[nH]c3c(F)cc(F)cc23)C1] | 5,7-difluoro-3-(1-methylpiperidin-3-yl)-1H- indole |
| 119 | 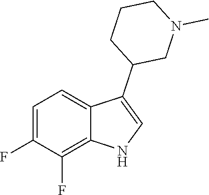 [SMILES: CN1CCCC(c2c[nH]c3c(F)c(F)ccc23)C1] | 6,7-difluoro-3-(1-methylpiperidin-3-yl)-1H- indole |
| 120 | 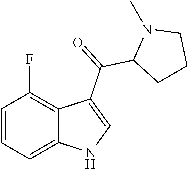 [SMILES: CN1CCCC1C(=O)c1c[nH]c2cccc(F)c12] | 4-fluoro-3-(methylprolyl)-1H-indole |
| 121 | 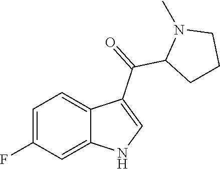 [SMILES: CN1CCCC1C(=O)c1c[nH]c2cc(F)ccc12] | 6-fluoro-3-(methylprolyl)-1H-indole |
| 122 | 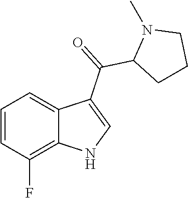 [SMILES: CN1CCCC1C(=O)c1c[nH]c2c(F)cccc12] | 7-fluoro-3-(mehtylprolyl)-1H-indole |
| 123 | 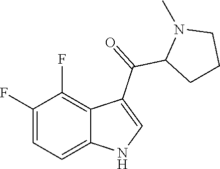 [SMILES: CN1CCCC1C(=O)c1c[nH]c2ccc(F)c(F)c12] | 4,5-difluoro-3-(methylprolyl)-1H-indole |
| 124 | 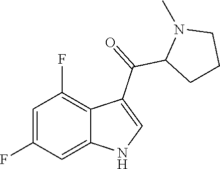 [SMILES: CN1CCCC1C(=O)c1c[nH]c2cc(F)cc(F)c12] | 4,6-difluoro-3-(methylprolyl)-1H-indole |
| 125 | 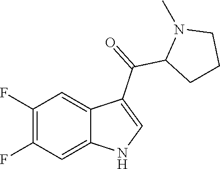 [SMILES: CN1CCCC1C(=O)c1c[nH]c2cc(F)c(F)cc12] | 5,6-difluoro-3-(methylprolyl)-1H-indole |
| 126 | 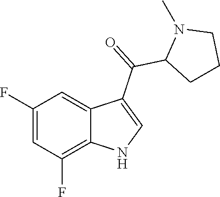 [SMILES: CN1CCCC1C(=O)c1c[nH]c2c(F)cc(F)cc12] | 5,7-difluoro-3-(methylprolyl)-1H-indole |
| 127 | 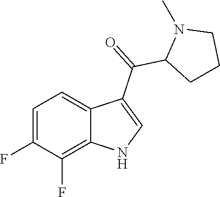 [SMILES: CN1CCCC1C(=O)c1c[nH]c2c(F)c(F)ccc12] | 6,7-difluoro-3-(methylprolyl)-1H-indole |
| 128 | 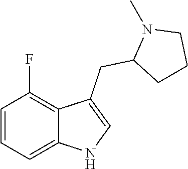 [SMILES: CN1CCCC1Cc1c[nH]c2cccc(F)c12] | 4-fluoro-3-((1-methylpyrrolidin-2- yl)methyl)-1H-indole |
| 129 | 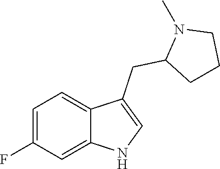 [SMILES: CN1CCCC1Cc1c[nH]c2cc(F)ccc12] | 6-fluoro-3-((1-methylpyrrolidin-2- yl)methyl)-1H-indole |
| 130 | 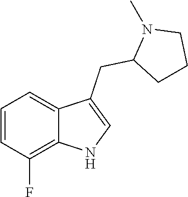 [SMILES: CN1CCCC1Cc1c[nH]c2c(F)cccc12] | 7-fluoro-3-((1-methylpyrrolidin-2- yl)methyl)-1H-indole |
| 131 | 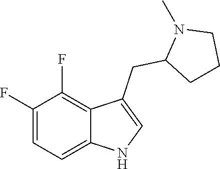 [SMILES: CN1CCCC1Cc1c[nH]c2ccc(F)c(F)c12] | 4,5-difluoro-3-((1-methylpyrrolidin-2- yl)methyl)-1H-indole |
| 132 | 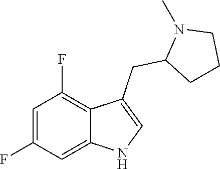 [SMILES: CN1CCCC1Cc1c[nH]c2cc(F)cc(F)c12] | 4,6-difluoro-3-((1-methylpyrrolidin-2- yl)methyl)-1H-indole |
| 133 | 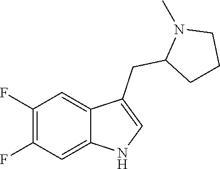 [SMILES: CN1CCCC1Cc1c[nH]c2cc(F)c(F)cc12] | 5,6-difluoro-3-((1-methylpyrrolidin-2- yl)methyl)-1H-indole |
| 134 | 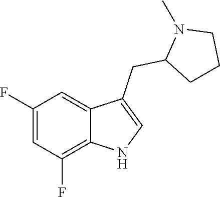 [SMILES: CN1CCCC1Cc1c[nH]c2c(F)cc(F)cc12] | 5,7-difluoro-3-((1-methylpyrrolidin-2- yl)methyl)-1H-indole |
| 135 | 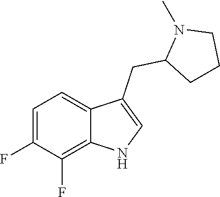 [SMILES: CN1CCCC1Cc1c[nH]c2c(F)c(F)ccc12] | 6,7-difluoro-3-((1-methylpyrrolidin-2- yl)methyl)-1H-indole |
| 136 | 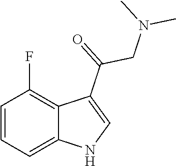 [SMILES: CN(C)CC(=O)c1c[nH]c2cccc(F)c12] | 2-(dimethylamino)-1-(4-fluoro-1H-indol-3- yl)ethan-1-one |
| 137 | 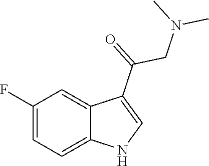 [SMILES: CN(C)CC(=O)c1c[nH]c2ccc(F)cc12] | 2-(dimethylamino)-1-(5-fluoro-1H-indol-3- yl)ethan-1-one |
| 138 | 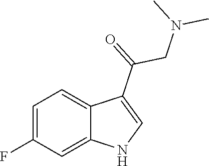 [SMILES: CN(C)CC(=O)c1c[nH]c2cc(F)ccc12] | 2-(dimethylamino)-1-(6-fluoro-1H-indol-3- yl)ethan-1-one |
| 139 | 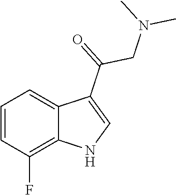 [SMILES: CN(C)CC(=O)c1c[nH]c2c(F)cccc12] | 2-(dimethyalmino)-1-(7-fluoro-1H-indol-3- yl)ethan-1-one |
| 140 | 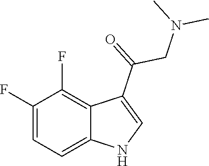 [SMILES: CN(C)CC(=O)c1c[nH]c2ccc(F)c(F)c12] | 1-(4,5-difluoro-1H-indol-3-yl)-2- (dimethylamino)ethan-1-one |
| 141 | 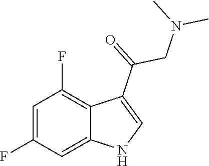 [SMILES: CN(C)CC(=O)c1c[nH]c2cc(F)cc(F)c12] | 1-(4,6-difluoro-1H-indol-3-yl)-2- (dimethylamino)ethan-1-one |
| 142 | 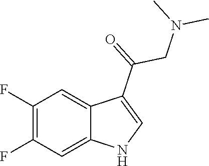 [SMILES: CN(C)CC(=O)c1c[nH]c2cc(F)c(F)cc12] | 1-(5,6-difluoro-1H-indol-3-yl)-2- (dimethylamino)ethan-1-one |
| 143 | 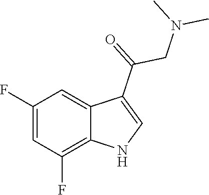 [SMILES: CN(C)CC(=O)c1c[nH]c2c(F)cc(F)cc12] | 1-(5,7-difluoro-1H-indol-3-yl)-2- (dimethylamino)ethan-1-one |
| 144 | 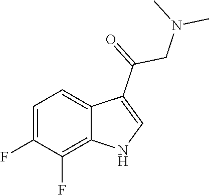 [SMILES: CN(C)CC(=O)c1c[nH]c2c(F)c(F)ccc12] | 1-(6,7-difluoro-1H-indol-3-yl)-2- (dimethylamino)ethan-1-one |
| 145 | 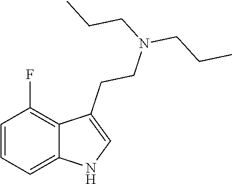 [SMILES: CCCN(CCC)CCc1c[nH]c2cccc(F)c12] | N-(2-(4-fluoro-1H-indol-3-yl)ethyl)-N- propylpropan-1-amine |
| 146 | 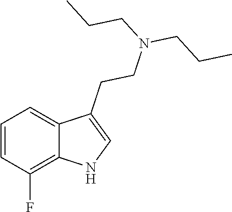 [SMILES: CCCN(CCC)CCc1c[nH]c2c(F)cccc12] | N-(2-(7-fluoro-1H-indol-3-yl)ethyl)-N- propylpropan-1-amine |
| 147 | 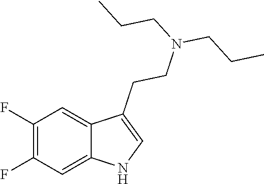 [SMILES: CCCN(CCC)CCc1c[nH]c2cc(F)c(F)cc12] | N-(2-(5,6-difluoro-1H-indol-3-yl)ethyl)-N- propylpropan-1-amine |
| 148 | 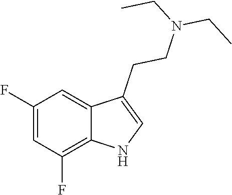 [SMILES: CCN(CC)CCc1c[nH]c2c(F)cc(F)cc12] | 2-(5,7-difluoro-1H-indol-3-yl)-N,N- diethylethan-1-amine |
| 149 | 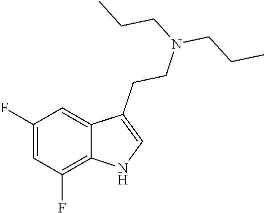 [SMILES: CCCN(CCC)CCc1c[nH]c2c(F)cc(F)cc12] | N-(2-(5,7-difluoro-1H-indol-3-yl)ethyl)-N- propylpropan-1-amine |
| 150 | 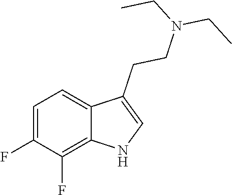 [SMILES: CCN(CC)CCc1c[nH]c2c(F)c(F)ccc12] | 2-(6,7-difluoro-1H-indol-3-yl)-N,N- diethylethan-1-amine |
| 151 | 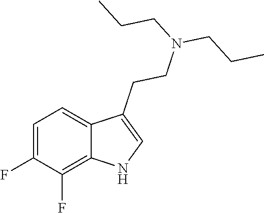 [SMILES: CCCN(CCC)CCc1c[nH]c2c(F)c(F)ccc12] | N-(2-(6,7-difluoro-1H-indol-3-yl)ethyl)-N- propylpropan-1-amine |
| 152 | 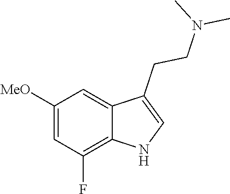 [SMILES: COc1cc(F)c2[nH]cc(CCN(C)C)c2c1] | 2-(7-fluoro-5-methoxy-1H-indol-3-yl)-N,N- dimethylethan-1-amine |
| 153 | 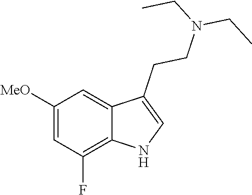 [SMILES: CCN(CC)CCc1c[nH]c2c(F)cc(OC)cc12] | N,N-diethyl-2-(7-fluoro-5-methoxy-1H- indol-3-yl)ethan-1-amine |
| 154 | 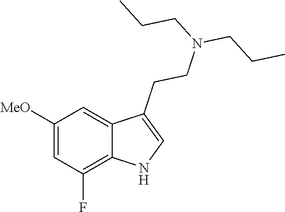 [SMILES: CCCN(CCC)CCc1c[nH]c2c(F)cc(OC)cc12] | N-(2-(7-fluoro-5-methoxy-1H-indol-3- yl)ethyl)-N-propylpropan-1-amine |
| 155 | 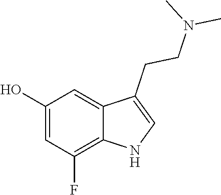 [SMILES: CN(C)CCc1c[nH]c2c(F)cc(O)cc12] | 3-(2-(dimethylamino)ethyl)-7-fluoro-1H- indol-5-ol |
| 156 | 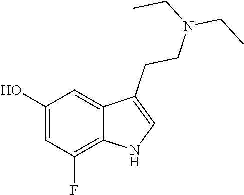 [SMILES: CCN(CC)CCc1c[nH]c2c(F)cc(O)cc12] | 3-(2-(diethylamino)ethyl)-7-fluoro-1H-indol- 5-ol |
| 157 | 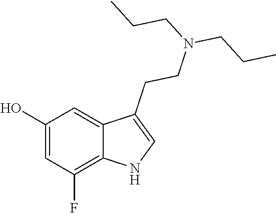 [SMILES: CCCN(CCC)CCc1c[nH]c2c(F)cc(O)cc12] | 3-(2-(dipropylamino)ethyl)-7-fluoro-1H- indol-5-ol |
| 158 | 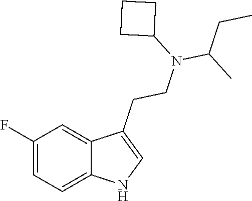 [SMILES: CCC(C)N(CCc1c[nH]c2ccc(F)cc12)C1CCC1] | N-(sec-butyl)-N-(2-(5-fluoro-1H-indol-3- yl)ethyl)cyclobutanamine |
| 159 | 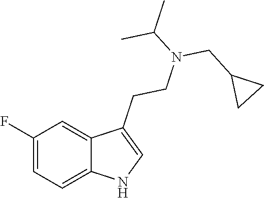 [SMILES: CC(C)N(CCc1c[nH]c2ccc(F)cc12)CC1CC1] | N-(cyclopropylmethyl)-N-(2-(5-fluoro-1H- indol-3-yl)ethyl)propan-2-amine |
| 160 | 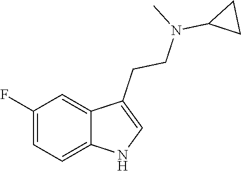 [SMILES: CN(CCc1c[nH]c2ccc(F)cc12)C1CC1] | N-(2-(5-fluoro-1H-indol-3-yl)ethyl)-N- methylcyclopropanamine |
| 161 | 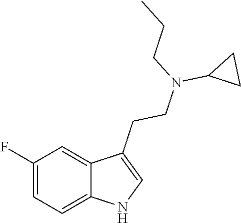 [SMILES: CCCN(CCc1c[nH]c2ccc(F)cc12)C1CC1] | N-(2-(5-fluoro-1H-indol-3-yl)ethyl)-N- propylcyclopropanamine |
| 162 | 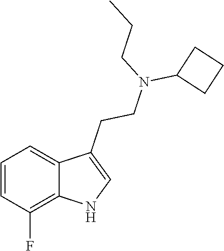 [SMILES: CCCN(CCc1c[nH]c2c(F)cccc12)C1CCC1] | N-(2-(7-fluoro-1H-indol-3-yl)ethyl)-N- propylcyclobutanamine |
| 163 | 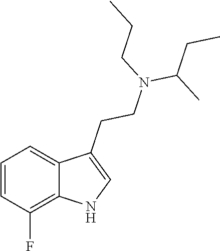 [SMILES: CCCN(CCc1c[nH]c2c(F)cccc12)C(C)CC] | N-(2-(7-fluoro-1H-indol-3-yl)ethyl)-N- propylbutan-2-amien |
| 164 | 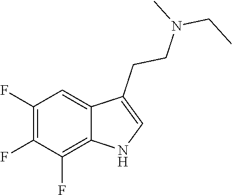 [SMILES: CCN(C)CCc1c[nH]c2c(F)c(F)c(F)cc12] | N-ethyl-N-methyl-2-(5,6,7-trifluoro-1H- indol-3-yl)ethan-1-amine |
| 165 | 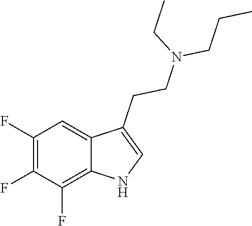 [SMILES: CCCN(CC)CCc1c[nH]c2c(F)c(F)c(F)cc12] | N-ethyl-N-(2-(5,6,7-trifluoro-1H-indol-3- yl)ethyl)propan-1-amine |
| 166 | 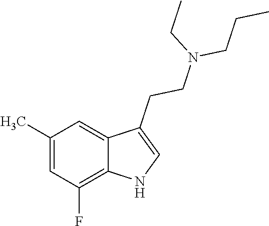 [SMILES: CCCN(CC)CCc1c[nH]c2c(F)cc(C)cc12] | N-ethyl-N-(2-(7-fluoro-5-methyl-1H-indol-3- yl)ethyl)propan-1-amine |
| 167 | 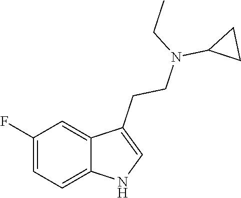 [SMILES: CCN(CCc1c[nH]c2ccc(F)cc12)C1CC1] | N-ethyl-N-(2-(5-fluoro-1H-indol-3- yl)ethyl)cyclopropanamine |
| 168 | 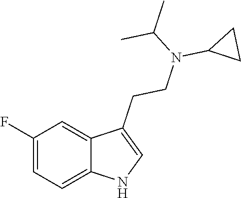 [SMILES: CC(C)N(CCc1c[nH]c2ccc(F)cc12)C1CC1] | N-(2-(5-fluoro-1H-indol-3-yl)ethyl)-N- isopropylcyclopropanamine |
| 169 | 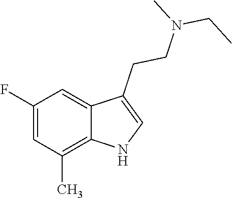 [SMILES: CCN(C)CCc1c[nH]c2c(C)cc(F)cc12] | N-ethyl-2-(5-fluoro-7-methyl-1H-indol-3- yl)-N-methylethan-1-amine |
| 170 | 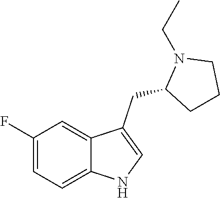 [SMILES: CCN1CCC[C@@H]1Cc1c[nH]c2ccc(F)cc12] | (R)-3-((1-ethylpyrrolidin-2-yl)methyl)-5- fluoro-1H-indole |
| 171 | 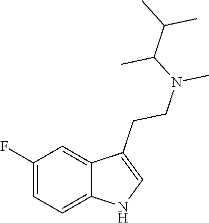 [SMILES: CC(C)C(C)N(C)CCc1c[nH]c2ccc(F)cc12] | N-(2-(5-fluoro-1H-indol-3-yl)ethyl)-N,3- dimethylbutan-2-amine |
| 172 | 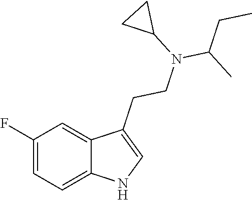 [SMILES: CCC(C)N(CCc1c[nH]c2ccc(F)cc12)C1CC1] | N-(sec-butyl)-N-(2-(5-fluoro-1H-indol-3- yl)ethyl)cyclopropanamine |
| 173 | 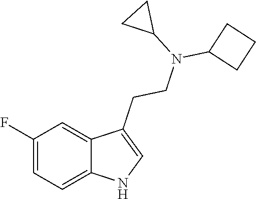 [SMILES: Fc1ccc2[nH]cc(CCN(C3CCC3)C3CC3)c2c1] | N-cyclopropyl-N-(2-(5-fluoro-1H-indol-3- yl)ethyl)cyclobutanamine |
| 174 | 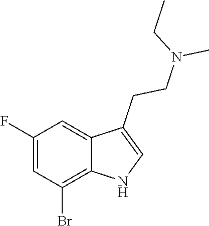 [SMILES: CCN(C)CCc1c[nH]c2c(Br)cc(F)cc12] | 2-(7-bromo-5-fluoro-1H-indol-3-yl)-N-ethyl- N-methylethan-1-amine |
| 175 | 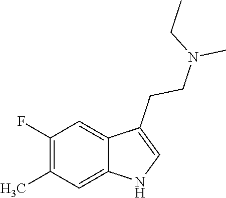 [SMILES: CCN(C)CCc1c[nH]c2cc(C)c(F)cc12] | N-ethyl-2-(5-fluoro-6-methyl-1H-indol-3- yl)-N-methylethan-1-amine |
| 176 | 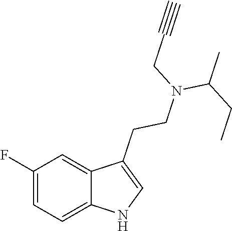 [SMILES: CCCN(CCc1c[nH]c2ccc(F)cc12)C(C)CC] | N-(2-(5-fluoro-1H-indol-3-yl)ethyl)-N-(prop- 2-yn-1-yl)butan-2-amine |
| 177 | 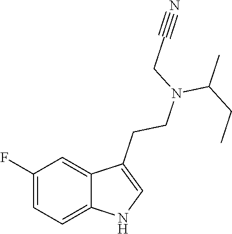 [SMILES: CCC(C)N(CCN)CCc1c[nH]c2ccc(F)cc12] | 2-(sec-butyl(2-(5-fluoro-1H-indol-3- yl)ethyl)amino)acetonitrile |
| 178 | 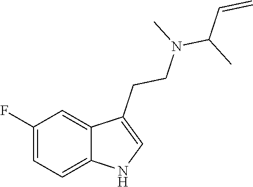 [SMILES: C=CC(C)N(C)CCc1c[nH]c2ccc(F)cc12] | N-(2-(5-fluoro-1H-indol-3-yl)ethyl)-N- methylbut-3-en-2-amine |
| 179 | 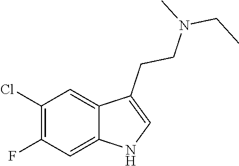 [SMILES: CCN(C)CCc1c[nH]c2cc(F)c(Cl)cc12] | 2-(5-chloro-6-fluoro-1H-indol-3-yl)-N-ethyl- N-methylethan-1-amine |
| 180 | 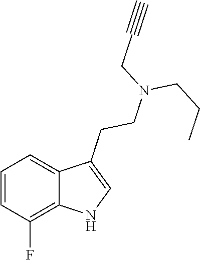 [SMILES: CCCN(CCC)CCc1c[nH]c2c(F)cccc12] | N-(2-(7-fluoro-1H-indol-3-yl)ethyl)-N- propylprop-2-yn-1-amine |
| 181 | 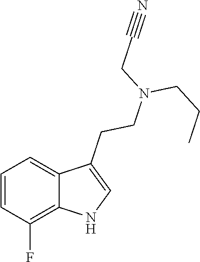 [SMILES: CCCN(CCN)CCc1c[nH]c2c(F)cccc12] | 2-((2-(7-fluoro-1H-indol-3- yl)ethyl)(propyl)amino)acetonitrile |
| 182 | 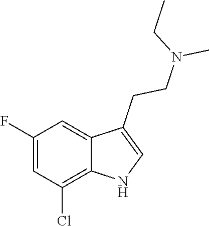 [SMILES: CCN(C)CCc1c[nH]c2c(Cl)cc(F)cc12] | 2-(7-chloro-5-fluoro-1H-indol-3-yl)-N-ethyl- N-methylethan-1-amine |
| 183 | 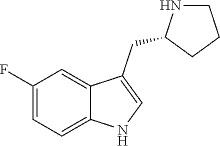 [SMILES: Fc1ccc2[nH]cc(C[C@H]3CCCN3)c2c1] | (R)-5-fluoro-3-(pyrrolidin-2-ylmethyl)-1H- indole |
| 184 | 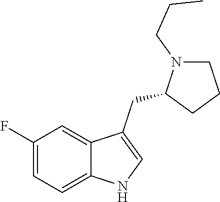 [SMILES: CCCN1CCC[C@@H]1Cc1c[nH]c2ccc(F)cc12] | (R)-5-fluoro-3-((1-propylpyrrolidin-2- yl)methyl)-1H-indole |
| 185 | 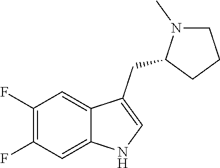 [SMILES: CN1CCC[C@@H]1Cc1c[nH]c2cc(F)c(F)cc12] | (R)-5,6-difluoro-3-((1-methylpyrrolidin-2- yl)methyl)-1H-indole |
| 186 | 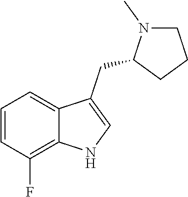 [SMILES: CN1CCC[C@@H]1Cc1c[nH]c2c(F)cccc12] | (R)-7-fluoro-3-((1-methylpyrrolidin-2- yl)methyl)-1H-indole |
| 187 | 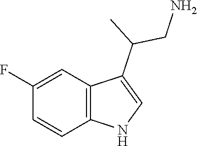 | 2-(5-fluoro-1H-indol-3-yl)propan-1-amine |
| 188 | 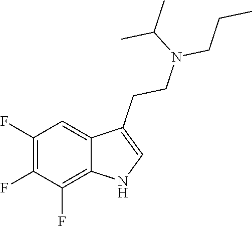 [SMILES: CCCN(CCc1c[nH]c2c(F)c(F)c(F)cc12)C(C)C] | N-isopropyl-N-(2-(5,6,7-trifluoro-1H-indol- 3-yl)ethyl)propan-1-amine |
| 189 | 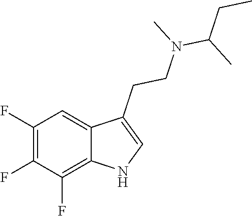 [SMILES: CCC(C)N(C)CCc1c[nH]c2c(F)c(F)c(F)cc12] | N-methyl-N-(2-(5,6,7-trifluroo-1H-indol-3- yl)ethyl)butan-2-amine |
| 190 | 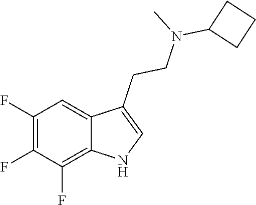 [SMILES: CN(CCc1c[nH]c2c(F)c(F)c(F)cc12)C1CCC1] | N-methyl-N-(2-(5,6,7-trifluoro-1H-indol-3- yl)ethyl)cyclobutanamine |
| 191 | 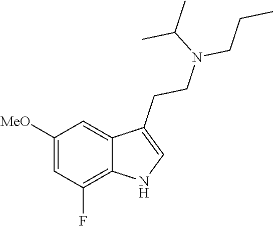 [SMILES: CCCN(CCc1c[nH]c2c(F)cc(OC)cc12)C(C)C] | N-(2-(7-fluoro-5-methoxy-1H-indol-3- yl)ethyl)-N-isopropylpropan-1-amine |
| 192 | 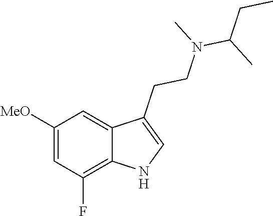 [SMILES: CCC(C)N(C)CCc1c[nH]c2c(F)cc(OC)cc12] | N-(2-(7-fluoro-5-methoxy-1H-indol-3- yl)ethyl)-N-methylbutan-2-amine |
| 193 | 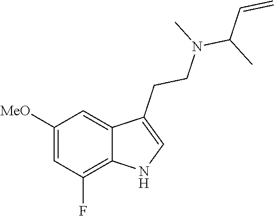 [SMILES: C=CC(C)N(C)CCc1c[nH]c2c(F)cc(OC)cc12] | N-(2-(7-fluoro-5-methoxy-1H-indol-3- yl)ethyl)-N-methylbut-3-en-2-amine |
| 194 | 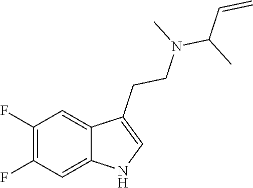 [SMILES: C=CC(C)N(C)CCc1c[nH]c2cc(F)c(F)cc12] | N-(2-(5,6-difluoro-1H-indol-3-yl)ethyl)-N- methylbut-3-en-2-amine |
| 195 | 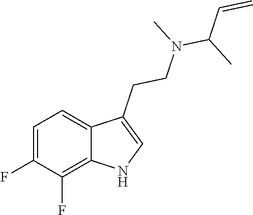 [SMILES: C=CC(C)N(C)CCc1c[nH]c2c(F)c(F)ccc12] | N-(2-(6,7-difluoro-1H-indol-3-yl)ethyl)-N- methylbut-3-en-2-amine |
| 196 | 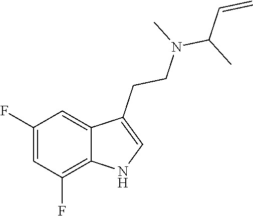 [SMILES: C=CC(C)N(C)CCc1c[nH]c2c(F)cc(F)cc12] | N-(2-(5,7-difluoro-1H-indol-3-yl)ethyl)-N- methylbut-3-en-2-amine |
| 197 | 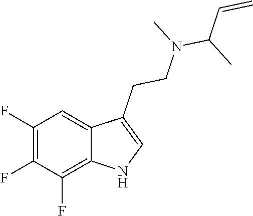 [SMILES: C=CC(C)N(C)CCc1c[nH]c2c(F)c(F)c(F)cc12] | N-methyl-N-(2-(5,6,7-trifluoro-1H-indol-3- yl)ethyl)but-3-en-2-amine |
| 198 | 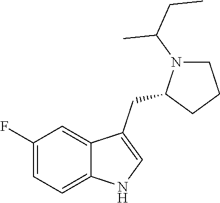 [SMILES: CCC(C)N1CCC[C@@H]1Cc1c[nH]c2ccc(F)cc12] | 3-(((2R)-1-(sec-butyl)pyrrolidin-2- yl)methyl)-5-fluoro-1H-indole |
| 199 | 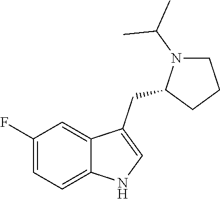 [SMILES: CC(C)N1CCC[C@@H]1Cc1c[nH]c2ccc(F)cc12] | (R)-5-fluoro-3-((1-isopropylpyrrolidin-2- yl)methyl)-1H-indole |
| 200 | 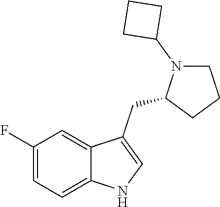 [SMILES: Fc1ccc2[nH]cc(C[C@H]3CCCN3C3CCC3)c2c1] | (R)-3-((1-cyclobutylpyrrolidin-2-yl)methyl)- 5-fluoro-1H-indole |
| 201 | 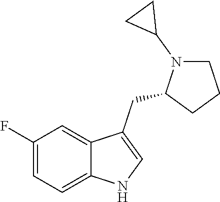 [SMILES: Fc1ccc2[nH]cc(C[C@H]3CCCN3C3CC3)c2c1] | (R)-3-((1-cyclopropylpyrrolidin-2- yl)methyl)-5-fluoro-1H-indole |
| 202 | 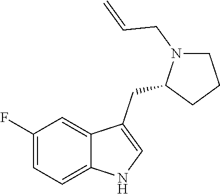 [SMILES: C=CCN1CCC[C@@H]1Cc1c[nH]c2ccc(F)cc12] | (R)-3-((1-allylpyrrolidin-2-yl)methyl)-5- fluoro-1H-indole |
| 203 | 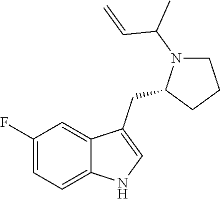 [SMILES: C=CC(C)N1CCC[C@@H]1Cc1c[nH]c2ccc(F)cc12] | 3-(((2R)-1-(but-3-en-2-yl)pyrrolidin-2- yl)methyl)-5-fluoro-1H-indole |
| 204 | 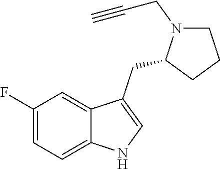 [SMILES: C#CCN1CCC[C@@H]1Cc1c[nH]c2ccc(F)cc12] | (R)-5-fluoro-3-((1-(prop-2-yn-1- yl)pyrrolidin-2-yl)methyl)-1H-indole |
| 205 | 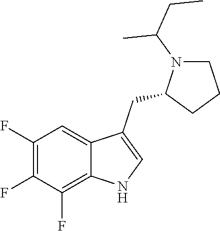 [SMILES: CCC(C)N1CCC[C@@H]1Cc1c[nH]c2c(F)c(F)c(F)cc12] | 3-(((2R)-1-(sec-butyl)pyrrolidin-2- yl)methyl)-5,6,7-trifluoro-1H-indole |
| 206 | 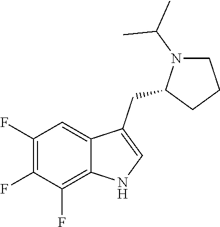 [SMILES: CC(C)N1CCC[C@@H]1Cc1c[nH]c2c(F)c(F)c(F)cc12] | (R)-5,6,7-trifluoro-3-((1-isopropylpyrrolidin- 2-yl)methyl)-1H-indole |
| 207 | 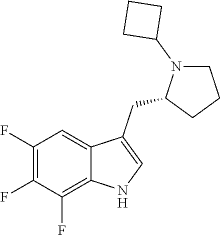 [SMILES: Fc1cc2c(C[C@H]3CCCN3C3CCC3)c[nH]c2c(F)c1F] | (R)-3-((1-cyclobutylpyrrolidin-2-yl)methyl)- 5,6,7-trifluoro-1H-indole |
| 208 | 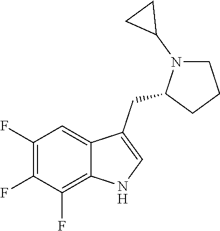 [SMILES: Fc1cc2c(C[C@H]3CCCN3C3CC3)c[nH]c2c(F)c1F] | (R)-3-((1-cyclopropylpyrrolidin-2- yl)methyl)-5,6,7-trifluoro-1H-indole |
| 209 | 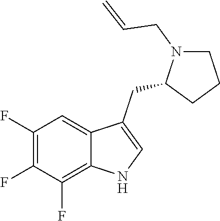 [SMILES: C=CCN1CCC[C@@H]1Cc1c[nH]c2c(F)c(F)c(F)cc12] | (R)-3-((1-allylpyrrolidin-2-yl)methyl)-5,6,7- trifluoro-1H-indole |
| 210 | 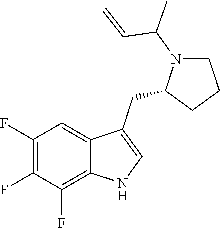 [SMILES: C=CC(C)N1CCC[C@@H]1Cc1c[nH]c2c(F)c(F)c(F)cc12] | 3-(((2R)-1-(but-3-en-2-yl)pyrrolidin-2- yl)methyl)-5,6,7-trifluoro-1H-indole |
| 211 | 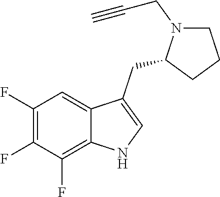 [SMILES: C#CCN1CCC[C@@H]1Cc1c[nH]c2c(F)c(F)c(F)cc12] | (R)-5,6,7-trifluoro-3-((1-(prop-2-yn-1- yl)pyrrolidin-2-yl)methyl)-1H-indole |
| 212 | 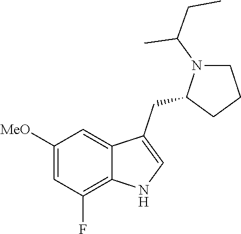 [SMILES: CCC(C)N1CCC[C@@H]1Cc1c[nH]c2c(F)cc(OC)cc12] | 3-(((2R)-1-(sec-butyl)pyrrolidin-2- yl)methyl)-7-fluoro-5-methoxy-1H-indole |
| 213 | 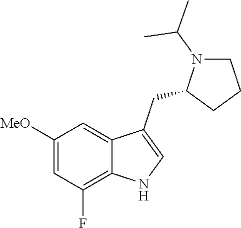 [SMILES: COc1cc(F)c2[nH]cc(C[C@H]3CCCN3C(C)C)c2c1] | (R)-7-fluoro-3-((1-isopropylpyrrolidin-2- yl)methyl)-5-methoxy-1H-indole |
| 214 | 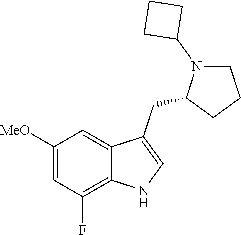 [SMILES: COc1cc(F)c2[nH]cc(C[C@H]3CCCN3C3CCC3)c2c1] | (R)-3-((1-cyclobutylpyrrolidin-2-yl)methyl)- 7-fluoro-5-methoxy-1H-indole |
| 215 | 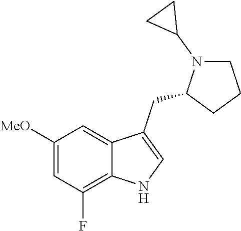 [SMILES: COc1cc(F)c2[nH]cc(C[C@H]3CCCN3C3CC3)c2c1] | (R)-3-((1-cyclopropylpyrrolidin-2- yl)methyl)-7-fluoro-5-methoxy-1H-indole |
| 216 | 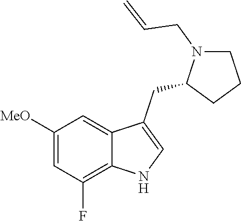 [SMILES: C=CCN1CCC[C@@H]1Cc1c[nH]c2c(F)cc(OC)cc12] | (R)-3-((1-allylpyrrolidin-2-yl)methyl)-7- fluoro-5-methoxy-1H-indole |
| 217 | 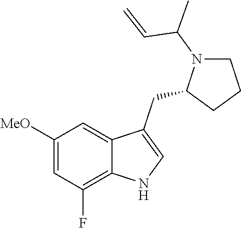 [SMILES: C=CC(C)N1CCC[C@@H]1Cc1c[nH]c2c(F)cc(OC)cc12] | 3-(((2R)-1-(but-3-en-2-yl)pyrrolidin-2- yl)methyl)-7-fluoro-5-methoxy-1H-indole |
| 218 | 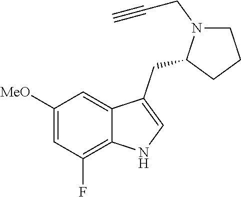 [SMILES: C#CCN1CCC[C@@H]1Cc1c[nH]c2c(F)cc(OC)cc12] | (R)-7-fluoro-5-methoxy-3-((1-(prop-2-yn-1- yl)pyrrolidin-2-yl)methyl)-1H-indole |

### Example 78: Molecular Docking Studies

Rigid docking using UCSD Autodock 4.2.6 was utilized. The receptor used was 6WGT (Kim et al. 2020). Receptor and ligand structures were prepared using Discovery Studio Visualizer. Docking was performed using AutodockTools 1.5.6. Receptor preparation included removing ligand, adding polar hydrogen atoms, merging non-polar hydrogen atoms, and adding Gasteiger charges. The grid was centered around the orthosteric LSD binding site. Genetic algorithm was used for search parameters and output was Lamarckian genetic algorithm. Docking results (top 10 poses per compound) were ranked by energy and exported (as .pdb files), then individually viewed in PyMol.

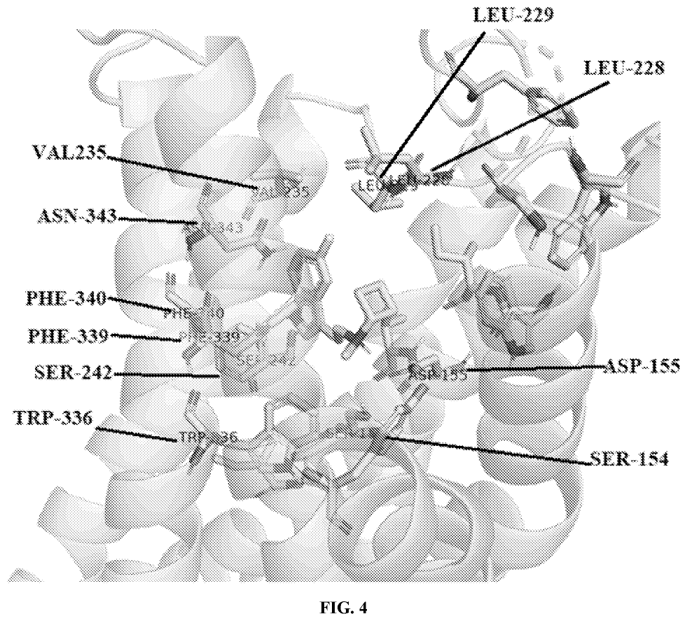

Docking studies were performed as described elsewhere herein, and demonstrated favorable binding of numerous examples of the target fluorinated tryptamines to the 5-HT_{2A} receptor (6WGT), as illustrated in FIG. 4, which shows binding of compound 51 to the 5-HT_{2A} receptor. Compound 51 shows close intermolecular interactions with several key residues of the orthosteric 5-HT_{2A} binding site. Docking was also performed on the endogenous ligand 5-hydroxytryptamine (5-HT or serotonin). Compound 51 shows a similar binding mode as serotonin, suggesting that the fluorine atom confers desirable interactions which underlie its ability to modulate the activity off the tryptamine scaffold.

Key interactions include an ionic interaction between the ammonium ion of protonated compound 51, which is expected to predominate under physiological conditions, and the highly conserved Asp155^{3.32}. Pi-stacking interactions are observed between the 5-fluoro-indole ring of compound 6 and the TM6 residues Phe339^{6.51}, Phe340^{6.52} and Trp336^{6.48}. Fluorination alters the electronics of the indole, resulting in differences in the strength and orientation of electrostatic interactions.

A predicted hydrogen bonding interaction between compound 51 and Asn343^{6.55} was also observed. Asn343^{6.55} is thought to engage in hydrogen bonding with the hydroxyl of serotonin (5-HT). Thus, without wishing to be bound by theory, the predicted hydrogen bonding interaction between 52 and Asn343^{6.55} suggests that the 5-fluoro-substituent can act as a hydrogen bonding acceptor comparable to a hydroxy or methoxy substituent, which are common in many reported tryptamine derivatives, including 5-methoxy-dimethyltryptamine (5-MeO-DMT) and 5-hydroxy-dimethyltryptamine (5-OH-DMT). This unexpected finding provides a basis for the high potency and agonism observed with the 5-fluoro-indole series, in contrast to prior reports of loss of activity with 6-F-tryptamine and fluorinated-psilocin analogs.

Close interactions involving N-alkyl substituents with Ile152^{3.29}, Trp151^{3.28}, Phe339^{6.51}, and Tyr370^{7.43}, were also observed, suggesting the importance of these substituents on binding affinity and functional activity. The N-alkyl substituents were also in close proximity to the important toggle switch Trp336^{6.48}. It is proposed herein that the functional selectivity observed in a number of cases may be due to differential interactions between these residues of TM3, TM5, TM6, and TM7 of the orthosteric site. Non-symmetrical N,N-dialkyl substitution of the 5-fluorotryptamine scaffold thus represents one approach to modulate binding affinities and functional activities of the 5-HT_{2A} receptor, as well as similar receptors including, but not limited to 5-HT_{2B}, 5-HT_{2C}, and 5-HT_{1A}, while simultaneously optimizing pharmacokinetic and physiochemical properties (e.g., absorption, distribution, metabolism, and excretion).

Additionally, close contact between compound 6 and Val235^{5.39} and Leu229^{ECL2} were observed, suggesting Van der Waals interactions (i.e., hydrophobic interactions). Leu229^{ECL2} is implicated in the high binding affinity of prototypical hallucinogenic LSD at the 5-HT₂ receptors. However, this interaction has not been previously reported with other tryptamines and may be unique to the compounds of the present invention.

Generally, the compounds of the present invention provided binding energy scores between −6 to −7 kcal/mol, which is consistent with the endogenous agonist 5-HT and known biologically active tryptamine derivatives (e.g., 5-MeO-DMT), suggesting favorable binding interactions.

Similar binding modes and scores were also observed with the disubstituted 5-fluoro containing compounds 61 and 164. This was in contrast to the binding modes predicted for the non-psychedelic 5-HT_{2A} agonist 6-F-DET. For 6-F-DET, close contact with Asn3436.55 was maintained, shifting the indole ring higher in the orthosteric pocket relative to 5-fluorotryptamine, 5-MeO-tryptamine, and 5-HT, leading to lose of the indole NH hydrogen bond to Ser242. This difference may underlie, in part, the major difference in pharmacology of 6-fluoro-substituted tryptamine compounds. Interestingly, compounds 61 and 164 showed a binding mode consistent with 5-fluoro-tryptamine compounds and 5-HT, maintaining contact between Asn343⁶⁵⁵ and the fluorine atom at the 5-position. This is consistent with the unexpected finding that a 6-fluoro-substitution is tolerated with respect to HTR activity and potent 5-HT_{2A} agonism so long as a fluorine substituent is present at the 5-position.

In certain embodiments, the fluorinated tryptamine derivatives of the present invention provide favorable interactions with the 5-HT_{2A} receptor, non-limiting examples including hydrogen-bonding, π-stacking, and Van der Waals interactions. In certain embodiments, the favorable interactions are a result of modified electronic properties of the compounds due to fluorine substitution (e.g., σ-withdrawing, π-donating, hydrogen bonding accepting). In certain embodiments, the favorable interactions are a result of the N,N-dialkyl substitution pattern. In certain embodiments, the favorable interactions are a result of synergistic properties of both the fluorine substitution and N,N-dialkyl substitution pattern. In certain embodiments, substituents at any position of the compound of the present invention may contribute to the favorable properties of the compounds of the present invention. In certain embodiments, the compounds of the present invention possess desirable pharmacokinetic and pharmacodynamic properties, non-limiting examples including absorption, metabolic stability, bioavailability, solubility, and half-life.

### Example 79: 5-HT_{2A} Receptor Binding Studies

ValiScreen Serotonin 5-HT_{2A} (human) cell line (product No: ES-313-C) grown in DMEM/F12 media augmented with 10% FBS, 4 mM GlutaMAX, 0.4 mg/mL Geneticin, 1% Penicillin-Streptomycin were utilized to prepare 5-HT_{2A} membrane fractions. Cells were grown in a 150 mm culture dishes and were harvested at between 70-90% confluency between passages 5-15. Cells were detached and lysed at room temperature with 1 mM HEPES buffer containing 2 mM EDTA at pH 7.4 and homogenized with a hand-held homogenizer. The lysate was then centrifuged (30 minutes at 30,000×G at 4° C.). The resultant pellet was resuspended in a storage buffer (20 mM HEPES, 10 mM MgCl₂, 0.1 mM EDTA, pH 7.4 at room temperature) and the suspension frozen and stored at −80° C. until use. Protein concentration was determined via the Bradford method using Coomassie protein assay reagent (Sigma, USA) with Bovine Serum albumin (Sigma, USA) as standard. Aliquots were resuspended in 10 mM HEPES immediately before the experiment.

Suspensions of 10 mM HEPES buffer (pH 7.4 at room temperature) containing 10 μg/mL protein, 1 nM (+)-[3H]-ketanserin (Perkin Elmer NET 1233, unlabeled competitor at various concentrations or 10 μM ketanserin for nonspecific binding in a total volume of 500 μL in a 96 well plate. Plates were then incubated in the dark while mixing on a mechanical rocker for 2 h at 37° C. Each plate also contained a dose response curve for ketanserin as a positive control.

Following incubation, membrane fractions were collected by vacuum filtration using a Unifilter-96 Cell Harvester (Perkin Elmer) over presoaked UniFilter-96 GF/C P Microplates (Perkin Elmer) and filters were washed with room temperature 10 mM HEPES buffer (pH 7.4 at room temperature) (3×1 mL). The filter plates were dried overnight in a fume hood and the trapped tritium trapped measured via liquid scintillation counting with MicroScint-O (Perkin Elmer), using a MicroBeta2 Plate Reader with 6-detectors scintillation counter (Perkin Elmer) at 55% efficiency. IC₅₀ value estimates were determined in GraphPad Prism 9.3.1 using non-linear regression (single site fit) with log-concentration plotted against percent specific binding. The percent specific binding for [3H]-ketanserin in a control experiment was ˜92%. Kᵢ values were calculated using the equation of Cheng and Prusoff. The K_{d} for ketanserin (7.79 nM), was determined via homologous binding experiments. Experiments were performed in duplicate. N=1-3 per compound.

TABLE 2
Receptor binding data

| Compound | 5-HT_{2A} pKᵢ ± SEM |
|---|---|
| 5-F-DMT^{a} | 6.65 |
| 5-F-DET^{b} | 6.12 |
| 1 | 5.95 ± 0.07 |
| 2 | 6.36 ± 0.003 |
| 3 | 6.23 ± 0.30 |
| 4 | 6.02 ± 0.07 |
| 5 | 6.20 |
| 6 | 6.06 ± 0.13 |
| 7 | 6.02 |
| 8 | 6.09 |
| 9 | 6.15 ± 0.21 |
| 10 | 6.51 ± 0.25 |
| 12 | 6.66 ± 0.12 |
| 13 | 6.72 ± 0.12 |
| 14 | 6.09 |
| 15 | 6.98 |
| 16 | 5.78 |
| 17 | 5.41 |
| 19 | 6.92 |
| 21 | 6.15 |
| 22 | 5.97 ± 0.02 |
| 23 | 6.43 |
| 27 | 5.57 |
| 28 | 7.19 ± 0.03 |
| 29 | 6.30 |
| 30 | 5.70 ± 0.39 |
| 31 | 6.26 ± 0.32 |
| 32 | 5.86 ± 0.16 |
| 33 | 6.25 ± 0.003 |
| 34 | 5.78 |
| 35 | 6.38 ± 0.29 |
| 36 | 6.13 ± 0.08 |
| 37 | 6.04 ± 0.05 |
| 39 | 6.12 |
| 40 | 6.06 ± 0.05 |
| 41 | 6.15 ± 0.34 |
| 51 | 6.80 ± 0.02 |
| 69 | 6.90 ± 0.002 |
| 72 | 6.30 ± 0.06 |
| 82 | 6.52 |
| 88 | 6.71 ± 0.31 |
| 158 | 6.08 ± 0.15 |
| 160 | 7.41 ± 0.33 |
| 161 | 6.50 |
| 162 | 5.91 |
| 163 | 5.65 ± 0.004 |
| 164 | 6.90 ± 0.13 |
| 165 | 6.69 |
| 166 | 5.96 ± 0.25 |
| 168 | 6.18 |
| 169 | 6.05 |
| 172 | 6.47 |
| 173 | 6.58 |
| 174 | 6.42 |
| 175 | 5.96 |
| 176 | 5.89 |
| 177 | 5.71 |
| 178 | 6.25 |
| 179 | 6.93 |
| 180 | 5.75 |
| 181 | 5.75 |
| 182 | 6.62 |
| 184 | 6.60 |
| 185 | 6.90 |
| 186 | 6.47 |
| 187 | 5.41 |

^{a}5-fluoro-dimethyltryptamine;
^{b}5-fluoro-diethyltryptamine

### Example 80: Serotonin Receptor Functional Assay

Assays performed as described in Kroeze et al. (Nat. Struct. Mol. Biol., 2015, 22:362-369), which is incorporated herein by reference in its entirety. Briefly functional assay screens at human isoforms of 5-HT_{2A}/2B/2c and 5-HT_{1A} receptors were performed in parallel using the same compound dilutions and 384-well format high-throughput assay platforms. Receptor constructs in pcDNA vectors were generated from the Presto-Tango GPCR library with minor modifications.

Compounds of the present disclosure were serially diluted in buffer (HBSS, 20 mM HEPES, pH 7.4 supplemented with 0.1% bovine serum albumin and 0.01% ascorbic acid) and dispensed into 384-well assay plates using a FLIPRTETRA (Molecular Devices). 5-HT was included as a positive control for each plate. For measurements of 5-HT₂ subtypes (5-HT_{2A}, 5-HT_{2B}, and 5-HT_{2C}) Gq-mediated calcium flux function, HEK Flp-In 293 T-Rex stable cell lines (Invitrogen) were loaded with Fluo-4 dye for 1 h, stimulated with desired concentration of compound and read for baseline (0-10 sec), and drug-induced effect (120 sec). Peak fold-over-basal fluorescence at 25° C. was then calculated using a FLIPRTETRA. For 5-HT_{1A}, Gi/o-mediated cAMP inhibition was measured using the split-luciferase GloSensor assay in HEKT cells in combination with 0.2 μM isoproterenol to stimulate cAMP detecting luminescence on a Microbeta Trilux (Perkin Elmer) with 15 min drug incubation at 25° C. Data were plotted and non-linear regression performed using “log(agonist) vs. response” in GraphPad Prism to yield EC₅₀ (nM) and Emax (% 5-HT) parameter estimates (Table 3).

TABLE 3
Functional Activity of Compounds at 5-HT_{2A} (Ca²⁺), 5-HT_{2B} (Ca²⁺), 5-HT_{2C} (Ca²⁺), and
5-HT_{1A} (cAMP inhibition)

| | 5-HT_{2A}-Ca²⁺ | | 5-HT_{2B}-Ca²⁺ | | 5-HT_{2C}-Ca²⁺ | | | |
|---|---|---|---|---|---|---|---|---|
| | FLUX | | FLUX | | FLUX | | 5-HT_{1A}-cAMP | |
| | | Eₘₐₓ | | Eₘₐₓ | | Eₘₐₓ | | Eₘₐₓ |
| Cmpd | pEC₅₀ | (% 5-HT) | pEC₅₀ | (% 5-HT) | pEC₅₀ | (% 5-HT) | pEC₅₀ | (% 5-HT) |
| 5-HT | 9.84 | 99.84 | 9.60 | 100.10 | 10.15 | 99.70 | 9.34 | 99.90 |
| 5-F- | 8.70 ± 0.03 | 96.6 ± 1.0 | 8.70 ± 0.11 | 61.1 ± 2.0 | 8.8 ± 0.06 | 100.3 ± 1.8 | 6.69 ± 0.08 | 94.2 ± 3.4 |
| DMT | | | | | | | | |
| 5-F- | 8.71 ± 0.03 | 103.6 ± 0.8 | 8.69 ± 0.04 | 92.3 ± 1.0 | 8.45 ± 0.06 | 109.6 ± 2.3 | 6.84 ± 0.05 | 92.8 ± 1.8 |
| DET | | | | | | | | |
| 1 | 8.87 ± 0.03 | 102.6 ± 0.8 | 8.88 ± 0.02 | 100.0 ± 0.6 | 8.18 ± 0.07 | 107.6 ± 2.3 | 6.23 ± 0.06 | 86.4 ± 2.6 |
| 2 | 8.71 ± 0.03 | 95.1 ± 0.8 | 8.48 ± 0.08 | 68.6 ± 1.7 | 7.66 ± 0.07 | 64.6 ± 1.7 | 5.74 ± 0.09 | 93.2 ± 4.7 |
| 3 | 8.43 ± 0.03 | 94.8 ± 0.8 | 8.04 ± 0.11 | 37.5 ± 1.5 | 7.31 ± 0.04 | 56.0 ± 0.9 | 5.41 ± 0.1 | 83.3 ± 5.7 |
| 4 | 9.33 ± 0.04 | 99.5 ± 1.1 | 9.12 ± 0.02 | 105.60 ± 0.7 | 8.04 ± 0.08 | 74.6 ± 2.0 | 6.48 ± 0.07 | 80.5 ± 2.5 |
| 5 | 9.14 ± 0.03 | 106.7 ± 0.8 | 9.12 ± 0.02 | 104.1 ± 0.7 | 8.47 ± 0.09 | 109.3 ± 3.1 | 7.13 ± 0.05 | 87.5 ± 1.6 |
| 6 | 9.11 ± 0.03 | 103.5 ± 0.8 | 8.96 ± 0.02 | 101.2 ± 0.7 | 8.75 ± 0.07 | 111.7 ± 2.5 | 7.31 ± 0.05 | 90.5 ± 1.8 |
| 9 | 8.99 ± 0.06 | 105.7 ± 1.9 | 8.77 ± 0.07 | 96.6 ± 2.0 | 7.72 ± 0.08 | 100.7 ± 3.0 | 6.64 ± 0.07 | 89.1 ± 2.9 |
| 10 | 9.32 ± 0.08 | 100.3 ± 2.2 | 9.02 ± 0.10 | 73.8 ± 2.3 | 8.52 ± 0.08 | 97.4 ± 2.6 | 7.05 ± 0.07 | 97.8 ± 2.7 |
| 11 | 9.21 ± 0.07 | 105.4 ± 2.1 | 9.10 ± 0.06 | 98.8 ± 1.6 | 7.69 ± 0.08 | 101.5 ± 2.8 | 6.82 ± 0.04 | 95.5 ± 1.7 |
| 12 | 8.76 ± 0.04 | 101.8 ± 1.1 | 8.64 ± 0.08 | 65.8 ± 1.6 | 8.80 ± 0.07 | 117.2 ± 2.5 | 7.12 ± 0.05 | 91.1 ± 1.7 |
| 13 | 8.80 ± 0.03 | 103.0 ± 1.1 | 8.58 ± 0.05 | 77.0 ± 1.2 | 8.82 ± 0.11 | 115.0 ± 4.0 | 6.61 ± 0.06 | 85.8 ± 2.5 |
| 16 | 9.06 ± 0.06 | 103.4 ± 1.8 | 8.68 ± 0.06 | 97.4 ± 1.7 | 7.31 ± 0.10 | 84.3 ± 3.4 | 6.78 ± 0.05 | 94.2 ± 2.2 |
| 19 | 8.41 ± 0.21 | 53.3 ± 3.6 | 8.82 ± 0.22 | 32.2 ± 2.2 | 8.44 ± 0.10 | 70.5 ± 2.3 | 5.53 ± 0.05 | 113.3 ± 4.0 |
| 20 | 7.54 ± 0.14 | 61.8 ± 3.2 | 8.62 ± 0.20 | 17.3 ± 1.1 | 6.33 ± 0.11 | 55.0 ± 2.8 | 6.10 ± 0.05 | 93.1 ± 2.5 |
| 22 | 9.05 ± 0.05 | 105.4 ± 1.6 | 9.09 ± 0.06 | 98.9 ± 1.6 | 7.30 ± 0.07 | 97.5 ± 2.6 | 6.49 ± 0.04 | 102.90 ± 1.9 |
| 23 | 8.07 ± 0.12 | 41.8 ± 1.6 | 8.36 ± 0.13 | 19.7 ± 0.8 | 7.68 ± 0.12 | 26.6 ± 1.1 | 7.65 ± 0.05 | 99.2 ± 1.7 |
| 24 | 5.85 ± 0.05 | 81.0 ± 2.4 | IA | IA | IA | IA | 9.31 ± 0.24 | 20.5 ± 1.4 |
| 26 | IA | IA | IA | IA | IA | IA | IA | IA |
| 27 | 7.23 ± 0.17 | 34.4 ± 2.3 | 7.85 ± 0.11 | 35.3 ± 1.3 | 7.25 ± 0.10 | 18.5 ± 0.7 | 6.25 ± 0.08 | 88.2 ± 3.6 |
| 28 | 8.63 ± 0.09 | 91.1 ± 2.5 | 9.14 ± 0.04 | 90.8 ± 1.1 | 8.76 ± 0.03 | 92.8 ± 0.9 | 7.14 ± 0.04 | 101.20 ± 1.7 |
| 29 | IA | IA | IA | IA | 7.31 ± 0.08 | 28.5 ± 0.8 | 6.07 ± 0.10 | 64.0 ± 3.5 |
| 30 | 8.61 ± 0.06 | 93.2 ± 1.7 | 8.46 ± 0.08 | 83.8 ± 2.1 | 6.83 ± 0.09 | 72.2 ± 2.9 | 6.33 ± 0.06 | 104.0 ± 3.1 |
| 31 | 8.69 ± 0.06 | 104.5 ± 1.8 | 8.60 ± 0.05 | 96.5 ± 1.4 | 7.24 ± 0.05 | 98.0 ± 1.9 | 7.65 ± 0.05 | 100.7 ± 1.8 |
| 32 | 8.95 ± 0.05 | 106.8 ± 1.7 | 9.02 ± 0.06 | 108.0 ± 1.8 | 7.19 ± 0.07 | 101.5 ± 2.7 | 5.90 ± 0.06 | 88.5 ± 2.8 |
| 33 | 9.31 ± 0.06 | 107.0 ± 1.9 | 9.23 ± 0.07 | 115.6 ± 2.2 | 7.72 ± 0.05 | 105.4 ± 1.8 | 7.84 ± 0.04 | 100.7 ± 1.3 |
| 33 | 9.31 ± 0.06 | 107.0 ± 1.9 | 9.23 ± 0.07 | 115.6 ± 2.2 | 7.72 ± 0.05 | 105.4 ± 1.8 | 7.84 ± 0.04 | 100.7 ± 1.3 |
| 36 | 8.92 ± 0.06 | 104.8 ± 2.0 | 8.76 ± 0.06 | 104.5 ± 2.0 | 7.77 ± 0.05 | 101.5 ± 1.9 | 7.25 ± 0.04 | 98.4 ± 1.5 |
| 40 | 9.05 ± 0.07 | 102.5 ± 2.0 | 8.96 ± 0.05 | 106.7 ± 1.5 | 6.95 ± 0.05 | 94.9 ± 2.0 | 7.18 ± 0.04 | 102.1 ± 1.7 |
| 82 | 7.98 ± 0.02 | 90.4 ± 0.7 | 7.18 ± 0.02 | 86.3 ± 0.8 | 6.71 ± 0.04 | 89.6 ± 1.7 | ND | ND |
| 88 | 8.71 ± 0.03 | 90.6 ± 0.8 | 8.25 ± 0.06 | 37.5 ± 0.7 | 7.69 ± 0.05 | 86.8 ± 1.4 | ND | ND |
| 164 | 8.19 ± 0.04 | 76.1 ± 1.1 | 8.21 ± 0.08 | 33.2 ± 0.9 | 7.73 ± 0.04 | 89.3 ± 1.3 | ND | ND |
| 165 | 8.34 ± 0.02 | 103.0 ± 0.8 | 8.13 ± 0.02 | 102.4 ± 0.8 | 7.41 ± 0.04 | 102.8 ± 1.7 | ND | ND |
| 166 | 7.74 ± 0.02 | 98.1 ± 0.7 | 7.48 ± 0.02 | 95.9 ± 0.7 | 6.70 ± 0.04 | 94.6 ± 1.7 | ND | ND |
| 172 | 8.40 ± 0.02 | 94.0 ± 0.6 | 8.19 ± 0.03 | 93.5 ± 0.8 | 6.82 ± 0.05 | 82.3 ± 1.7 | ND | ND |
| 173 | 8.14 ± 0.02 | 87.4 ± 0.7 | 7.75 ± 0.03 | 82.8 ± 0.8 | 6.52 ± 0.03 | 80.4 ± 1.2 | ND | ND |
| 174 | 7.73 ± 0.11 | 38.2 ± 1.4 | IA | IA | 7.88 ± 0.08 | 39.9 ± 1.1 | ND | ND |
| 175 | 8.17 ± 0.17 | 48.6 ± 2.8 | IA | IA | 6.99 ± 0.09 | 51.2 ± 2.0 | ND | ND |
| 176 | 7.39 ± 0.03 | 97.8 ± 1.1 | 7.54 ± 0.77 | 103.0 ± 0.6 | 6.72 ± 0.04 | 99.6 ± 1.5 | ND | ND |
| 178 | 8.27 ± 0.04 | 84.7 ± 1.2 | 8.26 ± 0.51 | 27.7 ± 0.5 | 7.80 ± 0.05 | 87.4 ± 1.5 | ND | ND |
| 179 | 7.97 ± 0.03 | 79.3 ± 0.7 | 7.97 ± 0.61 | 67.8 ± 0.6 | 7.64 ± 0.03 | 95.5 ± 1.0 | ND | ND |
| 180 | 7.10 ± 0.05 | 80.7 ± 1.6 | 7.42 ± 1.58 | 92.7 ± 1.2 | 6.20 ± 0.07 | 56.5 ± 2.1 | ND | ND |
| 181 | 7.23 ± 0.06 | 68.7 ± 1.7 | 6.53 ± 1.40 | 36.8 ± 0.7 | IA | IA | ND | ND |
| 187 | 7.44 ± 0.07 | 90.7 ± 2.5 | 8.90 ± 0.05 | 89.0 ± 1.4 | 7.73 ± 0.03 | 93.6 ± 0.9 | 6.03 ± 0.06 | 88.5 ± 2.8 |

IA (Inactive);
ND (not determined);
N = 1-3.

### Example 81: Head-Twitch Response (HTR) Studies

Male C57BL/6J mice (6-8 weeks old) were obtained from Jackson Laboratories (Bar Harbor, ME) and housed in a vivarium at the University of California San Diego, an AAALAC-approved animal facility that meets all federal and state requirements for care and treatment of laboratory animals. All animal experiments were carried out in accordance with NIH guidelines and were approved by the UCSD Institutional Animal Care and Use Committee. Mice were housed up to four per cage in a climate-controlled room on a reverse-light cycle (lights on at 1900 h, off at 0700 h). Animals were provided with ad libitum access to food and water, with the exception of during behavioral testing.

Testing was conducted between 1000 and 1800 h. Head movements were recorded using a head-mounted magnet and a magnetometer coil as described in Halberstadt et al. (Psychopharmacology, 2013, 227(4):727-739). For the head mounted magnet, mice were anesthetized, an incision was made in the scalp, and a neodymium magnet was attached to the dorsal surface of the cranium using dental cement. Following a 2-week recovery period, behavioral experiments were conducted in a well-lit room. At least 7 days occurred between sessions to avoid any carryover effects. Test substances (i.e., dimethyltryptamine (DMT), diethyltryptamine (DET), psilocybin, 6-fluoro-diethyltryptamine (6-F-DET), compounds 3-4, 6, 9, 12-13, 22, 33, 41, 69, 88, 166, and 178) were administered intraperitoneally (IP) at a volume of 5 mL/kg. Mice were treated with vehicle (saline) or 6 doses of the test compound and then placed in a glass cylinder surrounded by a magnetometer coil and tested for 30 min. Coil voltage was filtered (1 kHz lowpass), digitized (20 kHz sampling rate) and saved to disk using a Powerlab/8SP with LabChart v 7.3.2 (ADInstruments, Colorado Springs, CO).

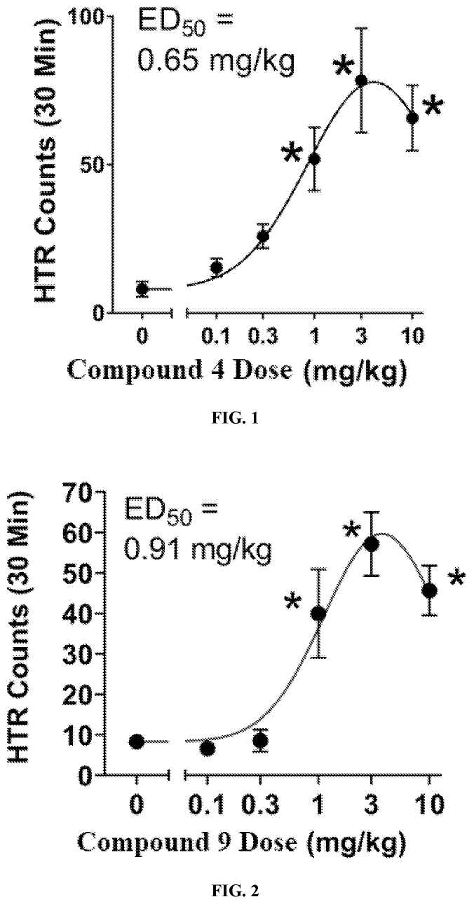

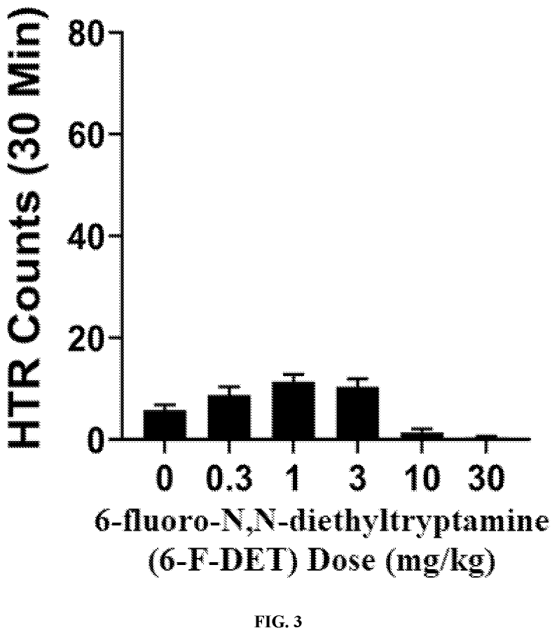

The entire 30 min recordings were examined for head twitches. Established procedures based on artificial intelligence were used to identify head twitches in the recordings (Scientific Reports, 2020, 10(1):1-12). HTR counts were analyzed using one-way analyses of variance (ANOVA), followed by post hoc pairwise comparisons between individual groups and vehicle using Dunnett's multiple comparisons test. Significance was demonstrated when an α-level of 0.05 was surpassed. Median effective doses (ED₅₀ values) and 95% confidence intervals (95% CI) were calculated using nonlinear regression (Prism 9.0.2, GraphPad Software, San Diego, CA) (Table 4 and FIGS. 1-3).

Results of the studies described herein indicate that the compounds of the present disclosure induce HTR in mice with a comparable and/or reduced ED₅₀, as compared to reported values of known 5HT_{2A} agonists run under comparable conditions (e.g., DMT, DET, psilocybin, and 6-F-DET) (Table 4) (Halberstadt et al. Neuropharmacology 2020, 167:107933). HTR has predictive validity for classical hallucinogen induced psychedelic activity in humans, as well as potency for inducing such effects. The 5-HT_{2A} agonist 6-F-DET lacks psychedelic effects in humans and was similarly inactive in the HTR study.

A significant increase in HTR counts, over baseline, was observed with administration of each of compounds 4 and 9, as compared to inactive compound 6-F-DET, as shown in the ED₅₀ plots of each respective compound (FIGS. 1-3). Compound 8 (4-F-DET) was also inactive in inducing HTR as compared to a control (i.e., vehicle). The lack of HTR with compound 8 and 6-F-DET are consistent with the literature regarding fluorinated derivatives of known psychedelics with reduced or attenuated psychedelic activity in rodents and humans.

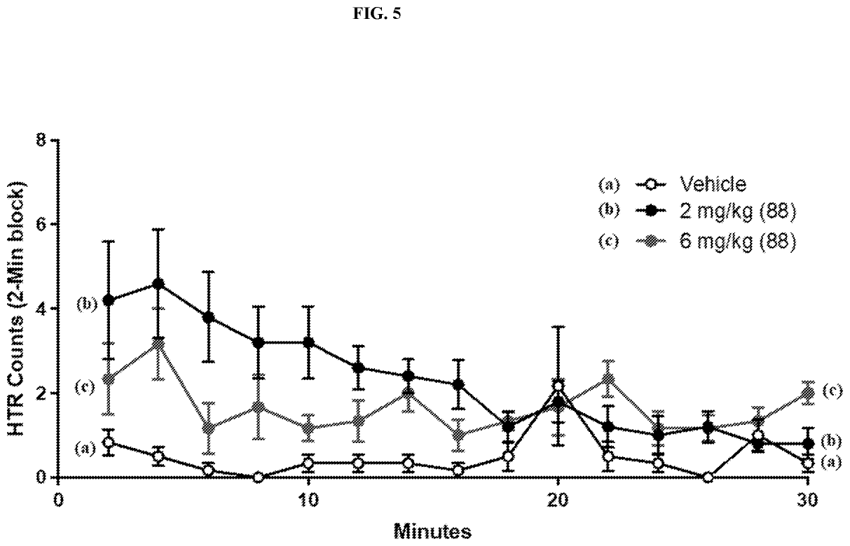

Duration of the HTR was also assessed using an ascending and descending arm dose relative to several psychedelic tryptamines of known durations of action in humans, including DMT, psilocin, 5-methoxy-α-methyltryptamine (5-MeO-AMT), and α-methyltryptamine (AMT). The compounds of the present disclosure typically had a duration of HTR above baseline that was longer than the short acting DMT (i.e., about 10 minutes), but shorter or comparable to psilocin (i.e., about 35 minutes), and substantially shorter than long acting compounds 5-MeO-AMT and AMT, in C57BL/6J mice. A time course group is provided for compound 88 (FIG. 5).

TABLE 4
Qualitative head-twitch response (HTR) potency data

| Compound | ED₅₀ mg/kg (95% CI) | ED₅₀ μmol/kg (95% CI) |
|---|---|---|
| DMT | 1.54 (1.08-2.19) | 5.05 (3.55-7.19) |
| DET | 2.28 (1.57-3.30) | 6.85 (4.72-9.93) |
| Psilocybin | 0.38 | 1.40 |
| 6-F-DET | —^{a} | —^{a} |
| 3 | 1.79 (0.93-2.88) | 6.28 (3.28-10.11) |
| 4 | 0.65 (0.27-1.25) | 2.18 (0.91-4.18) |
| 8 | —^{a} | —^{a} |
| 9 | 0.91 (0.53-1.54) | 3.00 (1.77-5.09) |
| 13 | 1.13 (0.72-1.77) | 4.81 (3.06-7.56) |
| 22 | 3.88 (2.92-5.17) | 12.41 (9.32-16.51) |
| 33 | 0.79 (0.51-1.08) | 2.55 (1.65-3.47) |
| 41 | 1.12 (0.85-1.47) | 3.56 (2.71-4.67) |
| 69 | 1.32 (0.96-2.10) | 4.81 (3.48-7.62) |
| 88 | 0.56 (0.29-1.10) | 1.95 (1-3.81) |
| 166 | 1.96 (1.19-3.22) | 6.56 (3.98-10.78) |
| 178 | 0.87 (0.55-1.29) | 3.53 (2.23-5.24) |

^{a}ED₅₀ could not be estimated using the regression model

### Example 82: Pharmacokinetic Studies

Compounds of the present disclosure (e.g., compounds 3 and/or 4) were subjected to one or more pharmacokinetic analyses in human hepatocytes (Table 5) and/or male Sprague Dawley mice (Table 6).

The metabolic stability of a test compounds were assessed by monitoring disappearance of each compound in the presence of cryopreserved hepatocytes for up to 1 hour at 37° C. Following protein precipitation and centrifugation, the samples were analyzed by UPLC-MS-MS. Test Compound stock solutions (10 mM) were diluted 1 in 100 (2 μL 10 mM DMSO stock+198 μL 50:50 acetonitrile:water) to provide a 100 μM working solution. Following addition of test compounds to buffer, samples were pre-incubated at 37° C. for 5 minutes prior to initiating the incubation by the addition of test compounds to hepatocytes (these were held at 37° C. during pre-incubation period). Aliquots were sampled at 0, 5, 10, 20, 40 and 60 min and added to ice cold acetonitrile to terminate the reaction and precipitate proteins. All samples were mixed, centrifuged, and the supernatants were analyzed by UPLC-MS-MS using a Phenomenex Luna Omega 1.6 μM, C18 100 Å, 50×2.1 mm column. The final incubation conditions were as follows: hepatocytes 0.5×106 viable cells/mL, test compound 1 μM (n=1), acetonitrile concentration<0.01% (v/v) and DMSO concentration<0.01% (v/v). The intrinsic clearance (Clint) and half-life (T_{1/2}) values are determined from the slope of the parent depletion curve. Positive control compounds with known metabolic clearance were included (diltiazem and naloxone).

For the in vivo stability study described herein, 1 mg/kg of drug was administered by intravenous injection (i.e., dose volume 2 mL/kg, vehicle 5% DMSO:95% hydroxypropyl-beta-cyclodextrin (20% w/v in water)) to three male Sprague Dawley rats (˜300 g at time of dosing). Blood samples (˜240 μL) were collected via venipuncture at various time points (5, 15, 30, 60 120 240, 480 and 1,440 min) into K2EDTA-coated tubes. The samples were spun at 10,000 rpm (9,600×G) for 2 minutes. 100 μL of the resulting plasma was then collected and stored at −80° C. until analyzed by HPLC as described above.

The results indicated that compounds 3 and 4 were rapidly metabolized by human hepatocytes consistent with rapid clearance and a short duration of action (Table 5). The in vitro T_{1/2} was only slightly greater than the short T_{1/2} control compound naloxone and substantially shorter than diltiazem which has a longer T_{1/2} of several hours in vivo. An in vivo kinetic study of intravenously administered compound 3 was then undertaken in rats and showed a clearance of CL=29.5 mL/min/kg and a short elimination T_{1/2} of 1.09 h (Table 6).

TABLE 5
Human hepatocyte kinetic data for compounds 3 and 4 as
compared to reference compounds diltiazem and naloxone

| Compound | Clint (μL/min/10⁶ cells) | T_{1/2} (min) |
|---|---|---|
| diltiazem | 32 | 44 |
| naloxone | 138 | 10 |
| 3 | 132 | 11 |
| 4 | 109 | 13 |

Concentrations were determined by UHPLC-MS-MS at various time points following compound administration

TABLE 6
Male Sprague Dawley rats kinetic data for
intravenously administered compound 3

| Parameter | Mean | CV % |
|---|---|---|
| Half-Life (h) | 1.09 | 48.6% |
| CL (mL/min/kg) | 29.5 | 28.5 |
| Vss (L/kg) | 0.999 | 40.3 |
| Vz (L/kg) | 3.04 | 69.4 |
| C0 (ng/mL) | 2490 | 23.2 |
| AUCall (ng/mL · h) | 594 | 29.2 |
| AUCinf (ng/mL · h) | 597 | 29.0 |
| AUC % Extrapolation to C0 | 27.7 | 9.45 |
| AUC % Extrapolation to Inf | 0.570 | 56.0 |

Compound 3 administered intravenously (1 mg/kg); Vehicle: 5% DMSO, 95% 2-hydroxypropyl-β-cyclodextrin (HPBCD) (20% w/v in water); Plasma concentrations were quantified at multiple time points by UHPLC-MS-MS; N=3.

### Enumerated Embodiments

The following exemplary embodiments are provided, the numbering of which is not to be construed as designating levels of importance:

Embodiment 1 provides a compound of formula (I), or a salt, prodrug, solvate, isotopologue, or stereoisomer thereof:

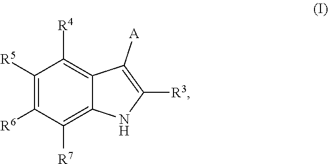

wherein:
   A is

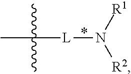

wherein * indicates the bond between L and N(R¹)(R²);
L is

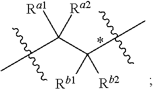

R^{a1} and R^{a2} are each independently selected from the group consisting of H, halogen, C₁-C₆ alkoxy, and C₁-C₆ alkyl,
   or R^{a1} and R² may combine to form a carbonyl (C═O);
R^{b1} and R^{b2} are each independently selected from the group consisting of H and C₁-C₆ alkyl;
R¹ and R² are each independently selected from the group consisting of optionally substituted C₁-C₈ alkyl, optionally substituted C₃-C₈ cycloalkyl, optionally substituted C₁-C₈ heteroalkyl, optionally substituted C₂-C₅ heterocycloalkyl, optionally substituted C₂-C₈ alkenyl, and optionally substituted C₂-C₈ alkynyl,
   wherein R¹ and R² are not identical, and
   wherein one of R¹ and R² may combine with one of R^{a1}, R^{a2}, R^{b1}, and R^{b2} to form an optionally substituted C₂-C₈ heterocyclyl,
   with the proviso that, if one of R¹ and R² combines with R^{b1} or R^{b2} to form a 5 membered ring and R⁵ is F, then at least one of R⁴, R⁶, and R⁷ is not H, or if one of R¹ and R² combine with R^{b1} or R^{b2} to form a stereocenter then the compound consists essentially of one stereoisomer;
R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
   wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl);
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl;
wherein the isotopologue does not comprise F¹⁸ in R¹ or R²; and
wherein the compound of formula (I) is not selected from the group consisting of:
N-ethyl-N-(2-(4-fluoro-1H-indol-3-yl)ethyl)propan-1-amine;
N-ethyl-N-(2-(5-fluoro-1H-indol-3-yl)ethyl)propan-1-amine;
N-ethyl-N-(2-(6-fluoro-1H-indol-3-yl)ethyl)propan-1-amine;
N-ethyl-N-(2-(7-fluoro-1H-indol-3-yl)ethyl)propan-1-amine;
N-ethyl-2-(4-fluoro-1H-indol-3-yl)-N-methylethan-1-amine;
N-ethyl-2-(5-fluoro-1H-indol-3-yl)-N-methylethan-1-amine;
N-ethyl-2-(6-fluoro-1H-indol-3-yl)-N-methylethan-1-amine;
N-ethyl-2-(7-fluoro-1H-indol-3-yl)-N-methylethan-1-amine;
(R)-4-fluoro-3-((1-(methyl-d3)pyrrolidin-2-yl)methyl-d2)-1H-indole;
(R)-4-fluoro-3-((1-(methyl-d3)pyrrolidin-2-yl)methyl)-1H-indole;
(R)-4-fluoro-3-(pyrrolidin-2-ylmethyl-d2)-1H-indole; and
(S)-4-fluoro-3-((1-(methyl-d3)pyrrolidin-2-yl)methyl-d2)-1H-indole.

Embodiment 2 provides the compound of Embodiment 1, wherein R¹ is optionally substituted C₁-C₃ alkyl and R² is selected from the group consisting of optionally substituted, branched C₃-C₈ alkyl and optionally substituted C₃-C₈ cycloalkyl.

Embodiment 3 provides the compound of Embodiment 1 or 2, wherein R¹ is selected from the group consisting of methyl, allyl, and n-propyl.

Embodiment 4 provides the compound of any one of Embodiments 1-3, wherein R² is optionally substituted, branched C₃-C₈ alkyl.

Embodiment 5 provides the compound of any one of Embodiments 1-4, wherein R² is selected from the group consisting of iso-propyl, sec-butyl, iso-butyl, 1,2-dimethylpropyl, methylallyl, and 2-methylallyl.

Embodiment 6 provides the compound of any one of Embodiments 1-3, wherein R² is optionally substituted C₃-C₈ cycloalkyl.

Embodiment 7 provides the compound of any one of Embodiments 1-3 and 6, wherein R² is selected from the group consisting of cyclopropyl and cyclobutyl.

Embodiment 8 provides the compound of Embodiment 1, wherein each of R¹ and R² are independently selected from the group consisting of methyl, ethyl, n-propyl, 1,2-dimethylpropyl, iso-propyl, sec-butyl, iso-butyl, n-butyl, cyclopropyl, cyclopropylmethyl, methylcyclopropyl, cyclopropylethyl, 2-cyclopropyleth-2-yl, cyclobutyl, 2-thietanyl, 3-thietanyl, allyl, methylallyl, 2-methylallyl, 3-methylallyl, propargyl, cyanomethyl, 2-hydroxyethyl, and 2-methoxyethyl.

Embodiment 9 provides the compound of any one of Embodiments 1-8, wherein R³ is H.

Embodiment 10 provides the compound of any one of Embodiments 1-9, wherein R⁴ is F and each of R⁵, R⁶, and R⁷ is H.

Embodiment 11 provides the compound of any one of Embodiments 1-9, wherein R⁵ is F, and each of R⁴, R⁶, and R⁷ is H.

Embodiment 12 provides the compound of any one of Embodiments 1-9, wherein R⁷ is F, and each of R⁴, R⁵, and R⁶ is H.

Embodiment 13 provides the compound of any one of Embodiments 1-9, wherein each of R⁴ and R⁵ is F, and each of R⁶ and R⁷ is H.

Embodiment 14 provides the compound of any one of Embodiments 1-9, wherein each of R⁴ and R⁶ is F, and each of R⁵ and R⁷ is H.

Embodiment 15 provides the compound of any one of Embodiments 1-9, wherein each of R⁴ and R⁷ is F, and each of R⁵ and R⁶ is H.

Embodiment 16 provides the compound of any one of Embodiments 1-9, wherein each of R⁵ and R⁶ is F, and each of R⁴ and R⁷ is H.

Embodiment 17 provides the compound of any one of Embodiments 1-9, wherein each of R⁵ and R⁷ is F, and each of R⁴ and R⁶ is H.

Embodiment 18 provides the compound of any one of Embodiments 1-9, wherein each of R⁶ and R⁷ is F, and each of R⁴ and R⁵ is H.

Embodiment 19 provides the compound of any one of Embodiments 1-9, wherein R⁵ is OMe, R⁷ is F, and each of R⁴ and R⁶ is H.

Embodiment 20 provides the compound of any one of Embodiments 1-9, wherein each of R⁵, R⁶, and R⁷ are F, and R⁴ is H.

Embodiment 21 provides the compound of any one of Embodiments 1-9, wherein R⁵ is selected from the group consisting of OR^{A}, N(R^{A})(R^{B}), and SR^{A}.

Embodiment 22 provides the compound of any one of Embodiments 1-9, wherein R⁵ is selected from the group consisting of H, F, C₁, OMe, OH, and Me.

Embodiment 23 provides the compound of Embodiment 22, wherein R⁵ is OMe.

Embodiment 24 provides the compound of Embodiment 22, wherein R⁵ is F.

Embodiment 25 provides the compound of any one of Embodiments 1-9 and 21-24, wherein R⁶ is selected from the group consisting of H, F, Me, C₁.

Embodiment 26 provides the compound of any one of Embodiments 1-9 and 21-25, wherein R⁷ is selected from the group consisting of H, F, Cl, Br, and Me.

Embodiment 27 provides the compound of any one of Embodiments 1-9 and 21-26, wherein each of R⁴ and R⁶ is H.

Embodiment 28 provides the compound of any one of Embodiments 1-27, wherein L is selected from the group consisting of

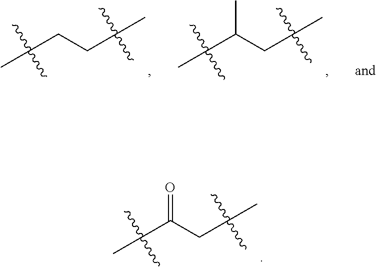

Embodiment 29 provides the compound of Embodiment 28, wherein L is

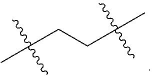 [SMILES: *CC*]

Embodiment 30 provides the compound of any one of Embodiments 1-27, wherein A is selected from the group consisting of:

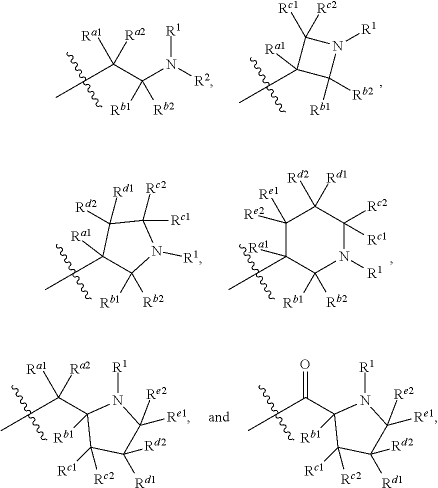

and
wherein:
   R^{c1}, R^{c2}, R^{d1}, R^{d2}, R^{c1}, and R^{c2}, if present, are each independently selected from the group consisting of H, C₁-C₃ alkyl, and C₁-C₃ haloalkyl.

Embodiment 31 provides the compound of any one of Embodiments 1-27 and 30, wherein each of R^{a1}, R^{a2}, R^{b1}, R^{b2}, R^{c1}, R^{c2}, R^{d1}, R^{d2}, R^{e1}, R^{e2}, R^{f1}, and R^{f2}, if present, is H.

Embodiment 32 provides the compound of any one of Embodiments 1-31, wherein A is selected from the group consisting of:

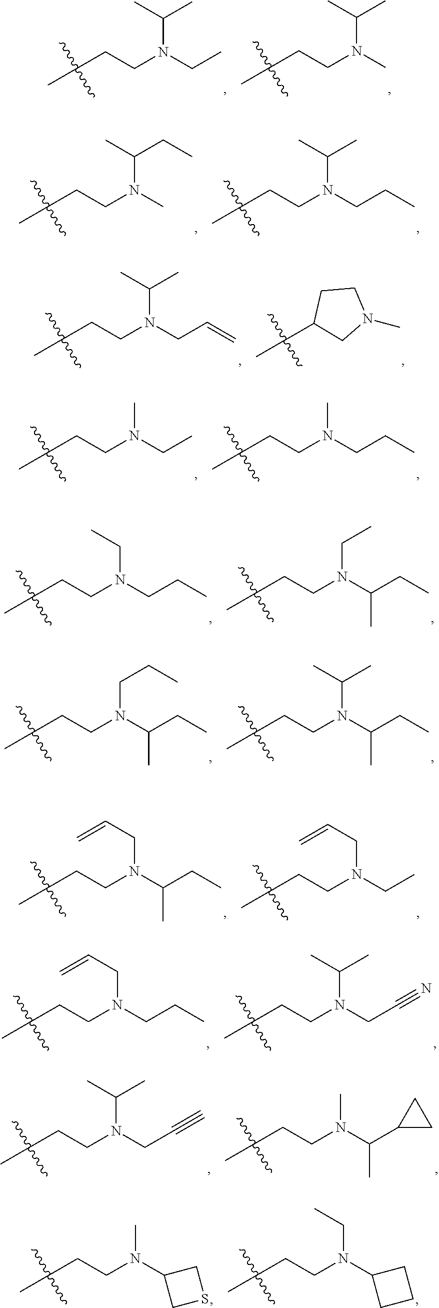

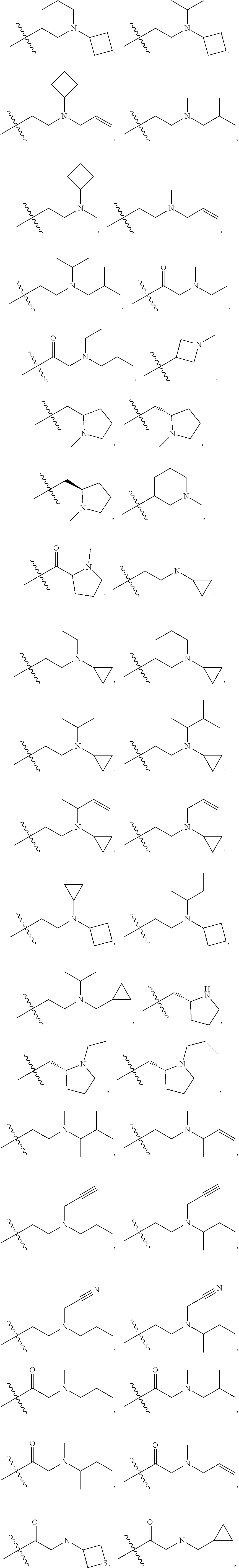

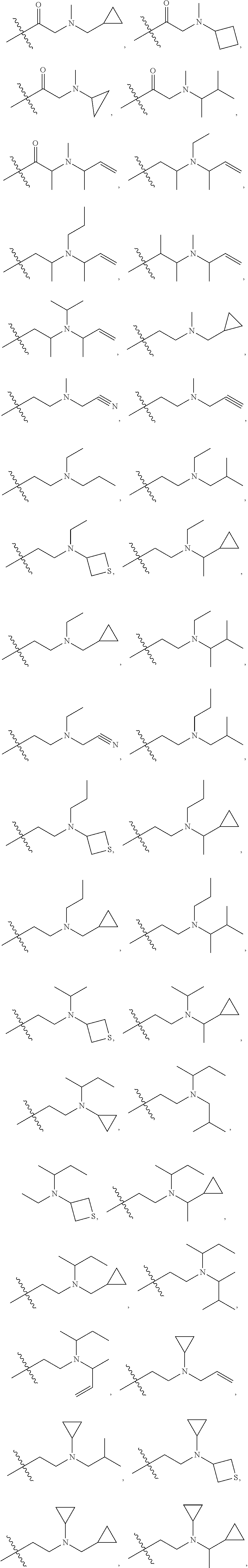

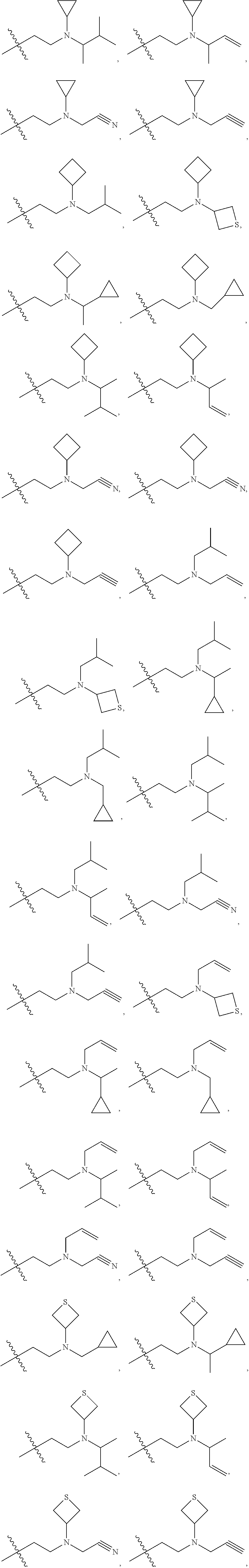

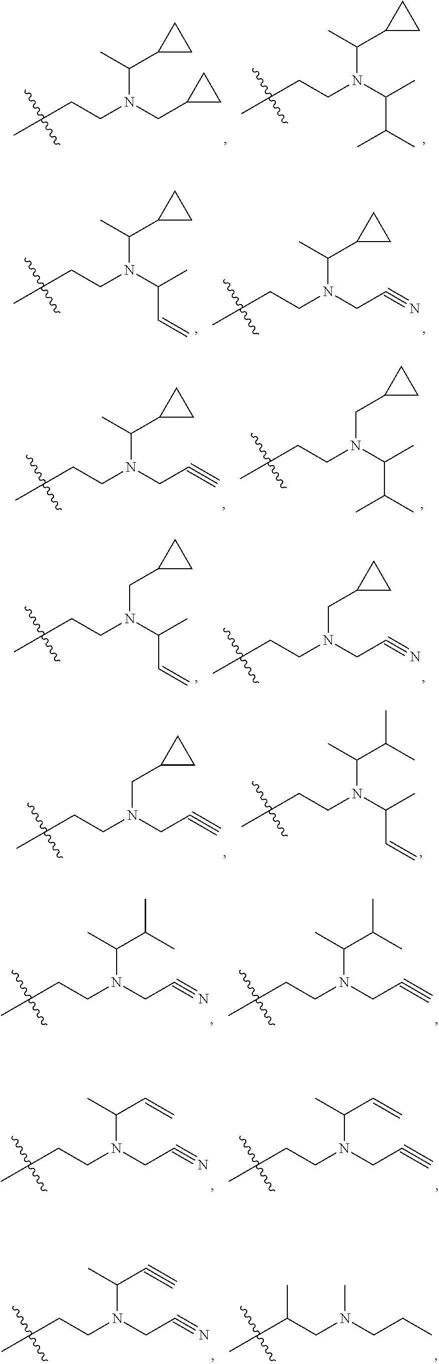

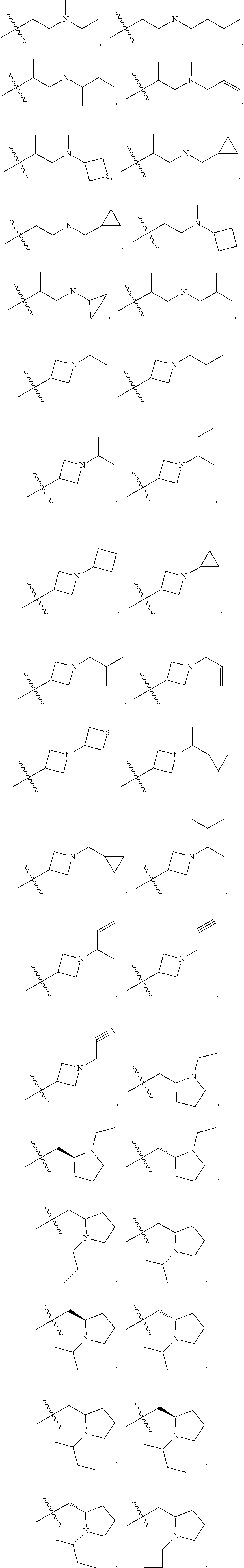

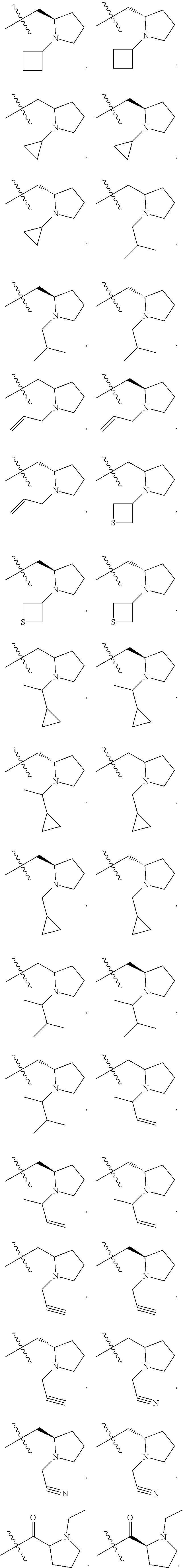

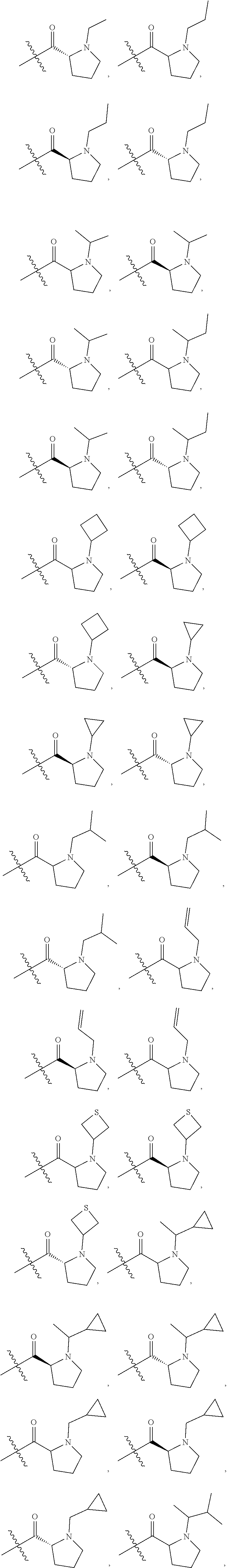

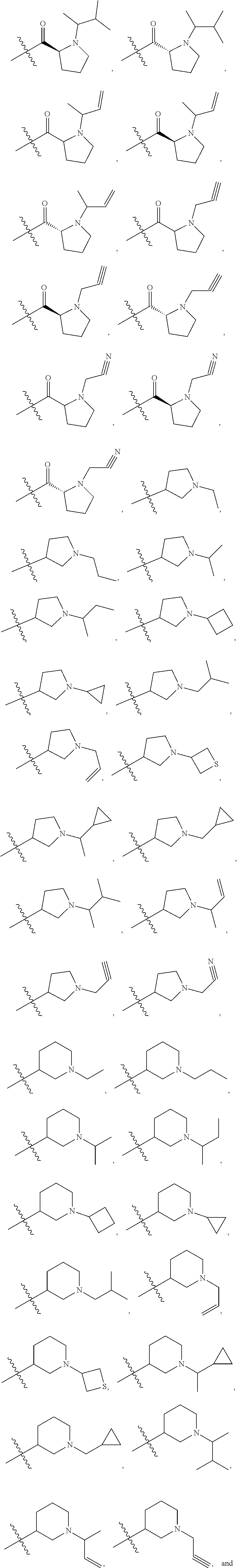

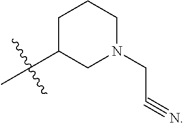 [SMILES: *C1CCCN(CC#N)C1]

Embodiment 33 provides the compound of any one of Embodiments 1-32, which is selected from the group consisting of:
N-ethyl-N-(2-(5-fluoro-1H-indol-3-yl)ethyl)propan-2-amine;
N-(2-(5-fluoro-1H-indol-3-yl)ethyl)-N-methylpropan-2-amine;
N-(2-(5-fluoro-1H-indol-3-yl)ethyl)-N-methylbutan-2-amine;
N-(2-(5-fluoro-1H-indol-3-yl)ethyl)-N-isopropylpropan-1-amine;
N-(2-(5,6-difluoro-1H-indol-3-yl)ethyl)-N-ethylpropan-1-amine;
N-(2-(5-fluoro-1H-indol-3-yl)ethyl)-N-methylprop-2-en-1-amine;
N-(2-(5-fluoro-1H-indol-3-yl)ethyl)-N-isopropylprop-2-en-1-amine;
N-(2-(5-fluoro-1H-indol-3-yl)ethyl)-N-methylpropan-1-amine;
N-allyl-N-(2-(5-fluoro-1H-indol-3-yl)ethyl)cyclobutanamine;
N-allyl-N-(2-(5-fluoro-1H-indol-3-yl)ethyl)butan-2-amine;
2-((2-(5-fluoro-1H-indol-3-yl)ethyl)(isopropyl)amino)acetonitrile;
5,6-difluoro-3-(1-methylpyrrolidin-3-yl)-1H-indole;
N-(2-(5-fluoro-1H-indol-3-yl)ethyl)-N-methylthietan-3-amine;
N-(2-(4,5-difluoro-1H-indol-3-yl)ethyl)-N-ethylpropan-1-amine;
N-(2-(5-fluoro-1H-indol-3-yl)ethyl)-N-propylbutan-2-amine;
7-fluoro-3-(1-methylpyrrolidin-3-yl)-1H-indole;
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
(S)-5-fluoro-3-((1-methylpyrrolidin-2-yl)methyl)-1H-indole;
(R)-5-fluoro-3-((1-methylpyrrolidin-2-yl)methyl)-1H-indole;
5-fluoro-3-(1-methylazetidin-3-yl)-1H-indole;
N-ethyl-N-(2-(5-fluoro-1H-indol-3-yl)ethyl)butan-2-amine;
N-ethyl-N-(2-(5-fluoro-1H-indol-3-yl)ethyl)cyclobutanamine;
N-(2-(5-fluoro-1H-indol-3-yl)ethyl)-N-isopropylbutan-2-amine;
N-(2-(5-fluoro-1H-indol-3-yl)ethyl)-N-propylcyclobutanamine;
N-(2-(5-fluoro-1H-indol-3-yl)ethyl)-N-isopropylprop-2-yn-1-amine;
N-(2-(5-fluoro-1H-indol-3-yl)ethyl)-N-propylprop-2-en-1-amine;
N-ethyl-N-(2-(5-fluoro-1H-indol-3-yl)ethyl)prop-2-en-1-amine;
N-(2-(6-chloro-5-fluoro-1H-indol-3-yl)ethyl)-N-ethylpropan-1-amine;
1-cyclopropyl-N-(2-(5-fluoro-1H-indol-3-yl)ethyl)-N-methylethan-1-amine;
N-(2-(5-fluoro-1H-indol-3-yl)ethyl)-N,2-dimethylpropan-1-amine;
N-(2-(5-fluoro-1H-indol-3-yl)ethyl)-N-isopropylcyclobutanamine;
N-ethyl-N-(2-(7-fluoro-5-methoxy-1H-indol-3-yl)ethyl)propan-1-amine;
N-(2-(4-fluoro-1H-indol-3-yl)ethyl)-N-methylpropan-1-amine;
N-(2-(4-fluoro-1H-indol-3-yl)ethyl)-N-methylpropan-2-amine;
N-ethyl-N-(2-(4-fluoro-1H-indol-3-yl)ethyl)propan-2-amine;
N-(2-(4-fluoro-1H-indol-3-yl)ethyl)-N,2-dimethylpropan-1-amine;
N-(2-(4-fluoro-1H-indol-3-yl)ethyl)-N-methylcyclobutanamine;
N-(2-(4-fluoro-1H-indol-3-yl)ethyl)-N-methylprop-2-en-1-amine;
N-(2-(4-fluoro-1H-indol-3-yl)ethyl)-N-isopropyl-2-methylpropan-1-amine;
N-(2-(5-fluoro-1H-indol-3-yl)ethyl)-N-methylcyclobutanamine;
N-(2-(5-fluoro-1H-indol-3-yl)ethyl)-N-isopropyl-2-methylpropan-1-amine;
N-(2-(7-fluoro-1H-indol-3-yl)ethyl)-N-methylpropan-1-amine;
N-(2-(7-fluoro-1H-indol-3-yl)ethyl)-N-methylpropan-2-amine;
N-ethyl-N-(2-(7-fluoro-1H-indol-3-yl)ethyl)propan-2-amine;
N-(2-(7-fluoro-1H-indol-3-yl)ethyl)-N,2-dimethylpropan-1-amine;
N-(2-(7-fluoro-1H-indol-3-yl)ethyl)-N-methylcyclobutanamine;
N-(2-(7-fluoro-1H-indol-3-yl)ethyl)-N-methylprop-2-en-1-amine;
N-(2-(7-fluoro-1H-indol-3-yl)ethyl)-N-isopropyl-2-methylpropan-1-amine;
2-(5,6-difluoro-1H-indol-3-yl)-N-ethyl-N-methylethan-1-amine;
N-(2-(5,6-difluoro-1H-indol-3-yl)ethyl)-N-methylpropan-1-amine;
N-(2-(5,6-difluoro-1H-indol-3-yl)ethyl)-N-methylpropan-2-amine;
N-(2-(5,6-difluoro-1H-indol-3-yl)ethyl)-N-ethylpropan-2-amine;
N-(2-(5,6-difluoro-1H-indol-3-yl)ethyl)-N,2-dimethylpropan-1-amine;
N-(2-(5,6-difluoro-1H-indol-3-yl)ethyl)-N-methylcyclobutanamine;
N-(2-(5,6-difluoro-1H-indol-3-yl)ethyl)-N-methylprop-2-en-1-amine;
N-(2-(5,6-difluoro-1H-indol-3-yl)ethyl)-N-isopropyl-2-methylpropan-1-amine;
2-(5,7-difluoro-1H-indol-3-yl)-N-ethyl-N-methylethan-1-amine;
N-(2-(5,7-difluoro-1H-indol-3-yl)ethyl)-N-methylpropan-1-amine;
N-(2-(5,7-difluoro-1H-indol-3-yl)ethyl)-N-methylpropan-2-amine;
N-(2-(5,7-difluoro-1H-indol-3-yl)ethyl)-N-ethylpropan-1-amine;
N-(2-(5,7-difluoro-1H-indol-3-yl)ethyl)-N-ethylpropan-2-amine;
N-(2-(5,7-difluoro-1H-indol-3-yl)ethyl)-N,2-dimethylpropan-1-amine;
N-(2-(5,7-difluoro-1H-indol-3-yl)ethyl)-N-methylcyclobutanamine;
N-(2-(5,7-difluoro-1H-indol-3-yl)ethyl)-N-methylprop-2-en-1-amine;
N-(2-(5,7-difluoro-1H-indol-3-yl)ethyl)-N-isopropyl-2-methylpropan-1-amine;
2-(6,7-difluoro-1H-indol-3-yl)-N-ethyl-N-methylethan-1-amine;
N-(2-(6,7-difluoro-1H-indol-3-yl)ethyl)-N-methylpropan-1-amine;
N-(2-(6,7-difluoro-1H-indol-3-yl)ethyl)-N-methylpropan-2-amine;
N-(2-(6,7-difluoro-1H-indol-3-yl)ethyl)-N-ethylpropan-1-amine;
N-(2-(6,7-difluoro-1H-indol-3-yl)ethyl)-N-ethylpropan-2-amine;
N-(2-(6,7-difluoro-1H-indol-3-yl)ethyl)-N,2-dimethylpropan-1-amine;
N-(2-(6,7-difluoro-1H-indol-3-yl)ethyl)-N-methylcyclobutanamine;
N-(2-(6,7-difluoro-1H-indol-3-yl)ethyl)-N-methylprop-2-en-1-amine;
N-(2-(6,7-difluoro-1H-indol-3-yl)ethyl)-N-isopropyl-2-methylpropan-1-amine;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
4-fluoro-3-(1-methylpyrrolidin-3-yl)-1H-indole;
6-fluoro-3-(1-methylpyrrolidin-3-yl)-1H-indole;
4,5-difluoro-3-(1-methylpyrrolidin-3-yl)-1H-indole;
4,6-difluoro-3-(1-methylpyrrolidin-3-yl)-1H-indole;
5,7-difluoro-3-(1-methylpyrrolidin-3-yl)-1H-indole;
6,7-difluoro-3-(1-methylpyrrolidin-3-yl)-1H-indole;
4-fluoro-3-(1-methylpiperidin-3-yl)-1H-indole;
5-fluoro-3-(1-methylpiperidin-3-yl)-1H-indole;
6-fluoro-3-(1-methylpiperidin-3-yl)-1H-indole;
7-fluoro-3-(1-methylpiperidin-3-yl)-1H-indole;
4,5-difluoro-3-(1-methylpiperidin-3-yl)-1H-indole;
4,6-difluoro-3-(1-methylpiperidin-3-yl)-1H-indole;
5,6-difluoro-3-(1-methylpiperidin-3-yl)-1H-indole;
5,7-difluoro-3-(1-methylpiperidin-3-yl)-1H-indole;
6,7-difluoro-3-(1-methylpiperidin-3-yl)-1H-indole;
4-fluoro-3-(methylprolyl)-1H-indole;
6-fluoro-3-(methylprolyl)-1H-indole;
7-fluoro-3-(methylprolyl)-1H-indole;
4,5-difluoro-3-(methylprolyl)-1H-indole;
4,6-difluoro-3-(methylprolyl)-1H-indole;
5,6-difluoro-3-(methylprolyl)-1H-indole;
5,7-difluoro-3-(methylprolyl)-1H-indole;
6,7-difluoro-3-(methylprolyl)-1H-indole;
4-fluoro-3-((1-methylpyrrolidin-2-yl)methyl)-1H-indole;
6-fluoro-3-((1-methylpyrrolidin-2-yl)methyl)-1H-indole;
7-fluoro-3-((1-methylpyrrolidin-2-yl)methyl)-1H-indole;
4,5-difluoro-3-((1-methylpyrrolidin-2-yl)methyl)-1H-indole;
4,6-difluoro-3-((1-methylpyrrolidin-2-yl)methyl)-1H-indole;
5,6-difluoro-3-((1-methylpyrrolidin-2-yl)methyl)-1H-indole;
5,7-difluoro-3-((1-methylpyrrolidin-2-yl)methyl)-1H-indole;
6,7-difluoro-3-((1-methylpyrrolidin-2-yl)methyl)-1H-indole;
N-(sec-butyl)-N-(2-(5-fluoro-1H-indol-3-yl)ethyl)cyclobutanamine;
N-(cyclopropylmethyl)-N-(2-(5-fluoro-1H-indol-3-yl)ethyl)propan-2-amine;
N-(2-(5-fluoro-1H-indol-3-yl)ethyl)-N-methylcyclopropanamine;
N-(2-(5-fluoro-1H-indol-3-yl)ethyl)-N-propylcyclopropanamine;
N-(2-(7-fluoro-1H-indol-3-yl)ethyl)-N-propylcyclobutanamine;
N-(2-(7-fluoro-1H-indol-3-yl)ethyl)-N-propylbutan-2-amine;
N-ethyl-N-methyl-2-(5,6,7-trifluoro-1H-indol-3-yl)ethan-1-amine;
N-ethyl-N-(2-(5,6,7-trifluoro-1H-indol-3-yl)ethyl)propan-1-amine;
N-ethyl-N-(2-(7-fluoro-5-methyl-1H-indol-3-yl)ethyl)propan-1-amine;
N-ethyl-N-(2-(5-fluoro-1H-indol-3-yl)ethyl)cyclopropanamine;
N-(2-(5-fluoro-1H-indol-3-yl)ethyl)-N-isopropylcyclopropanamine;
N-ethyl-2-(5-fluoro-7-methyl-1H-indol-3-yl)-N-methylethan-1-amine;
N-(2-(5-fluoro-1H-indol-3-yl)ethyl)-N,3-dimethylbutan-2-amine;
N-(sec-butyl)-N-(2-(5-fluoro-1H-indol-3-yl)ethyl)cyclopropanamine;
N-cyclopropyl-N-(2-(5-fluoro-1H-indol-3-yl)ethyl)cyclobutanamine;
2-(7-bromo-5-fluoro-1H-indol-3-yl)-N-ethyl-N-methylethan-1-amine;
N-ethyl-2-(5-fluoro-6-methyl-1H-indol-3-yl)-N-methylethan-1-amine;
N-(2-(5-fluoro-1H-indol-3-yl)ethyl)-N-(prop-2-yn-1-yl)butan-2-amine;
2-(sec-butyl(2-(5-fluoro-1H-indol-3-yl)ethyl)amino)acetonitrile;
N-(2-(5-fluoro-1H-indol-3-yl)ethyl)-N-methylbut-3-en-2-amine;
2-(5-chloro-6-fluoro-1H-indol-3-yl)-N-ethyl-N-methylethan-1-amine;
N-(2-(7-fluoro-1H-indol-3-yl)ethyl)-N-propylprop-2-yn-1-amine;
2-((2-(7-fluoro-1H-indol-3-yl)ethyl)(propyl)amino)acetonitrile;
2-(7-chloro-5-fluoro-1H-indol-3-yl)-N-ethyl-N-methylethan-1-amine;
(R)-3-((1-ethylpyrrolidin-2-yl)methyl)-5-fluoro-1H-indole;
(R)-5-fluoro-3-(pyrrolidin-2-ylmethyl)-1H-indole;
(R)-5-fluoro-3-((1-propylpyrrolidin-2-yl)methyl)-1H-indole;
(R)-5,6-difluoro-3-((1-methylpyrrolidin-2-yl)methyl)-1H-indole;
(R)-7-fluoro-3-((1-methylpyrrolidin-2-yl)methyl)-1H-indole;
2-(ethyl(propyl)amino)-1-(5-fluoro-1H-indol-3-yl)ethan-1-one;
N-isopropyl-N-(2-(5,6,7-trifluoro-1H-indol-3-yl)ethyl)propan-1-amine;
N-methyl-N-(2-(5,6,7-trifluoro-1H-indol-3-yl)ethyl)butan-2-amine;
N-methyl-N-(2-(5,6,7-trifluoro-1H-indol-3-yl)ethyl)cyclobutanamine;
N-(2-(7-fluoro-5-methoxy-1H-indol-3-yl)ethyl)-N-isopropylpropan-1-amine;
N-(2-(7-fluoro-5-methoxy-1H-indol-3-yl)ethyl)-N-methylbutan-2-amine;
N-(2-(7-fluoro-5-methoxy-1H-indol-3-yl)ethyl)-N-methylbut-3-en-2-amine;
N-(2-(5,6-difluoro-1H-indol-3-yl)ethyl)-N-methylbut-3-en-2-amine;
N-(2-(6,7-difluoro-1H-indol-3-yl)ethyl)-N-methylbut-3-en-2-amine;
N-(2-(5,7-difluoro-1H-indol-3-yl)ethyl)-N-methylbut-3-en-2-amine;
N-methyl-N-(2-(5,6,7-trifluoro-1H-indol-3-yl)ethyl)but-3-en-2-amine;
3-(((2R)-1-(sec-butyl)pyrrolidin-2-yl)methyl)-5-fluoro-1H-indole;
(R)-5-fluoro-3-((1-isopropylpyrrolidin-2-yl)methyl)-1H-indole;
(R)-3-((1-cyclobutylpyrrolidin-2-yl)methyl)-5-fluoro-1H-indole;
(R)-3-((1-cyclopropylpyrrolidin-2-yl)methyl)-5-fluoro-1H-indole;
(R)-3-((1-allylpyrrolidin-2-yl)methyl)-5-fluoro-1H-indole;
3-(((2R)-1-(but-3-en-2-yl)pyrrolidin-2-yl)methyl)-5-fluoro-1H-indole;
(R)-5-fluoro-3-((1-(prop-2-yn-1-yl)pyrrolidin-2-yl)methyl)-1H-indole;
3-(((2R)-1-(sec-butyl)pyrrolidin-2-yl)methyl)-5,6,7-trifluoro-1H-indole;
(R)-5,6,7-trifluoro-3-((1-isopropylpyrrolidin-2-yl)methyl)-1H-indole;
(R)-3-((1-cyclobutylpyrrolidin-2-yl)methyl)-5,6,7-trifluoro-1H-indole;
(R)-3-((1-cyclopropylpyrrolidin-2-yl)methyl)-5,6,7-trifluoro-1H-indole;
(R)-3-((1-allylpyrrolidin-2-yl)methyl)-5,6,7-trifluoro-1H-indole;
3-(((2R)-1-(but-3-en-2-yl)pyrrolidin-2-yl)methyl)-5,6,7-trifluoro-1H-indole;
(R)-5,6,7-trifluoro-3-((1-(prop-2-yn-1-yl)pyrrolidin-2-yl)methyl)-1H-indole;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

Embodiment 34 provides a compound of formula (II), or a salt, prodrug, solvate, isotopologue, or stereoisomer thereof:

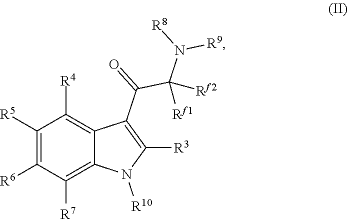

wherein:
   R³ is selected from the group consisting of H, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
   R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted C₁-C₈ heteroalkyl, optionally substituted benzyl, optionally substituted phenyl, and optionally substituted C₂-C₉ heteroaryl,
      wherein at least one of R⁴, R⁵, R⁶, and R⁷ is F;
   R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
   R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R^{f1} and R^{f2} are each independently selected from the group consisting of H and C₁-C₆ alkyl;
   each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂₀(C₆-C₁₀ aryl), —S(═O)₂₀(C₁-C₆ alkyl), —S(═O)₂₀(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
   each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

Embodiment 35 provides the compound of Embodiment 34, wherein R³ is H.

Embodiment 36 provides the compound of Embodiment 34 or 35, wherein R⁸ and R⁹ are each independently selected from the group consisting of methyl, ethyl, and n-propyl.

Embodiment 37 provides the compound of any one of Embodiments 34-36, wherein R¹⁰ is H.

Embodiment 38 provides the compound of any one of Embodiments 34-37, wherein R¹ and R are H.

Embodiment 39 provides the compound of any one of Embodiments 34-38, wherein:
(a) R⁴ is F, and each of R⁵, R⁶, and R⁷ is H;
(b) R⁵ is F, and each of R⁴, R⁶, and R⁷ is H;
(c) R⁶ is F, and each of R⁴, R⁵, and R⁷ is H;
(d) R⁷ is F, and each of R⁴, R⁵, and R⁶ is H;
(e) each of R⁴ and R⁵ is F, and each of R⁶ and R⁷ is H;
(f) each of R⁴ and R⁶ is F, and each of R⁵ and R⁷ is H;
(g) each of R⁴ and R⁷ is F, and each of R⁵ and R⁶ is H;
(h) each of R⁵ and R⁶ is F, and each of R⁴ and R⁷ is H;
(i) each of R⁵ and R⁷ is F, and each of R⁴ and R⁶ is H; or
(j) each of R⁶ and R⁷ is F, and each of R⁴ and R⁵ is H.

Embodiment 40 provides the compound of any one of Embodiments 34-39, which is selected from the group consisting of:
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
2-(dimethylamino)-1-(4-fluoro-1H-indol-3-yl)ethan-1-one;
2-(dimethylamino)-1-(5-fluoro-1H-indol-3-yl)ethan-1-one;
2-(dimethylamino)-1-(6-fluoro-1H-indol-3-yl)ethan-1-one;
2-(dimethylamino)-1-(7-fluoro-1H-indol-3-yl)ethan-1-one;
1-(4,5-difluoro-1H-indol-3-yl)-2-(dimethylamino)ethan-1-one;
1-(4,6-difluoro-1H-indol-3-yl)-2-(dimethylamino)ethan-1-one;
1-(5,6-difluoro-1H-indol-3-yl)-2-(dimethylamino)ethan-1-one;
1-(5,7-difluoro-1H-indol-3-yl)-2-(dimethylamino)ethan-1-one; and
1-(6,7-difluoro-1H-indol-3-yl)-2-(dimethylamino)ethan-1-one.

Embodiment 41 provides a compound of formula (III), or a salt, prodrug, solvate, isotopologue, or stereoisomer thereof:

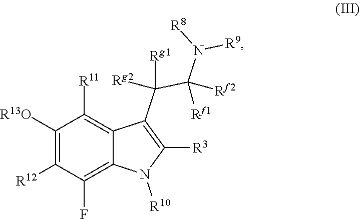

wherein:
   R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
   R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R¹¹ and R¹² are each independently selected from the group consisting of H, F, Cl, Br, I, OR^{A}, N(R^{A})(R^{B}), optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
   R¹³ is selected from the group consisting of H, optionally substituted C₁-C₆ alkyl, optionally substituted C₁-C₆ alkenyl, optionally substituted C₁-C₆ alkynyl, optionally substituted C₁-C₃ haloalkyl, optionally substituted benzyl, optionally substituted phenyl, and optionally substituted C₂-C₉ heteroaryl;
   R^{f1} and R^{f2} are each independently selected from the group consisting of H and C₁-C₆ alkyl;
   R^{g1} and R^{g2} are each independently selected from the group consisting of H and C₁-C₆ alkyl;
   each occurrence of R^{A} is independently selected from the group consisting of H, C₁-C₆ alkyl, C₂-C₆ alkenyl, C₁-C₆ haloalkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂₀(C₆-C₁₀ aryl), —S(═O)₂₀(C₁-C₆ alkyl), —S(═O)₂₀(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
   each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

Embodiment 42 provides the compound of Embodiment 41, wherein R³ is H.

Embodiment 43 provides the compound of Embodiment 41 or 42, wherein each of R⁸ and R⁹ are independently selected from the group consisting of methyl, ethyl, n-propyl, iso-propyl, sec-butyl, iso-butyl, n-butyl, cyclopropyl, cyclopropylmethyl, methylcyclopropyl, cyclopropylethyl, cyclobutyl, allyl, methylallyl, 2-methylallyl, 3-methylallyl, allylmethyl, propargyl, cyanomethyl, 2-hydroxyethyl, and 2-methoxyethyl.

Embodiment 44 provides the compound of any one of Embodiments 41-43, wherein R¹⁰ is H.

Embodiment 45 provides the compound of any one of Embodiments 41-44, wherein each of R¹¹ and R¹² is H.

Embodiment 46 provides the compound of any one of Embodiments 41-45, wherein R¹³ is H or methyl.

Embodiment 47 provides the compound of any one of Embodiments 41-46, wherein each of R^{f1}, R^{f2}, R^{g1}, and R^{g2} is H.

Embodiment 48 provides the compound of any one of Embodiments 41-47, which is selected from the group consisting of:
N-ethyl-N-(2-(7-fluoro-5-methoxy-1H-indol-3-yl)ethyl)propan-1-amine;
2-(7-fluoro-5-methoxy-1H-indol-3-yl)-N,N-dimethylethan-1-amine;
N,N-diethyl-2-(7-fluoro-5-methoxy-1H-indol-3-yl)ethan-1-amine;
N-(2-(7-fluoro-5-methoxy-1H-indol-3-yl)ethyl)-N-propylpropan-1-amine;
3-(2-(dimethylamino)ethyl)-7-fluoro-1H-indol-5-ol;
3-(2-(diethylamino)ethyl)-7-fluoro-1H-indol-5-ol;
3-(2-(dipropylamino)ethyl)-7-fluoro-1H-indol-5-ol;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

Embodiment 49 provides the compound of any one of Embodiments 1-48, wherein each occurrence of alkyl, heteroalkyl, alkenyl, alkynyl, cycloalkyl, and heterocyclyl is independently optionally substituted with at least one substituent selected from the group consisting of C₁-C₆ alkyl, C₃-C₈ cycloalkyl, halo, cyano, OR^{A}, optionally substituted benzyl, optionally substituted phenyl, optionally substituted C₂-C₉ heteroaryl, C(═O)OR^{A}, OC(═O)OR^{A}, OC(═O)R^{A}, SR^{A}S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}) and N(R^{A})(R^{B}).

Embodiment 50 provides the compound of any one of Embodiments 1-49, wherein each occurrence of benzyl, phenyl, and heteroaryl is independently optionally substituted with at least one substituent selected from the group consisting of C₁-C₆ alkyl, C₃-C₈ cycloalkyl, halo, cyano, OR^{A}, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}).

Embodiment 51 provides a compound selected from the group consisting of:
N-(2-(5-fluoro-1H-indol-3-yl)ethyl)prop-2-en-1-amine;
2-(5-fluoro-1H-indol-3-yl)-N,N-dimethylpropan-1-amine;
N-(2-(5-fluoro-1H-indol-3-yl)ethyl)-N-propylpropan-1-amine;
N,N-diethyl-2-(4-fluoro-1H-indol-3-yl)ethan-1-amine;
N-(2-(4-fluoro-1H-indol-3-yl)ethyl)-N-propylpropan-1-amine;
N-(2-(7-fluoro-1H-indol-3-yl)ethyl)-N-propylpropan-1-amine;
N-(2-(5,6-difluoro-1H-indol-3-yl)ethyl)-N-propylpropan-1-amine;
2-(5,7-difluoro-1H-indol-3-yl)-N,N-diethylethan-1-amine;
N-(2-(5,7-difluoro-1H-indol-3-yl)ethyl)-N-propylpropan-1-amine;
2-(6,7-difluoro-1H-indol-3-yl)-N,N-diethylethan-1-amine;
N-(2-(6,7-difluoro-1H-indol-3-yl)ethyl)-N-propylpropan-1-amine; and
2-(5-fluoro-1H-indol-3-yl)propan-1-amine.

Embodiment 52 provides a pharmaceutical composition comprising at least one compound of any one of Embodiments 1-51 and a pharmaceutically acceptable carrier.

Embodiment 53 provides a method of treating, preventing, and/or ameliorating a psychiatric disease or disorder in a subject in need thereof, the method comprising administering to the subject a therapeutically effective amount of at least one compound of any of Embodiments 1-51, at least one compound of any of compounds 1-218, or the pharmaceutical composition of Embodiment 52.

Embodiment 54 provides the method of Embodiment 53, wherein the psychiatric disease or disorder is selected from the group consisting of a depressive disorder, anxiety disorder, and eating disorder.

Embodiment 55 provides the method of Embodiment 53 or 54, wherein the psychiatric disease or disorder is selected from the group consisting of attention deficit disorder (ADD), attention deficit hyperactivity disorder (ADHD), treatment resistant depression, major depressive disorder (MDD), bipolar I disorder, bipolar II disorder, cyclothymic disorder, anti-social personality disorder, pain, sleep-wake disorders, disruptive mood dysregulation disorder, persistent depressive disorder (dysthymia), premenstrual dysphoric disorder, substance/medication-induced depressive disorder, post-partum depression, depressive disorder due to another medical condition, separation anxiety disorder, specific phobia, social anxiety disorder, panic disorder, panic attack, agoraphobia, generalized anxiety disorder, substance-medication induced anxiety disorder, anxiety disorder due to another medical condition, somatic symptom disorder, illness anxiety disorder, obsessive-compulsive disorder (OCD), obsessive-compulsive and related disorder (OCRD), OCRD due to another medical condition, substance-related disorders, alcohol-related disorders, cannabis-related disorders, hallucinogen-related disorders, inhalant-related disorders, cocaine-related disorders, opioid-related disorders, sedative-, hypnotic-, and/or anxiolytic-related disorders, stimulant-related disorders, tobacco-related disorders, non-substance-related disorders (gambling and/or gaming disorder), anorexia nervosa, bulimia nervosa, and binge-eating disorder.

Embodiment 56 provides the method of any one of Embodiments 53-55, wherein the subject is further administered at least one additional agent useful for treating, preventing, and/or ameliorating the psychiatric disease or disorder.

Embodiment 57 provides the method of Embodiment 56, wherein the at least one additional agent is selected from the group consisting of a selective serotonin reuptake inhibitor, triple reuptake inhibitor, serotonin and norepinephrine reuptake inhibitor, tricyclic antidepressant, tetracyclic antidepressant, dopamine reuptake inhibitor, mood stabilizer, anticonvulsant, antipsychotic, anxiolytics, benzodiazepines, monoamine releasers, dopamine receptor agonist, cannabinoids, triptans, anti-migraine medications, analgesics, anti-inflammatory, immune modulator, 5-HT_{1A} receptor antagonist, 5-HT₂ receptor antagonist, 5-HT₃ receptor antagonist, monoamine oxidase inhibitor, and noradrenergic antagonist.

Embodiment 58 provides the method of Embodiment 56 or 57, wherein the subject is co-administered the at least one compound and the at least one additional agent.

Embodiment 59 provides the method of any one of Embodiments 56-58, wherein the at least one compound and the at least one additional agent are co-formulated.

Embodiment 60 provides the method of any one of Embodiments 53-59, wherein the subject is a mammal.

Embodiment 61 provides the method of Embodiment 60, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of formula (I), or a salt, prodrug, solvate, isotopologue, or stereoisomer thereof: 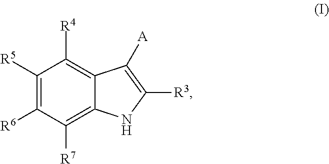
wherein:
A is 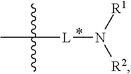
wherein * indicates the bond between L and N(R¹)(R²);
L is 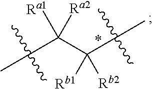
R^{a1} and R^{a2} are each independently selected from the group consisting of H, halogen, C₁-C₆ alkoxy, and C₁-C₆ alkyl,
or R^{a1} and R^{a2} may combine to form a carbonyl (C═O);
R^{b1} and R^{b2} are each independently selected from the group consisting of H and C₁-C₆ alkyl;
R¹ and R² are each independently selected from the group consisting of optionally substituted C₁-C₈ alkyl, optionally substituted C₃-C₈ cycloalkyl, optionally substituted C₁-C₈ heteroalkyl, optionally substituted C₂-C₈ heterocycloalkyl, optionally substituted C₂-C₈ alkenyl, and optionally substituted C₂-C₈ alkynyl,
wherein R¹ and R² are not identical, and
wherein one of R¹ and R² may combine with one of R^{a1}, R^{a2}, R^{b1}, and R^{b2} to form an optionally substituted C₂-C₈ heterocyclyl,
with the proviso that,
if one of R¹ and R² combines with R^{b1} or R^{b2} to form a 5 membered ring and R⁵ is F, then at least one of R⁴, R⁶, and R⁷ is not H, or
if one of R¹ and R² combine with R^{b1} or R^{b2} to form a stereocenter then the compound consists essentially of one stereoisomer;
R³ is selected from the group consisting of H, halogen, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl,
wherein at least one of R⁴, R⁵, R⁶, and R⁷ is F;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl);
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl;
wherein the isotopologue does not comprise F¹⁸ in R¹ or R²; and
wherein the compound of formula (I) is not selected from the group consisting of:
N-ethyl-N-(2-(4-fluoro-1H-indol-3-yl)ethyl)propan-1-amine;
N-ethyl-N-(2-(5-fluoro-1H-indol-3-yl)ethyl)propan-1-amine;
N-ethyl-N-(2-(6-fluoro-1H-indol-3-yl)ethyl)propan-1-amine;
N-ethyl-N-(2-(7-fluoro-1H-indol-3-yl)ethyl)propan-1-amine;
N-ethyl-2-(4-fluoro-1H-indol-3-yl)-N-methylethan-1-amine;
N-ethyl-2-(5-fluoro-1H-indol-3-yl)-N-methylethan-1-amine;
N-ethyl-2-(6-fluoro-1H-indol-3-yl)-N-methylethan-1-amine;
N-ethyl-2-(7-fluoro-1H-indol-3-yl)-N-methylethan-1-amine;
(R)-4-fluoro-3-((1-(methyl-d3)pyrrolidin-2-yl)methyl-d2)-1H-indole;
(R)-4-fluoro-3-((1-(methyl-d3)pyrrolidin-2-yl)methyl)-1H-indole;
(R)-4-fluoro-3-(pyrrolidin-2-ylmethyl-d2)-1H-indole; and
(S)-4-fluoro-3-((1-(methyl-d3)pyrrolidin-2-yl)methyl-d2)-1H-indole.

2. The compound of claim 1, wherein R¹ is optionally substituted C₁-C₃ alkyl and R² is selected from the group consisting of optionally substituted-branched C₃-C₈ alkyl and optionally substituted C₃-C₈ cycloalkyl.

3. The compound of claim 1, wherein R¹ is selected from the group consisting of methyl, allyl, and n-propyl.

4. The compound of claim 1, wherein R² is optionally substituted-branched C₃-C₈ alkyl or optionally substituted C₃-C₈ cycloalkyl.

5. The compound of claim 4, wherein
(a) R² is selected from the group consisting of iso-propyl, sec-butyl, iso-butyl, 1,2-dimethylpropyl, methylallyl, and 2-methylallyl; or
(b) R² is selected from the group consisting of cyclopropyl and cyclobutyl.

6-7. (canceled)

8. The compound of claim 1, wherein each of R¹ and R² are independently selected from the group consisting of methyl, ethyl, n-propyl, 1,2-dimethylpropyl, iso-propyl, sec-butyl, iso-butyl, n-butyl, cyclopropyl, cyclopropylmethyl, methylcyclopropyl, cyclopropylethyl, 2-cyclopropyleth-2-yl, cyclobutyl, 2-thietanyl, 3-thietanyl, allyl, methylallyl, 2-methylallyl, 3-methylallyl, propargyl, cyanomethyl, 2-hydroxyethyl, and 2-methoxyethyl.

9. The compound of claim 1, wherein R³ is H.

10. The compound of claim 1, wherein R⁴ is F and each of R⁵, R⁶, and R⁷ is H.

11. The compound of claim 1, wherein at least one of the following applies:
(a) R⁵ is F, and each of R⁴, R⁶, and R⁷ is H;
(b) R⁷ is F, and each of R⁴, R⁵, and R⁶ is H;
(c) each of R⁴ and R⁵ is F, and each of R⁶ and R⁷ is H;
(d) each of R⁴ and R⁶ is F, and each of R⁵ and R⁷ is H;
(e) each of R⁴ and R⁷ is F, and each of R⁵ and R⁶ is H;
(f) each of R⁵ and R⁶ is F, and each of R⁴ and R⁷ is H;
(g) each of R⁵ and R⁷ is F, and each of R⁴ and R⁶ is H;
(h) each of R⁶ and R⁷ is F, and each of R⁴ and R⁵ is H;
(i) R⁵ is OMe, R⁷ is F, and each of R⁴ and R⁶ is H;
(i) R⁵ is OMe, R⁷ is F, and each of R⁴ and R⁶ is H;
(k) each of R⁵, R⁶, and R⁷ are F, and R⁴ is H;
(l) R⁶ is selected from the group consisting of H, F, Me, and Cl;
(m) R⁷ is selected from the group consisting of H, F, Cl, Br, and Me;
(n) each of R⁴ and R⁶ is H.

12-20. (canceled)

21. The compound of claim 1, wherein R⁵ is selected from the group consisting of OR^{A}, N(R^{A})(R^{B}), and SR^{A}.

22. The compound of claim 1, wherein R⁵ is selected from the group consisting of H, F, Cl, OMe, OH, and Me, optionally wherein R⁵ is OMe or F.

23-27. (canceled)

28. The compound of claim 1, wherein L is selected from the group consisting of 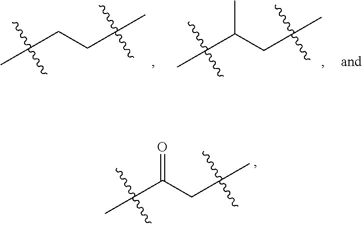
optionally wherein L is

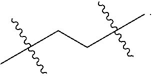 [SMILES: *CC*]

29. (canceled)

30. The compound of claim 1, wherein A is selected from the group consisting of: 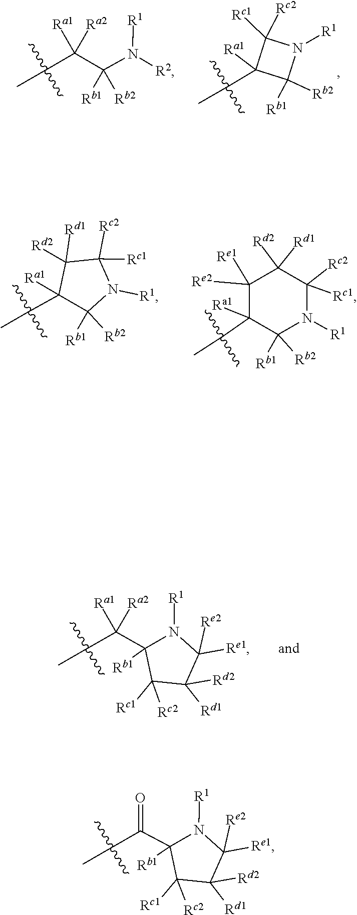
wherein:
R^{c1}, R^{c2}, R^{d1}, R^{d2}, R^{e1}, and R^{e2}, if present, are each independently selected from the group consisting of H, C₁-C₃ alkyl, and C₁-C₃ haloalkyl.

31. The compound of claim 1, wherein each of R^{a1}, R^{a2}, R^{b1}, R^{b2}, R^{c1}, R^{c2}, R^{d1}, R^{d2}, R^{e1}, R^{e2}, R^{f1}, and R^{f2}, if present, is H.

32. The compound of claim 1, wherein A is selected from the group consisting of:

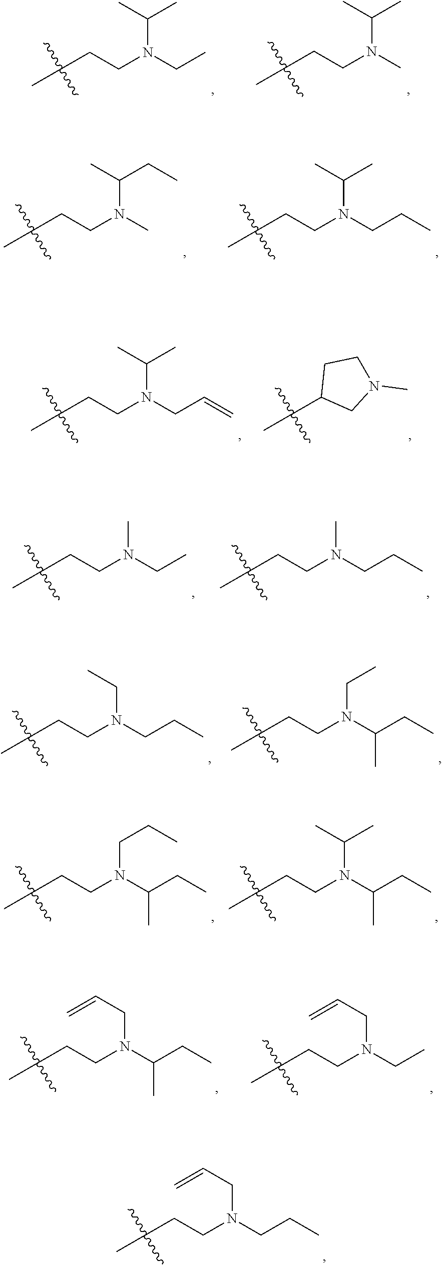

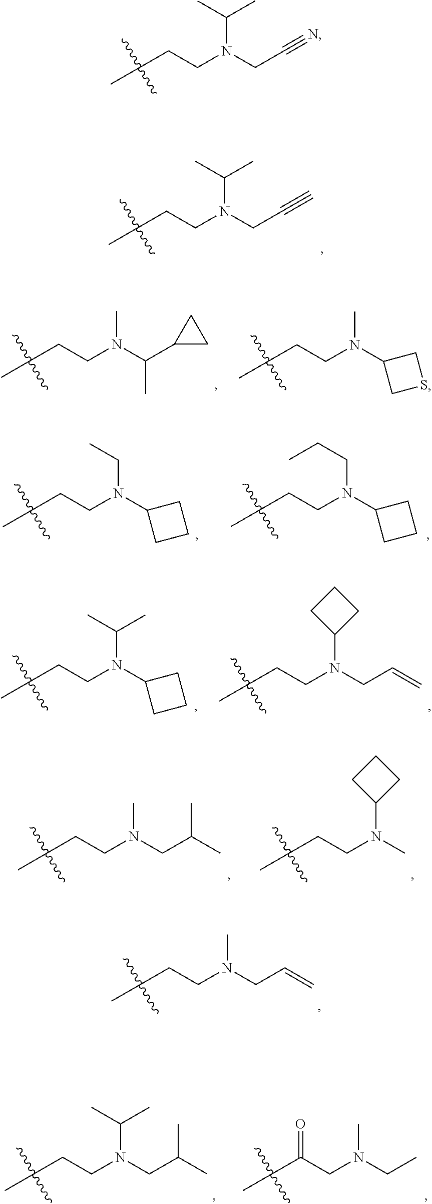

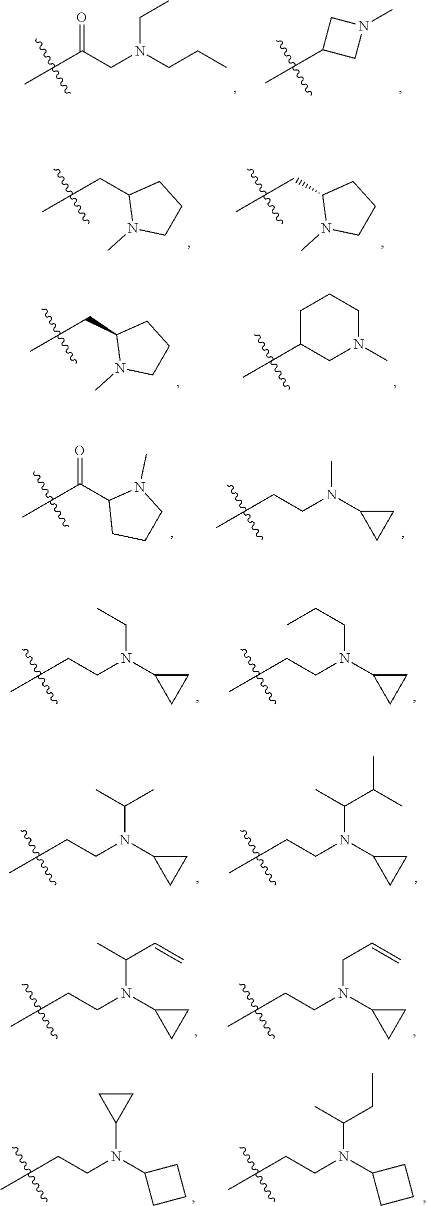

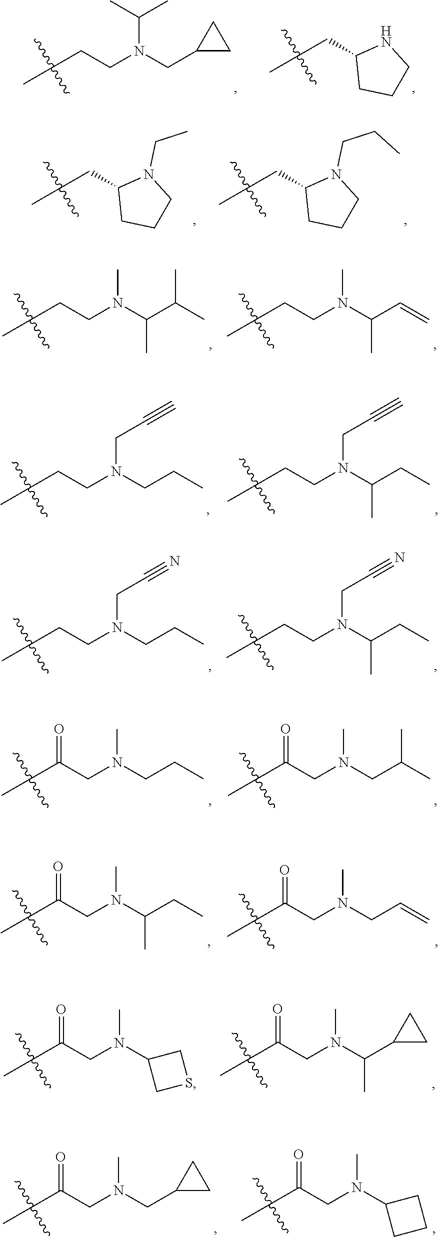

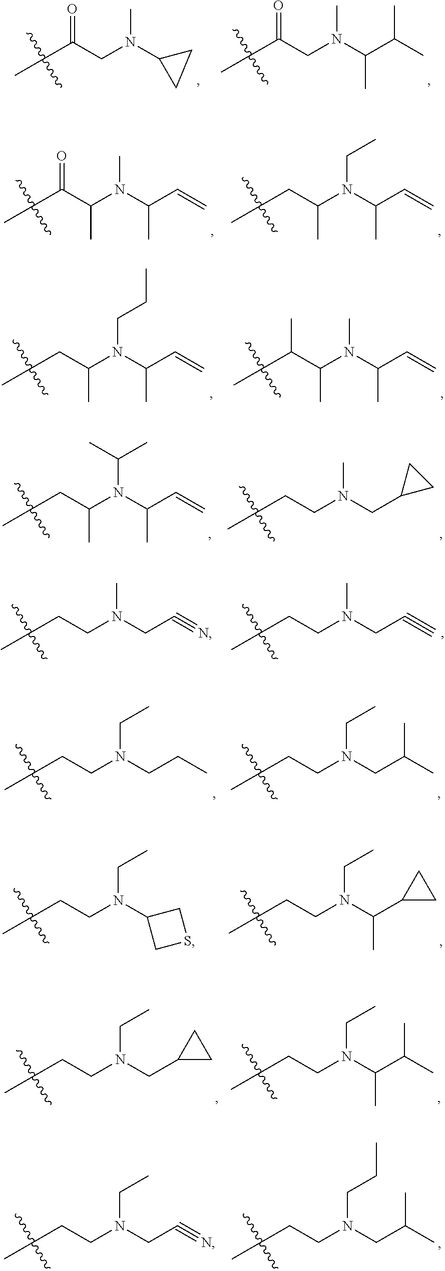

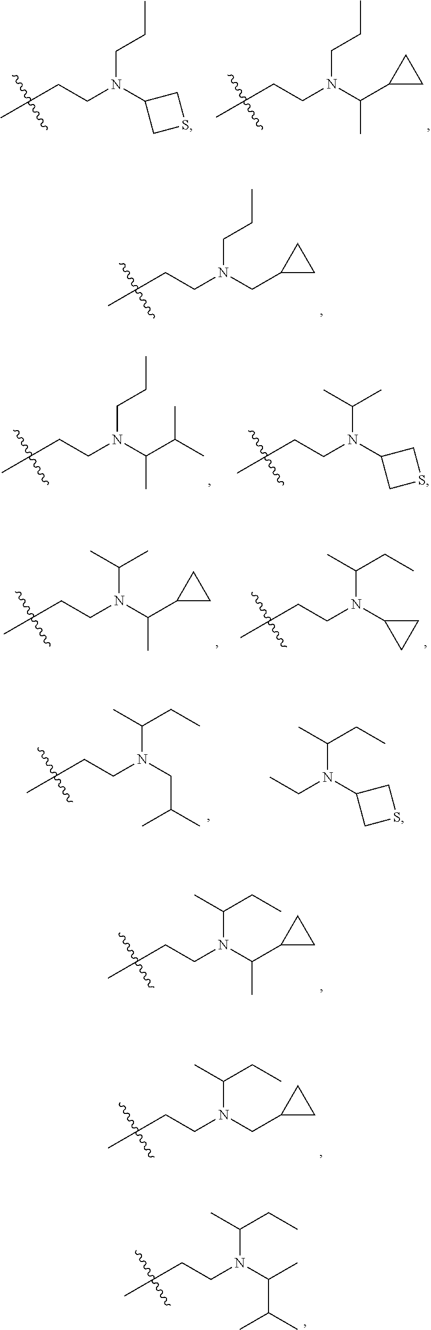

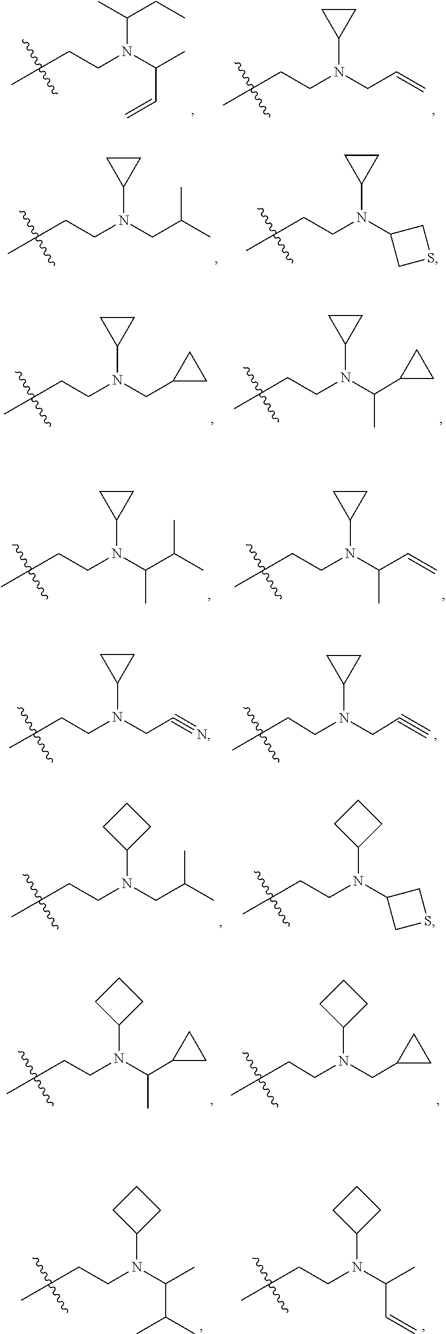

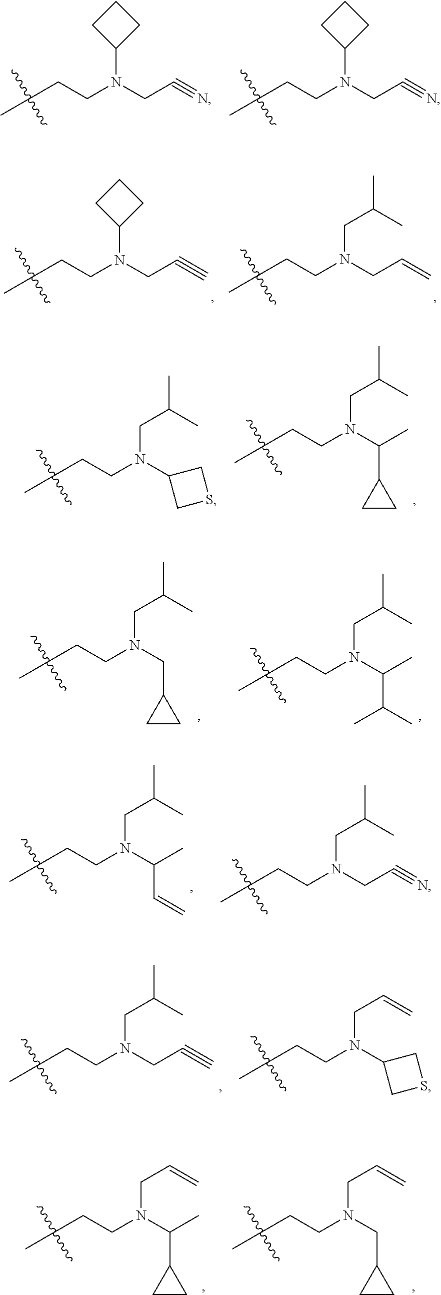

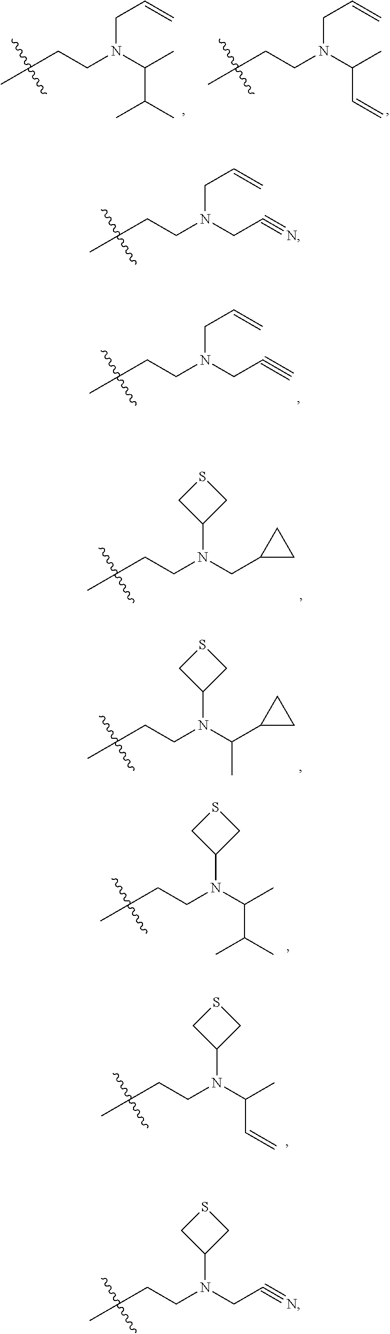

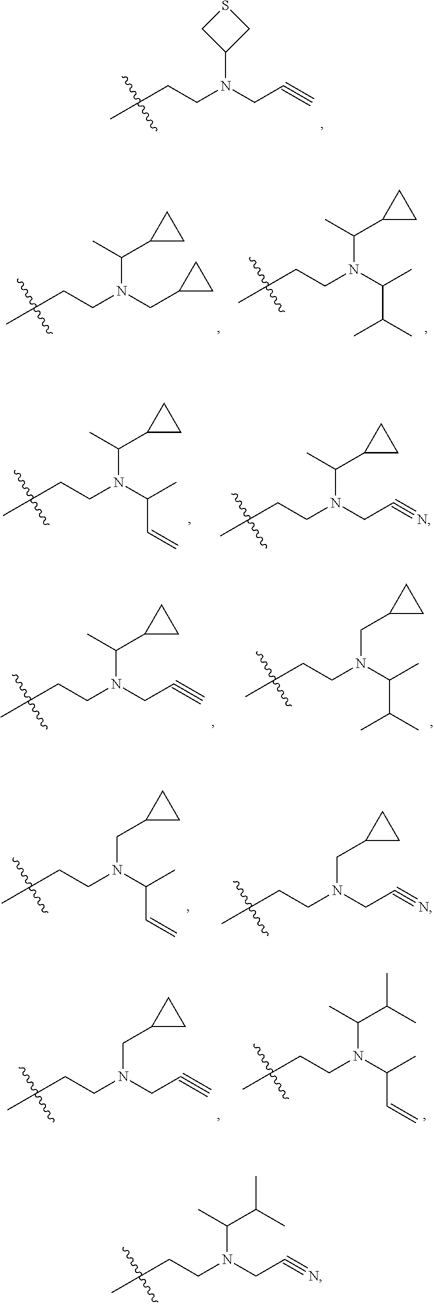

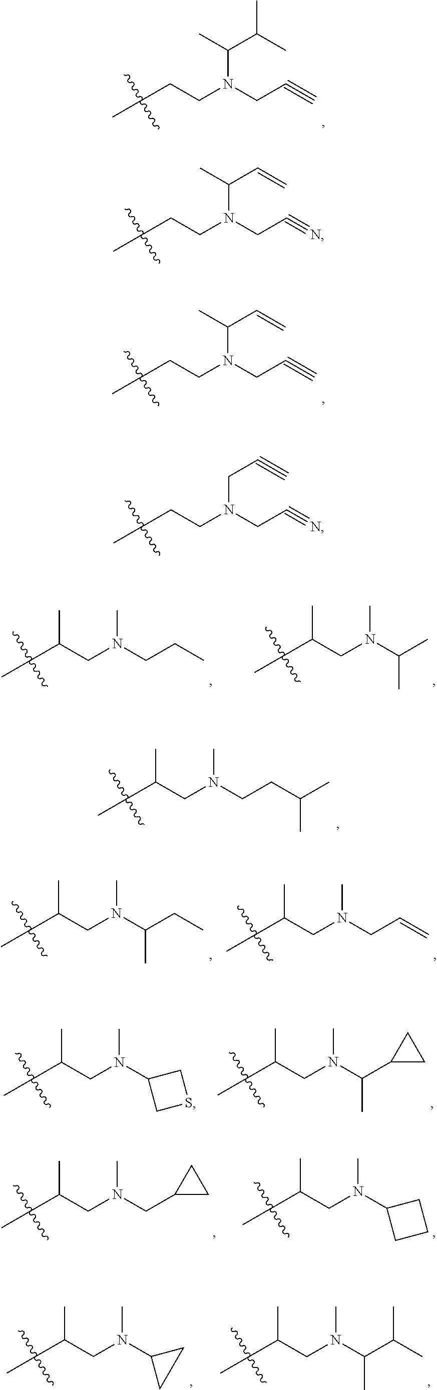

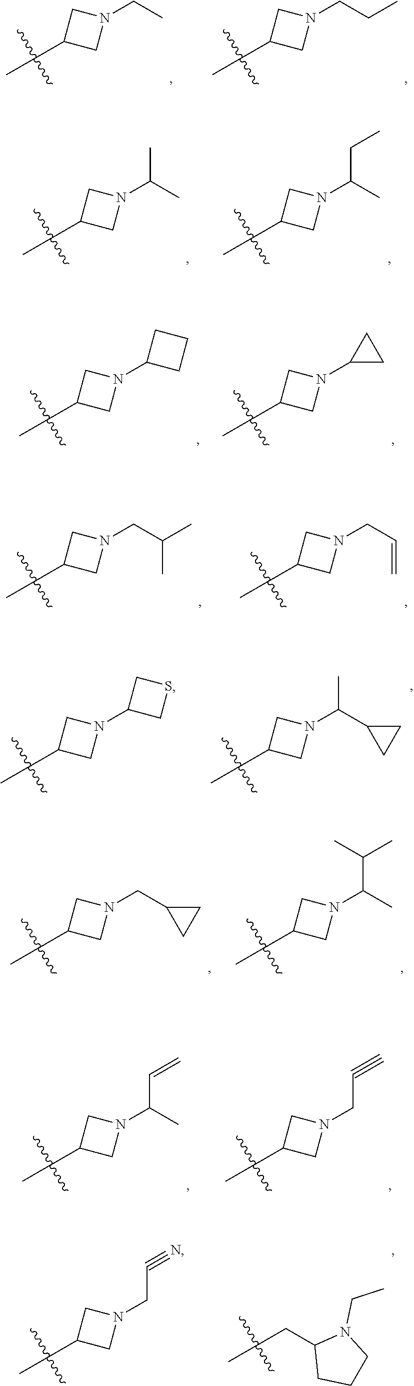

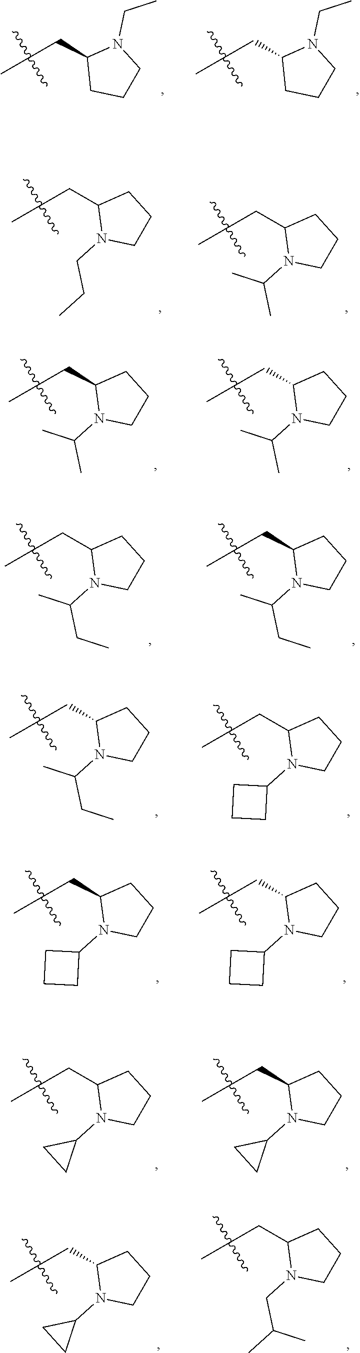

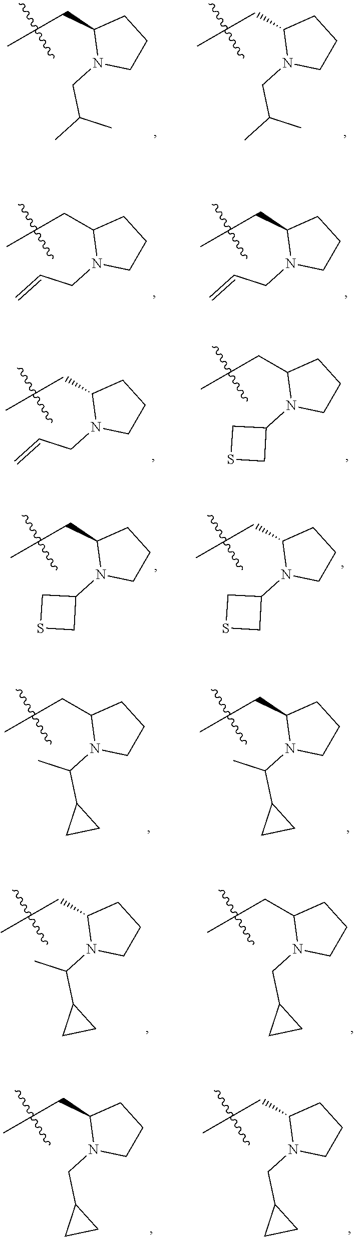

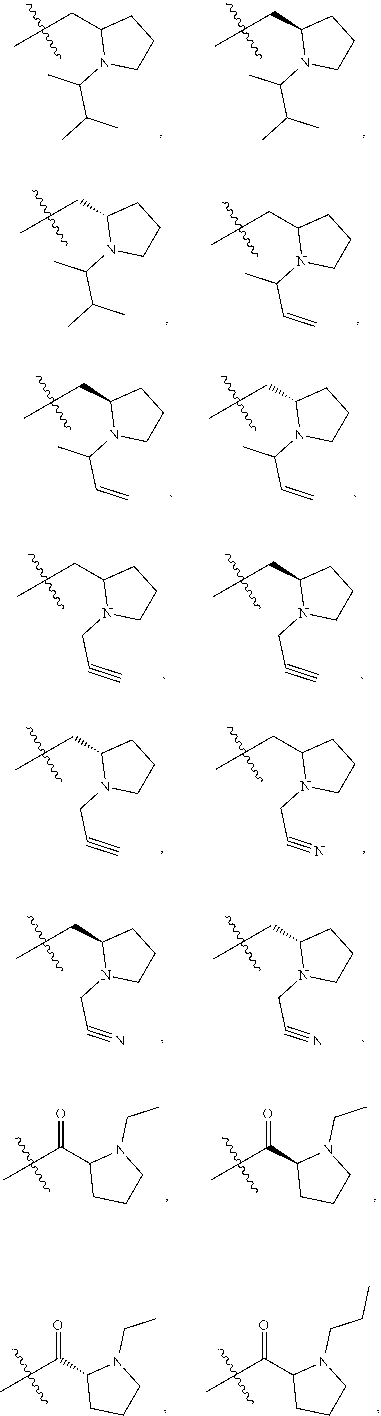

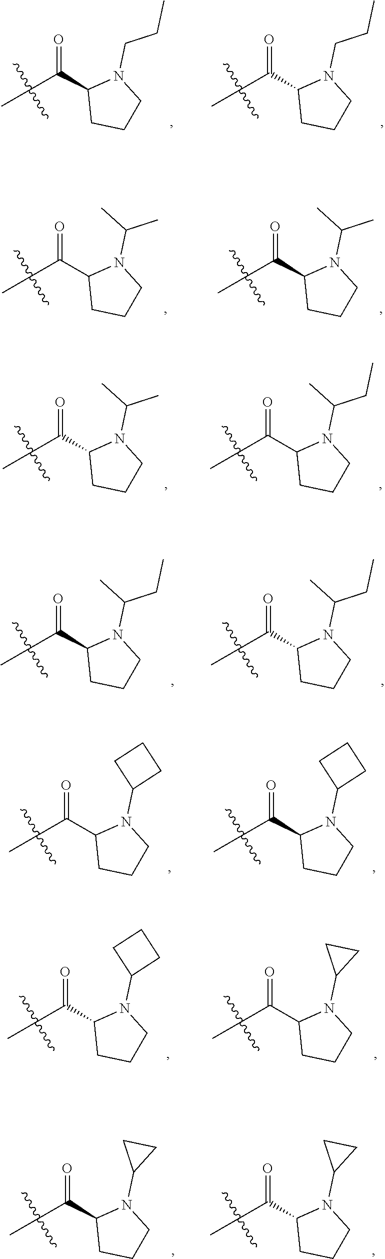

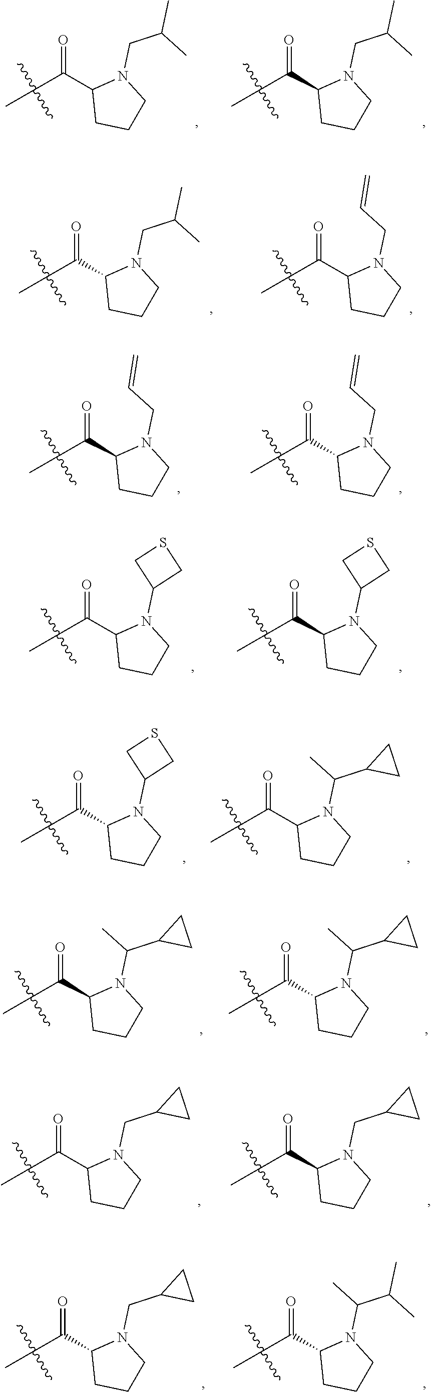

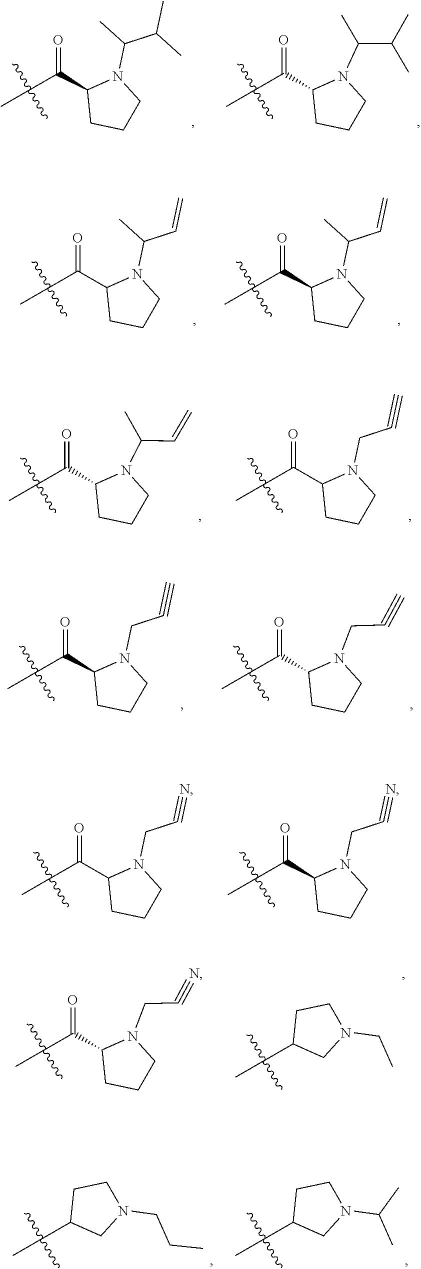

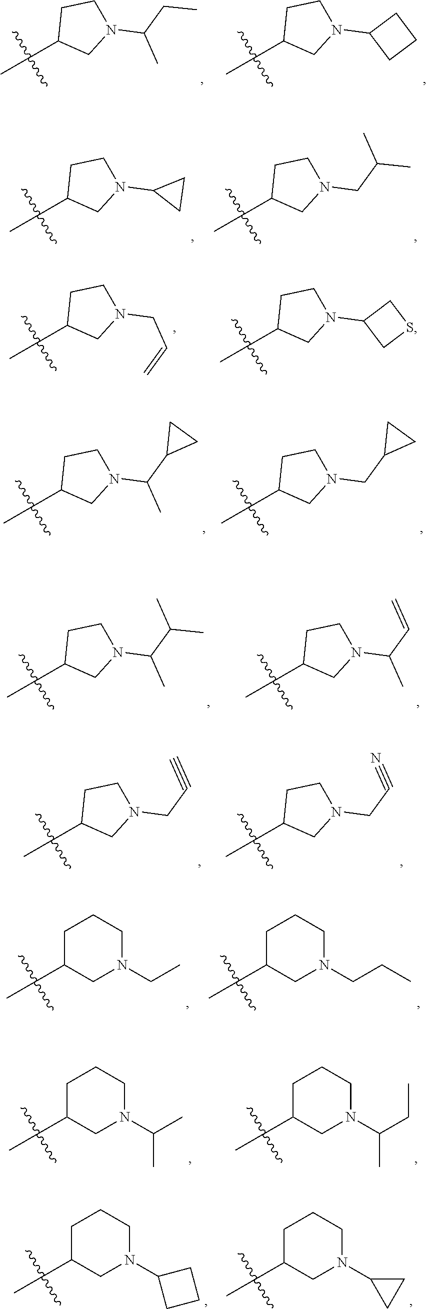

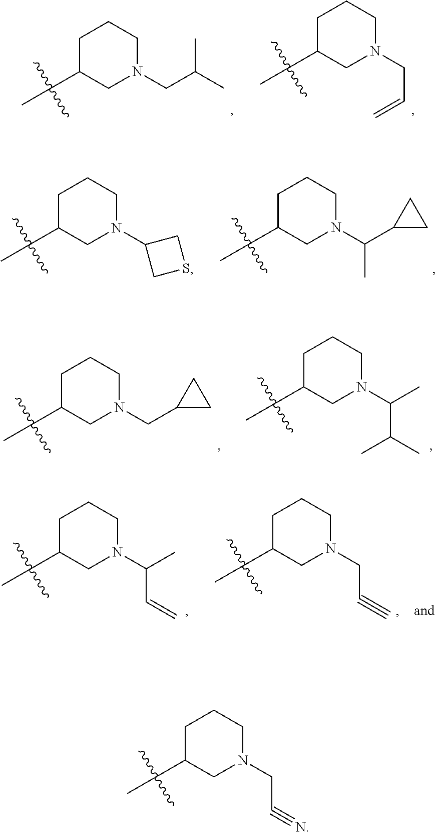

33. The compound of claim 1, which is selected from the group consisting of:
N-ethyl-N-(2-(5-fluoro-1H-indol-3-yl)ethyl)propan-2-amine;
N-(2-(5-fluoro-1H-indol-3-yl)ethyl)-N-methylpropan-2-amine;
N-(2-(5-fluoro-1H-indol-3-yl)ethyl)-N-methylbutan-2-amine;
N-(2-(5-fluoro-1H-indol-3-yl)ethyl)-N-isopropylpropan-1-amine;
N-(2-(5,6-difluoro-1H-indol-3-yl)ethyl)-N-ethylpropan-1-amine;
N-(2-(5-fluoro-1H-indol-3-yl)ethyl)-N-methylprop-2-en-1-amine;
N-(2-(5-fluoro-1H-indol-3-yl)ethyl)-N-isopropylprop-2-en-1-amine;
N-(2-(5-fluoro-1H-indol-3-yl)ethyl)-N-methylpropan-1-amine;
N-allyl-N-(2-(5-fluoro-1H-indol-3-yl)ethyl)cyclobutanamine;
N-allyl-N-(2-(5-fluoro-1H-indol-3-yl)ethyl)butan-2-amine;
2-((2-(5-fluoro-1H-indol-3-yl)ethyl)(isopropyl)amino)acetonitrile;
5,6-difluoro-3-(1-methylpyrrolidin-3-yl)-1H-indole;
N-(2-(5-fluoro-1H-indol-3-yl)ethyl)-N-methylthietan-3-amine;
N-(2-(4,5-difluoro-1H-indol-3-yl)ethyl)-N-ethylpropan-1-amine;
N-(2-(5-fluoro-1H-indol-3-yl)ethyl)-N-propylbutan-2-amine;
7-fluoro-3-(1-methylpyrrolidin-3-yl)-1H-indole;
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
(S)-5-fluoro-3-((1-methylpyrrolidin-2-yl)methyl)-1H-indole;
(R)-5-fluoro-3-((1-methylpyrrolidin-2-yl)methyl)-1H-indole;
5-fluoro-3-(1-methylazetidin-3-yl)-1H-indole;
N-ethyl-N-(2-(5-fluoro-1H-indol-3-yl)ethyl)butan-2-amine;
N-ethyl-N-(2-(5-fluoro-1H-indol-3-yl)ethyl)cyclobutanamine;
N-(2-(5-fluoro-1H-indol-3-yl)ethyl)-N-isopropylbutan-2-amine;
N-(2-(5-fluoro-1H-indol-3-yl)ethyl)-N-propylcyclobutanamine;
N-(2-(5-fluoro-1H-indol-3-yl)ethyl)-N-isopropylprop-2-yn-1-amine;
N-(2-(5-fluoro-1H-indol-3-yl)ethyl)-N-propylprop-2-en-1-amine;
N-ethyl-N-(2-(5-fluoro-1H-indol-3-yl)ethyl)prop-2-en-1-amine;
N-(2-(6-chloro-5-fluoro-1H-indol-3-yl)ethyl)-N-ethylpropan-1-amine;
1-cyclopropyl-N-(2-(5-fluoro-1H-indol-3-yl)ethyl)-N-methylethan-1-amine;
N-(2-(5-fluoro-1H-indol-3-yl)ethyl)-N,2-dimethylpropan-1-amine;
N-(2-(5-fluoro-1H-indol-3-yl)ethyl)-N-isopropylcyclobutanamine;
N-ethyl-N-(2-(7-fluoro-5-methoxy-1H-indol-3-yl)ethyl)propan-1-amine;
N-(2-(4-fluoro-1H-indol-3-yl)ethyl)-N-methylpropan-1-amine;
N-(2-(4-fluoro-1H-indol-3-yl)ethyl)-N-methylpropan-2-amine;
N-ethyl-N-(2-(4-fluoro-1H-indol-3-yl)ethyl)propan-2-amine;
N-(2-(4-fluoro-1H-indol-3-yl)ethyl)-N,2-dimethylpropan-1-amine;
N-(2-(4-fluoro-1H-indol-3-yl)ethyl)-N-methylcyclobutanamine;
N-(2-(4-fluoro-1H-indol-3-yl)ethyl)-N-methylprop-2-en-1-amine;
N-(2-(4-fluoro-1H-indol-3-yl)ethyl)-N-isopropyl-2-methylpropan-1-amine;
N-(2-(5-fluoro-1H-indol-3-yl)ethyl)-N-methylcyclobutanamine;
N-(2-(5-fluoro-1H-indol-3-yl)ethyl)-N-isopropyl-2-methylpropan-1-amine;
N-(2-(7-fluoro-1H-indol-3-yl)ethyl)-N-methylpropan-1-amine;
N-(2-(7-fluoro-1H-indol-3-yl)ethyl)-N-methylpropan-2-amine;
N-ethyl-N-(2-(7-fluoro-1H-indol-3-yl)ethyl)propan-2-amine;
N-(2-(7-fluoro-1H-indol-3-yl)ethyl)-N,2-dimethylpropan-1-amine;
N-(2-(7-fluoro-1H-indol-3-yl)ethyl)-N-methylcyclobutanamine;
N-(2-(7-fluoro-1H-indol-3-yl)ethyl)-N-methylprop-2-en-1-amine;
N-(2-(7-fluoro-1H-indol-3-yl)ethyl)-N-isopropyl-2-methylpropan-1-amine;
2-(5,6-difluoro-1H-indol-3-yl)-N-ethyl-N-methylethan-1-amine;
N-(2-(5,6-difluoro-1H-indol-3-yl)ethyl)-N-methylpropan-1-amine;
N-(2-(5,6-difluoro-1H-indol-3-yl)ethyl)-N-methylpropan-2-amine;
N-(2-(5,6-difluoro-1H-indol-3-yl)ethyl)-N-ethylpropan-2-amine;
N-(2-(5,6-difluoro-1H-indol-3-yl)ethyl)-N,2-dimethylpropan-1-amine;
N-(2-(5, 6-difluoro-1H-indol-3-yl)ethyl)-N-methylcyclobutanamine;
N-(2-(5, 6-difluoro-1H-indol-3-yl)ethyl)-N-methylprop-2-en-1-amine;
N-(2-(5, 6-difluoro-1H-indol-3-yl)ethyl)-N-isopropyl-2-methylpropan-1-amine;
2-(5,7-difluoro-1H-indol-3-yl)-N-ethyl-N-methylethan-1-amine;
N-(2-(5,7-difluoro-1H-indol-3-yl)ethyl)-N-methylpropan-1-amine;
N-(2-(5,7-difluoro-1H-indol-3-yl)ethyl)-N-methylpropan-2-amine;
N-(2-(5,7-difluoro-1H-indol-3-yl)ethyl)-N-ethylpropan-1-amine;
N-(2-(5,7-difluoro-1H-indol-3-yl)ethyl)-N-ethylpropan-2-amine;
N-(2-(5,7-difluoro-1H-indol-3-yl)ethyl)-N,2-dimethylpropan-1-amine;
N-(2-(5,7-difluoro-1H-indol-3-yl)ethyl)-N-methylcyclobutanamine;
N-(2-(5,7-difluoro-1H-indol-3-yl)ethyl)-N-methylprop-2-en-1-amine;
N-(2-(5,7-difluoro-1H-indol-3-yl)ethyl)-N-isopropyl-2-methylpropan-1-amine;
2-(6,7-difluoro-1H-indol-3-yl)-N-ethyl-N-methylethan-1-amine;
N-(2-(6,7-difluoro-1H-indol-3-yl)ethyl)-N-methylpropan-1-amine;
N-(2-(6,7-difluoro-1H-indol-3-yl)ethyl)-N-methylpropan-2-amine;
N-(2-(6,7-difluoro-1H-indol-3-yl)ethyl)-N-ethylpropan-1-amine;
N-(2-(6,7-difluoro-1H-indol-3-yl)ethyl)-N-ethylpropan-2-amine;
N-(2-(6,7-difluoro-1H-indol-3-yl)ethyl)-N,2-dimethylpropan-1-amine;
N-(2-(6,7-difluoro-1H-indol-3-yl)ethyl)-N-methylcyclobutanamine;
N-(2-(6,7-difluoro-1H-indol-3-yl)ethyl)-N-methylprop-2-en-1-amine;
N-(2-(6,7-difluoro-1H-indol-3-yl)ethyl)-N-isopropyl-2-methylpropan-1-amine;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
4-fluoro-3-(1-methylpyrrolidin-3-yl)-1H-indole;
6-fluoro-3-(1-methylpyrrolidin-3-yl)-1H-indole;
4,5-difluoro-3-(1-methylpyrrolidin-3-yl)-1H-indole;
4,6-difluoro-3-(1-methylpyrrolidin-3-yl)-1H-indole;
5,7-difluoro-3-(1-methylpyrrolidin-3-yl)-1H-indole;
6,7-difluoro-3-(1-methylpyrrolidin-3-yl)-1H-indole;
4-fluoro-3-(1-methylpiperidin-3-yl)-1H-indole;
5-fluoro-3-(1-methylpiperidin-3-yl)-1H-indole;
6-fluoro-3-(1-methylpiperidin-3-yl)-1H-indole;
7-fluoro-3-(1-methylpiperidin-3-yl)-1H-indole;
4,5-difluoro-3-(1-methylpiperidin-3-yl)-1H-indole;
4,6-difluoro-3-(1-methylpiperidin-3-yl)-1H-indole;
5,6-difluoro-3-(1-methylpiperidin-3-yl)-1H-indole;
5,7-difluoro-3-(1-methylpiperidin-3-yl)-1H-indole;
6,7-difluoro-3-(1-methylpiperidin-3-yl)-1H-indole;
4-fluoro-3-(methylprolyl)-1H-indole;
6-fluoro-3-(methylprolyl)-1H-indole;
7-fluoro-3-(methylprolyl)-1H-indole;
4,5-difluoro-3-(methylprolyl)-1H-indole;
4,6-difluoro-3-(methylprolyl)-1H-indole;
5,6-difluoro-3-(methylprolyl)-1H-indole;
5,7-difluoro-3-(methylprolyl)-1H-indole;
6,7-difluoro-3-(methylprolyl)-1H-indole;
4-fluoro-3-((1-methylpyrrolidin-2-yl)methyl)-1H-indole;
6-fluoro-3-((1-methylpyrrolidin-2-yl)methyl)-1H-indole;
7-fluoro-3-((1-methylpyrrolidin-2-yl)methyl)-1H-indole;
4,5-difluoro-3-((1-methylpyrrolidin-2-yl)methyl)-1H-indole;
4,6-difluoro-3-((1-methylpyrrolidin-2-yl)methyl)-1H-indole;
5,6-difluoro-3-((1-methylpyrrolidin-2-yl)methyl)-1H-indole;
5,7-difluoro-3-((1-methylpyrrolidin-2-yl)methyl)-1H-indole;
6,7-difluoro-3-((1-methylpyrrolidin-2-yl)methyl)-1H-indole;
N-(sec-butyl)-N-(2-(5-fluoro-1H-indol-3-yl)ethyl)cyclobutanamine;
N-(cyclopropylmethyl)-N-(2-(5-fluoro-1H-indol-3-yl)ethyl)propan-2-amine;
N-(2-(5-fluoro-1H-indol-3-yl)ethyl)-N-methylcyclopropanamine;
N-(2-(5-fluoro-1H-indol-3-yl)ethyl)-N-propylcyclopropanamine;
N-(2-(7-fluoro-1H-indol-3-yl)ethyl)-N-propylcyclobutanamine;
N-(2-(7-fluoro-1H-indol-3-yl)ethyl)-N-propylbutan-2-amine;
N-ethyl-N-methyl-2-(5,6,7-trifluoro-1H-indol-3-yl)ethan-1-amine;
N-ethyl-N-(2-(5, 6,7-trifluoro-1H-indol-3-yl)ethyl)propan-1-amine;
N-ethyl-N-(2-(7-fluoro-5-methyl-1H-indol-3-yl)ethyl)propan-1-amine;
N-ethyl-N-(2-(5-fluoro-1H-indol-3-yl)ethyl)cyclopropanamine;
N-(2-(5-fluoro-1H-indol-3-yl)ethyl)-N-isopropylcyclopropanamine;
N-ethyl-2-(5-fluoro-7-methyl-1H-indol-3-yl)-N-methylethan-1-amine;
N-(2-(5-fluoro-1H-indol-3-yl)ethyl)-N,3-dimethylbutan-2-amine;
N-(sec-butyl)-N-(2-(5-fluoro-1H-indol-3-yl)ethyl)cyclopropanamine;
N-cyclopropyl-N-(2-(5-fluoro-1H-indol-3-yl)ethyl)cyclobutanamine;
2-(7-bromo-5-fluoro-1H-indol-3-yl)-N-ethyl-N-methylethan-1-amine;
N-ethyl-2-(5-fluoro-6-methyl-1H-indol-3-yl)-N-methylethan-1-amine;
N-(2-(5-fluoro-1H-indol-3-yl)ethyl)-N-(prop-2-yn-1-yl)butan-2-amine;
2-(sec-butyl(2-(5-fluoro-1H-indol-3-yl)ethyl)amino)acetonitrile;
N-(2-(5-fluoro-1H-indol-3-yl)ethyl)-N-methylbut-3-en-2-amine;
2-(5-chloro-6-fluoro-1H-indol-3-yl)-N-ethyl-N-methylethan-1-amine;
N-(2-(7-fluoro-1H-indol-3-yl)ethyl)-N-propylprop-2-yn-1-amine;
2-((2-(7-fluoro-1H-indol-3-yl)ethyl)(propyl)amino)acetonitrile;
2-(7-chloro-5-fluoro-1H-indol-3-yl)-N-ethyl-N-methylethan-1-amine;
(R)-3-((1-ethylpyrrolidin-2-yl)methyl)-5-fluoro-1H-indole;
(R)-5-fluoro-3-(pyrrolidin-2-ylmethyl)-1H-indole;
(R)-5-fluoro-3-((1-propylpyrrolidin-2-yl)methyl)-1H-indole;
(R)-5,6-difluoro-3-((1-methylpyrrolidin-2-yl)methyl)-1H-indole;
(R)-7-fluoro-3-((1-methylpyrrolidin-2-yl)methyl)-1H-indole;
2-(ethyl(propyl)amino)-1-(5-fluoro-1H-indol-3-yl)ethan-1-one;
N-isopropyl-N-(2-(5, 6,7-trifluoro-1H-indol-3-yl)ethyl)propan-1-amine;
N-methyl-N-(2-(5,6,7-trifluoro-1H-indol-3-yl)ethyl)butan-2-amine;
N-methyl-N-(2-(5,6,7-trifluoro-1H-indol-3-yl)ethyl)cyclobutanamine;
N-(2-(7-fluoro-5-methoxy-1H-indol-3-yl)ethyl)-N-isopropylpropan-1-amine;
N-(2-(7-fluoro-5-methoxy-1H-indol-3-yl)ethyl)-N-methylbutan-2-amine;
N-(2-(7-fluoro-5-methoxy-1H-indol-3-yl)ethyl)-N-methylbut-3-en-2-amine;
N-(2-(5, 6-difluoro-1H-indol-3-yl)ethyl)-N-methylbut-3-en-2-amine;
N-(2-(6,7-difluoro-1H-indol-3-yl)ethyl)-N-methylbut-3-en-2-amine;
N-(2-(5,7-difluoro-1H-indol-3-yl)ethyl)-N-methylbut-3-en-2-amine;
N-methyl-N-(2-(5,6,7-trifluoro-1H-indol-3-yl)ethyl)but-3-en-2-amine;
3-(((2R)-1-(sec-butyl)pyrrolidin-2-yl)methyl)-5-fluoro-1H-indole;
(R)-5-fluoro-3-((1-isopropylpyrrolidin-2-yl)methyl)-1H-indole;
(R)-3-((1-cyclobutylpyrrolidin-2-yl)methyl)-5-fluoro-1H-indole;
(R)-3-((1-cyclopropylpyrrolidin-2-yl)methyl)-5-fluoro-1H-indole;
(R)-3-((1-allylpyrrolidin-2-yl)methyl)-5-fluoro-1H-indole;
3-(((2R)-1-(but-3-en-2-yl)pyrrolidin-2-yl)methyl)-5-fluoro-1H-indole;
(R)-5-fluoro-3-((1-(prop-2-yn-1-yl)pyrrolidin-2-yl)methyl)-1H-indole;
3-(((2R)-1-(sec-butyl)pyrrolidin-2-yl)methyl)-5,6,7-trifluoro-1H-indole;
(R)-5, 6,7-trifluoro-3-((1-isopropylpyrrolidin-2-yl)methyl)-1H-indole;
(R)-3-((1-cyclobutylpyrrolidin-2-yl)methyl)-5,6,7-trifluoro-1H-indole;
(R)-3-((1-cyclopropylpyrrolidin-2-yl)methyl)-5,6,7-trifluoro-1H-indole;
(R)-3-((1-allylpyrrolidin-2-yl)methyl)-5,6,7-trifluoro-1H-indole;
3-(((2R)-1-(but-3-en-2-yl)pyrrolidin-2-yl)methyl)-5,6,7-trifluoro-1H-indole;
(R)-5,6,7-trifluoro-3-((1-(prop-2-yn-1-yl)pyrrolidin-2-yl)methyl)-1H-indole;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

34. A compound of formula (II), or a salt, prodrug, solvate, isotopologue, or stereoisomer thereof: 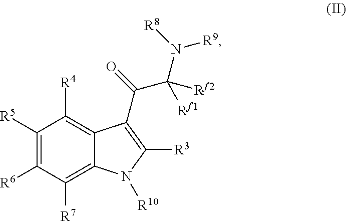
wherein:
R³ is selected from the group consisting of H, optionally substituted C₁-C₈ alkyl, optionally substituted benzyl, optionally substituted C₃-C₈ cycloalkyl, optionally substituted C₂-C₈ alkenyl, and optionally substituted C₂-C₈ alkynyl;
R⁴, R⁵, R⁶, and R⁷ are each independently selected from the group consisting of H, F, Cl, Br, I, OR^{A}, N(R^{A})(R^{B}), SR^{A}, optionally substituted C₁-C₆ alkyl, optionally substituted C₂-C₆ alkenyl, optionally substituted C₂-C₆ alkynyl, optionally substituted C₁-C₈ heteroalkyl, optionally substituted benzyl, optionally substituted phenyl, and optionally substituted C₂-C₉ heteroaryl,
wherein at least one of R⁴, R⁵, R⁶, and R⁷ is F;
R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
R^{f1} and R^{f2} are each independently selected from the group consisting of H and C₁-C₆ alkyl;
each occurrence of R^{A} is independently selected from the group consisting of H, C₁-C₆ haloalkyl, C₂-C₆ alkynyl, C₁-C₆ alkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂₀(C₆-C₁₀ aryl), —S(═O)₂₀(C₁-C₆ alkyl), —S(═O)₂₀(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

35. The compound of claim 34, wherein at least one of the following applies:
(a) R³ is H,
(b) R⁸ and R⁹ are each independently selected from the group consisting of methyl, ethyl, and n-propyl;
(c) R¹⁰ is H;
(d) R^{f1} and R^{f2} are H.

36-38. (canceled)

39. The compound of claim 34, wherein:
(a) R⁴ is F, and each of R⁵, R⁶, and R⁷ is H;
(b) R⁵ is F, and each of R⁴, R⁶, and R⁷ is H;
(c) R⁶ is F, and each of R⁴, R⁵, and R⁷ is H;
(d) R⁷ is F, and each of R⁴, R⁵, and R⁶ is H;
(e) each of R⁴ and R⁵ is F, and each of R⁶ and R⁷ is H;
(f) each of R⁴ and R⁶ is F, and each of R⁵ and R⁷ is H;
(g) each of R⁴ and R⁷ is F, and each of R⁵ and R⁶ is H;
(h) each of R⁵ and R⁶ is F, and each of R⁴ and R⁷ is H;
(i) each of R⁵ and R⁷ is F, and each of R⁴ and R⁶ is H; or
(j) each of R⁶ and R⁷ is F, and each of R⁴ and R⁵ is H.

40. The compound of claim 34, which is selected from the group consisting of:
2-(ethyl(propyl)amino)-1-(5-fluoro-1H-indol-3-yl)ethan-1-one;
2-(ethyl(methyl)amino)-1-(5-fluoro-1H-indol-3-yl)ethan-1-one;
2-(dimethylamino)-1-(4-fluoro-1H-indol-3-yl)ethan-1-one;
2-(dimethylamino)-1-(5-fluoro-1H-indol-3-yl)ethan-1-one;
2-(dimethylamino)-1-(6-fluoro-1H-indol-3-yl)ethan-1-one;
2-(dimethylamino)-1-(7-fluoro-1H-indol-3-yl)ethan-1-one;
1-(4,5-difluoro-1H-indol-3-yl)-2-(dimethylamino)ethan-1-one;
1-(4,6-difluoro-1H-indol-3-yl)-2-(dimethylamino)ethan-1-one;
1-(5,6-difluoro-1H-indol-3-yl)-2-(dimethylamino)ethan-1-one;
1-(5,7-difluoro-1H-indol-3-yl)-2-(dimethylamino)ethan-1-one; and
1-(6,7-difluoro-1H-indol-3-yl)-2-(dimethylamino)ethan-1-one.

41. A compound of formula (III), or a salt, prodrug, solvate, isotopologue, or stereoisomer thereof: 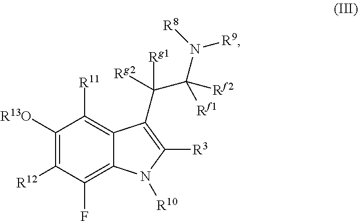
wherein:
R⁸ and R⁹ are each independently selected from the group consisting of optionally substituted C₁-C₈ alkyl, optionally substituted C₃-C₈ cycloalkyl, optionally substituted C₂-C₈ alkenyl, and optionally substituted C₂-C₈ alkynyl;
R¹⁰ is selected from the group consisting of H, optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
R¹¹ and R¹² are each independently selected from the group consisting of H, F, Cl, Br, I, OR^{A}, N(R^{A})(R^{B}), optionally substituted C₁-C₆ alkyl, optionally substituted C₂-C₆ alkenyl, optionally substituted C₂-C₆ alkynyl, optionally substituted benzyl, optionally substituted phenyl, and optionally substituted C₂-C₉ heteroaryl;
R¹³ is selected from the group consisting of H, optionally substituted C₁-C₆ alkyl, optionally substituted C₁-C₆ alkenyl, optionally substituted C₁-C₆ alkynyl, optionally substituted C₁-C₃ haloalkyl, optionally substituted benzyl, optionally substituted phenyl, and optionally substituted C₂-C₉ heteroaryl;
R^{f1} and R^{f2} are each independently selected from the group consisting of H and C₁-C₆ alkyl;
R^{g1} and R^{g2} are each independently selected from the group consisting of H and C₁-C₆ alkyl;
each occurrence of R^{A} is independently selected from the group consisting of H, C₁-C₆ alkyl, C₂-C₆ alkenyl, C₁-C₆ haloalkyl, —C(═O)C₁-C₆ alkyl, —C(═O)C₆-C₁₀ aryl, —C(═O)NH(C₁-C₆ alkyl), —C(═O)NH(C₆-C₁₀ aryl), —C(═O)N(C₁-C₆ alkyl)₂, —C(═O)N(C₁-C₆ alkyl)(C₆-C₁₀ aryl), —C(═O)O(C₁-C₆ alkyl), —C(═O)O(C₆-C₁₀ aryl), —P(═O)(O(C₁-C₆ alkyl))₂, —P(═O)(O(C₁-C₆ alkyl))(OH), —P(═O)(OH)₂, —S(═O)₂O(C₆-C₁₀ aryl), —S(═O)₂O(C₁-C₆ alkyl), —S(═O)₂O(C₆-C₁₀ aryl), —S(═O)₂NH(C₁-C₆ alkyl), —S(═O)₂NH(C₆-C₁₀ aryl), —S(═O)₂N(C₁-C₆ alkyl)(C₁-C₆ alkyl), and —S(═O)₂N(C₁-C₆ alkyl)(C₆-C₁₀ aryl); and
each occurrence of R^{B} is independently selected from the group consisting of H, C₁-C₆ alkyl, C₁-C₃ haloalkyl, C₂-C₆ alkenyl, benzyl, naphthyl, C₂-C₉ heteroaryl, and phenyl.

42. The compound of claim 41, wherein at least one of the following applies:
(a) R³ is H;
(b) each of R⁸ and R⁹ are independently selected from the group consisting of methyl, ethyl, n-propyl, iso-propyl, sec-butyl, iso-butyl, n-butyl, cyclopropyl, cyclopropylmethyl, methylcyclopropyl, cyclopropylethyl, cyclobutyl, allyl, methylallyl, 2-methylallyl, 3-methylallyl, allylmethyl, propargyl, cyanomethyl, 2-hydroxyethyl, and 2-methoxyethyl;
(c) R¹⁰ is H;
(d) each of R¹¹ and R¹² is H;
(e) R¹³ is H or methyl;
(f) each of R^{f1}, R^{f2}, R^{g1}, and R^{g2} is H.

43-47. (canceled)

48. The compound of claim 41, which is selected from the group consisting of:
N-ethyl-N-(2-(7-fluoro-5-methoxy-1H-indol-3-yl)ethyl)propan-1-amine;
2-(7-fluoro-5-methoxy-1H-indol-3-yl)-N,N-dimethylethan-1-amine;
N,N-diethyl-2-(7-fluoro-5-methoxy-1H-indol-3-yl)ethan-1-amine;
N-(2-(7-fluoro-5-methoxy-1H-indol-3-yl)ethyl)-N-propylpropan-1-amine;
3-(2-(dimethylamino)ethyl)-7-fluoro-1H-indol-5-ol;
3-(2-(diethylamino)ethyl)-7-fluoro-1H-indol-5-ol;
3-(2-(dipropylamino)ethyl)-7-fluoro-1H-indol-5-ol;
N-ethyl-2-(7-fluoro-5-methoxy-1H-indol-3-yl)-N-methylethan-1-amine;
N-(2-(7-fluoro-5-methoxy-1H-indol-3-yl)ethyl)-N-methylpropan-1-amine;
N-(2-(7-fluoro-5-methoxy-1H-indol-3-yl)ethyl)-N-methylpropan-2-amine;
N-ethyl-N-(2-(7-fluoro-5-methoxy-1H-indol-3-yl)ethyl)propan-2-amine;
N-(2-(7-fluoro-5-methoxy-1H-indol-3-yl)ethyl)-N,2-dimethylpropan-1-amine;
N-(2-(7-fluoro-5-methoxy-1H-indol-3-yl)ethyl)-N-methylcyclobutanamine;
N-(2-(7-fluoro-5-methoxy-1H-indol-3-yl)ethyl)-N-methylprop-2-en-1-amine;
N-(2-(7-fluoro-5-methoxy-1H-indol-3-yl)ethyl)-N-isopropyl-2-methylpropan-1-amine;
3-(2-(ethyl(methyl)amino)ethyl)-7-fluoro-1H-indol-5-ol;
7-fluoro-3-(2-(methyl(propyl)amino)ethyl)-1H-indol-5-ol;
7-fluoro-3-(2-(isopropyl(methyl)amino)ethyl)-1H-indol-5-ol;
3-(2-(ethyl(propyl)amino)ethyl)-7-fluoro-1H-indol-5-ol;
3-(2-(ethyl(isopropyl)amino)ethyl)-7-fluoro-1H-indol-5-ol;
7-fluoro-3-(2-(isobutyl(methyl)amino)ethyl)-1H-indol-5-ol;
3-(2-(cyclobutyl(methyl)amino)ethyl)-7-fluoro-1H-indol-5-ol;
3-(2-(allyl(methyl)amino)ethyl)-7-fluoro-1H-indol-5-ol;
7-fluoro-3-(2-(isobutyl(isopropyl)amino)ethyl)-1H-indol-5-ol;
3-(((2R)-1-(sec-butyl)pyrrolidin-2-yl)methyl)-7-fluoro-5-methoxy-1H-indole;
(R)-7-fluoro-3-((1-isopropylpyrrolidin-2-yl)methyl)-5-methoxy-1H-indole;
(R)-3-((1-cyclobutylpyrrolidin-2-yl)methyl)-7-fluoro-5-methoxy-1H-indole;
(R)-3-((1-cyclopropylpyrrolidin-2-yl)methyl)-7-fluoro-5-methoxy-1H-indole;
(R)-3-((1-allylpyrrolidin-2-yl)methyl)-7-fluoro-5-methoxy-1H-indole;
3-(((2R)-1-(but-3-en-2-yl)pyrrolidin-2-yl)methyl)-7-fluoro-5-methoxy-1H-indole; and
(R)-7-fluoro-5-methoxy-3-((1-(prop-2-yn-1-yl)pyrrolidin-2-yl)methyl)-1H-indole.

49. The compound of claim 1, wherein each occurrence of alkyl, heteroalkyl, alkenyl, alkynyl, cycloalkyl, and heterocyclyl is independently optionally substituted with at least one substituent selected from the group consisting of C₁-C₆ alkyl, C₃-C₈ cycloalkyl, halo, cyano, OR^{A}, optionally substituted benzyl, optionally substituted phenyl, optionally substituted C₂-C₉ heteroaryl, C(═O)OR^{A}, OC(═O)OR^{A}OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}).

50. The compound of claim 1, wherein each occurrence of benzyl, phenyl, and heteroaryl is independently optionally substituted with at least one substituent selected from the group consisting of C₁-C₆ alkyl, C₃-C₈ cycloalkyl, halo, cyano, OR^{A}, C(═O)OR^{A}, OC(═O)OR^{A}, OC(═O)R^{A}, SR^{A}, S(═O)R^{A}, S(═O)₂R^{A}, S(═O)₂N(R^{A})(R^{B}), N(R^{A})S(═O)₂R^{A}, N(R^{A})C(═O)R^{A}, C(═O)N(R^{A})(R^{B}), and N(R^{A})(R^{B}).

51. A compound selected from the group consisting of:
N-(2-(5-fluoro-1H-indol-3-yl)ethyl)prop-2-en-1-amine;
2-(5-fluoro-1H-indol-3-yl)-N,N-dimethylpropan-1-amine;
N-(2-(5-fluoro-1H-indol-3-yl)ethyl)-N-propylpropan-1-amine;
N,N-diethyl-2-(4-fluoro-1H-indol-3-yl)ethan-1-amine;
N-(2-(4-fluoro-1H-indol-3-yl)ethyl)-N-propylpropan-1-amine;
N-(2-(7-fluoro-1H-indol-3-yl)ethyl)-N-propylpropan-1-amine;
N-(2-(5,6-difluoro-1H-indol-3-yl)ethyl)-N-propylpropan-1-amine;
2-(5,7-difluoro-1H-indol-3-yl)-N,N-diethylethan-1-amine;
N-(2-(5,7-difluoro-1H-indol-3-yl)ethyl)-N-propylpropan-1-amine;
2-(6,7-difluoro-1H-indol-3-yl)-N,N-diethylethan-1-amine;
N-(2-(6,7-difluoro-1H-indol-3-yl)ethyl)-N-propylpropan-1-amine; and
2-(5-fluoro-1H-indol-3-yl)propan-1-amine,
or a salt, prodrug, solvate, isotopologue, or stereoisomer thereof.

52. A pharmaceutical composition comprising at least one compound of claim 1 and a pharmaceutically acceptable carrier.

53. A method of treating, preventing, or ameliorating a psychiatric disease or disorder in a subject in need thereof, the method comprising administering to the subject a therapeutically effective amount of at least one compound of claim 1 or at least one compound of any of compounds 1-218.

54. The method of claim 53, wherein the psychiatric disease or disorder is selected from the group consisting of a depressive disorder, anxiety disorder, and eating disorder.

55. The method of claim 53, wherein the psychiatric disease or disorder is selected from the group consisting of attention deficit disorder (ADD), attention deficit hyperactivity disorder (ADHD), treatment resistant depression, major depressive disorder (MDD), bipolar I disorder, bipolar II disorder, cyclothymic disorder, anti-social personality disorder, pain, sleep-wake disorders, disruptive mood dysregulation disorder, persistent depressive disorder (dysthymia), premenstrual dysphoric disorder, substance/medication-induced depressive disorder, post-partum depression, depressive disorder due to another medical condition, separation anxiety disorder, specific phobia, social anxiety disorder, panic disorder, panic attack, agoraphobia, generalized anxiety disorder, substance-medication induced anxiety disorder, anxiety disorder due to another medical condition, somatic symptom disorder, illness anxiety disorder, obsessive-compulsive disorder (OCD), obsessive-compulsive and related disorder (OCRD), OCRD due to another medical condition, substance-related disorders, alcohol-related disorders, cannabis-related disorders, hallucinogen-related disorders, inhalant-related disorders, cocaine-related disorders, opioid-related disorders, sedative-, hypnotic-, and/or anxiolytic-related disorders, stimulant-related disorders, tobacco-related disorders, non-substance-related disorders (gambling and/or gaming disorder), anorexia nervosa, bulimia nervosa, and binge-eating disorder.

56. The method of claim 53, wherein the subject is further administered at least one additional agent useful for treating, preventing, and/or ameliorating the psychiatric disease or disorder.

57. The method of claim 56, wherein the at least one additional agent is selected from the group consisting of a selective serotonin reuptake inhibitor, triple reuptake inhibitor, serotonin and norepinephrine reuptake inhibitor, tricyclic antidepressant, tetracyclic antidepressant, dopamine reuptake inhibitor, mood stabilizer, anticonvulsant, antipsychotic, anxiolytics, benzodiazepines, monoamine releasers, dopamine receptor agonist, cannabinoids, triptans, anti-migraine medications, analgesics, anti-inflammatory, immune modulator, 5-HT_{1A} receptor antagonist, 5-HT₂ receptor antagonist, 5-HT₃ receptor antagonist, monoamine oxidase inhibitor, and noradrenergic antagonist.

58. The method of claim 56, wherein the subject is co-administered the at least one compound and the at least one additional agent, optionally wherein the at least one compound and the at least one additional agent are co-formulated.

59. (canceled)

60. The method of claim 53, wherein the subject is a mammal, optionally wherein the mammal is human.

61. (canceled)
